(19) United States
(12) Patent Application Publication
(10) Pub. No.: US 2014/0042411 A1
(43) Pub. Date: Feb. 13, 2014

(54) METHOD FOR PURIFYING ORGANIC MATERIAL, MATERIAL FOR ORGANIC ELECTRONICS, PHOTOELECTRIC CONVERSION DEVICE, OPTICAL SENSOR, IMAGING DEVICE, AND ORGANIC ELECTROLUMINESCENCE DEVICE

(71) Applicant: FUJIFILM CORPORATION, Tokyo (JP)

(72) Inventor: Eiji FUKUZAKI, Ashigarakami-gun Kanagawa (JP)

(73) Assignee: FUJIFILM CORPORATION, Tokyo (JP)

(21) Appl. No.: 14/047,908

(22) Filed: Oct. 7, 2013

Related U.S. Application Data
(63) Continuation of application No. PCT/JP2012/058993, filed on Apr. 2, 2012.

(30) Foreign Application Priority Data
Apr. 8, 2011 (JP) 2011-086506
Mar. 28, 2012 (JP) 2012-074554

(51) Int. Cl. H01L 51/00; H01L 51/44

(52) U.S. Cl. CPC H01L 51/006; H01L 51/0072; H01L 51/0059; H01L 51/0071; H01L 51/44
USPC 257/40; 548/440; 546/102; 564/434; 564/427; 548/418; 544/38; 438/57

(57) ABSTRACT

Provided is a method for purifying an organic material having a 10% weight reduction temperature of 250° C. or more as measured by thermogravimetry at a vacuum degree of 1×10⁻² Pa or less, which may sublime and purify the organic material having high heat resistance at high sublimation temperature with high purity and high yield in a short period of time, in which the organic material is subjected sublimation purification after a concentration of inorganic impurities in the organic material is adjusted to 5,000 ppm or less.

### CROSS REFERENCE TO RELATED APPLICATION

This is a continuation of International Application No. PCT/JP2012/058993 filed on Apr. 2, 2012, and claims priority from Japanese Patent Application Nos. 2011-086506 filed on Apr. 8, 2011 and 2012-074554 filed on Mar. 28, 2012, the entire disclosures of which are incorporated by reference.

### TECHNICAL FIELD

The present invention relates to a method for purifying an organic material, a material for organic electronics, a photoelectric conversion device, an optical sensor, an imaging device, and an organic electroluminescence device. Particularly, the present invention relates to a purification method which is effective in improving purity of an organic material used as a constituent material of an organic semiconductor device, such as a photoelectric conversion device, an organic electroluminescence device and an organic thin film transistor, and a material for organic electronics whose purity is improved. Further, the present invention also relates to a material for organic electronics, which is useful as a material for a photoelectric conversion device, and a photoelectric conversion device, an optical sensor, an imaging device and an organic electroluminescence device using the material.

### BACKGROUND ART

An organic electronics device represented by an organic electroluminescence (EL) device, an organic thin film transistor or a photoelectric conversion device is expected to be developed for various uses such as electronic paper or display, and illumination due to characteristics, such as lightweight, an area, flexibility and printability.

For example, a device using an organic material has been considered as an imaging device. In general, a plane type light receiving device in which photoelectric conversion sites are two-dimensionally arranged in a semiconductor to form pixels and a signal generated by photoelectric conversion in each pixel is charge-transferred and read by a CCD circuit or a CMOS circuit is widely used as an imaging device. As photoelectric conversion sites in the related art, sites in which a photodiode part using the PN junction in a semiconductor such as Si is formed are generally used. In recent years, while the fabrication of a multipixel device is proceeding, due to a decrease in a pixel size and a reduction in area of the photodiode part, problems of a reduction in aperture ratio, reduction in light collection efficiency, and the resulting reduction in sensitivity have emerged. A solid-state imaging device having a photoelectric conversion film using an organic material has been examined as a method of improving an aperture ratio and the like.

A solar cell using an organic semiconductor is easily manufactured compared to an inorganic solar cell represented by silicon and the like, and thus has a benefit of achieving a large area at low costs and has been widely examined, but fails to reach a practical use level due to low energy conversion efficiency.

Since an organic electroluminescence (EC) device is capable of obtaining light emission with high luminance intensity at low voltage, the device has been highlighted as a display device and a light emitting device. Since the organic EL device greatly reduces electric power consumption and easily leads to miniaturization and large area thereof, practical application studies thereof have been actively performed as a next generation display device and light emitting device.

Typically, an organic compound includes large amounts of impurities such as unreacted materials•intermediates•inorganic salts derived from the synthetic process thereof, and it is known that when the organic compound is used as it is as a material for organic electronics, the impurities serve as a trap to disturb hole or electron conduction or a trap to disturb recombination of holes and electrons, and a quencher of excitons, and thus adversely affects device performances, such as an increase in driving voltage and reduction in light emission efficiency or photoelectric conversion.

Accordingly, as a method of removing impurities included in the material for organic electronics, for example, purification methods such as column chromatography, recrystallization, reprecipitation purification and sublimation purification have been used. In particular, since the sublimation purification is performed in the absence of a solvent, and thus may suppress incorporation of impurities included in the solvent or the solvent from remaining in the material (responsible for a reduction in vacuum degree during vacuum deposition performed in the device manufacture), the sublimation purification has been widely used as a purification method for obtaining a high-purity material for organic electronics.

For example, in Patent Document 1, a carbazole derivative used in an organic EL device is sublimed by sublimation purification.

However, in a typical sublimation purification method, it takes time for sublimation and a yield thereof is also low, and thus the improvement thereof is needed.

Further, since sublimation temperature of a material is increased for sublimation purification of the material having high heat resistance, it is known that more time is taken for sublimation and the material itself decomposes. When incorporated into the device, the material decomposition product may serve as a charge trap or an exciton quencher to thereby be responsible for making the device performance deteriorate, and thus it is required that a sublimation purification method which does not cause a material to decompose has been demanded.

Patent Documents 2 to 5 describe attempts made to enhance a sublimation rate and a yield by improving a sublimation purification device, but do not sufficiently describe a material which triggers sublimation.

Patent Documents 6 and 7 describe that efficiency (high purity, high yield, and short period of time) is enhanced by stirring and vibrating a material or promoting nucleus growth (addition of quartz wool), but do not sufficiently describe an amount of impurities included in the material before sublimation.

### RELATED ART

### Patent Document

Patent Document 1: Japanese Patent Application Laid-Open No. 2007-284411
Patent Document 2: International Publication No. WO01/070364
Patent Document 3: Japanese Patent No. 2706936
Patent Document 4: Japanese Patent Application Laid-Open No. 2007-44592
Patent Document 5: Japanese Patent Application Laid-Open No. 2003-88704
Patent Document 6: Japanese Patent Application Laid-Open No. 2000-203988
Patent Document 7: Japanese Patent Application Laid-Open No. H11-171801

### DISCLOSURE OF INVENTION

### Problems to be Solved by the Invention

As described above, since impurities included in an organic compound used as a material for organic electronics adversely affect the device performance, the impurities are generally removed from a material for organic electronics by sublimation purification, but there is a problem in sublimation efficiency (high purity, high yield and sublimation time).

In particular, in a material having high sublimation temperature, a difference between thermal decomposition temperature and sublimation temperature of the material is so small that the material while being subjected to sublimation purification is likely to thermally decompose. Since thermal decomposition of the material reduces the purity and yield, it was difficult to efficiently sublime the material having high sublimation temperature.

Further, a material having high heat resistance (high glass transition temperature Tg) has high van der Waals force and a high molecular weight in many cases, and molecules having a large molecular weight have high sublimation temperature and the difference between sublimation temperature and thermal decomposition temperature of the material is easily decreased, and thus it is difficult to find efficient sublimation conditions.

For this reason, it is difficult to efficiently subject an organic material having high heat resistance to sublimation purification, and high-purity organic materials fail to be obtained even though other purification methods are used.

Meanwhile, even among the materials for organic electronics, a material for a photoelectric conversion device needs to have high heat resistance for application to a manufacturing process having a heating step, such as provision of a color filter, provision of a protection film, and soldering of a device, or enhancement of preserving property.

Even in an organic photoelectric luminescence device, a material having high heat resistance is needed in use of a display for car navigation, an outdoor type display, and illumination.

As described above, in the material for organic electronics, it is required that impurities are removed from the viewpoint of device performance, and materials having high heat resistance and high purity are needed.

In consideration of the aforementioned circumstances, an object of the present invention is to provide a method for purifying an organic material, which may sublime and purify the organic material having high heat resistance at high sublimation temperature with high purity and high yield in a short period of time.

Another object of the present invention is to provide a material for organic electronics, which has high heat resistance at high purification temperature and high purity. Yet another object of the present invention is to provide a photoelectric conversion device, an optical sensor, an imaging device and an organic electroluminescence device, using the material for organic electronics.

### Means for Solving the Problem

The present inventors have intensively studied, and as a result, found that by adjusting an amount of a specific impurity, which is included in a material before being subjected to sublimation purification to a predetermined amount or less, the sublimation efficiency in the sublimation purification of the material (high purity, high yield, and sublimation time) may be significantly enhanced, thereby completing the present invention.

That is, a specific means for solving the problem is as follows.

[1] A method for purifying an organic material having a 10% weight reduction temperature of 250° C. or more as measured by thermogravimetry at a vacuum degree of 1×10⁻² Pa or less,

in which the organic material is subjected to sublimation purification after a concentration of inorganic impurities in the organic material is adjusted to 5,000 ppm or less.

[2] The method described in [1], in which the inorganic impurities having a concentration of 5,000 ppm or less are atoms and ions of a metal belonging to alkali metals, alkaline earth metals, transition metals, or typical metals.

[3] The method described in [2], in which the inorganic impurities having a concentration of 5,000 ppm or less are atoms and ions of a metal belonging to alkali metals, or transition metals.

[4] A material for organic electronics having a 10% weight reduction temperature of 250° C. or more as measured by thermogravimetry at a vacuum degree of 1×10⁻² Pa or less, in which a purity of the material for organic electronics is 98.5% or more.

[5] The material for organic electronics described in [4], in which the material for organic electronics is a compound represented by the following Formula (1).

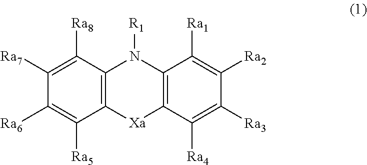

(In the formula, R₁ represents an alkyl group, an aryl group or a heterocyclic group, which may have a substituent. Ra₁ to Ra₈ independently represent a hydrogen atom or a substituent. At least two of R₁ and Ra₁ to Ra₈ may be bound with each other to form a ring. Xa represents a single bond, an oxygen atom, a sulfur atom, or an alkylene group, a silylene group, an alkenylene group, a cycloalkylene group, a cycloalkenylene group, an arylene group, a divalent heterocyclic group or an imino group, which may have a substituent.)

[6] The material for organic electronics described in [5], in which the compound represented by Formula (1) is a compound represented by the following Formula (F-1).

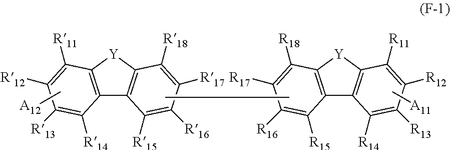

(In Formula (F-1), R₁₁ to R₁₈ and R′₁₁ to R′₁₈ independently represent a hydrogen atom, a halogen atom, an alkyl group, an aryl group, a heterocyclic group, a hydroxyl group, an amino group or a mercapto group, and these groups may further have a substituent. However, any one of R₁₅ to R₁₈ is linked to any one of R′₁₅ to R′₁₈ to form a single bond. A₁₁ and A₁₂ each independently represent a substituent represented by the following Formula (A-1), and are substituted as one of R₁₁ to R₁₄ and one of R′₁₁ to R′₁₄. Y independently represents a carbon atom, a nitrogen atom, an oxygen atom, a sulfur atom or a silicon atom, and these groups may further have a substituent.

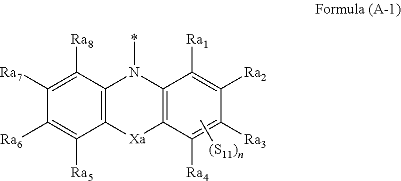

(In Formula (A-1), Ra₁ to Ra₈ independently represent a hydrogen atom, a halogen atom, an alkyl group, an aryl group, a heterocyclic group or an alkoxy group, and these groups may further have a substituent. At least two of Ra₁ to Ra₈ may be bound with each other to form a ring. * represents a bonding position. Xa represents a single bond, an oxygen atom, a sulfur atom, or an alkylene group, a silylene group, an alkenylene group, a cycloalkylene group, a cycloalkenylene group, an arylene group, a divalent heterocyclic group or an imino group, which may have a substituent. S₁₁ independently represents the following substituent (S₁₁), and is substituted as one of Ra₁ to Ra₈. n independently represents an integer of 1 to 4.)

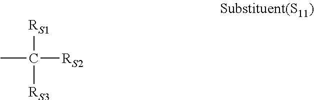

(R_{S1} to R_{S3} independently represent a hydrogen atom or an alkyl group. At least two of R_{S1} to R_{S3} may be bound with each other to form a ring.)

[7] The material for organic electronics described in [6], in which the compound represented by Formula (F-1) is a compound represented by the following Formula (F-2).

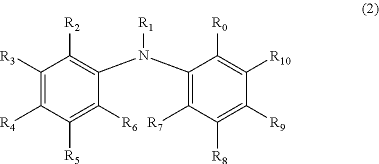

(In Formula (F-2), R₁₁ to R₁₆, R₁₈, R′₁₁ to R′₁₆ and R′₁₈ each independently represent a hydrogen atom, a halogen atom, an alkyl group, an aryl group, a heterocyclic group, a hydroxyl group, an amino group or a mercapto group, and these groups may further have a substituent. A₁₁ and A₁₂ each independently represent the substituent represented by Formula (A-1), and are substituted as one of R₁₁ to R₁₄ and one of R′₁₁ to R′₁₄. Y independently represents a carbon atom, a nitrogen atom, an oxygen atom, a sulfur atom or a silicon atom, and these groups may further have a substituent.)

[8] The material for organic electronics described in [6] or [7], in which in Formulae (F-1) and (F-2), the substituent represented by Formula (A-1) is independently substituted with R₁₂ and R′₁₂.

[9] The material for organic electronics described in any one of [6] to [8], in which n in Formula (A-1) represents 1 or 2.

[10] The material for organic electronics described in any one of [6] to [9], in which at least one of Ra₁ and Ra_{b} in Formula (A-1) each independently represents the substituent (S₁₁).

[11] The material for organic electronics described in any one of [6] to [10], in which Y in Formulae (F-1) and (F-2) represents —N(R₂₀)—, and R₂₀ represents an alkyl group, an aryl group or a heterocyclic group.

[12] The material for organic electronics described in any one of [6] to [10], in which Y in Formulae (F-1) and (F-2) represents —C(R₂₁)(R₂₂)—, and R₂₁ and R₂₂ each independently represent an alkyl group, an aryl group or a heterocyclic group.

[13] The material for organic electronics described in [4], in which the material for organic electronics is a material represented by the following Formula (2).

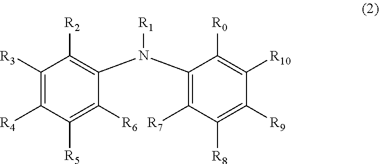

(In the formula, R₁ represents an alkyl group, an aryl group or a heterocyclic group, which may have a substituent. R₀ and R₂ to R₁₀ each independently represent a hydrogen atom or a substituent.)

[14] The material for organic electronics described in [13], in which in Formula (2), R₁ which may have a substituent group is an aryl group.

[15] The material for organic electronics described in any one of [4] to [14], in which a glass transition temperature (Tg) of the material for organic electronics is 130° C. or more.

[16] The material for organic electronics described in any one of [4] to [15], in which a molecular weight of the material for organic electronics is from 500 to 2,000.

[17] A photoelectric conversion device including a transparent conductive film, a photoelectric conversion film and a conductive film in this order, in which the photoelectric conversion film includes a photoelectric conversion layer and a charge blocking layer, and the charge blocking layer contains the material for organic electronics described in any one of [4] to [16].

[18] The photoelectric conversion device described in [17], in which the photoelectric conversion layer includes an n-type organic semiconductor.

[19] The photoelectric conversion device described in [18], in which the n-type organic semiconductor is fullerene or a fullerene derivative.

[20] The photoelectric conversion device described in any one of [17] to [19], in which the photoelectric conversion film includes a compound of the following Formula (I).

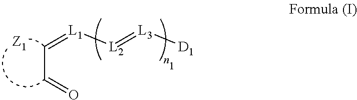

(In the formula, Z₁ is a ring including at least two carbon atoms, and represents a 5-membered ring, a 6-membered ring or a condensed ring including at least one of the 5-membered ring and the 6-membered ring. L₁, L₂ and L₃ each independently represent an unsubstituted methine group or a substituted methine group. D₁ represents an atom group. n₁ represents an integer of 0 or more.)

[21] A method for manufacturing the photoelectric conversion device described in any one of [17] to [20], the method including: film-forming each of the photoelectric conversion layer and the charge blocking layer by vacuum thermal deposition.

[22] An optical sensor including the photoelectric conversion device described in any one of [17] to [20].

[23] An imaging device including the photoelectric conversion device described in any one of [17] to [20].

[24] An organic electroluminescence device including at least one organic layer including a light emitting layer between a pair of electrodes, in which the organic layer contains the material for organic electronics described in any one of [4] to [16].

### Effects of Invention

According to the present invention, an organic material having high sublimation temperature and high heat resistance may be subjected to sublimation purification with high efficiency (high purity, high yield and short period of time) by reducing an amount of inorganic impurities included in the material before the sublimation purification.

Further, it is possible to obtain a high-performance organic electronics device by using a high-purity organic material purified by the method of the present invention as the material for organic electronics. In particular, when applying the organic material to a photoelectric conversion device, it is also possible to provide a photoelectric conversion device which exhibits low dark current, and has a small increase in dark current even when the device is subjected to heat treatment, and an imaging device equipped with the photoelectric conversion device. In addition, when applying the organic material to an organic electroluminescence device, it is possible to provide an organic electroluminescence device having high external quantum efficiency and low driving voltage.

### BRIEF DESCRIPTION OF DRAWINGS

FIGS. 1(a) and (b) each are schematic cross-sectional views illustrating a configuration example of a photoelectric conversion device according to the present invention.

FIG. 2 is a schematic cross-sectional view of one pixel of an imaging device according to the present invention.

FIG. 3 is a schematic cross-sectional view illustrating an example of a layer configuration of an organic electroluminescent device according to the present invention.

### EMBODIMENTS FOR CARRYING OUT THE INVENTION

Hereinafter, the present invention will be described in detail. Further, in the present specification, “to” indicates a range including the numerical values described before and after “to” as a minimum value and a maximum value, respectively.

[Method for Purifying Organic Material]

A method for purifying an organic material according to the present invention is a method for purifying an organic material having a 10% weight reduction temperature of 250° C. or more as measured by thermogravimetry at a vacuum degree of 1×10⁻² Pa or less, in which the organic material is subjected to sublimation purification after a concentration of inorganic impurities in the organic material is adjusted to 5,000 ppm or less.

Here, the 10% weight reduction temperature as measured by thermogravimetry at a vacuum degree of 1×10⁻² Pa or less is an index of the sublimation temperature of a material, and in the present invention, a material having a 10% weight reduction temperature of 250° C. or more means a material having high sublimation purification temperature.

The 10% weight reduction temperature is more preferably 300° C. or more, and particularly preferably 350° C. or more. Since an organic material having high heat resistance (an organic material having a high glass transition temperature (Tg)) has high van der Waals force and a high molecular weight in many cases such that the sublimation temperature thereof is also increased, the 10% weight reduction temperature thereof is also increased.

Further, in the thermogravimetry, the mass of a material is measured while the temperature of the material is changed at a predetermined vacuum degree. The 10% weight reduction temperature may also be measured by a so-called differential heat-thermogravimetry simultaneous measurement (Thermo Gravimetric and Differential Thermal Analysis: TG-DTA) which simultaneously perform thermogravimetry and differential heat analysis (measurement by which a temperature difference between a material to be measured and a reference material is detected).

In the present invention, even an organic material having high sublimation temperature and high heat resistance may be subjected to sublimation purification with high sublimation efficiency (high purity, high yield, and short period of time) by adjusting the concentration of inorganic impurities in the material before the sublimation purification to 5,000 ppm or less. The detailed reason is not clear, but is considered as follows.

In general, in the sublimation purification, only a target compound is separated (since each of compounds has an inherent sublimation temperature, impurities and a target product may be separated by creating a temperature gradient on the collection unit) by a collection unit at low temperature by heating and subliming a material under reduced pressure (about 0.2 Pa or less), and as the sublimation purification proceeds, impurities having high sublimation temperature (particularly, inorganic impurities) included in the material before sublimation are concentrated as a residue in an unsublimed material.

It is assumed that since the impurities concentrated as a residue form a hard shell on the surface of the unsublimed material to cause a reduction in heat conduction to a target product, thereby significantly reducing the sublimation efficiency, or inhibiting molecules entrapped inside the shell from being sublimed, it takes a long time for the sublimation purification. When the sublimation efficiency deteriorates, the heating time is prolonged, and thus the material is easily thermally decomposed. Further, it is assumed that even impurities having high sublimation temperature, particularly inorganic impurities generally do not have a sublimation temperature, so as to easily remain as a residue, and thus the sublimation efficiency of the material is reduced, or inorganic impurities themselves promote thermal decomposition of the material.

It is thought that in the present invention, a shell derived from inorganic impurities formed on the surface of the unsublimed material is difficult to be formed during the sublimation purification by adjusting the concentration of inorganic impurities included before the sublimation purification to 5,000 ppm or less, thereby preventing the sublimation of the material from being inhibited or the material from being thermally decomposed by maintaining the heat conduction efficiency to the material at a good level, and as a result, sublimation purification may be achieved at high purity and high yield for a short purification time.

Further, a high-performance organic electronics device may be obtained by using an organic material purified with high purity as the material for organic electronics.

The concentration of inorganic impurities in the organic material before the sublimation purification is more preferably 2,000 ppm or less, still more preferably 1,000 ppm or less, further more preferably 500 ppm, and particularly preferably 200 ppm or less from the viewpoint of obtaining an organic material having high sublimation efficiency and high purity.

The method of quantifying the content of inorganic impurities in the organic material is not particularly limited, but examples of a quantitative analysis method include ICP atomic emission spectrometry (ICP-AES), atomic absorption spectrometry (AAS), ICP mass spectrometry (ICP-MS), glow discharge mass spectrometry (GDMS), X-ray fluorescence spectrometry (XRF), ion chromatography (IC), capillary electrophoresis (CE) and the like. It is preferred that measurement is performed by ICP atomic emission spectrometry (ICP-AES), atomic absorption spectrometry (AAS), and ICP mass spectrometry (ICP-MS) from the viewpoint of the type of analyzed element, quantitativity, and sensitivity.

(Inorganic Impurities)

Examples of inorganic impurities, which may be contained in an organic material, include the following atoms and ions.

Lithium, sodium, potassium, rubidium, cesium, beryllium, magnesium, calcium, strontium, barium, titanium, zirconium, hafnium, vanadium, niobium, tantalum, chromium, molybdenum, tungsten, manganese, technetium, rhenium, iron, ruthenium, osmium, cobalt, rhodium, iridium, nickel, palladium, platinum, copper, silver, gold, zinc, cadmium, mercury, boron, aluminum, gallium, indium, thallium, silicon, tin, lead, phosphorus, arsenic, antimony, bismuth, selenium, tellurium, fluorine, chlorine, bromine and iodine (Further, in the present invention, when the elements are included as a substituent of an organic material to be sublimed, an atom that constitutes the substituent, or a counter ion, the elements are not considered as inorganic impurities.)

In terms of the effects of the present invention, as inorganic impurities having a concentration of 5,000 ppm or less included in the organic material before the sublimation purification, inorganic impurities which are atoms and ions belonging to alkali metals, alkaline earth metals, transition metals and typical metals are preferred, and inorganic impurities which are atoms and ions belonging to alkali metals and transition metals are more preferred. More specifically, the inorganic impurities are preferably lithium, sodium, potassium, rubidium, cesium, iron, nickel, palladium, platinum, copper, and an ion thereof, more preferably sodium, potassium, rubidium, cesium, nickel, palladium, copper, and an ion thereof, and particularly preferably are rubidium, cesium, nickel, palladium, copper, and an ion thereof.

The metal atoms and ions thereof are easily included in an organic material during the synthesis process thereof, and thus easily promote thermal decomposition during heating in the sublimation purification. In particular, alkali metals and transition metals are used in the catalyst reaction process in many cases, and thus are easily included as impurities in the organic material and also easily promote the thermal decomposition of the material catalytically. Among them, rubidium and cesium having a large atomic (ionic) radius as an alkali metal has high reactivity, and thus easily promote the decomposition thereof. Further, palladium and copper atoms also have catalytic activity, and thus easily promote the decomposition thereof.

For this reason, it is preferred that the metal atoms and the ions thereof are not included as inorganic impurities in an organic material before the sublimation purification.

(Purification Process of Inorganic Impurities)

The method of adjusting the concentration of inorganic impurities included in the organic material before the sublimation purification to 5,000 ppm or less is not particularly limited, but examples thereof include recrystallization purification; reprecipitation purification; column chromatography purification; liquid separation; washing with water or solvents; reslurrying; filtration; separation by filtration; ion exchange resin chromatography; adsorption by activated carbon, diatomaceous earth, ion exchange resin or resin, and the like.

In consideration of simplicity of manipulation and manufacturing suitability, it is preferred that the purification method is recrystallization purification; washing with water or solvents; reslurrying; separation by filtration of impurities and precipitates after a solvent is dissolved; and adsorption by activated carbon, diatomaceous earth, ion exchange resin or resin.

Further, inorganic metal elements and ions may be solubilized by adding an oxidizer, a reducer, a solubilizer, such as an acid (for example, hydrochloric acid, sulfuric acid, phosphoric acid, trifluoroacetic acid, methanesulfonic acid, acetic acid, tetrafluoroboric acid, hexafluorophosphoric acid, perchloric acid, ammonium chloride and the like), a base (potassium hydroxide, sodium hydroxide, sodium butoxide, potassium butoxide, sodium methoxide, sodium ethoxide, cesium hydroxide, rubidium hydroxide, thallium hydroxide, calcium hydroxide, strontium hydroxide, barium hydroxide, triethylamine, potassium carbonate, sodium carbonate, sodium bicarbonate, tripotassium phosphate, cesium carbonate and the like), a salt (lithium chloride, potassium chloride and sodium chloride), a chelate (an azobenzene compound, a naphthylazo compound, a pyridylazo compound, oxalic acid, ethylenediamine, bipyridine, ethylenediaminetetraacetic acid, phenanthroline, porphyrin, crown ether, oxalic acid, Rochelle salt, malic acid and citric acid), a monodentate ligand (N-heterocyclic carbene ligand, a phosphine ligand (triphenylphosphine and tributylphosphine), pyridine, acetonitrile and norbornadiene), and a precipitant, and inorganic impurities may be removed by precipitation.

(Organic Material)

An organic material used in the purification method of the present invention is not limited as long as the organic material has a 10% weight reduction temperature of 250° C. or more as measured by thermogravimetry at a vacuum degree of 1×10⁻² Pa or less, but is preferably a material for organic electronics, such as a photoelectric conversion device, an organic EL device and an organic thin film transistor, in which high purity is required.

The organic material having a 10% weight reduction temperature of 250° C. or more tends to be an organic material having a large molecular weight, and the molecular weight of the organic material is preferably 500 to 2,000, more preferably 500 to 1,500, still more preferably 700 to 1,500, preferably 800 to 1,500 among them, particularly preferably 900 to 1,500, and most preferably 940 to 1,500.

Further, the organic material having a 10% weight reduction temperature of 250° C. or more tends to have high heat resistance, and the glass transition temperature (Tg) thereof is preferably 130° C. or more, more preferably 160° C. or more, still more preferably 175° C. or more, further more preferably 200° C. or more, and particularly preferably 220° C. or more. It is possible to enhance heat resistance of an organic electronic device by using the organic material having a glass transition temperature of 130° C. or more as a material for organic electronic.

[Material for Organic Electronics]

A material for organic electronics of the present invention is a material for organic electronics having a 10% weight reduction temperature of 250° C. or more as measured by thermogravimetry at a vacuum degree of 1×10⁻² Pa or less, in which a purity of the material for organic electronics is 98.5% or more.

The purity of the material for organic electronics is preferably 99.0% or more, more preferably 99.5% or more, and particularly preferably 99.9% or more. Such high purity may be obtained by purifying the material for organic electronics having high sublimation temperature by the purification method of the present invention.

By using the aforementioned high-purity material as the material for organic electronics having high sublimation temperature and high heat resistance in an organic electronics device, the device performance of the device may be enhanced.

Examples of the material for organic electronics include compounds represented by the following Formula (1) or compounds represented by the following Formula (2).

Since the moving velocity of electric charge is high in the compound represented by Formula (2), enhancement in device performance may be realized while heat resistance of the device is maintained. Specifically, it is possible to realize high electric charge collection efficiency and fast response in a photoelectric conversion device, light emission with high efficiency in an organic electroluminescence device, and a high on/off ratio in an organic transistor.

Further, since free rotation of molecules by thermal motion is suppressed in a compound having a condensed ring diarylamine structure and represented by Formula (1), the glass transition temperature is increased, and thus heat resistance of the device is increased.

A compound represented by the following Formula (F-1), in which a condensed ring diarylamine (a substituent represented by the following Formula (A-1)) is linked by the following divalent linking group (D-1), is useful as a charge blocking material of a photoelectric conversion device. A compound linked with the linking group (D-1) and represented by the following Formula (F-1) is polymerized when compared to a material linked with (D-2), thereby enhancing heat resistance. Further, it is assumed that since the bonding between structures is twisted such that the conjugated system is cut off, a layer (for example, a charge blocking layer) using the material and an adjacent layer thereto (for example, a photoelectric conversion layer) do not interact with each other, and thus the dark current of the photoelectric conversion device is maintained at a low level. In addition, it is thought that the diarylamine structure as a charge transporting unit is in introduced into both ends of the molecule instead of the internal side thereof, and thus has high charge transportability.

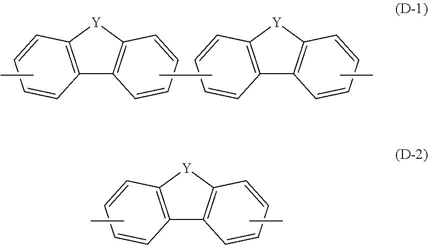

(Y each independently represents —C(R₂₁)(R₂₂)—, —Si(R₂₃)(R₂₄)—, —N(R₂₀)—, an oxygen atom or a sulfur atom, and R₂₀ to R₂₄ independently represent a hydrogen atom, a halogen atom, an alkyl group, an aryl group, a heterocyclic group, a hydroxyl group, an amino group or a mercapto group.)

It could be found by studies of the present inventors that in Formula (F-1), the charge blocking layer may be highly heat resistant without causing a reduction in response speed of the photoelectric conversion device by selecting the linking position of the linking group (D-1), the bonding position of the substituent represented by Formula (A-1), the substitution position of the following substituent (S₁₁) and the type of substituent (S₁₁). It is thought that by finding out the linking position of the linking group (D-1), the bonding position of the substituent represented by Formula (A-1), the substitution position of the substituent (S₁₁) and the optimal point of substituent (S₁₁), effects of suppressing interaction with the photoelectric conversion layer and increasing intermolecular force among compounds represented by Formula (F-1) due to polymerization are strongly exhibited, thereby making the device highly heat resistant.

Hereinafter, a compound represented by each Formula will be described.

First, a compound represented by Formula (1) will be described.

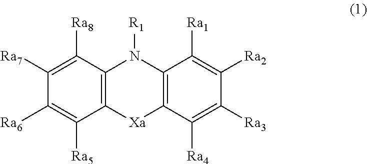

(In the formula, R₁ represents an alkyl group, an aryl group or a heterocyclic group, which may have a substituent. Ra₁ to Ra₈ independently represent a hydrogen atom or a substituent. At least two of R₁ and Ra₁ to Ra₈ may be bound with each other to form a ring. Xa represents a single bond, an oxygen atom, a sulfur atom, or an alkylene group, a silylene group, an alkenylene group, a cycloalkylene group, a cycloalkenylene group, an arylene group, a divalent heterocyclic group or an imino group, which may have a substituent.)

R₁ represents an alkyl group, an aryl group or a heterocyclic group, and may have a substituent. Specific examples of the substituent include a substituent W to be described below, and are preferably a halogen atom, an alkyl group, an aryl group, a heterocyclic group, a hydroxyl group, an amino group or a mercapto group, more preferably a halogen atom, an alkyl group, an aryl group and a heterocyclic group, still more preferably a fluorine atom, an alkyl group and an aryl group, particularly preferably an alkyl group and an aryl group, and most preferably an alkyl group. In the case of having a plurality of the substituents, the substituents may be linked to each other to form a ring. Examples of the ring formed include a ring R to be described below.

When R₁ is an alkyl group, the alkyl group may be a straight•branched alkyl group, and a cyclic alkyl group (a cycloalkyl group), but is preferably a cycloalkyl group. When a carbazole structure is not included in R₁, the carbon number thereof is preferably 4 to 20, and more preferably 5 to 16, and when a carbazole structure is included in R₁, the carbon number thereof is preferably 19 to 35, and more preferably 20 to 31. Specifically, examples of the cycloalkyl group include a cycloalkyl group (a cyclopropyl group, a cyclopentyl group, a cyclohexyl group and the like), a cycloalkenyl group (a 2-cyclohexen-1-yl group and the like).

When R₁ is an aryl group, the aryl group is a substituted or unsubstituted aryl group having preferably 6 to 20 carbon atoms and more preferably 6 to 16 carbon atoms in the case where a carbazole structure is not included in R₁, and a substituted or unsubstituted aryl group having preferably 21 to 35 carbon atoms, and more preferably 21 to 31 carbon atoms in the case where a carbazole structure is included in R₁. More specific examples thereof include a phenyl group, a naphthyl group, an anthryl group, a fluorenyl group and the like.

When R₁ is a heterocyclic group, examples of the heterocyclic group include a 5-membered or 6-membered heterocyclic group, and specific examples thereof include a furyl group, a thienyl group, a pyridyl group, a quinolyl group, a thiazolyl group, an oxazolyl group, an azepinyl group, a carbazolyl group and the like. The aryl group or heterocyclic group may include a condensed ring composed of 2 to 4 monocycles.

R₁ is preferably an aryl group or a heterocyclic group, more preferably an aryl group, and most preferably a phenyl group.

Further, another preferred aspect of R₁ is an aryl group or a heterocyclic group, which has a structure represented by the following Formula (F).

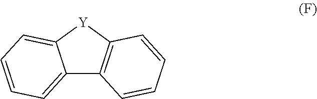

(Y represents —C(R₂₁)(R₂₂)—, —Si(R₂₃)(R₂₄)—, —N(R₂₀)—, an oxygen atom or a sulfur atom, and R₂₀ to R₂₄ independently represent a hydrogen atom, a halogen atom, an alkyl group, an aryl group, a heterocyclic group, a hydroxyl group, an amino group or a mercapto group.)

The group having the structure represented by Formula (F) may further have a substituent, and specific examples of the substituent include the substituent W to be described below. It is also preferred that the substituent further has an aryl group or a heterocyclic group (these groups may further have the substituent W to be described below), which has a structure represented by Formula (F). In addition, the substituents may be linked to each other to form a ring, and examples of the ring formed include the ring R to be described below.

Another more preferred aspect of R₁ is an aspect in which two or more of an aryl group or a heterocyclic group, which has the structure represented by Formula (F), are linked through a single bond or a substituent (still more preferably, an aspect in which two are linked), and particularly preferred aspect is an aspect in which two of an aryl group or a heterocyclic group having the structure represented by Formula (F) are linked through a single bond.

In Formula (1), Ra₁ to Ra₈ independently represent a hydrogen atom or a substituent, and specific examples of the substituent include the substituent W to be described below. The substituent is preferably a halogen atom, an alkyl group, an aryl group, a heterocyclic group, a hydroxyl group, an amino group, a mercapto group or an alkoxy group, more preferably a halogen atom, an alkyl group, an aryl group, a heterocyclic group and an alkoxy group, still more preferably a halogen atom, an alkyl group, an aryl group and a heterocyclic group, further more preferably a fluorine atom, an alkyl group and an aryl group, particularly preferably an alkyl group and an aryl group, and most preferably an alkyl group.

At least two of R₁ and Ra₁ to Ra₈ may be bound with each other to form a ring. Examples of the ring formed include the ring R to be described below.

Xa represents a single bond, an oxygen atom, or a sulfur atom, an alkylene group, a silylene group, an alkenylene group, a cycloalkylene group, a cycloalkenylene group, an arylene group, a divalent heterocyclic group or an imino group, which may have a substituent. Specific examples of the substituent include the substituent W, and are preferably an alkyl group or an aryl group.

Xa is preferably a single bond, an alkylene group having 1 to 12 carbon atoms, an alkenylene group having 2 to 12 carbon atoms, an arylene group having 6 to 14 carbon atoms, a heterocyclic group having 4 to 13 carbon atoms, an oxygen atom, a sulfur atom, an imino group (for example, a phenylimino group, a methylimino group, and a t-butylimino group) having a hydrocarbon group having 1 to 12 carbon atoms (preferably an aryl group or alkyl group) and a silylene group, more preferably a single bond, an oxygen atom, an alkylene group having 1 to 6 carbon atoms (for example, a methylene group, a 1,2-ethylene group, and a 1,1-dimethylmethylene group), an alkenylene group having 2 carbon atoms (for example, —CH₂═CH₂—), an arylene group having 6 to 10 carbon atoms (for example, a 1,2-phenylene group and a 2,3-naphthylene group) and a silylene group, and still more preferably a single bond, an oxygen atom and an alkylene group having 1 to 6 carbon atoms (for example, a methylene group, a 1,2-etheylene group and a 1,1-dimethylmethylene group).

A preferred form of the compound represented by Formula (1) is a compound represented by the following Formula (F-1).

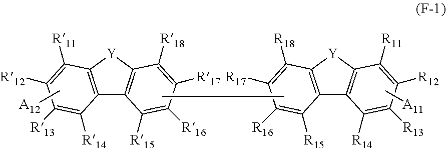

(In Formula (F-1), R₁₁ to R₁₈ and R′₁₁ to R′₁₈ independently represent a hydrogen atom, a halogen atom, an alkyl group, an aryl group, a heterocyclic group, a hydroxyl group, an amino group or a mercapto group, and these groups may further have a substituent. However, any one of R₁₅ to R₁₈ is linked to any one of R′₁₅ to R′₁₈ to form a single bond. A₁₁ and A₁₂ each independently represent a substituent represented by the following Formula (A-1), and are substituted as one of R₁₁ to R₁₄ and one of R′₁₁ to R′₁₄. Y each independently represents a carbon atom, a nitrogen atom, an oxygen atom, a sulfur atom or a silicon atom, and these groups may further have a substituent.)

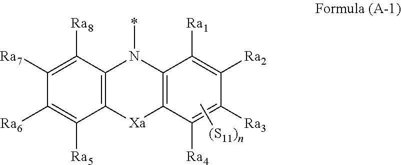

(In Formula (A-1), Ra₁ to Ra₈ independently represent a hydrogen atom, a halogen atom, an alkyl group, an aryl group, a heterocyclic group or an alkoxy group, and these groups may further have a substituent. At least two of Ra₁ to Ra₈ may be bound with each other to form a ring. * represents a bonding position. Xa represents a single bond, an oxygen atom, a sulfur atom, or an alkylene group, a silylene group, an alkenylene group, a cycloalkylene group, a cycloalkenylene group, an arylene group, a divalent heterocyclic group or an imino group, which may have a substituent. S₁₁ each independently represents the following substituent (S₁₁), and is substituted as one of Ra₁ to Ra₈. n each independently represents an integer of 1 to 4.)

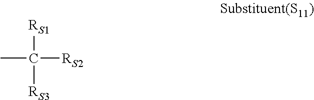

(R_{S1} to R_{S3} independently represent a hydrogen atom or an alkyl group. At least two of R_{S1} to R_{S3} may be bound with each other to form a ring.)

In Formula (F-1), R₁₁ to R₁₈ and R′₁₁ to R′₁₈ independently represent a hydrogen atom, a halogen atom, an alkyl group, an aryl group, a heterocyclic group, a hydroxyl group, an amino group or a mercapto group, and these groups may further have a substituent. More specific examples of the substituent include the substituent W to be described below, and are preferably a halogen atom, an alkyl group, an aryl group, a heterocyclic group, a hydroxyl group, an amino group or a mercapto group, more preferably a halogen atom, an alkyl group, an aryl group and a heterocyclic group, still more preferably a fluorine atom, an alkyl group and an aryl group, particularly preferably an alkyl group and an aryl group, and most preferably an alkyl group.

R₁₁ to R₁₈ and R′₁₁ to R′₁₈ are preferably a hydrogen atom, and an alkyl group, an aryl group and a heterocyclic group, which may have a substituent, and more preferably a hydrogen atom, and an alkyl group having 1 to 18 carbon atoms, an aryl group having 6 to 18 carbon atoms or a heterocyclic group having 4 to 16 carbon atoms, which may have a substituent, from the viewpoint of chemical stability, electric charge mobility and heat resistance. Among them, from the viewpoint of electric charge mobility and heat resistance, it is preferred that the substituent represented by Formula (A-1) is each independently substituted with R₁₂ and R′₁₂, it is more preferred that the substituent represented by Formula (A-1) is each independently substituted with R₁₂ and R′₁₂, and R₁₁, R₁₃ to R₁₈, R′₁₁ and R′₁₃ to R′₁₈ are a hydrogen atom, or an alkyl group having 1 to 18 carbon atoms, which may have a substituent, and it is particularly preferred that the substituent represented by Formula (A-1) is each independently substituted with R₁₂ and R′₁₂, and R₁₁, R₁₃ to R₁₈, R′₁₁ and R′₁₃ to R′₁₈ are a hydrogen atom.

Y each independently represents a carbon atom, a nitrogen atom, an oxygen atom, a sulfur atom or a silicon atom, and these groups may further have a substituent. That is, Y represents a divalent linking group composed of a carbon atom, a nitrogen atom, an oxygen atom, a sulfur atom or a silicon atom. Examples of the substituent include the substituent W to be described below.

It is preferred that Y each independently represents —C(R₂₁)(R₂₂)—, —Si(R₂₃)(R₂₄)—, —N(R₂₀)—, an oxygen atom or a sulfur atom, and R₂₀ to R₂₄ independently represent a hydrogen atom, a halogen atom, an alkyl group, an aryl group, a heterocyclic group, a hydroxyl group, an amino group or a mercapto group. Among them, from the viewpoint of chemical stability, electric charge mobility and heat resistance, —C(R₂₁)(R₂₂)—, —Si(R₂₃)(R₂₄)— and —N(R₂₀)— are preferred, —C(R₂₁)(R₂₂)— and —N(R₂₀)— are more preferred, and —C(R₂₁)(R₂₂)— is particularly preferred.

In the —C(R₂₁)(R₂₂)—, R₂₁ and R₂₂ each independently represent a hydrogen atom, a halogen atom, an alkyl group, an aryl group, a heterocyclic group, a hydroxyl group, an amino group or a mercapto group. R₂₁ and R₂₂ may further have a substituent, and still more specific examples of the substituent include the substituent W, and are preferably an alkyl group, an aryl group or an alkoxy group.

R₂₁ and R₂₂ are preferably a hydrogen atom, and an alkyl group, an aryl group and a heterocyclic group, which may have a substituent, more preferably, a hydrogen atom, and an alkyl group having 1 to 18 carbon atoms, an aryl group having 6 to 18 carbon atoms or a heterocyclic group having 4 to 16 carbon atoms, which may have a substituent, still more preferably a hydrogen atom, and an alkyl group having 1 to 18 carbon atoms, which may have a substituent, and particularly preferably an alkyl group having 1 to 18 carbon atoms.

In the —C(R₂₃)(R₂₄)—, R₂₃ and R₂₄ each independently represent a hydrogen atom, a halogen atom, an alkyl group, an aryl group, a heterocyclic group, a hydroxyl group, an amino group or a mercapto group. R₂₃ and R₂₄ may further have a substituent, and still more specific examples of the substituent include the substituent W, and are preferably an alkyl group, an aryl group or an alkoxy group.

R₂₃ and R₂₄ are preferably a hydrogen atom, and an alkyl group, an aryl group and a heterocyclic group, which may have a substituent, more preferably, a hydrogen atom, and an alkyl group having 1 to 18 carbon atoms, an aryl group having 6 to 18 carbon atoms or a heterocyclic group having 4 to 16 carbon atoms, which may have a substituent, still more preferably a hydrogen atom, and an alkyl group having 1 to 18 carbon atoms, which may have a substituent, and particularly preferably an alkyl group having 1 to 18 carbon atoms.

Further, R₂₃ and R₂₄ may be bound with each other to form a ring, and the ring is preferably an aliphatic hydrocarbon ring, and more preferably an aliphatic hydrocarbon ring having 4 to 10 carbon atoms.

In the —N(R₂₀)—, R₂₀ preferably represents an alkyl group, an aryl group and a heterocyclic group. R₂₀ may further have a substituent, and still more specific examples of the substituent include the substituent W, and are preferably an alkyl group or an aryl group.

R₂₀ is more preferably a hydrogen atom, and an alkyl group having 1 to 18 carbon atoms, an aryl group having 6 to 18 carbon atoms or a heterocyclic group having 4 to 16 carbon atoms, which may have a substituent, still more preferably a hydrogen atom, and an alkyl group having 1 to 18, which may have a substituent, and particularly preferably an alkyl group having 1 to 18 carbon atoms.

(In Formula (A-1), Ra₁ to Ra₈ independently represent a hydrogen atom, a halogen atom, an alkyl group, an aryl group, a heterocyclic group or an alkoxy group. Ra₁ to Ra₈ may further have a substituent, and still more specific examples of the substituent include the substituent W, and are preferably an alkyl group. Further, at least two of Ra₁ to Ra₈ may be bound with each other to form a ring. Examples of the ring formed include a cycloalkyl ring having 5 to 18 carbon atoms, a benzene ring, a naphthalene ring, an indane ring, an anthracene ring, a pyrene ring, a phenanthrene ring, a perylene ring, a pyridine ring, a quinoline ring, an isoquinoline ring, phenanthridine ring, a pyrimidine ring, a pyrazine ring, a pyridazine ring, a triazine ring, a cinnoline ring, an acridine ring, a phthalazine ring, a quinazoline ring, a quinoxaline ring, a naphthyridine ring, a pteridine ring, a pyrrole ring, a pyrazole ring, a triazole ring, an indole ring, a carbazole ring, an indazole ring, a benzimidazole ring, an oxazole ring, a thiazole ring, an oxadiazole ring, a thiadiazole ring, a benzoxazole ring, a benzothiazole ring, an imidazopyridine ring, a thiophene ring, a benzothiophene ring, a furan ring, a benzofuran ring, a phosphole ring, a phosphinine ring, a silole ring and the like. The ring is preferably a cycloalkyl ring having 5 to 18 carbon atoms, a benzene ring, a naphthalene ring, an indane ring, an anthracene ring, a pyrene ring, a phenanthrene ring, a perylene ring, a pyrrole ring, an indole ring, a carbazole ring, an indazole ring, a thiophene ring, a benzothiophene ring, a furan ring and a benzofuran ring, more preferably a cycloalkyl ring having 5 to 18 carbon atoms, a benzene ring, a naphthalene ring, an indane ring, an indole ring, a carbazole ring and an indazole ring, particularly preferably a cycloalkyl ring having 5 to 10 carbon atoms, a benzene ring, a naphthalene ring, an indane ring and an anthracene ring, and among them, the ring is preferably a cycloalkyl ring having 5 to 10 carbon atoms, a benzene ring, a naphthalene ring and an indane ring, and most preferably a cycloalkyl ring having 5 and 6 carbon atoms, a benzene ring and an indane ring. These rings may further have the substituent W to be described below.

From the viewpoint of chemical stability, electric charge mobility and heat resistance, Ra₁ to Ra₈ are preferably a hydrogen atom, a halogen atom, an alkyl group having 1 to 18 carbon atoms, an aryl group having 6 to 18 carbon atoms, a heterocyclic group having 4 to 16 carbon atoms and an alkoxy group having 1 and 2 carbon atoms, more preferably a hydrogen atom, an alkyl group having 1 to 12 carbon atoms, and an aryl group having 6 to 14 carbon atoms, and still more preferably a hydrogen atom, an alkyl group having 1 to 6 carbon atoms and an aryl group having 6 to 10 carbon atoms. The alkyl group may be branched.

Preferred examples of Ra₁ to Ra₈ include a hydrogen atom, a fluorine atom, a methyl group, an ethyl group, a propyl group, a butyl group, a hexyl group, a cyclohexyl group, a phenyl group, a naphthyl group and the like.

Further, it is preferred that at least one of Ra₃ and Ra₆ is a hydrogen atom or an alkyl group having 1 to 10 carbon atoms, and Ra₁, Ra₂, Ra₄, Ra₅, Ra₇ and Ra₈ are a hydrogen atom, or that at least one of Ra₂ and Ra₇ is a hydrogen atom or an alkyl group having 1 to 10 carbon atoms, and Ra₁, Ra₃, Ra₄, Ra₅, Ra₆ and Ra₈ are a hydrogen atom, and it is particularly preferred that Ra₃ and Ra₆ are a hydrogen atom or an alkyl group having 1 to 6 carbon atoms, and Ra₁, Ra₂, Ra₄, Ra₅, Ra₇ and Ra₈ are a hydrogen atom.

Xa represents a single bond, an oxygen atom, a sulfur atom, an alkylene group, a silylene group, an alkenylene group, a cycloalkylene group, a cycloalkenylene group, an arylene group, a divalent heterocyclic group or an imino group, and these groups may further have a substituent group. Still more specific examples include the substituent W, and are preferably an alkyl group or an aryl group.

Xa is preferably a single bond, an alkylene group having 1 to 12 carbon atoms, an alkenylene group having 2 to 12 carbon atoms, an arylene group having 6 to 14 carbon atoms, a heterocyclic group having 4 to 13 carbon atoms, an oxygen atom, a sulfur atom, and an imino group (for example, a phenylimino group, a methylimino group and a t-butylimino group) having a hydrocarbon group having 1 to 12 carbon atoms (preferably an aryl group or alkyl group), more preferably a single bond, an oxygen atom, an alkylene group having 1 to 6 carbon atoms (for example, a methylene group, a 1,2-ethylene group and a 1,1-dimethylmethylene group), an alkenylene group having 2 carbon atoms (for example, —CH₂═CH₂—), an arylene group having 6 to 10 carbon atoms (for example, a 1,2-perylene group and a 2,3-naphthylene group) and a silylene group, and still more preferably a single bond, an oxygen atom and an alkylene group having 1 to 6 carbon atoms (for example, a methylene group, a 1,2-etheylene group and a 1,1-dimethylmethylene group).

In the substituent (S₁₁), R_{S1} represents a hydrogen atom or an alkyl group. From the viewpoint of chemical stability, electric charge mobility and heat resistance, R_{S1} is preferably an alkyl group having 1 to 10 carbon atoms, and more preferably an alkyl group having 1 to 6 carbon atoms, and specifically, R_{S1} is preferably a methyl group, an ethyl group, a propyl group, an isopropyl group, a butyl group or a tert-butyl group, more preferably a methyl group, an ethyl group, a propyl group, an isopropyl group or a tert-butyl group, still more preferably a methyl group, an ethyl group, an isopropyl group or a tert-butyl group, and particularly preferably a methyl group, an ethyl group or a tert-butyl group.

R_{S2} represents a hydrogen atom or an alkyl group. From the viewpoint of chemical stability, electric charge mobility and heat resistance, R_{S2} is preferably a hydrogen atom or an alkyl group having 1 to 10 carbon atoms, and more preferably a hydrogen atom or an alkyl group having 1 to 6 carbon atom, and specifically, R_{S2} is preferably a hydrogen atom, a methyl group, an ethyl group, a propyl group, an isopropyl group, a butyl group or a tert-butyl group, more preferably a hydrogen atom, a methyl group, an ethyl group or a propyl group, still more preferably a hydrogen atom and a methyl group, and particularly preferably a methyl group.

R_{S3} represents a hydrogen atom or an alkyl group. From the viewpoint of chemical stability, electric charge mobility and heat resistance, R_{S3} is preferably a hydrogen atom or an alkyl group having 1 to 10 carbon atom, and more preferably a hydrogen atom or an alkyl group having 1 to 6 carbon atoms, and specifically, R_{S3} is a hydrogen atom or a methyl group, and more preferably a methyl group.

At least two of R_{S1} to R_{S3} may be bound with each other to form a ring. The ring is preferably an aliphatic hydrocarbon ring. The number of ring members is not particularly limited, but is preferably a 5-membered to 12-membered ring, more preferably a 5-membered or 6-membered ring, and still more preferably a 6-membered ring. Specific examples of the ring include a cyclopentane ring, a cyclohexane ring, an adamantane ring and the like.

S₁₁ represents the substituent (S₁₁), and is substituted with one of Ra₁ to Ra₈. It is preferred that at least one of Ra₁ and Ra₆ in Formula (A-1) each independently represents the substituent (S₁₁).

Preferred examples of the substituent (S₁₁) include the following (a) to (x), (a) to (j) are more preferred, (a) to (h) are still more preferred, (a) to (f) are particularly preferred, (a) to (c) are further more preferred, and (a) is most preferred. In the following (a) to (x), “*” represents a position substituted with Formula (A-1).

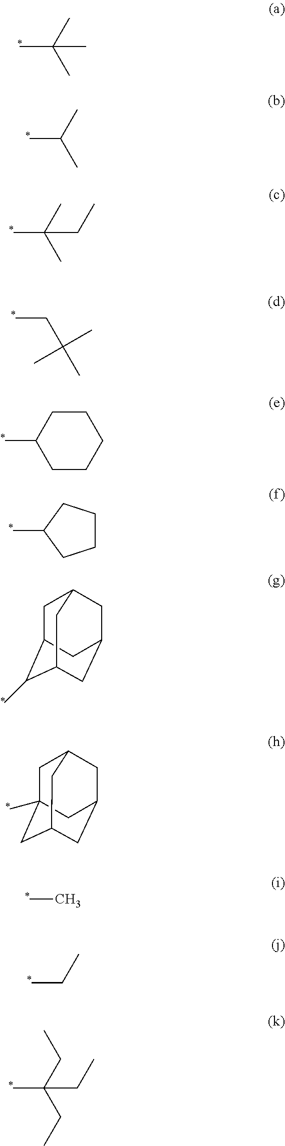

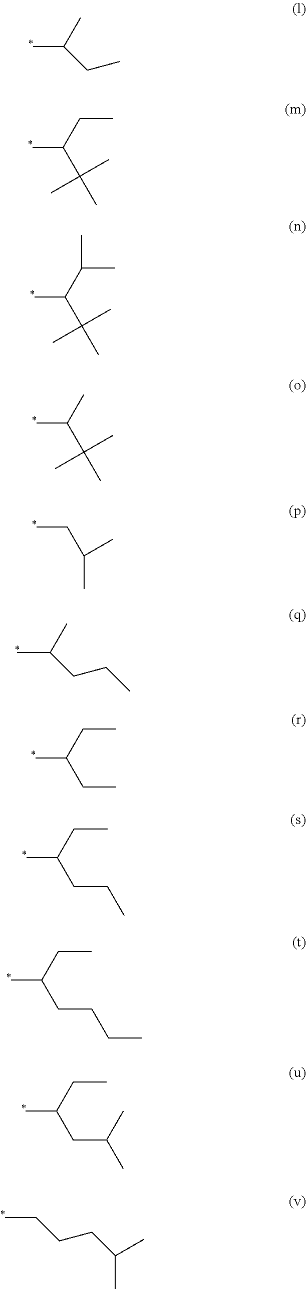

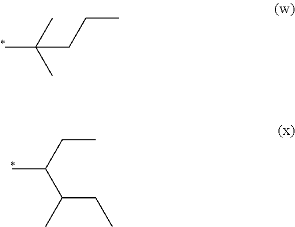

n each independently represents an integer of 1 to 4, and is preferably 1 to 3, more preferably 1 or 2, and particularly preferably 2. When the substituent represented by S₁₁ is introduced, and the compound represented by Formula (F-1) is used in a charge blocking layer of a photoelectric conversion device, interaction with the photoelectric conversion layer is suppressed, the dark current is decreased and intermolecular force among compounds represented by Formula (F-1) is increased due to polymerization, thereby making the device highly heat resistant.

One of preferred aspects in the present invention includes the case where in the group represented by Formula (A-1), Ra₁ to Ra₈ independently represent a hydrogen atom, a halogen atom or an alkyl group.

In the group represented by Formula (A-1), when Ra₁ to Ra₈ independently represent a hydrogen atom, a halogen atom or an alkyl group, one of preferred forms is a group in which Formula (A-1) is represented by the following Formulae (A-3) to (A-5).

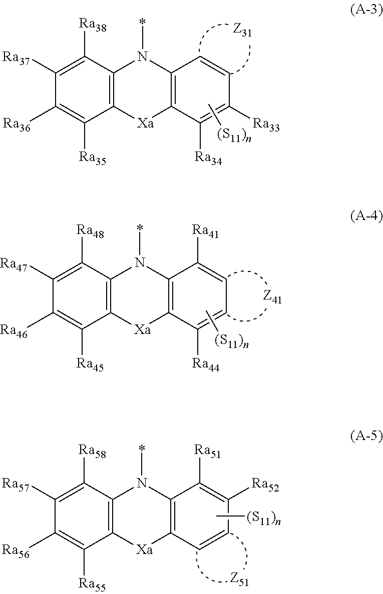

(In Formulae (A-3) to (A-5), Ra₃₃ to Ra₃₈, Ra₄₁, Ra₄₄ to Ra₄₈, Ra₅₁, Ra₅₂ and Ra₅₅ to Ra₅₈ each independently represent a hydrogen atom, a halogen atom or an alkyl group. * represents a bonding position. Xa represents a single bond, an oxygen atom, a sulfur atom, an alkylene group, a silylene group, an alkenylene group, a cycloalkylene group, a cycloalkenylene group, an arylene group, a divalent heterocyclic group or an imino group. S₁₁ each independently represents the substituent (S₁₁), and is substituted as one of Ra₃₃ to Ra₃₈, Ra₄₁, Ra₄₄ to Ra₄₈, Ra₅₁, Ra₅₂ and Ra₅₅ to Ra₅₈. Z₃₁, Z₄₁ and Z₅₁ represent a cycloalkyl ring, an aromatic hydrocarbon ring or an aromatic heterocyclic ring. n represents an integer of 1 to 4.)

Xa, S₁₁ and n in Formulae (A-3) to (A-5) have the same meaning as Xa, S₁₁ and n in Formula (A-1), and preferred examples are also the same. Ra₃₃ to Ra₃₈, Ra₄₁, Ra₄₄ to Ra₄₈, Ra₅₁, Ra₅₂ and Ra₅₅ to Ra₅₈ in Formulae (A-3) to (A-5) have the same meaning as a hydrogen atom, a halogen atom or an alkyl group, which Ra₂₁ to Ra₂₈ represent, in Formula (A-1), and preferred examples are also the same.

Z₃₁, Z₄₁ and Z₅₁ represent a cycloalkyl ring, an aromatic hydrocarbon ring or an aromatic heterocyclic ring. Preferred examples of the ring represented by Z₃₁, Z₄₁ and Z₅₁ include a cycloalkyl ring having 5 to 18 carbon atoms, a benzene ring, a naphthalene ring, an indane ring, an anthracene ring, a pyrene ring, a phenanthrene ring, a perylene ring, a pyridine ring, a quinoline ring, an isoquinoline ring, phenanthridine ring, a pyrimidine ring, a pyrazine ring, a pyridazine ring, a triazine ring, a cinnoline ring, an acridine ring, a phthalazine ring, a quinazoline ring, a quinoxaline ring, a naphthyridine ring, a pteridine ring, a pyrrole ring, a pyrazole ring, a triazole ring, an indole ring, a carbazole ring, an indazole ring, a benzimidazole ring, an oxazole ring, a thiazole ring, an oxadiazole ring, a thiadiazole ring, a benzoxazole ring, a benzothiazole ring, an imidazopyridine ring, a thiophene ring, a benzothiophene ring, a furan ring, a benzofuran ring, a phosphole ring, a phosphinine ring, a silole ring and the like. The ring is more preferably a cycloalkyl ring having 5 to 18 carbon atoms, a benzene ring, a naphthalene ring, an indane ring, an anthracene ring, a pyrene ring, a phenanthrene ring, a perylene ring, a pyrrole ring, an indole ring, a carbazole ring, an indazole ring, a thiophene ring, a benzothiophene ring, a furan ring and a benzofuran ring, still more preferably a cycloalkyl ring having 5 to 18 carbon atoms, a benzene ring, a naphthalene ring, an indane ring, an indole ring, a carbazole ring and an indazole ring, particularly preferably a cycloalkyl ring having 5 to 10 carbon atoms, a benzene ring, a naphthalene ring, an indane ring and an anthracene ring, and among them, the ring is preferably a cycloalkyl ring having 5 to 10 carbon atoms, a benzene ring, a naphthalene ring and an indane ring, and most preferably a cycloalkyl ring having 5 and 6 carbon atoms, a benzene ring and an indane ring. These rings may have the substituent W to be described below.

Specific examples of the group represented by Formula (A-1) include groups represented by the following N-1 to N-135. However, the present invention is not limited thereto. The group represented by Formula (A-1) is preferably N-1 to N-93, more preferably N-1 to N-72, still more preferably N-1 to N-37, and among them, the group is preferably N-1 to N-3, N-12 to N-22 and N-24 to N-35, particularly preferably N-1 to N-3, N-17 to N-22 and N-30 to N-35, and most preferably N-1 to N-3, N-17 to N-19 and N-30 to N-32. (S) in the drawing represents the aforementioned substituent (S₁₁), n′ and n″ each independently represent an integer of 1 to 4, and n′+n″ is an integer of 1 to 4.

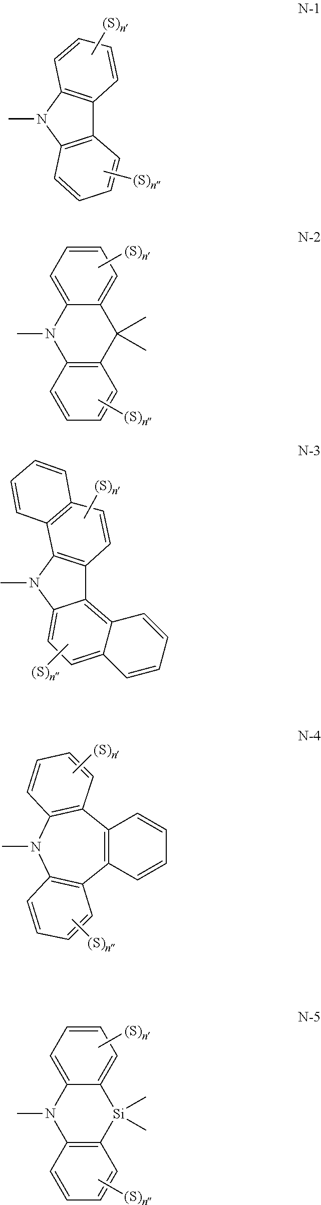

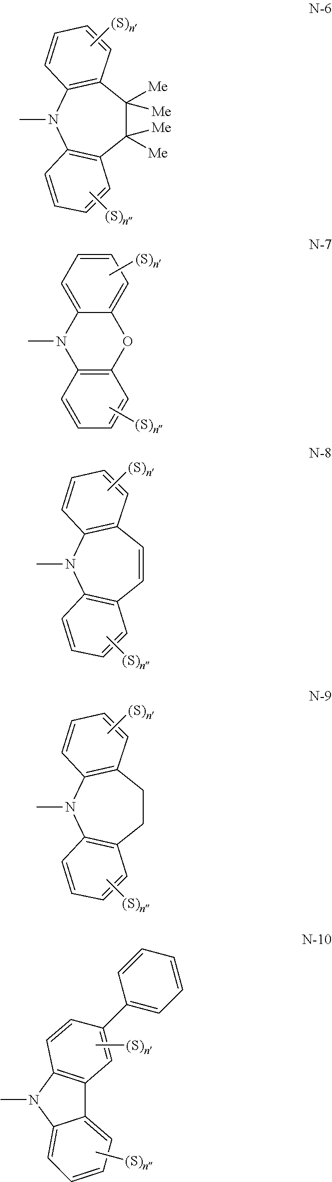

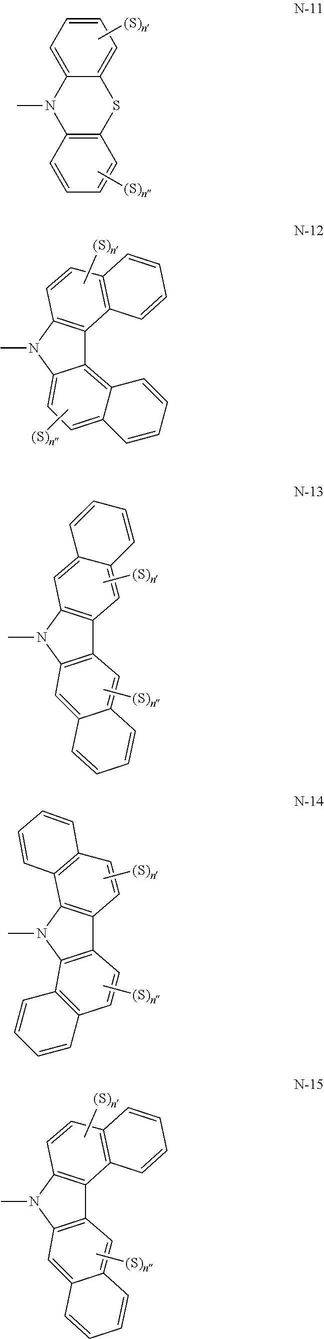

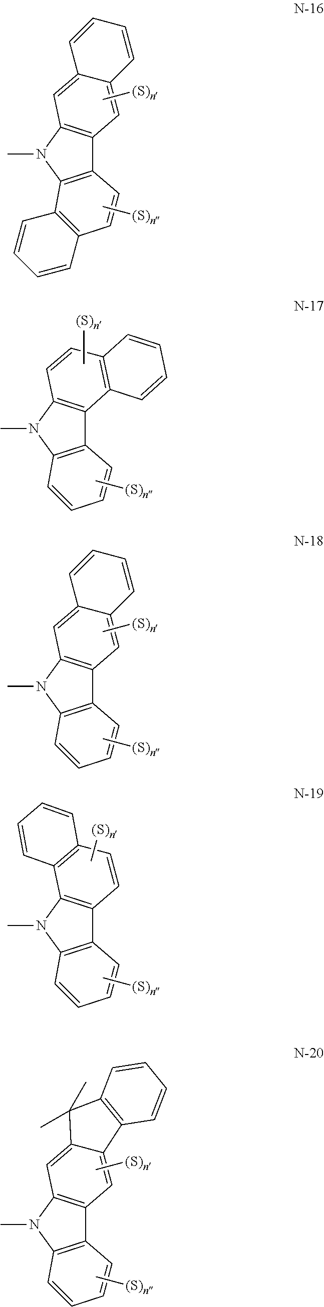

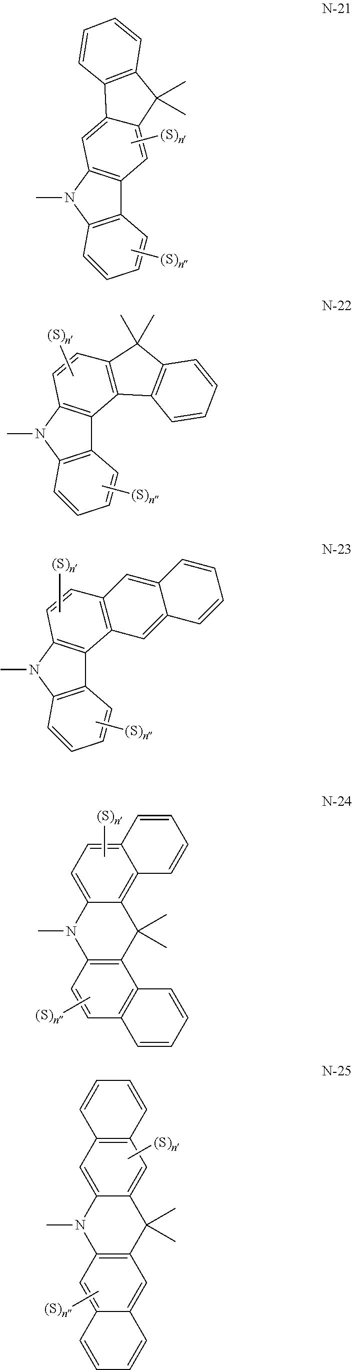

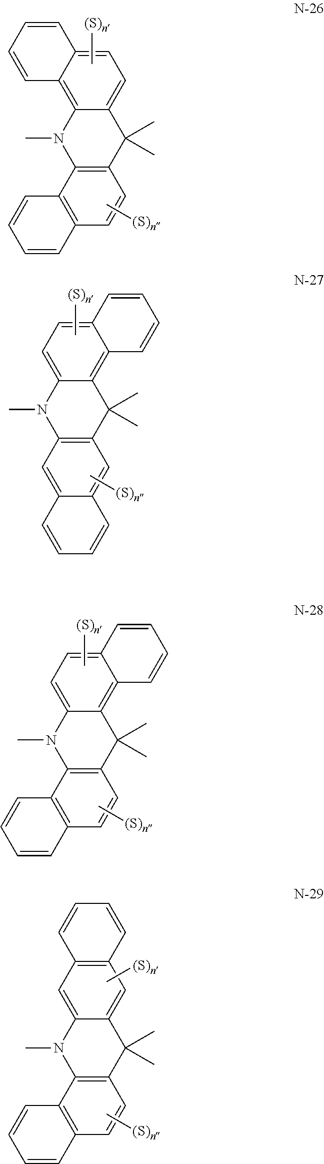

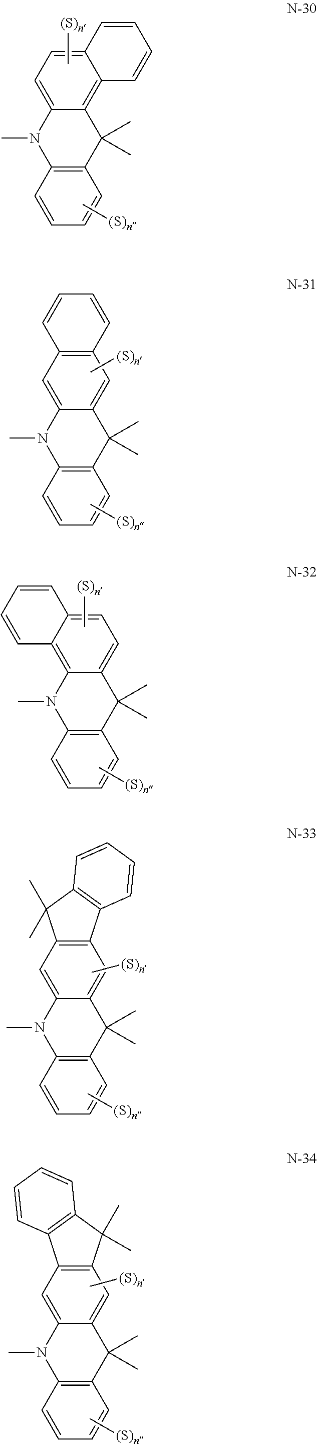

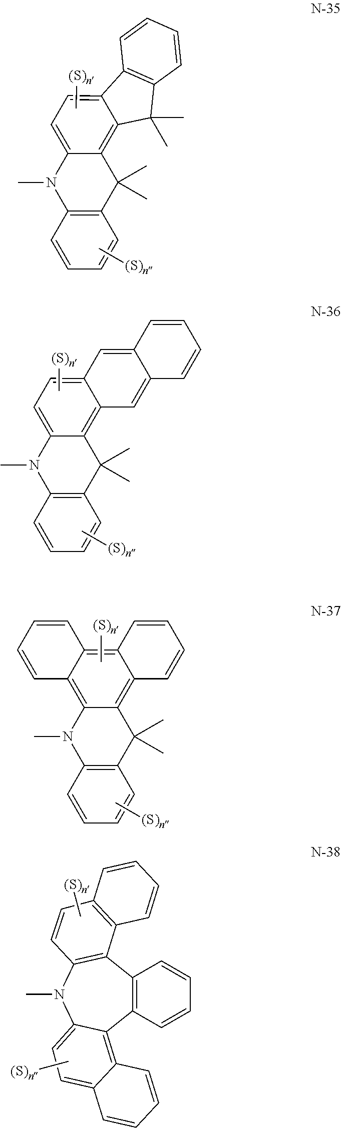

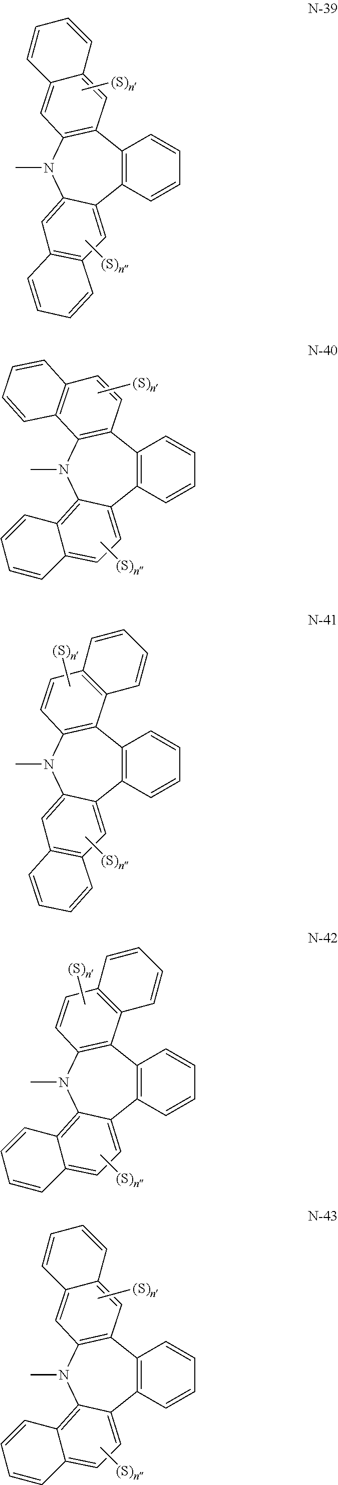

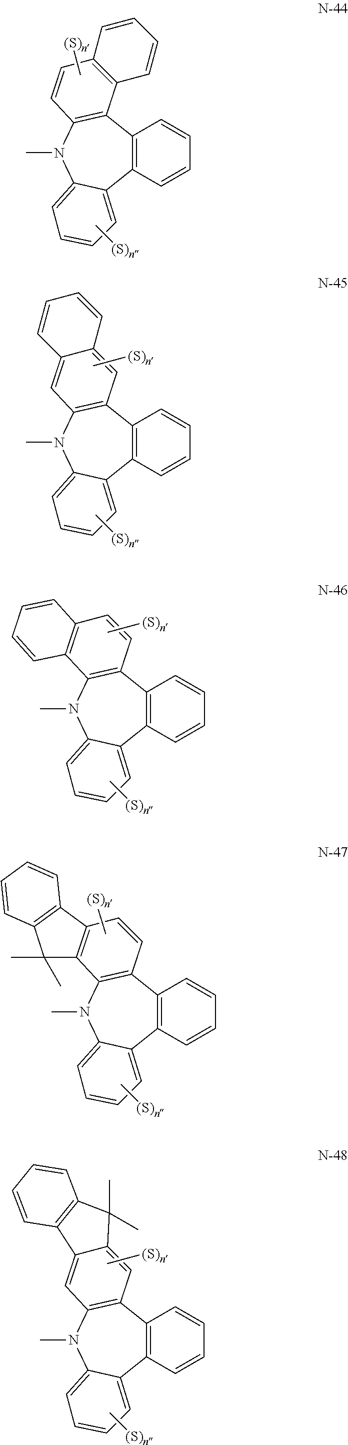

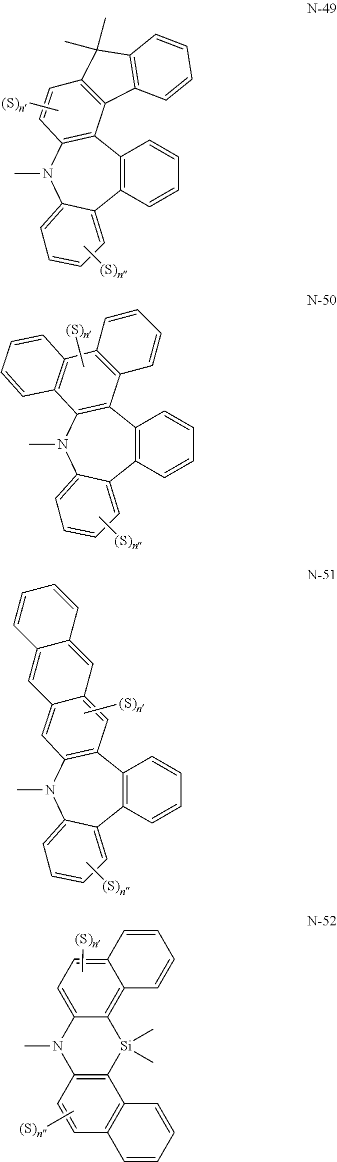

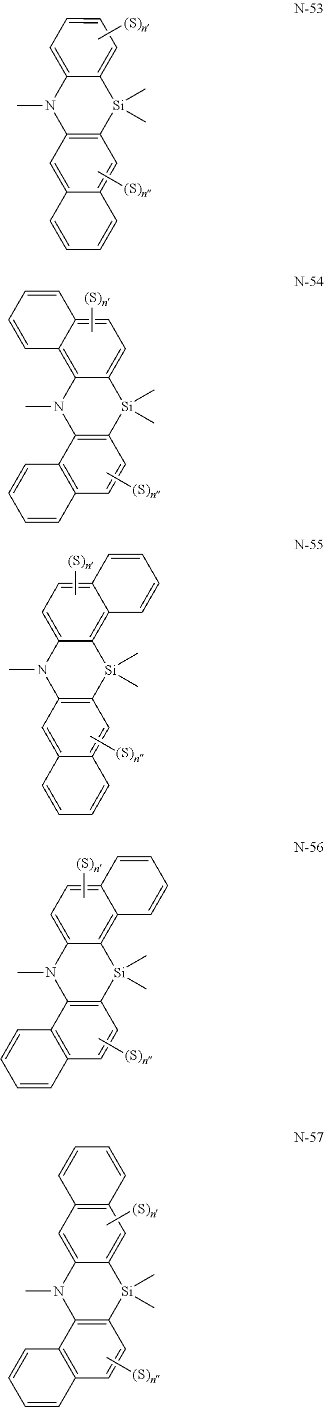

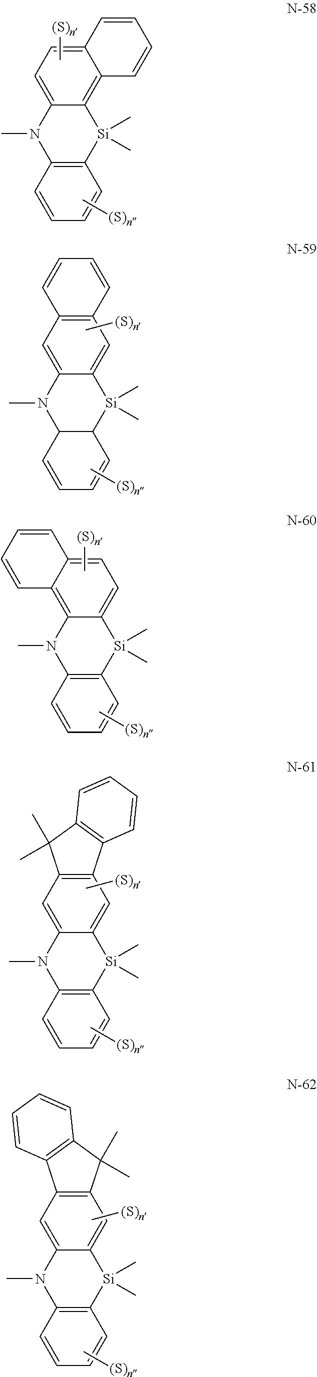

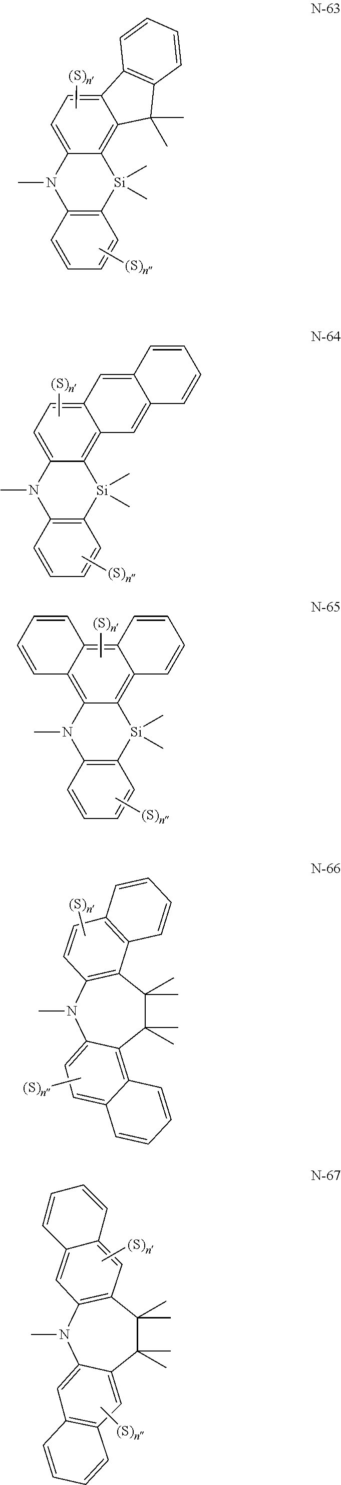

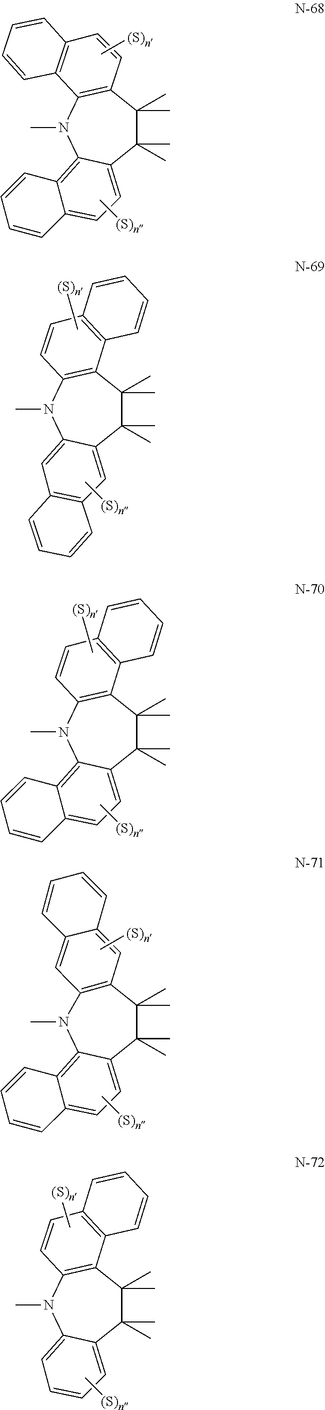

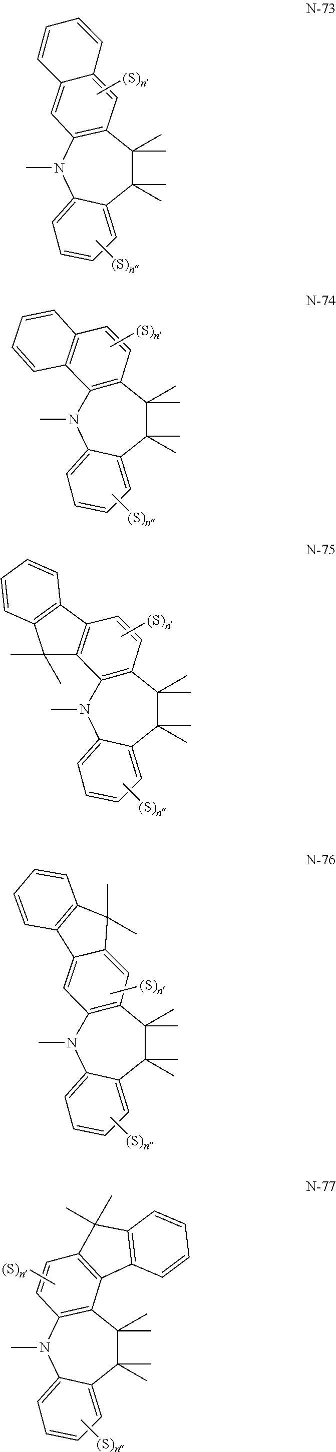

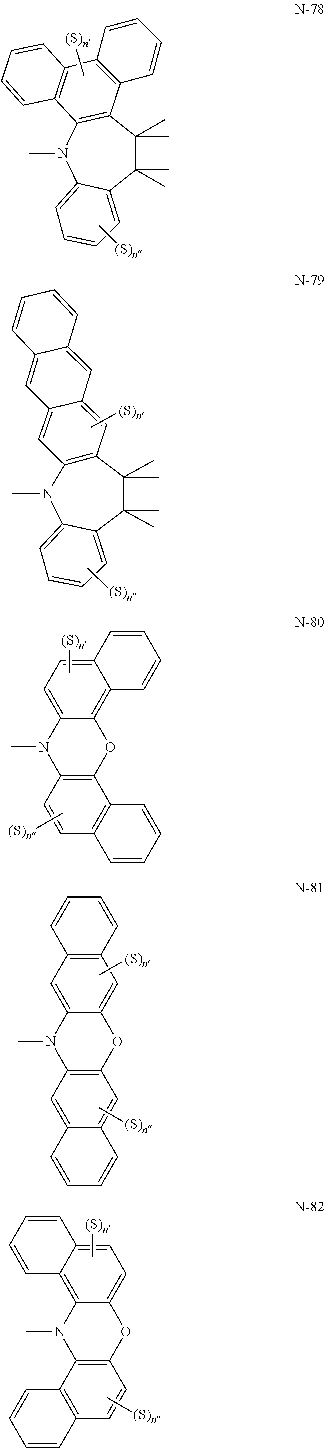

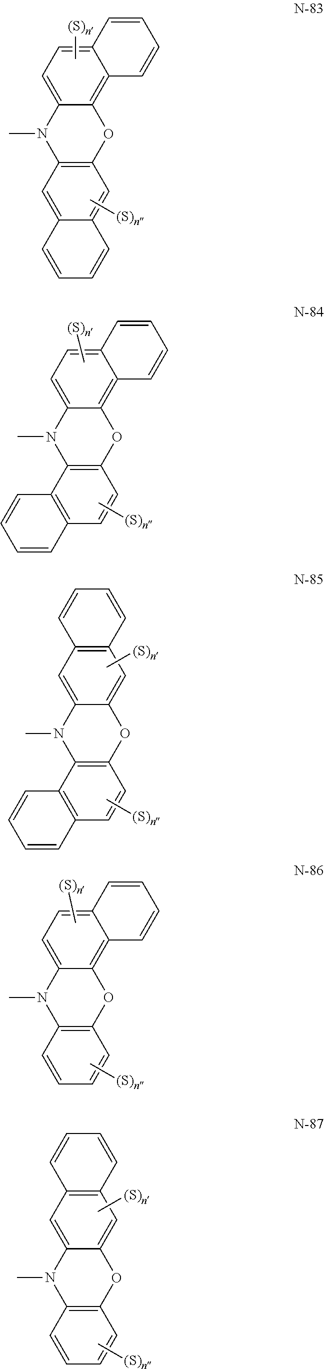

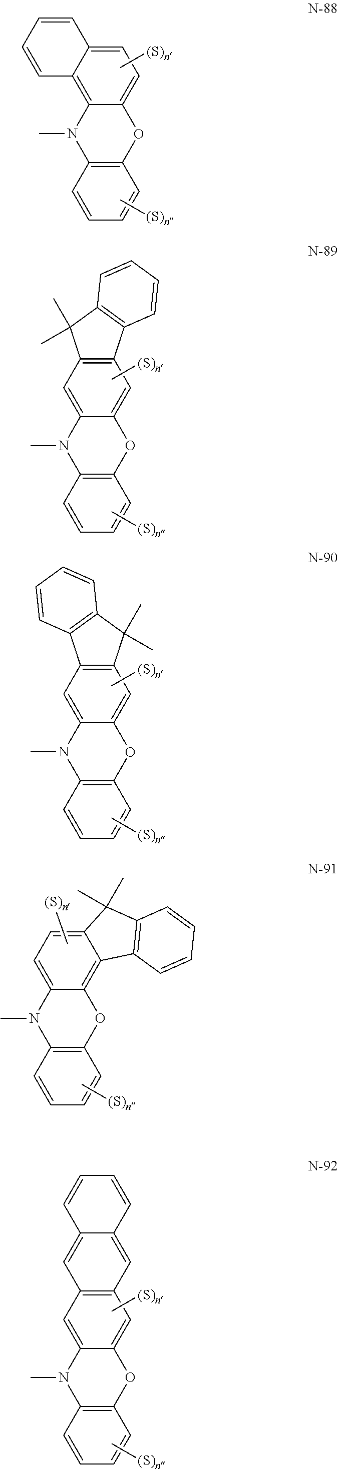

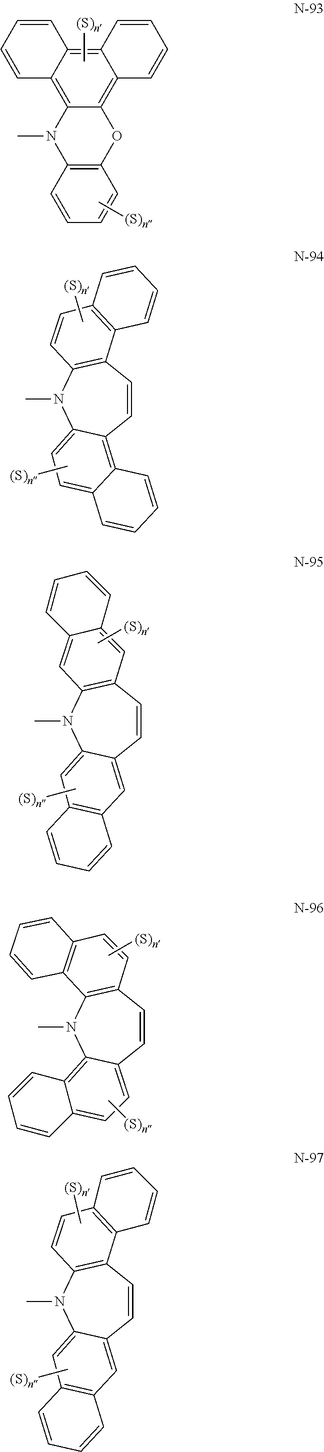

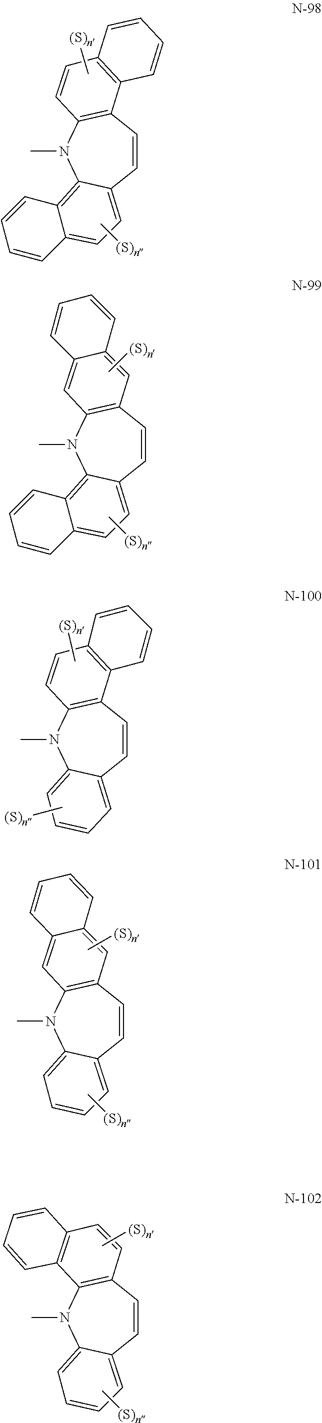

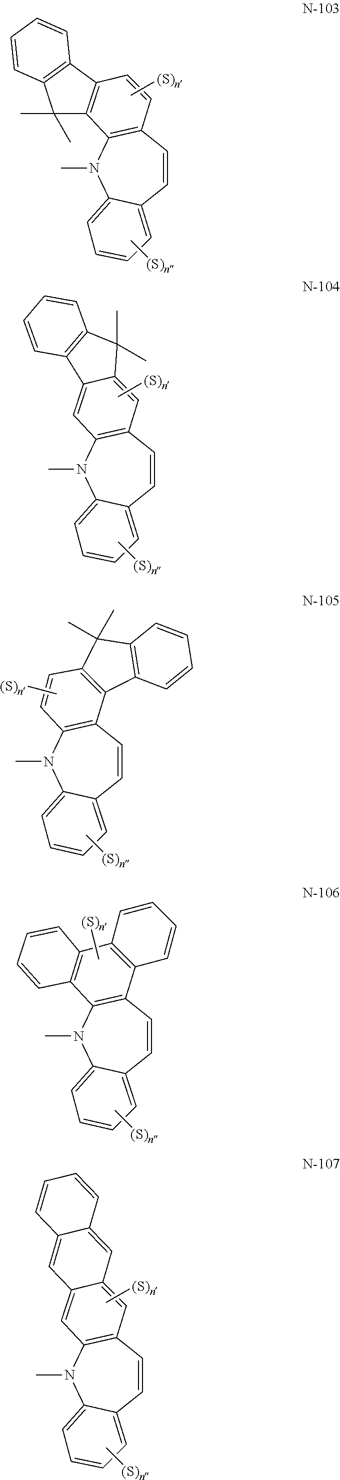

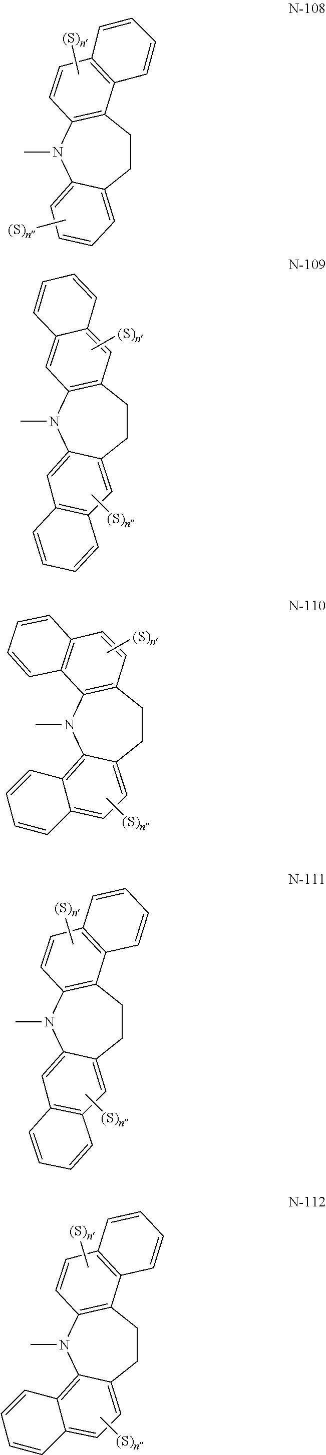

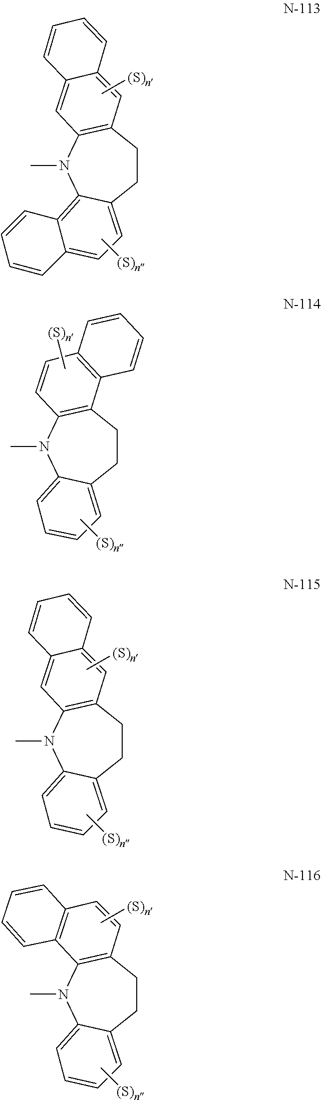

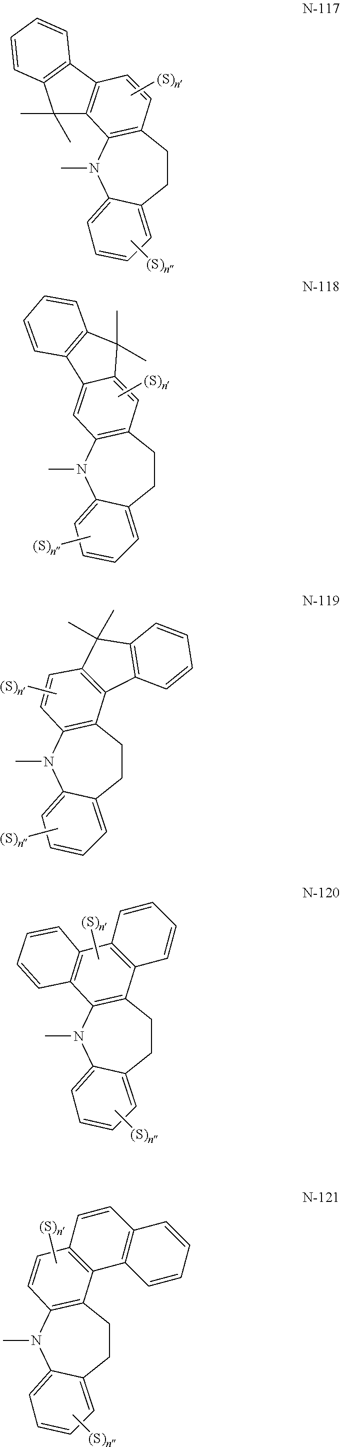

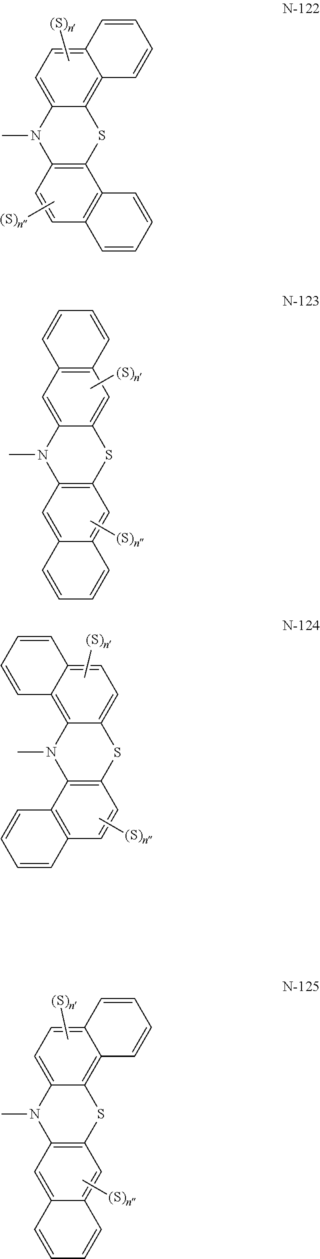

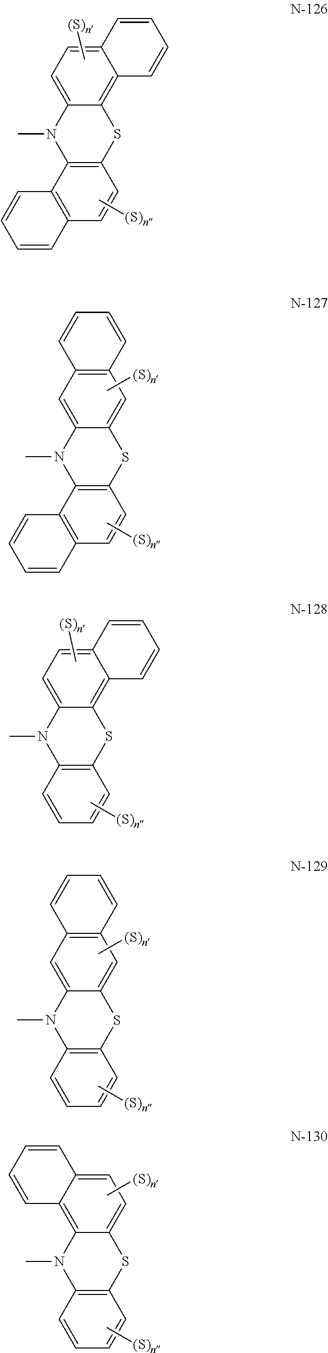

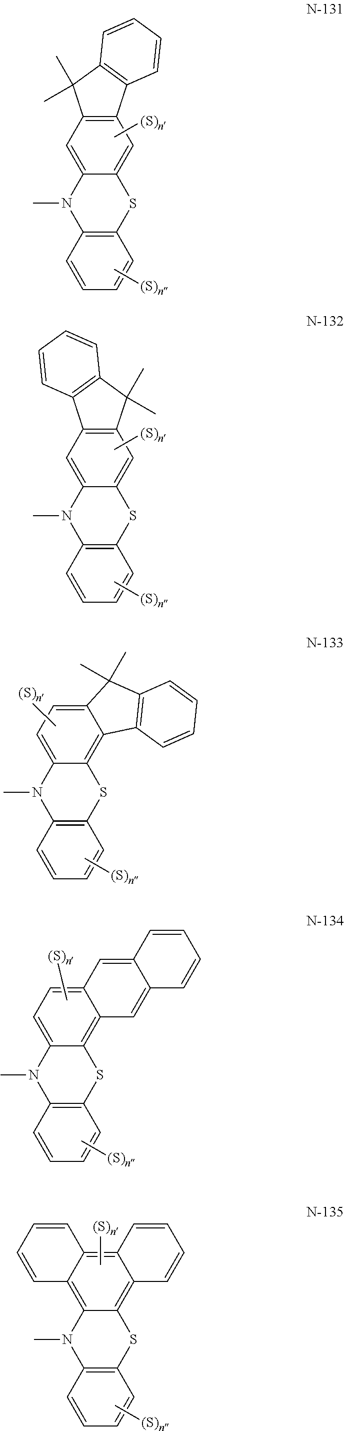

A preferred form of the compound represented by Formula (F-1) is a compound represented by the following Formula (F-2). When the compound having the structure like Formula (F-2) is used in a charge blocking layer of a photoelectric conversion device, interaction with the photoelectric conversion layer is suppressed, the dark current is decreased, and intermolecular force is increased due to polymerization, thereby making the device highly heat resistant.

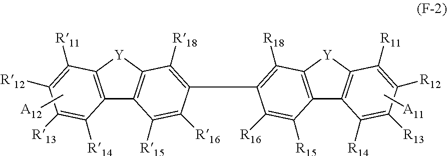

(In Formula (F-2), R₁₁ to R₁₆, R₁₈, R′₁₁ to R′₁₆ and R′₁₈ each independently represent a hydrogen atom, a halogen atom, an alkyl group, an aryl group, a heterocyclic group, a hydroxyl group, an amino group or a mercapto group, and these groups may further have a substituent. A₁₁ and A₁₂ each independently represent the substituent represented by Formula (A-1), and are substituted as one of R₁₁ to R₁₄ and one of R′₁₁ to R′₁₄. Y each independently represents a carbon atom, a nitrogen atom, an oxygen atom, a sulfur atom or a silicon atom, and these groups may further have a substituent.)

In Formula (F-2), R₁₁ to R₁₆, R₁₈, R′₁₁ to R′₁₆, R′₁₈, Y, A₁₁ and A₁₂ have the same meaning as R₁₁ to R₁₆, R₁₈, R′₁₁ to R′₁₆, R′₁₈, Y, A₁₁ and A₁₂ in Formula (F-1), and preferred ranges are also the same.

A preferred form of the compound represented by Formula (F-1) and the compound represented by Formula (F-2) is the case where Y in Formulae (F-1) and (F-2) each independently, represents —C(R₂₁)(R₂₂)—, —Si(R₂₃)(R²⁴)—, an oxygen atom or a sulfur atom, and Ra₁ to Ra₈ in the group represented by Formula (A-1) independently represent an oxygen, a halogen atom or an alkyl group. By using the compound of this aspect in a charge blocking layer of the photoelectric conversion device, interaction with the photoelectric conversion layer is suppressed, dark current is decreased, and intermolecular force is increased due to polymerization, thereby making the device highly heat resistant. The case where Y each independently represents —C(R₂₁)(R₂₂)—, and R₂₁ and R₂₂ each independently represent an alkyl group, an aryl group or a heterocyclic group is particularly preferred.

As another aspect of the compound represented by Formula (F-1) and the compound represented by Formula (F-2), the case where Y in Formulae (F-1) and (F-2) each independently represents —N(R₂₀)—, and R₂₀ represents an alkyl group, an aryl group or a heterocyclic group is also preferred. It is possible to obtain an effect of obtaining a device having a fast response speed by using the compound of this aspect in the charge blocking layer.

Further, a preferred form of the compound represented by Formula (F-1) and the compound represented by Formula (F-2) is the case where the substituent represented by Formula (A-1) is independently substituted with R₁₂ and R′₁₂. The symmetry of molecule is enhanced, and the melting temperature and the glass transition temperature are increased.

The case where n in Formula (A-1) is 1 or 2 is preferred. By using the compound of this aspect in a charge blocking layer of the photoelectric conversion device, interaction with the photoelectric conversion layer is suppressed, dark current is decreased, and intermolecular force is increased due to polymerization, thereby making the device highly heat resistant.

In particular, the case where at least one of Ra₁ and Ra₆ in Formula (A-1) each independently represents the substituent (S₁₁) is particularly preferred. The active site is protected, thereby enhancing the chemical stability of the compound.

An ionization potential (Ip) of the compound represented by Formula (F-1) and the compound represented by Formula (F-2) needs to be smaller than the Ip of a material responsible for transporting holes in the photoelectric conversion layer because holes need to be received from the material responsible for transporting holes in the photoelectric conversion layer without a barrier when the compounds are used in the charge blocking layer. In particular, when selecting an absorbing material having sensitivity in a visible light range, it is preferred that the compound according to the present invention has an ionization potential of 5.8 eV or less in order to be suitable for more materials. It is possible to obtain an effect of exhibiting high electric charge collection efficiency and fast responsiveness without generating a barrier for charge transport by having an Ip of 5.8 eV or less.

Further, the Ip is preferably 4.9 eV or more, and more preferably 5.0 eV or more. It is possible to obtain an effect of highly suppressing dark current by having an Ip of 4.9 eV or more.

In addition, the Ip of each compound may be measured by ultraviolet photoelectron spectroscopy (UPS) or a photoelectron spectrometer in air (for example, AC-2 and the like manufactured by RIKEN KEIKI Co., Ltd.).

The Ip of the compound according to the present invention may be adjusted to the range by changing a substituent which is bonded to the structure, and the like.

Next, compounds represented by Formula (2) will be described.

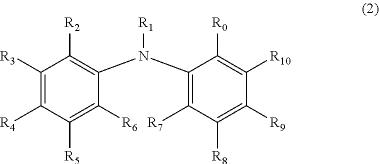

(In the formula, R₁ represents an alkyl group, an aryl group or a heterocyclic group, which may have a substituent. R₀ and R₂ to R₁₀ independently represent a hydrogen atom or a substituent.)

R₁ represents an alkyl group, an aryl group or a heterocyclic group, and may have a substituent. Specific examples of the substituent include the substituent W to be described below, and are preferably a halogen atom, an alkyl group, an aryl group, a heterocyclic group, a hydroxyl group, an amino group or a mercapto group, more preferably a halogen atom, an alkyl group, an aryl group, a heterocyclic group and an amino group, still more preferably a fluorine atom, an alkyl group, an aryl group and an amino group, particularly preferably an alkyl group, an aryl group and an amino group, and most preferably an aryl group and an amino group, which have a substituent (as the substituent, an alkyl group, an aryl group and a heterocyclic group are preferred).

Further, in the case of having a plurality of substituents, the substituents may be linked to each other to form a ring. Examples of the ring formed include the ring R to be described below.

When R₁ is an alkyl group, the alkyl group may be a straight•branched alkyl group, and a cyclic alkyl group (a cycloalkyl group), but is preferably a cycloalkyl group. When a carbazole structure is not included in R₁, the carbon number thereof is preferably 4 to 20, and more preferably 5 to 16, and when a carbazole structure is included in R₁, the carbon number thereof is preferably 19 to 35, and more preferably 20 to 31. Specifically, examples of the cycloalkyl group include a cycloalkyl group (a cyclopropyl group, a cyclopentyl group, a cyclohexyl group and the like), a cycloalkenyl group (a 2-cyclohexen-1-yl group and the like), and the like.

When R₁ is an aryl group, the aryl group is a substituted or unsubstituted aryl group having preferably 6 to 20 carbon atoms and more preferably 6 to 16 carbon atoms in the case where a carbazole structure is not included in R₁, and a substituted or unsubstituted aryl group having preferably 21 to 35 carbon atoms, and more preferably 21 to 31 carbon atoms in the case where a carbazole structure is included in R₁. More specific examples thereof include a phenyl group, a naphthyl group, an anthryl group, a fluorenyl group and the like.

When R₁ is a heterocyclic group, examples of the heterocyclic group include a 5-membered or 6-membered heterocyclic group, and specific examples thereof include a furyl group, a thienyl group, a pyridyl group, a quinolyl group, a thiazolyl group, an oxazolyl group, an azepinyl group, a carbazolyl group and the like. The aryl group or heterocyclic group may include a condensed ring composed of 2 to 4 monocycles.

R₁ is preferably an aryl group or a heterocyclic group, more preferably an aryl group, and most preferably a phenyl group.

R₀ and R₂ to R₁₀ each independently represent a hydrogen atom or a substituent, and specific examples of the substituent include the substituent W to be described below. The substituent is preferably a halogen atom, an alkyl group, an aryl group, a heterocyclic group, a hydroxyl group, an amino group or a mercapto group, more preferably a halogen atom, an alkyl group, an aryl group and a heterocyclic group, still more preferably a fluorine atom, an alkyl group and an aryl group, particularly preferably an alkyl group and an aryl group, and most preferably an alkyl group.

At least two of R₀ and R₂ to R₁₀ may be bound with each other to form a ring. Examples of the ring formed include the ring R to be described below.

Hereinafter, specific examples of the compound represented by Formula (1), (2), (F-1) or (F-2) according to the present invention will be described, but the present invention is not limited to the following specific examples.

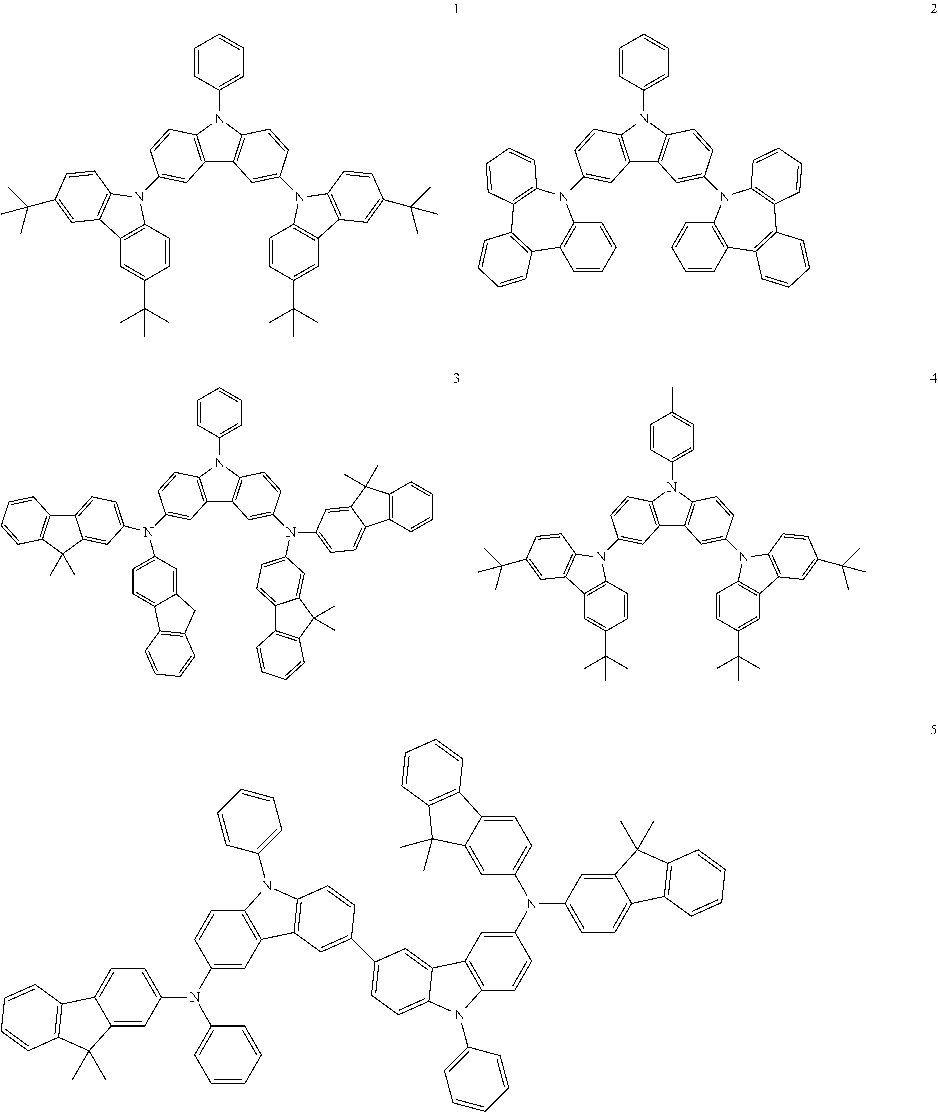

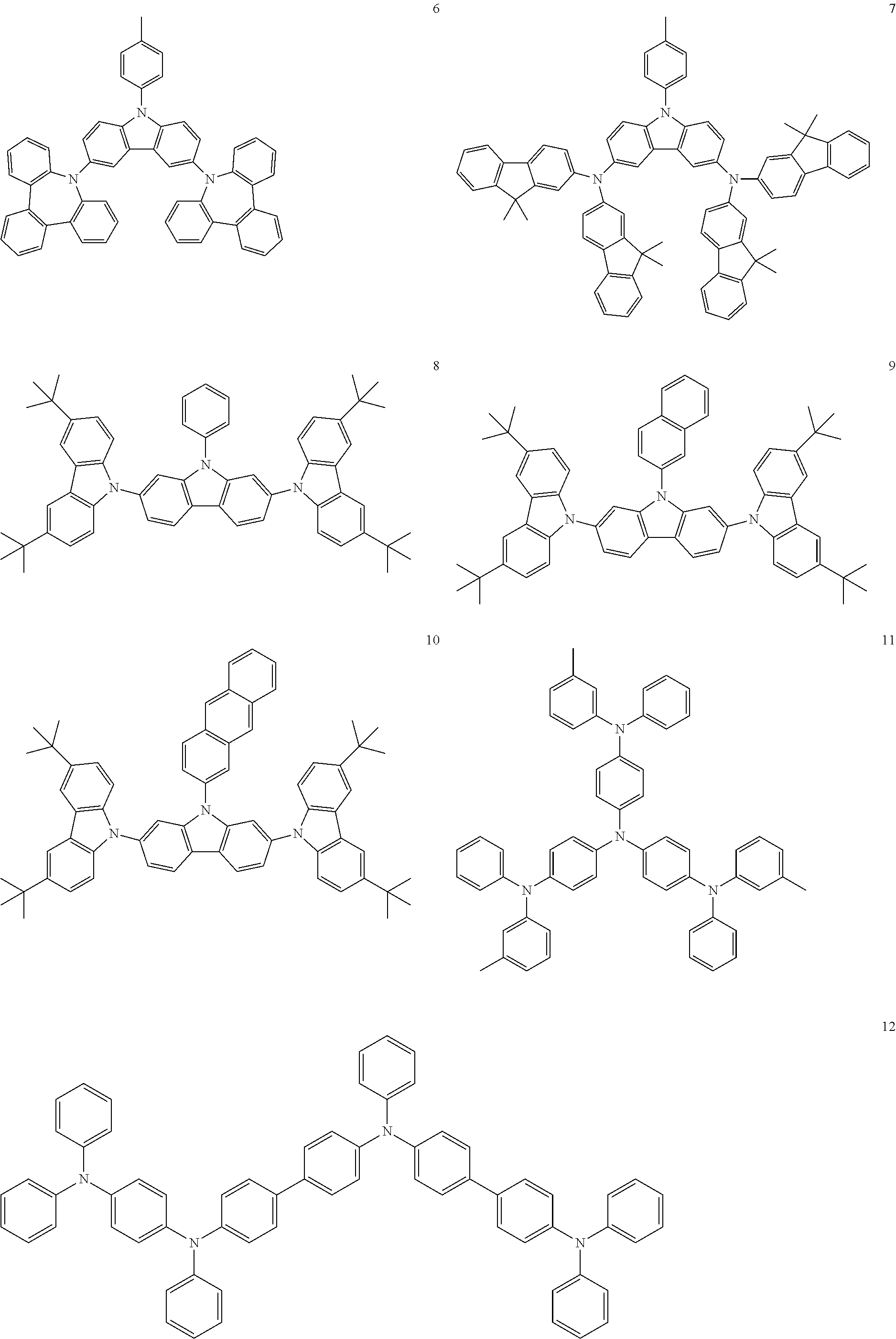

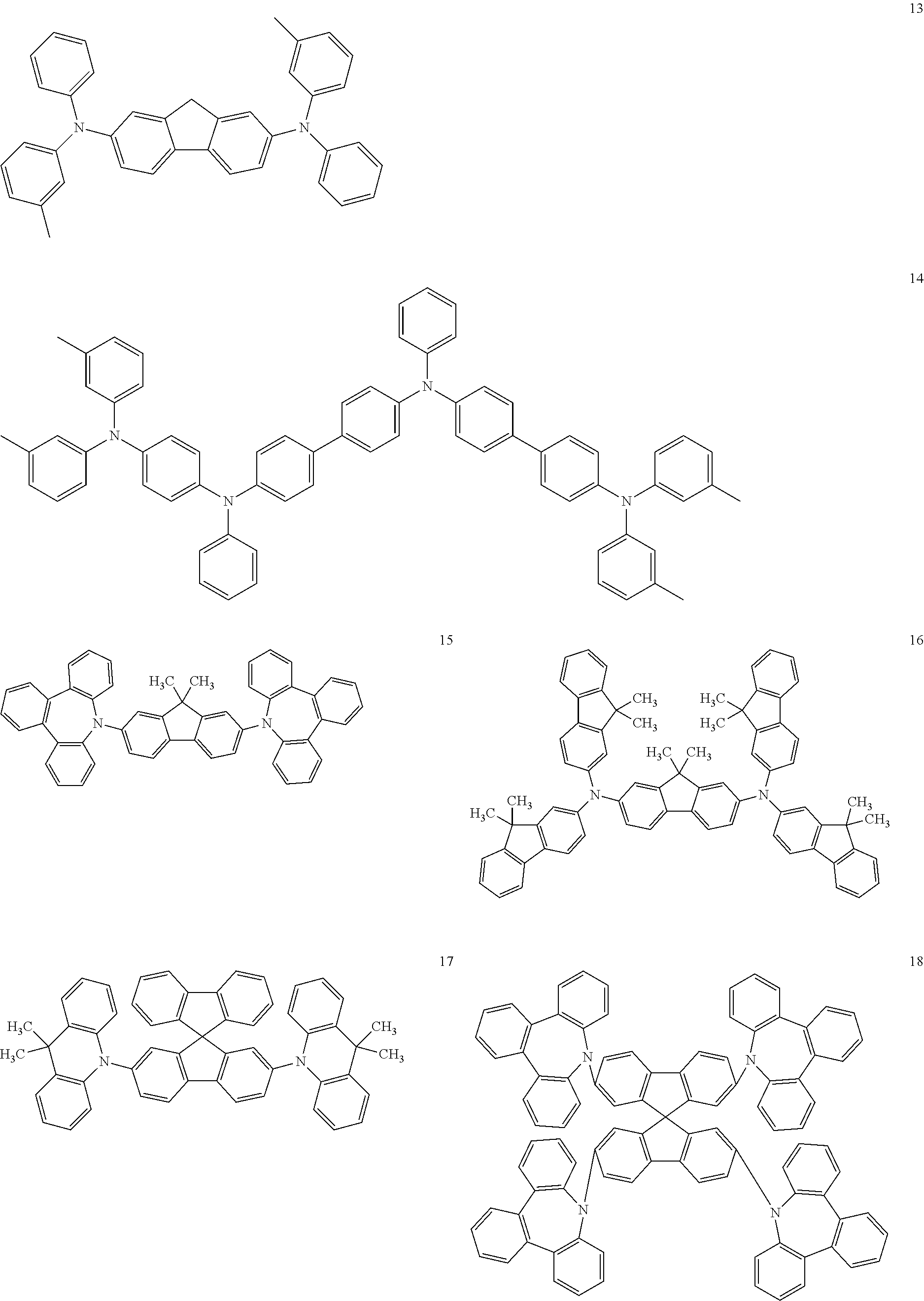

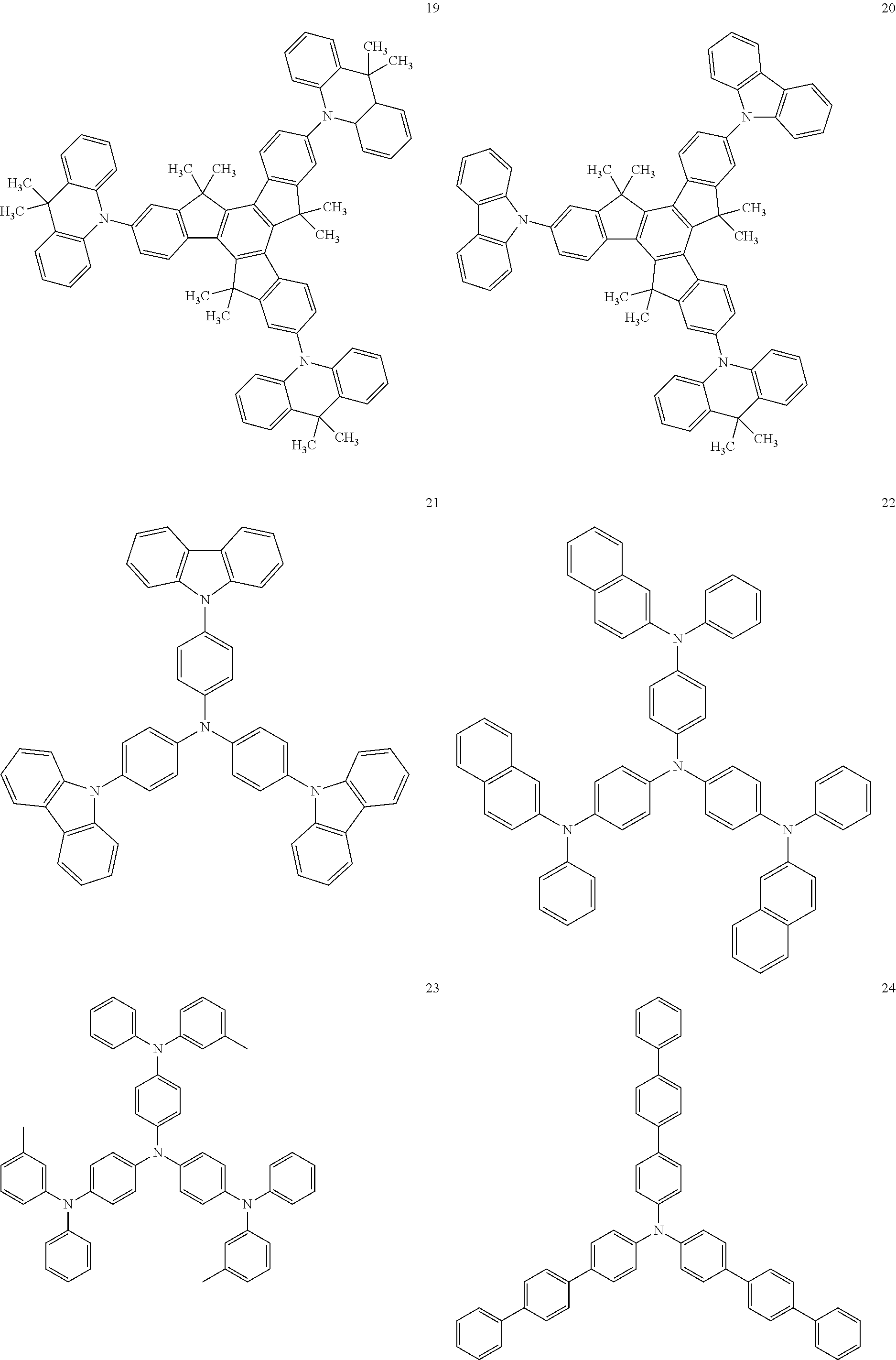

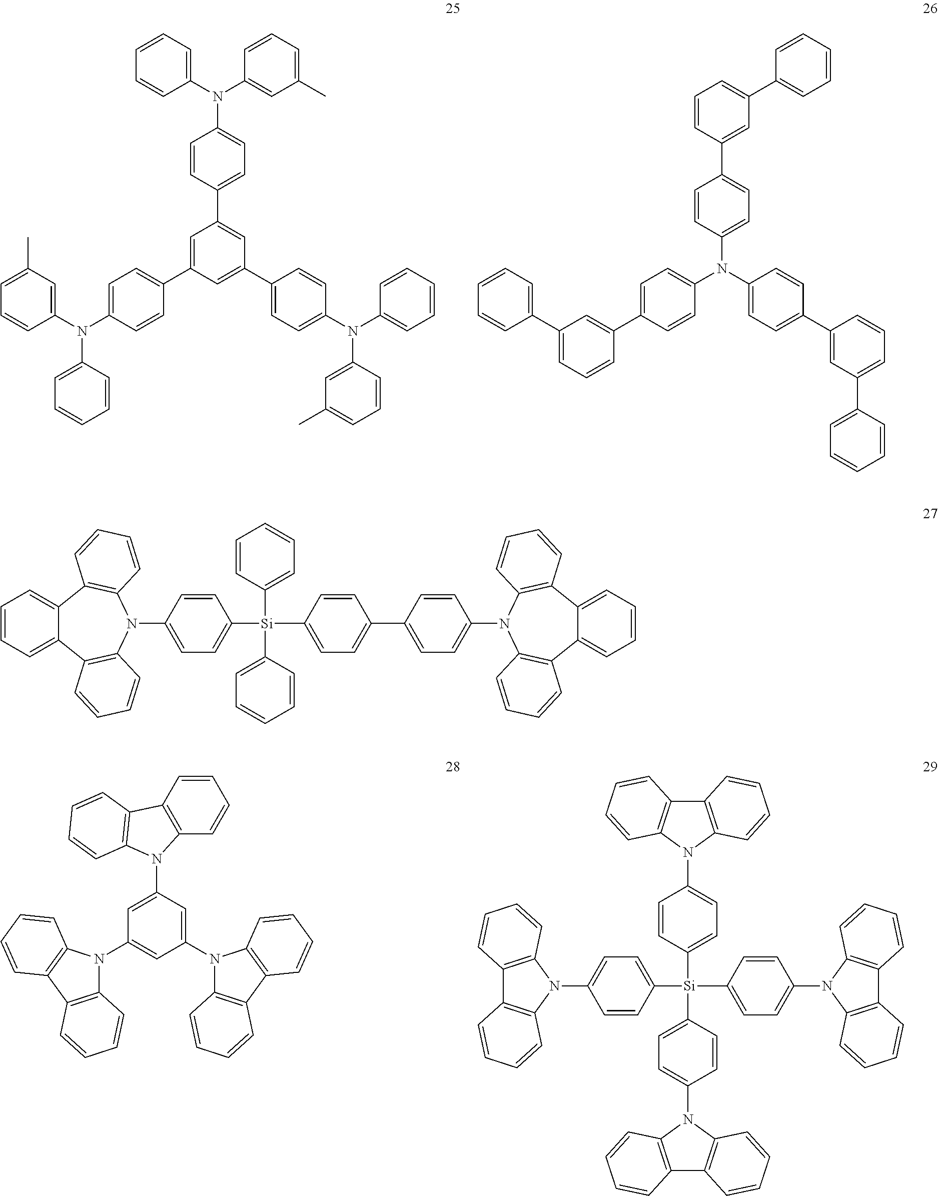

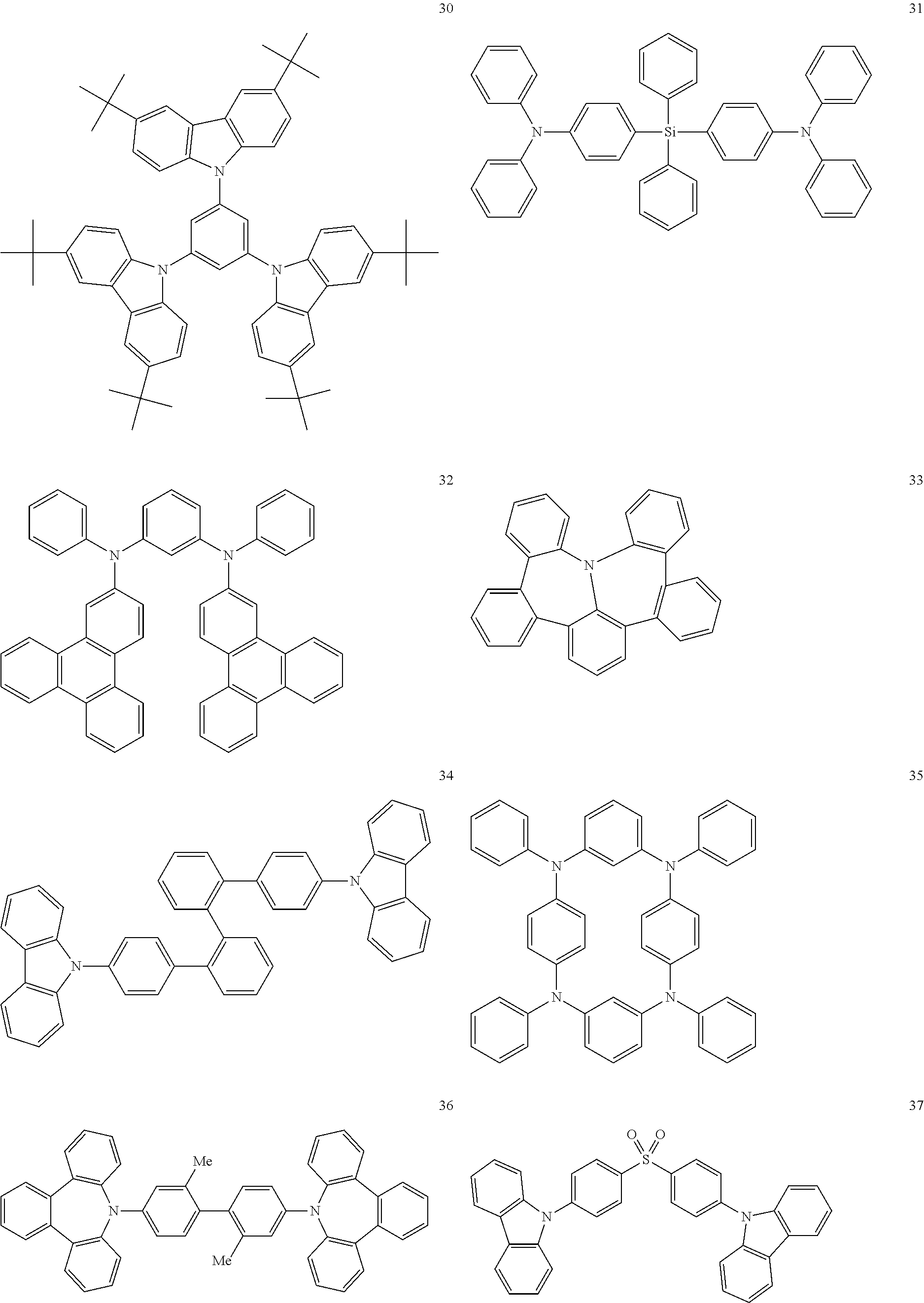

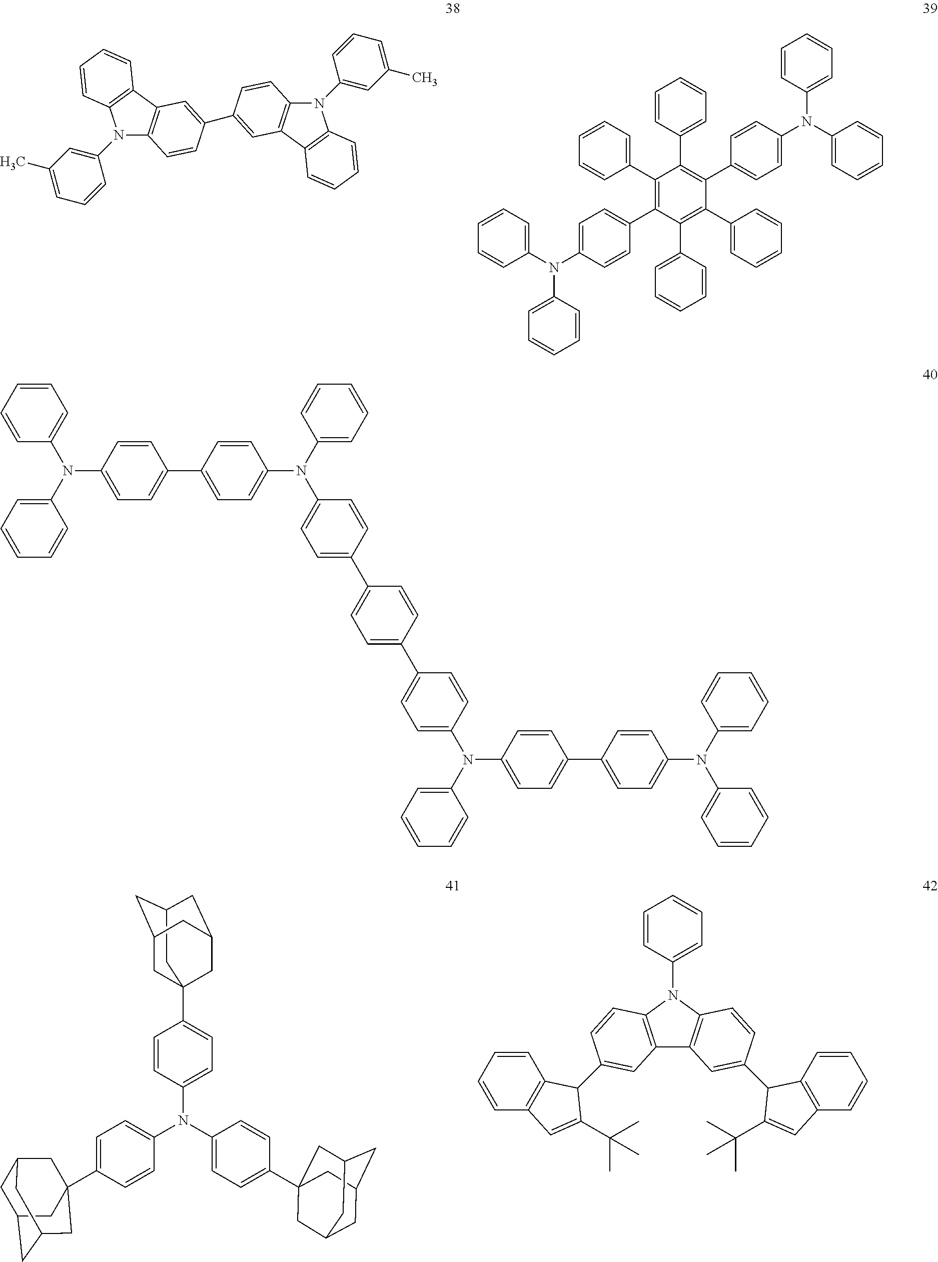

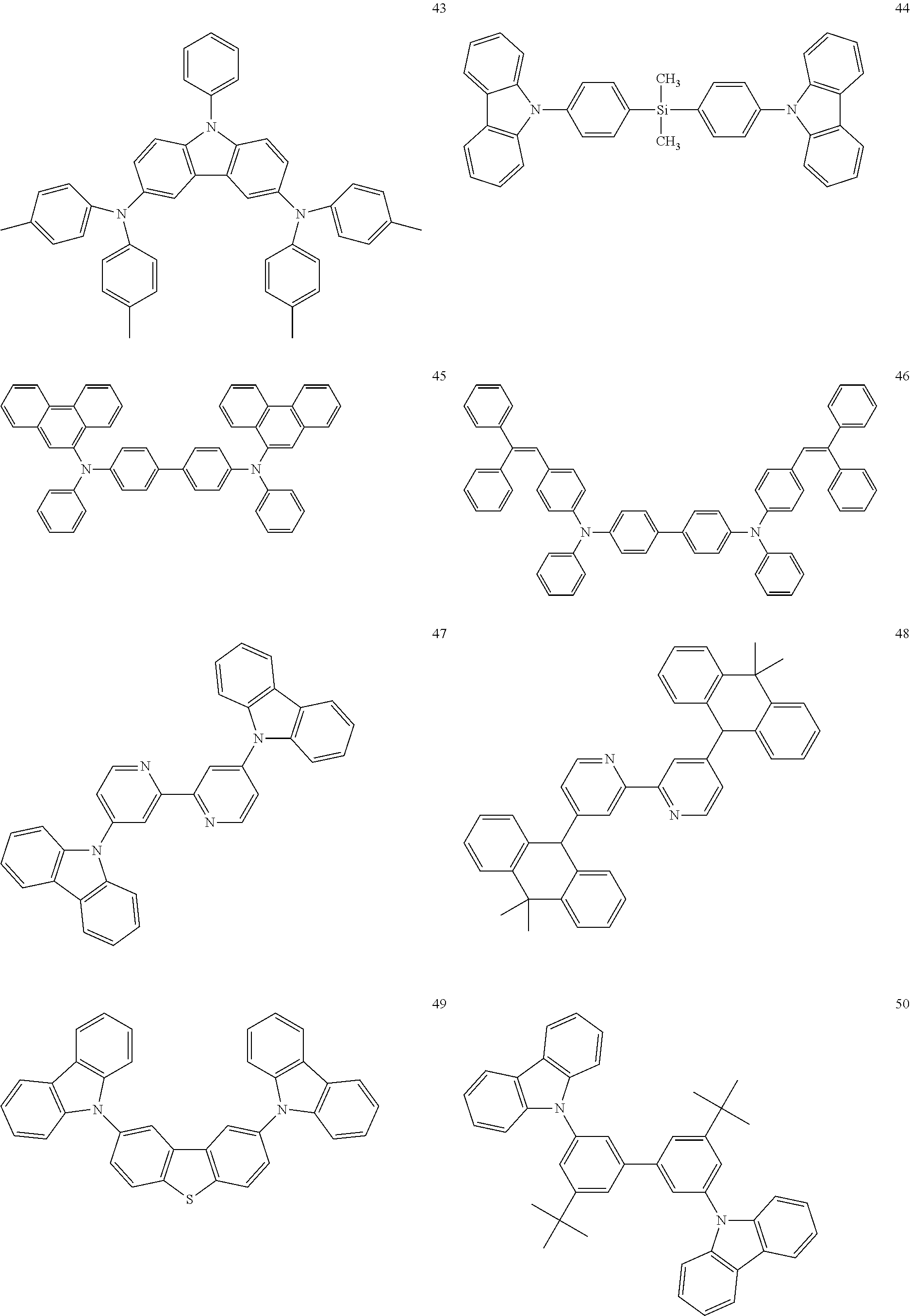

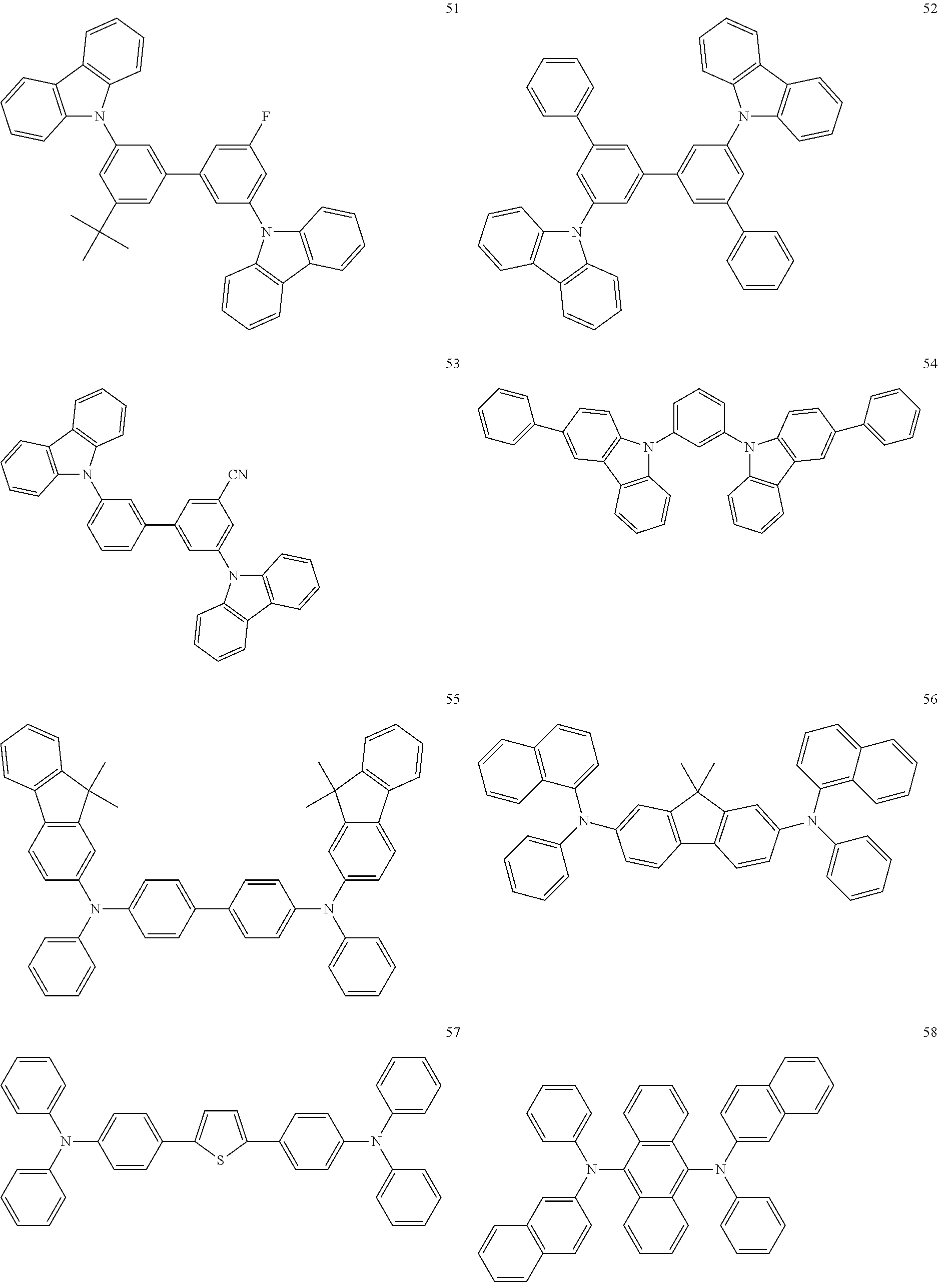

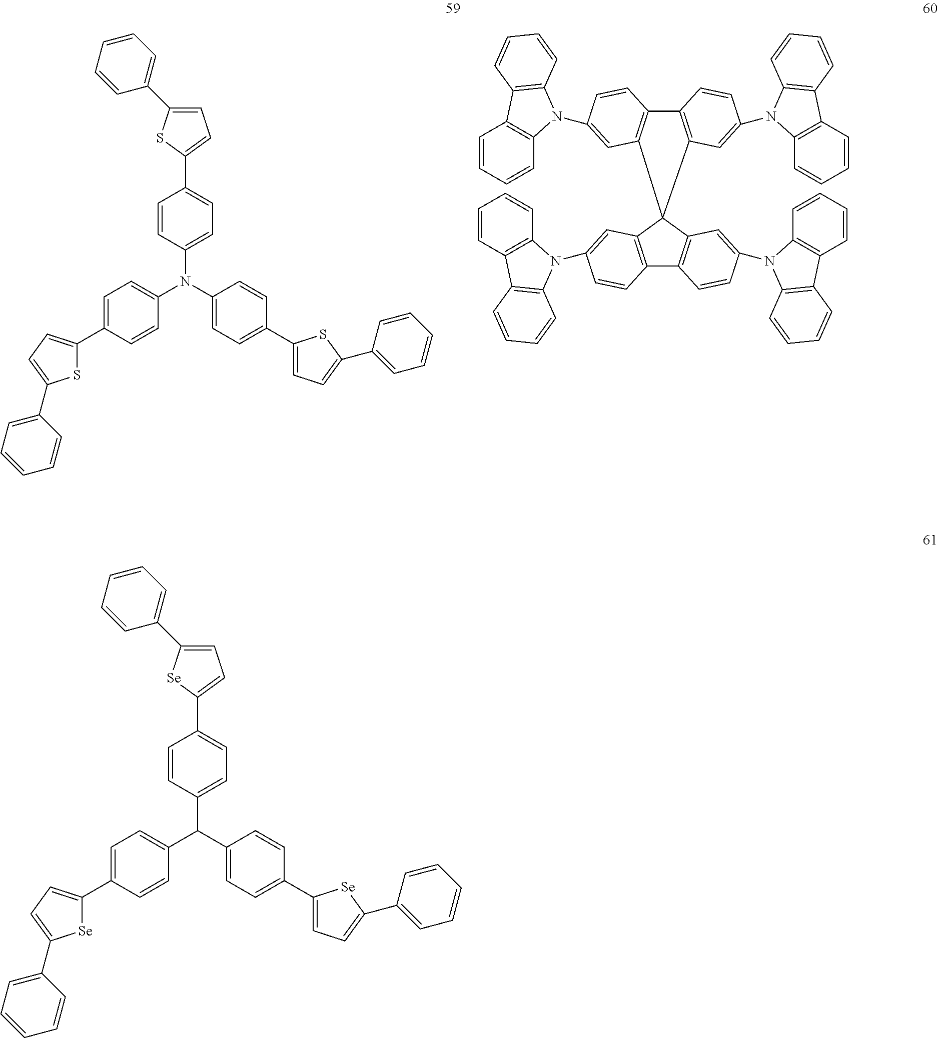 [SMILES: c1ccc(-c2ccc(-c3ccc(N(c4ccc(-c5ccc(-c6ccccc6)s5)cc4)c4ccc(-c5ccc(-c6ccccc6)s5)cc4)cc3)s2)cc1 ; c1ccc2c(c1)c1ccccc1n2-c1ccc2c(c1)C1(c3cc(-n4c5ccccc5c5ccccc54)ccc3-2)c2cc(-n3c4ccccc4c4ccccc43)ccc2-c2ccc(-n3c4ccccc4c4ccccc43)cc21 ; c1ccc(-c2ccc(-c3ccc(C(c4ccc(-c5ccc(-c6ccccc6)[se]5)cc4)c4ccc(-c5ccc(-c6ccccc6)[se]5)cc4)cc3)[se]2)cc1]

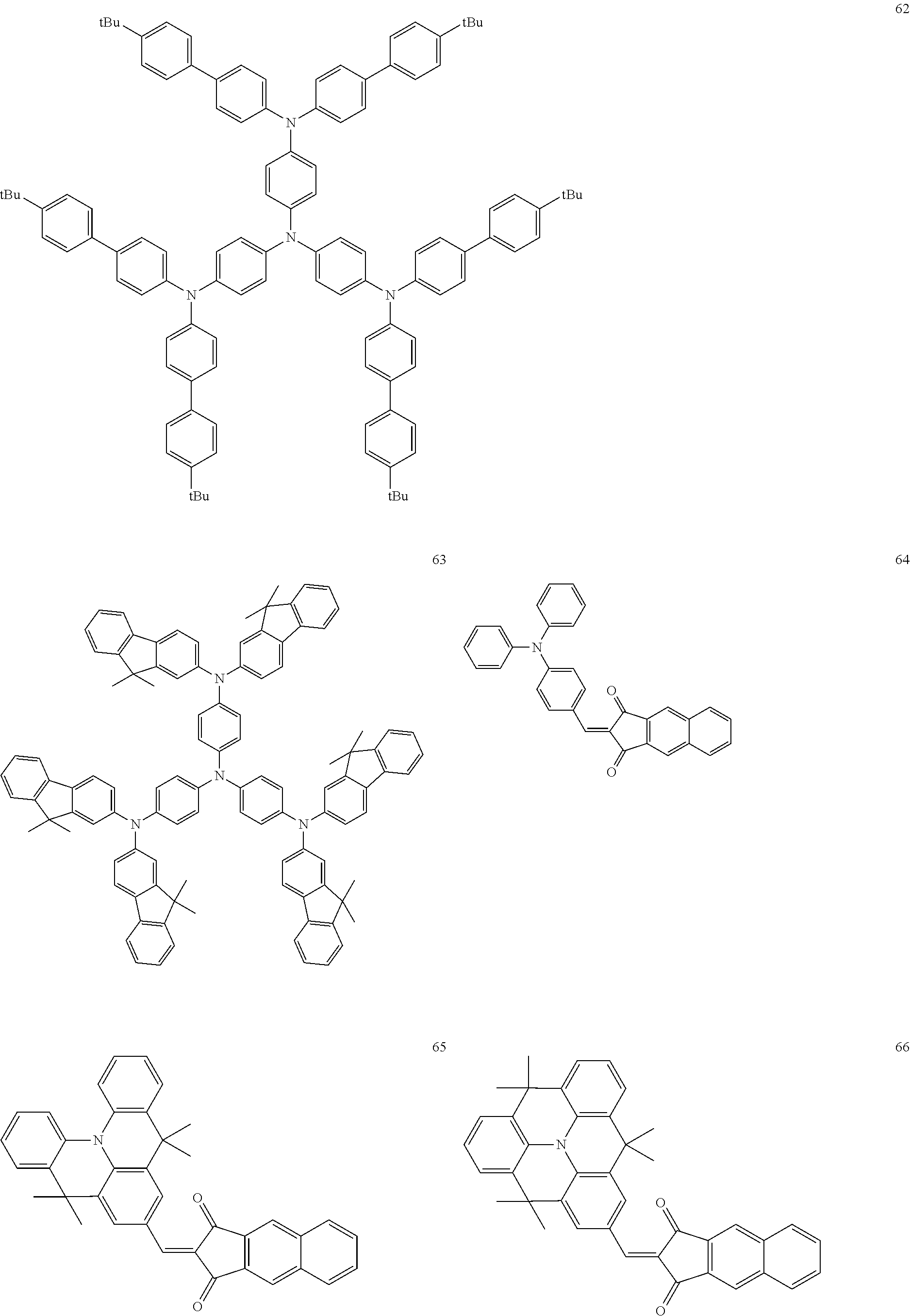

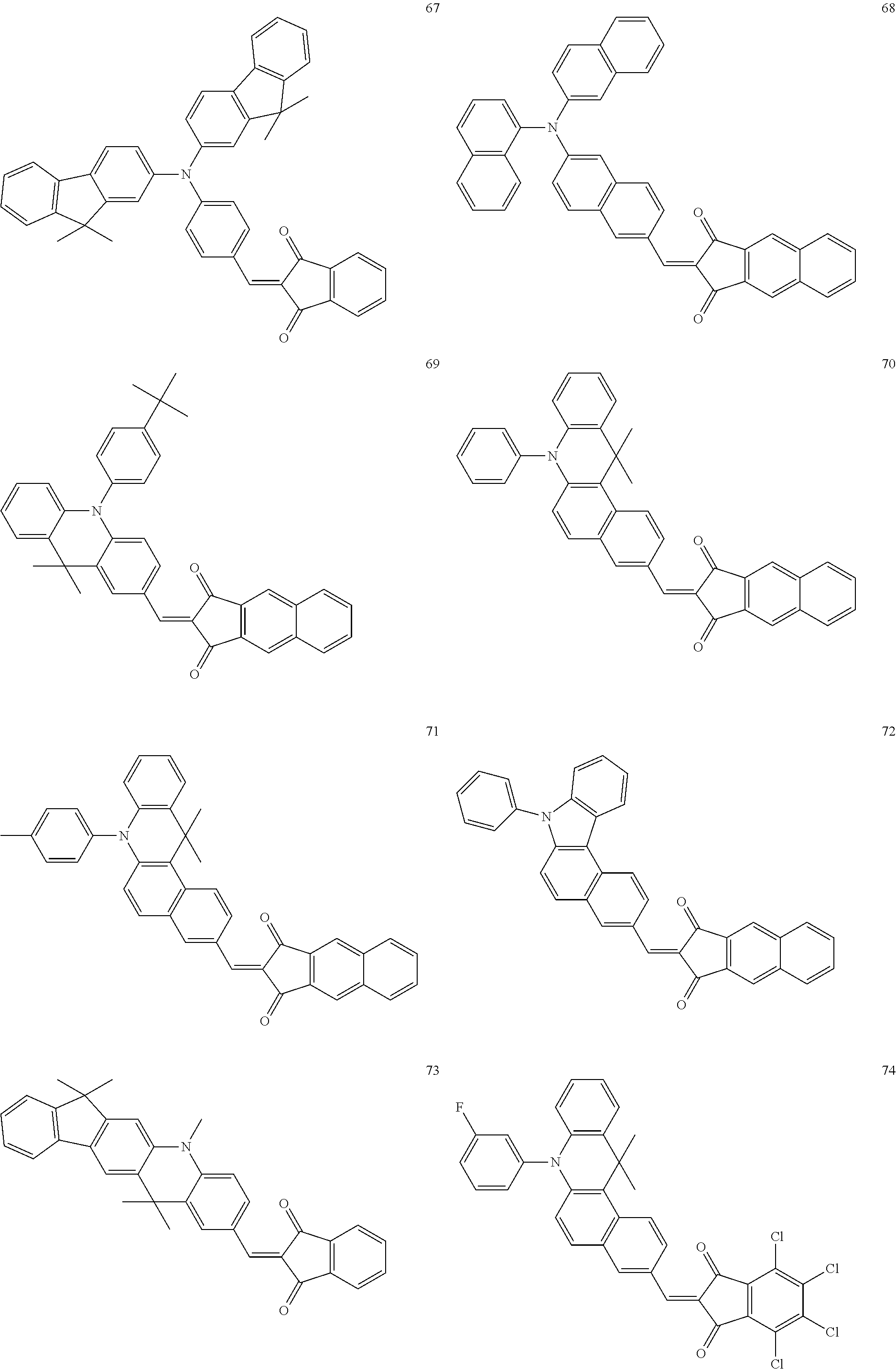

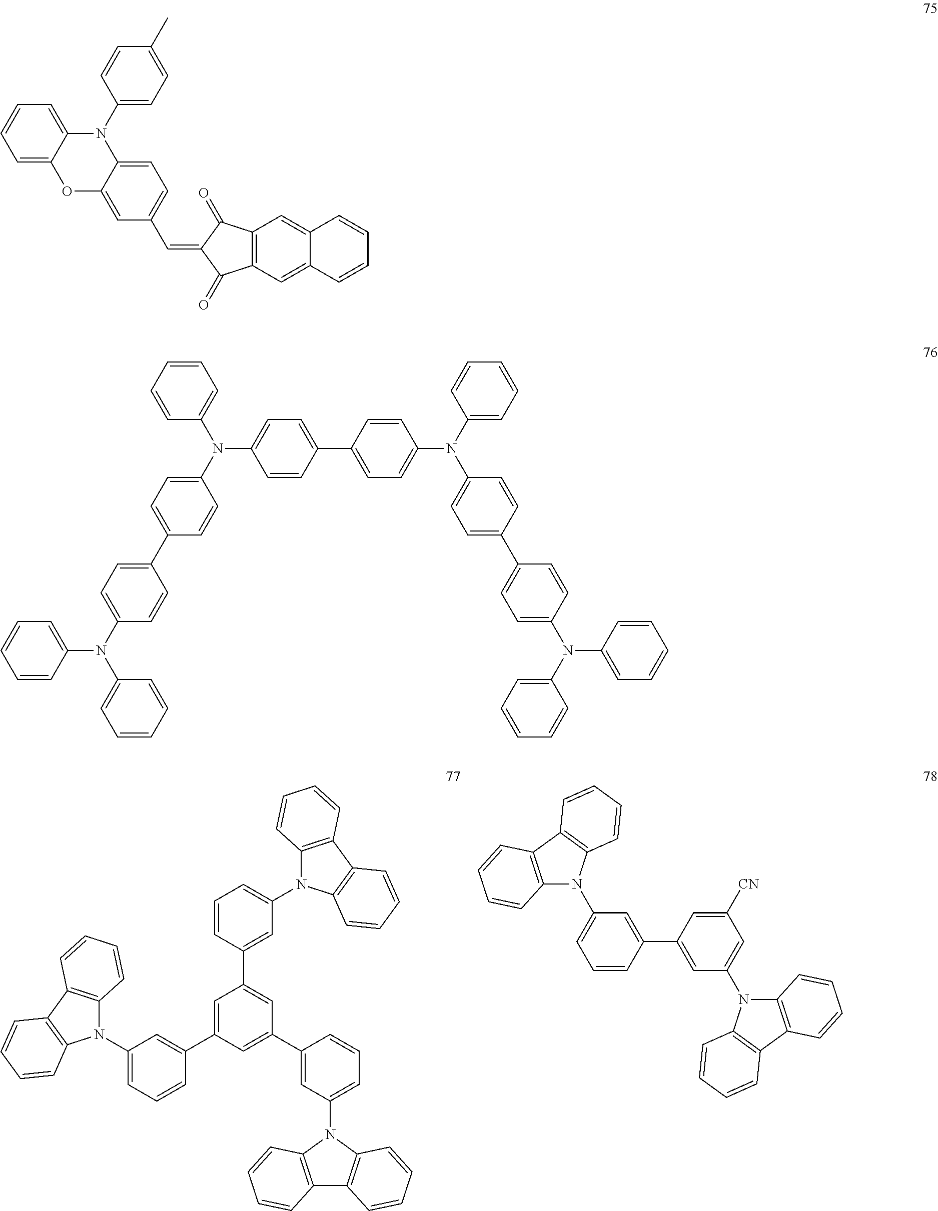 [SMILES: Cc1ccc(N2c3ccccc3Oc3cc(C=C4C(=O)c5cc6ccccc6cc5C4=O)ccc32)cc1 ; c1ccc(N(c2ccccc2)c2ccc(-c3ccc(N(c4ccccc4)c4ccc(-c5ccc(N(c6ccccc6)c6ccc(-c7ccc(N(c8ccccc8)c8ccccc8)cc7)cc6)cc5)cc4)cc3)cc2)cc1 ; c1cc(-c2cc(-c3cccc(-n4c5ccccc5c5ccccc54)c3)cc(-c3cccc(-n4c5ccccc5c5ccccc54)c3)c2)cc(-n2c3ccccc3c3ccccc32)c1 ; N#Cc1cc(-c2cccc(-n3c4ccccc4c4ccccc43)c2)cc(-n2c3ccccc3c3ccccc32)c1]

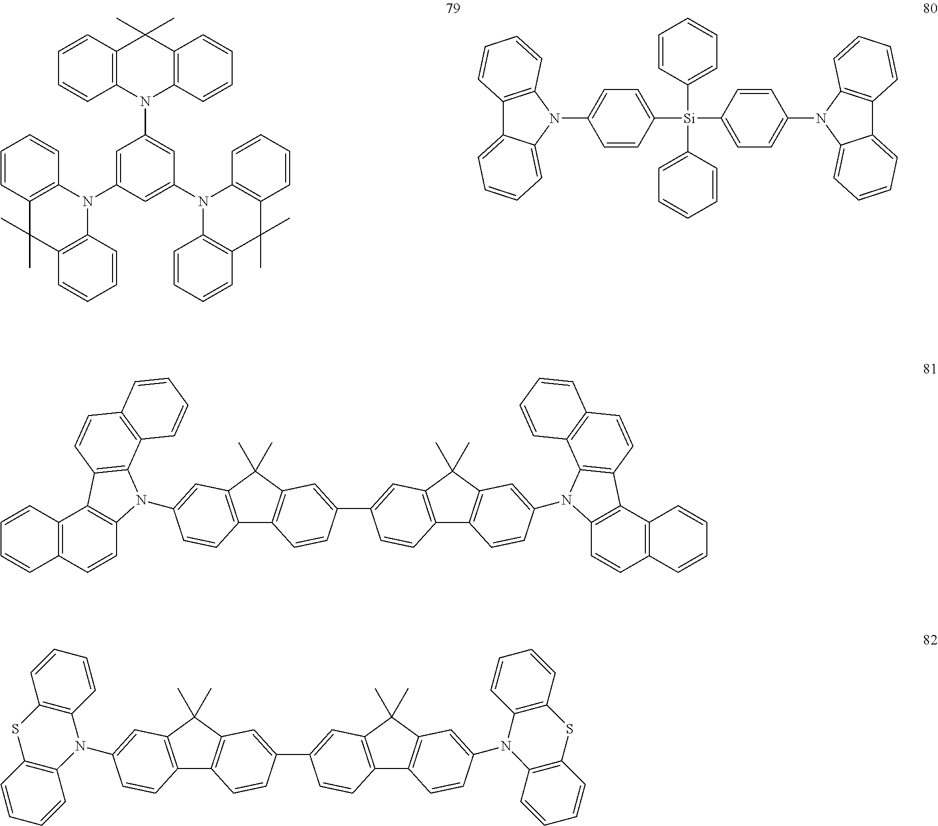 [SMILES: CC1(C)c2ccccc2N(c2cc(N3c4ccccc4C(C)(C)c4ccccc43)cc(N3c4ccccc4C(C)(C)c4ccccc43)c2)c2ccccc21 ; c1ccc([Si](c2ccccc2)(c2ccc(-n3c4ccccc4c4ccccc43)cc2)c2ccc(-n3c4ccccc4c4ccccc43)cc2)cc1 ; CC1(C)c2cc(-c3ccc4c(c3)C(C)(C)c3cc(-n5c6ccc7ccccc7c6c6ccc7ccccc7c65)ccc3-4)ccc2-c2ccc(-n3c4ccc5ccccc5c4c4ccc5ccccc5c43)cc21 ; CC1(C)c2cc(-c3ccc4c(c3)C(C)(C)c3cc(N5c6ccccc6Sc6ccccc65)ccc3-4)ccc2-c2ccc(N3c4ccccc4Sc4ccccc43)cc21]

Hereinafter, in particular, specific examples ((B-1) to (B-136)) of the structure represented by Formula (A-1), (2) and specific examples of the compound represented by Formula (F-1) or (F-2) according to the present invention will be described, but the present invention is not limited to the following specific examples. In the following Formulae (a) to (t), for the case where “A₁₁ and A₁₂”, “R₂₀ and R′₂₀”, “R₂₃ and R₂₄, and R′₂₃ and R′₂₄”, and the like are not the same as each other, a combination other than the exemplified structure is also possible.

Further, in examples of the following compound, Me: a methyl group, Et: an ethyl group, i-Pr: an isopropyl group, n-Bu: an n-butyl group, t-Bu: a tert-butyl group, Ph: a phenyl group, 2-tol: a 2-toluoyl group, 3-tol: a 3-toluoyl group, 4-tol: a 4-toluoyl group, 1-Np: a 1-naphthyl group, 2-Np: a 2-naphthyl group, 2-An: a 2-anthryl group, and 2-Fn: a 2-fluorenyl group.

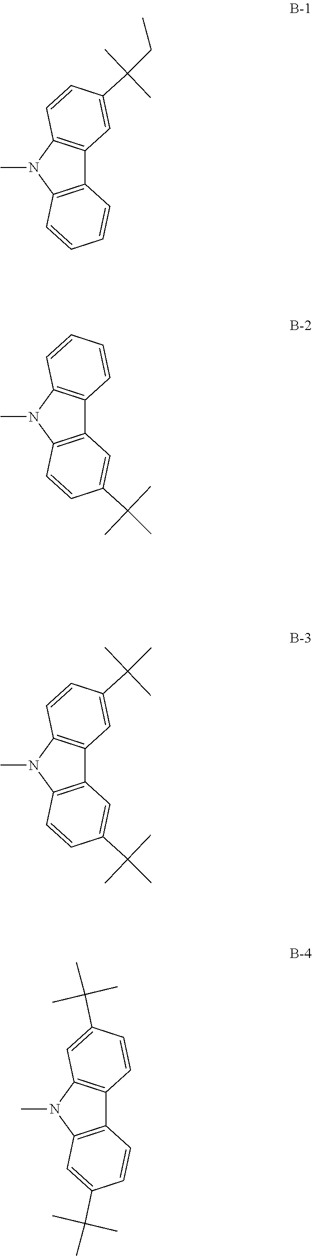

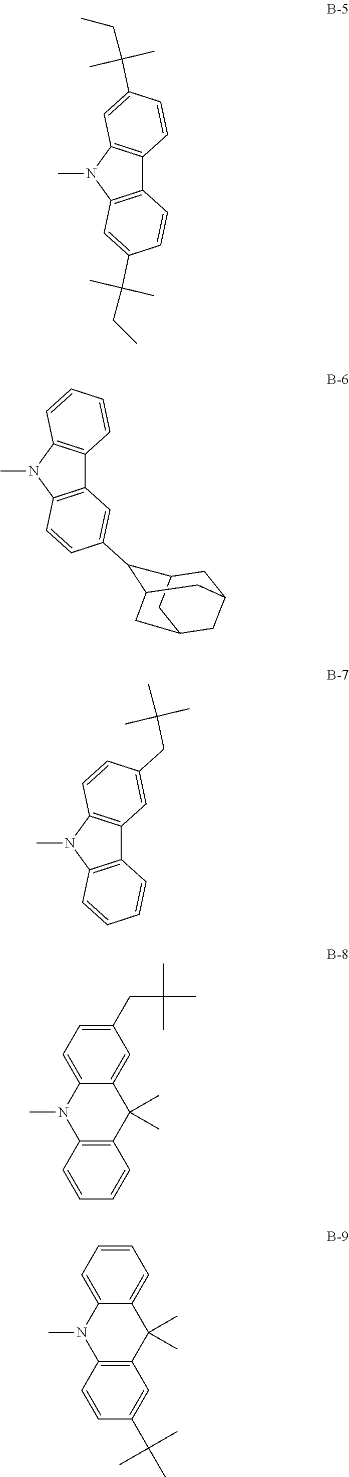

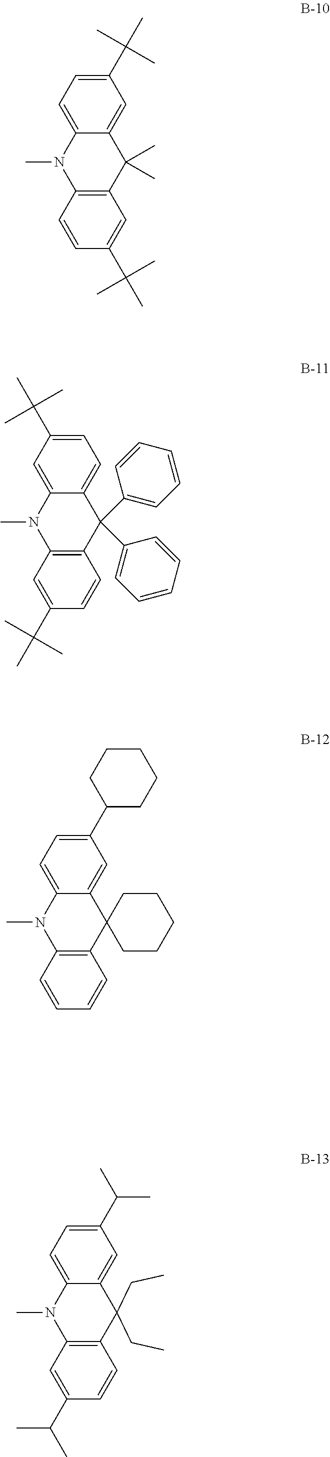

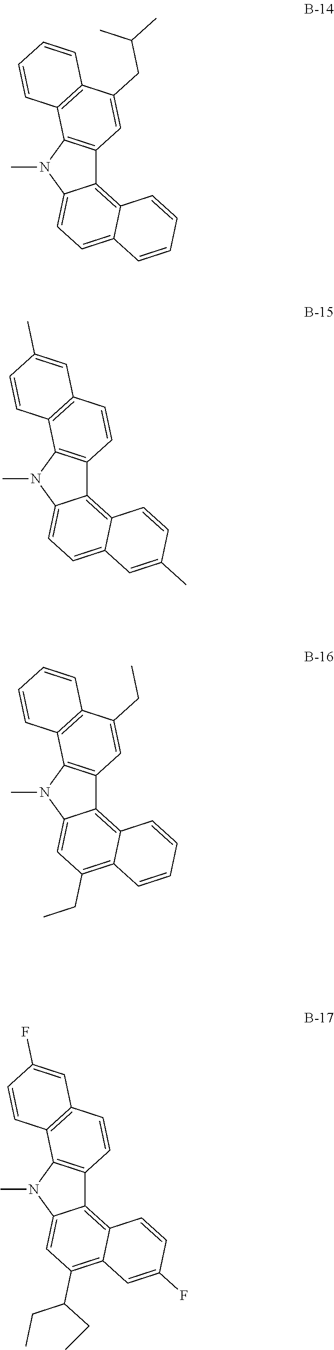

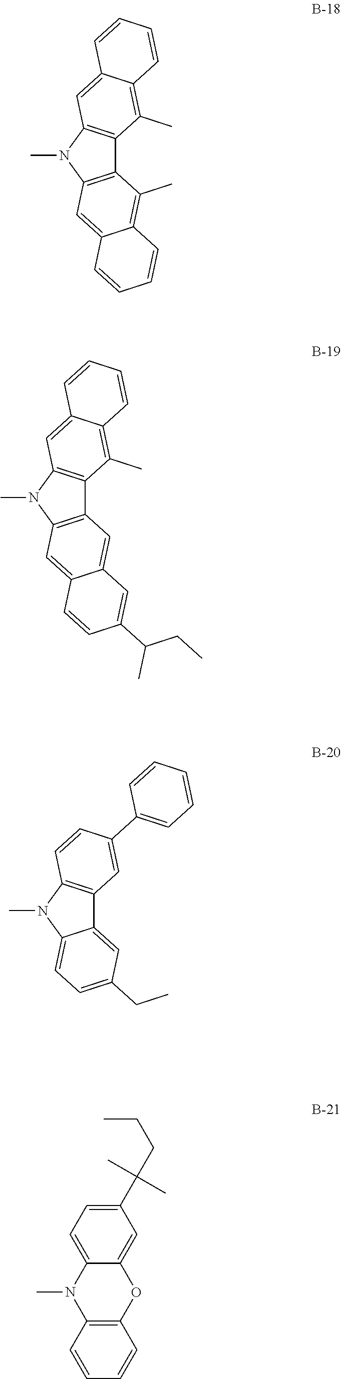

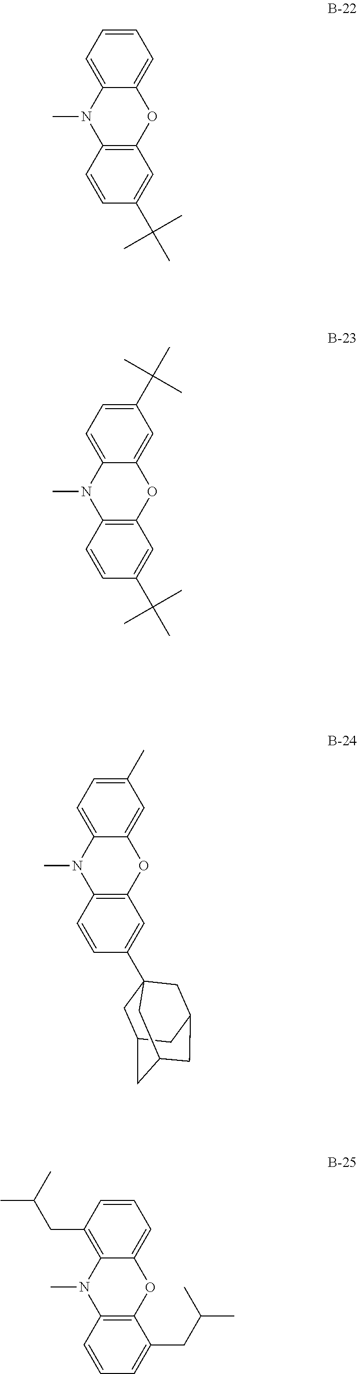

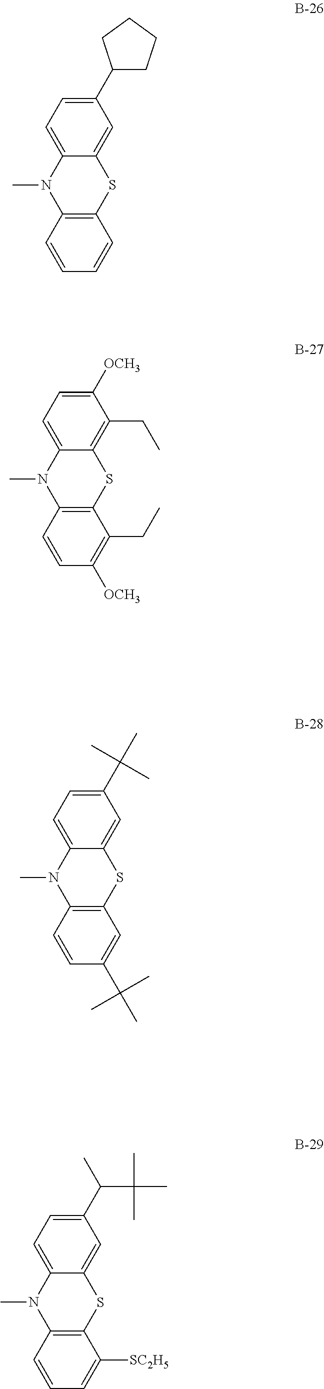

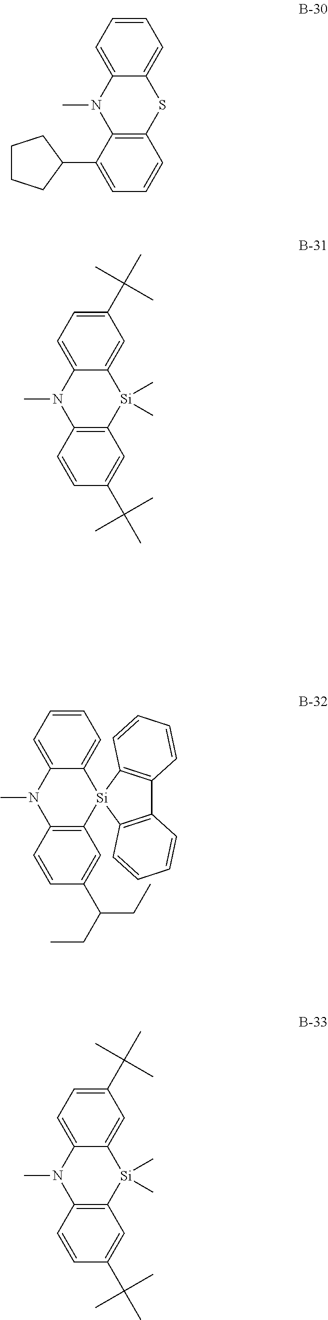

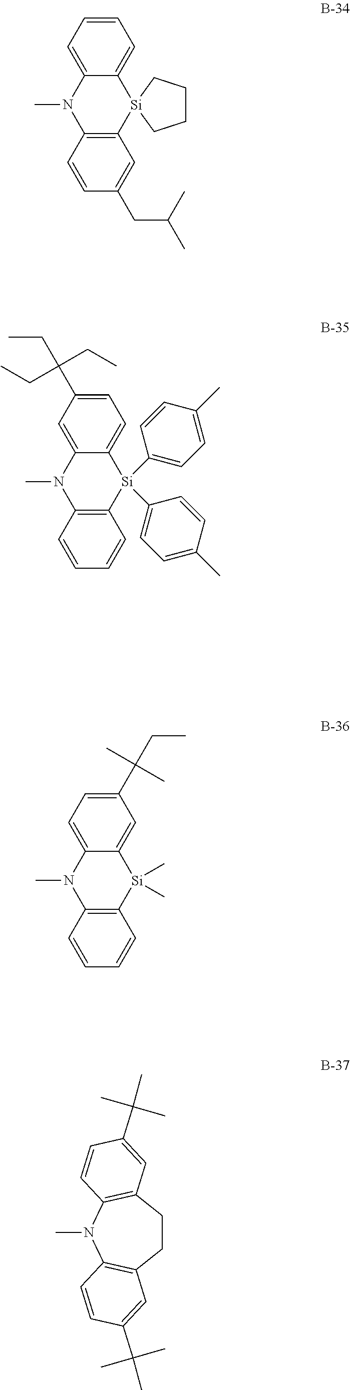

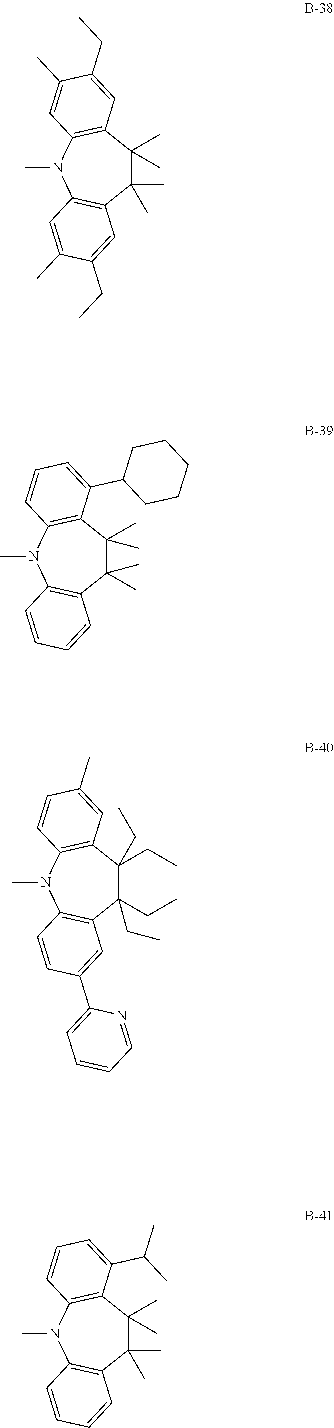

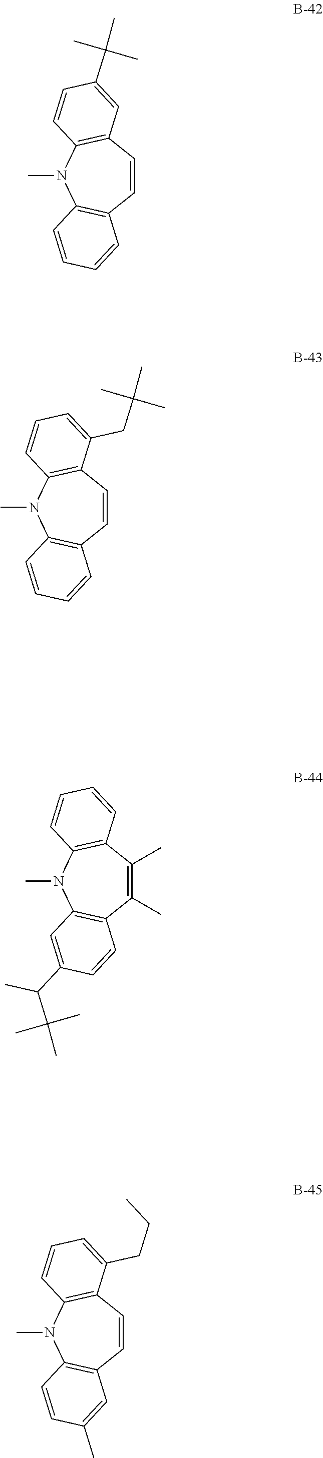

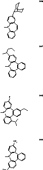

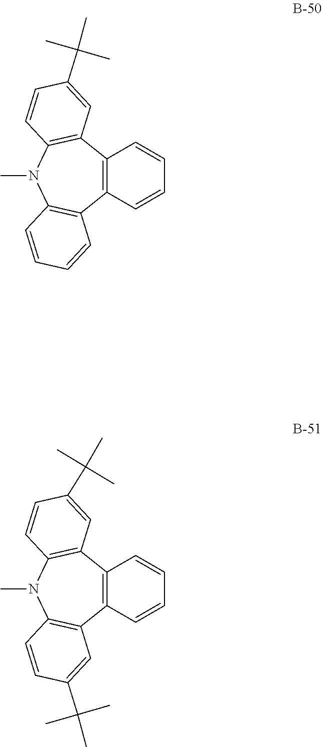 [SMILES: CN1c2ccccc2-c2ccccc2-c2cc(C(C)(C)C)ccc21 ; CN1c2ccc(C(C)(C)C)cc2-c2ccccc2-c2cc(C(C)(C)C)ccc21]

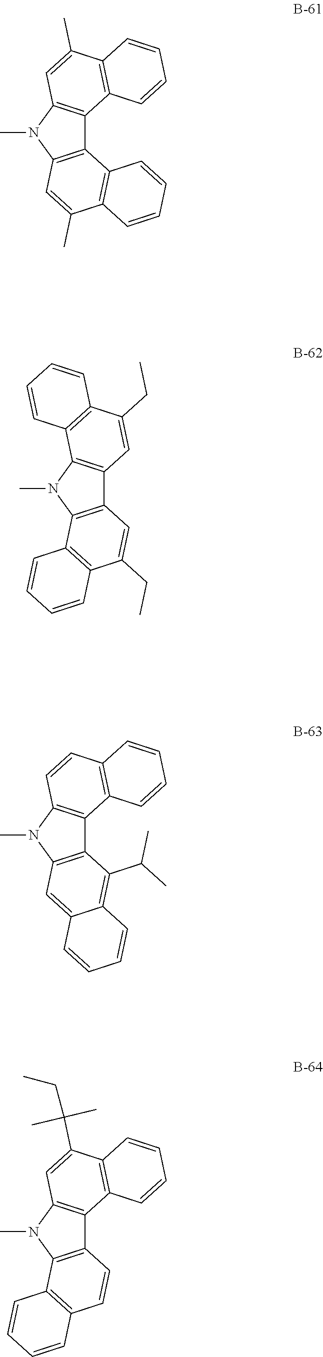

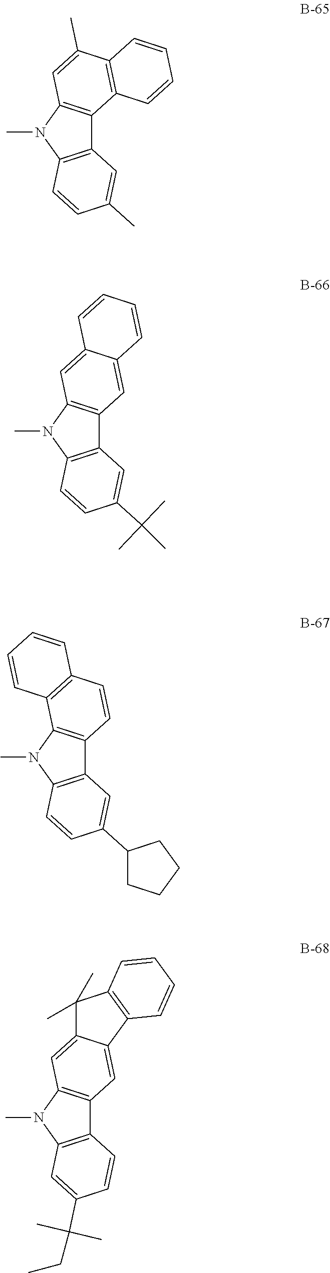

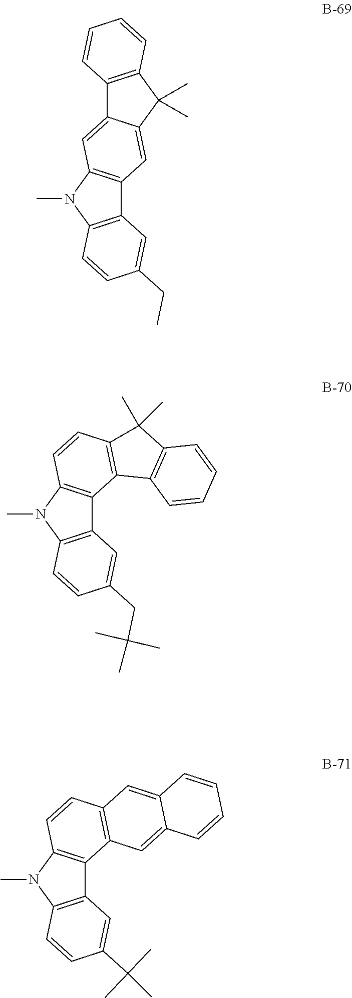

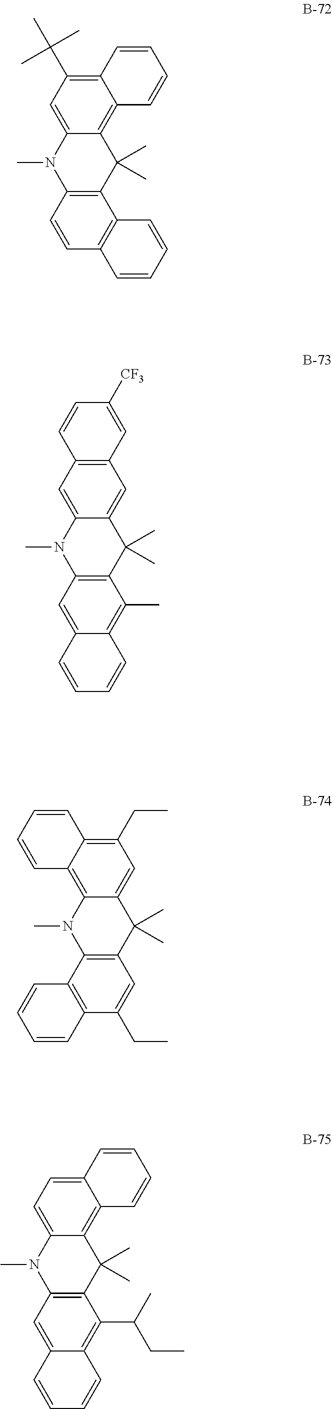

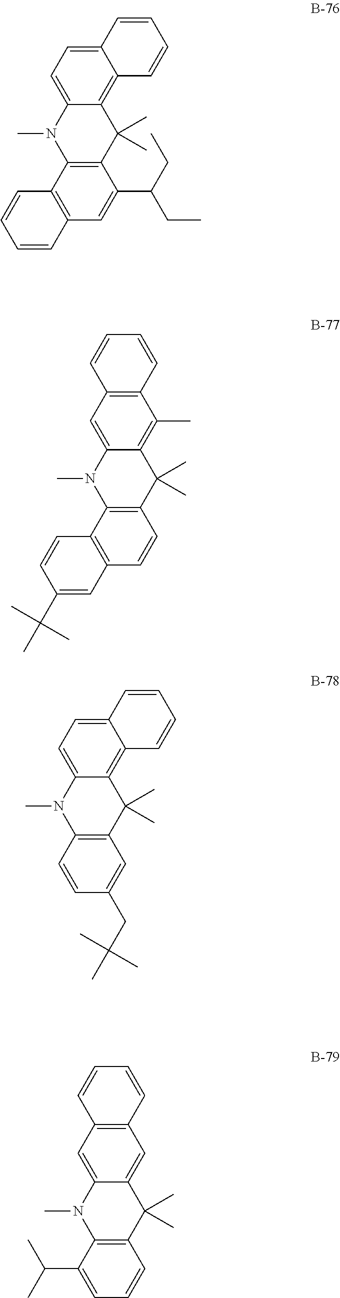

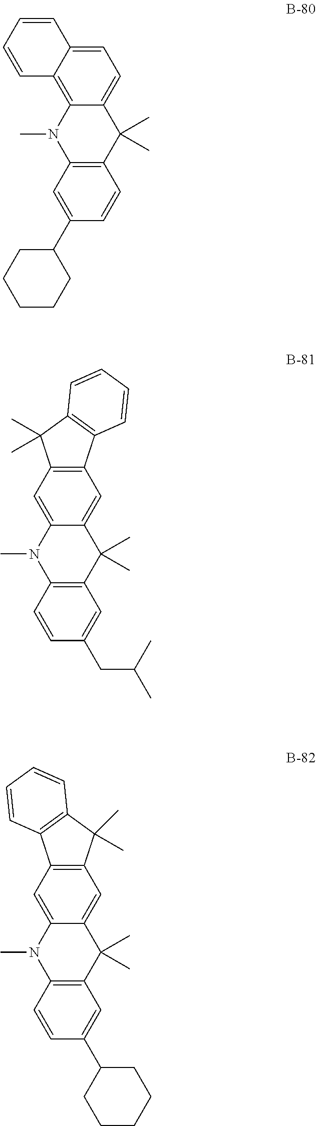

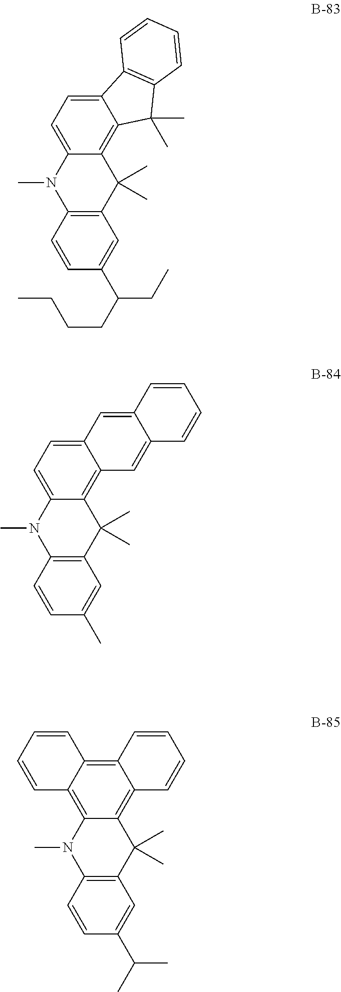

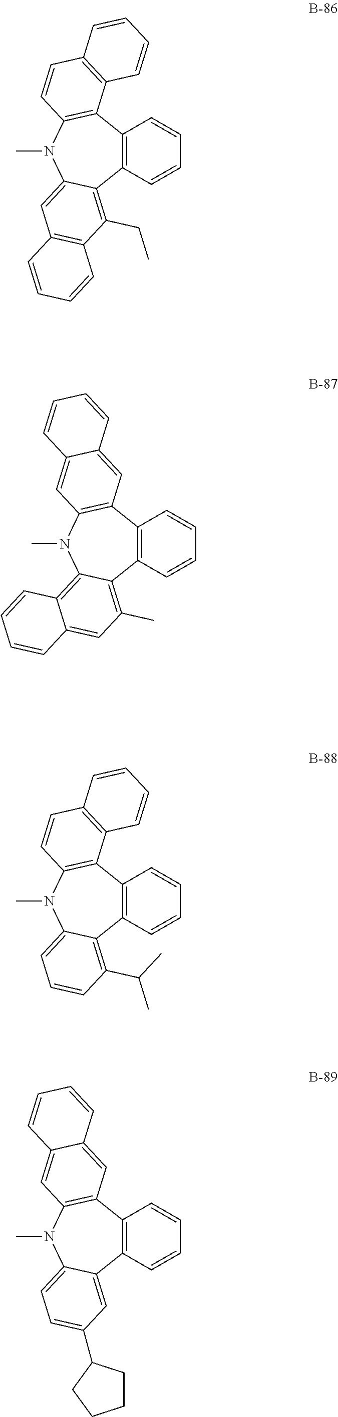 [SMILES: CCc1c2c(cc3ccccc13)N(C)c1ccc3ccccc3c1-c1ccccc1-2 ; Cc1cc2ccccc2c2c1-c1ccccc1-c1cc3ccccc3cc1N2C ; CC(C)c1cccc2c1-c1ccccc1-c1c(ccc3ccccc13)N2C ; CN1c2ccc(C3CCCC3)cc2-c2ccccc2-c2cc3ccccc3cc21]

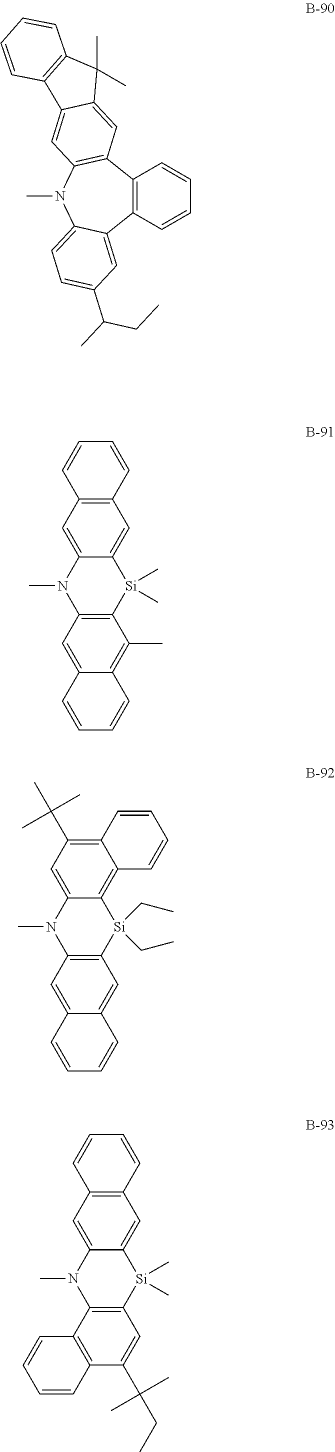

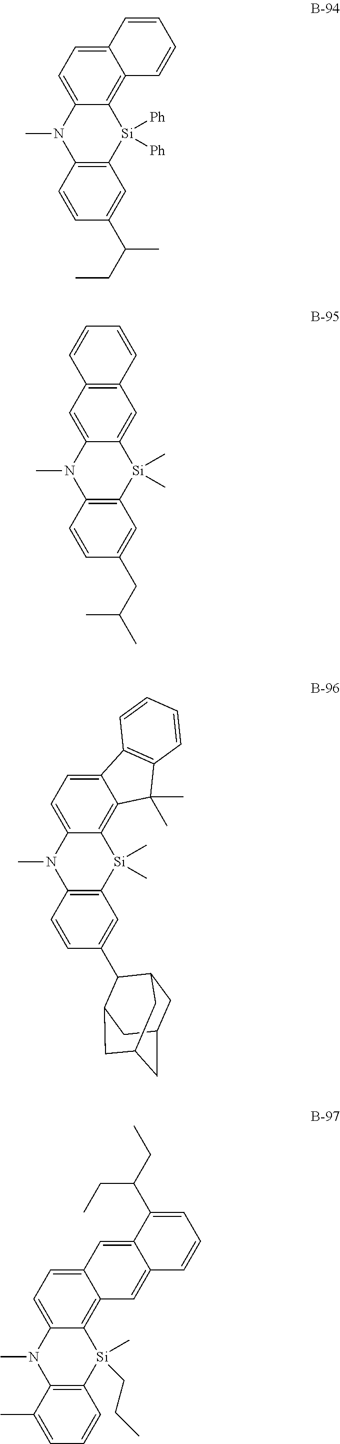

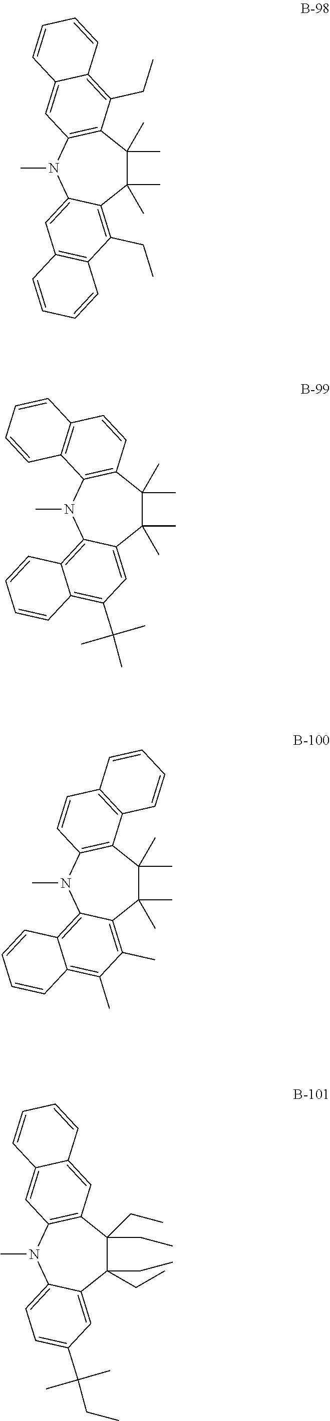

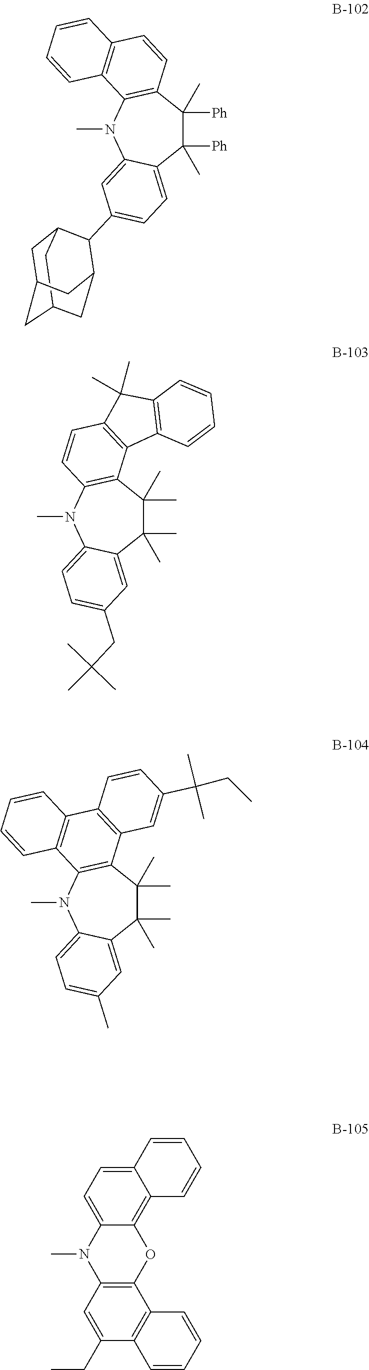

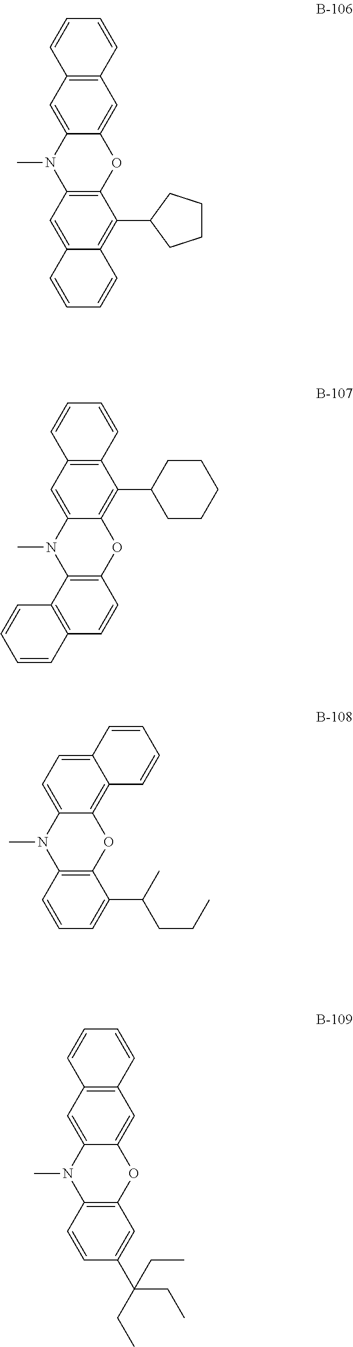

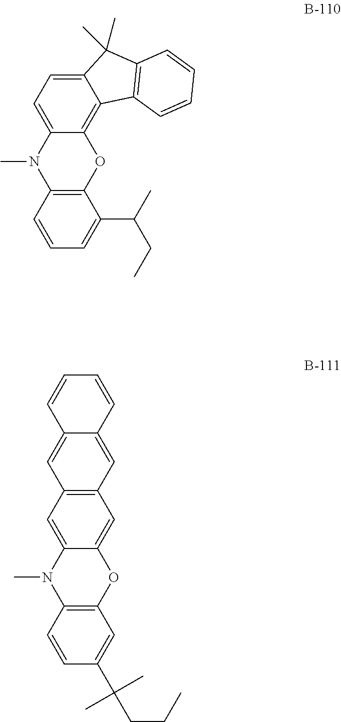 [SMILES: CCC(C)c1cccc2c1Oc1c(ccc3c1-c1ccccc1C3(C)C)N2C ; CCCC(C)(C)c1ccc2c(c1)Oc1cc3cc4ccccc4cc3cc1N2C]

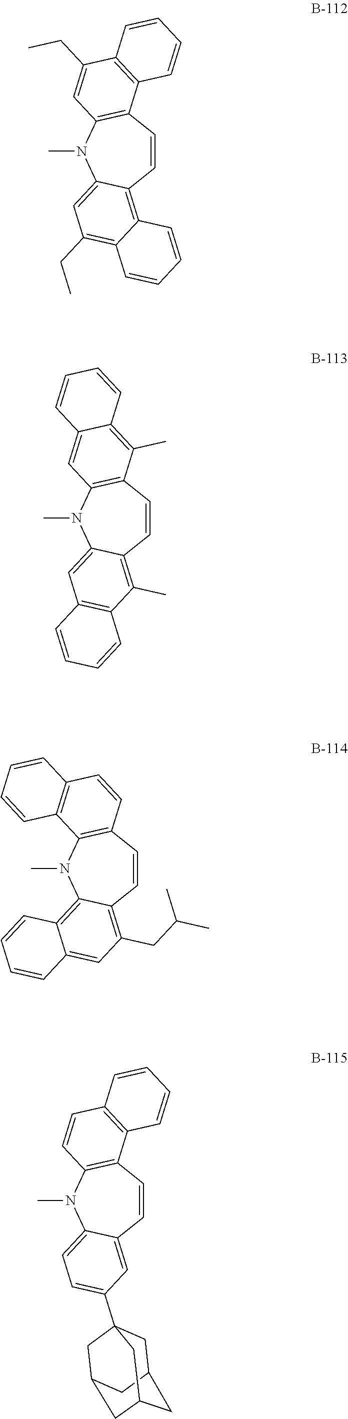

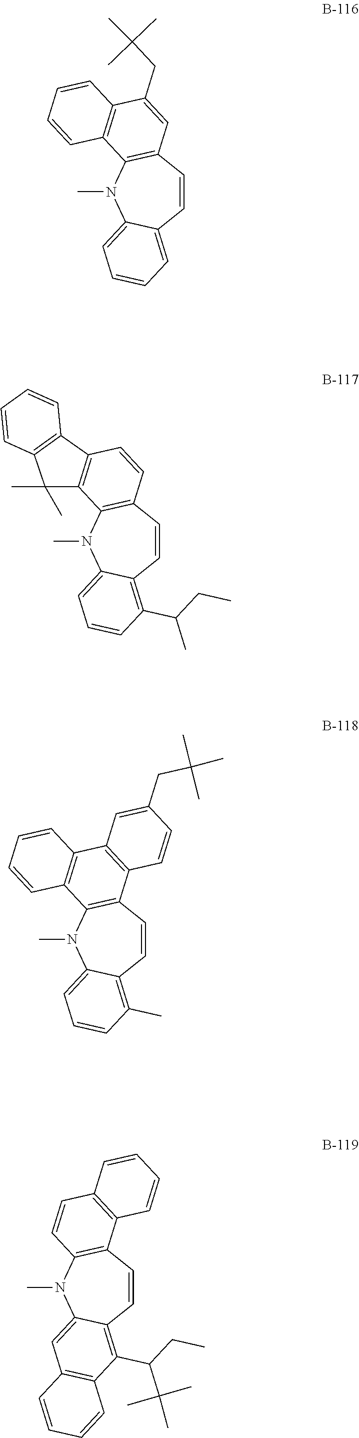

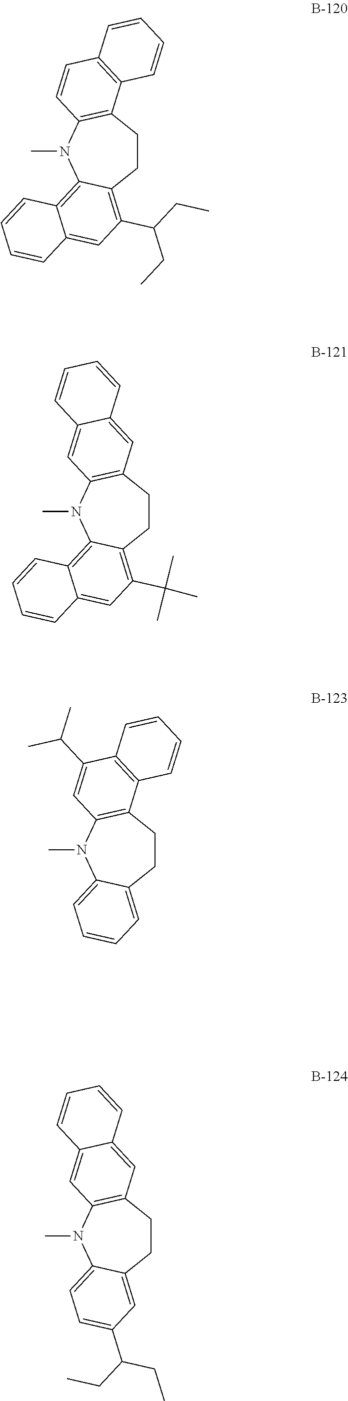

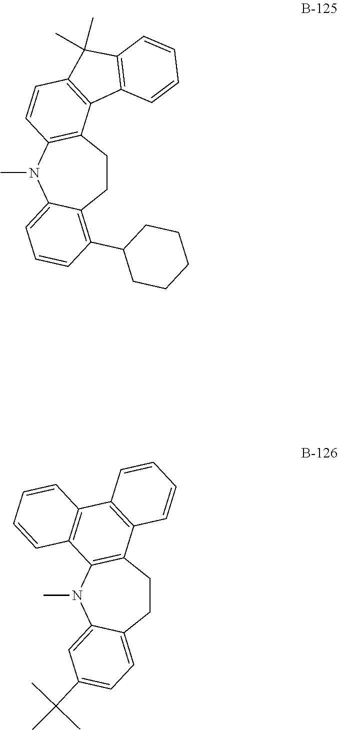 [SMILES: CN1c2cccc(C3CCCCC3)c2CCc2c1ccc1c2-c2ccccc2C1(C)C ; CN1c2cc(C(C)(C)C)ccc2CCc2c1c1ccccc1c1ccccc21]

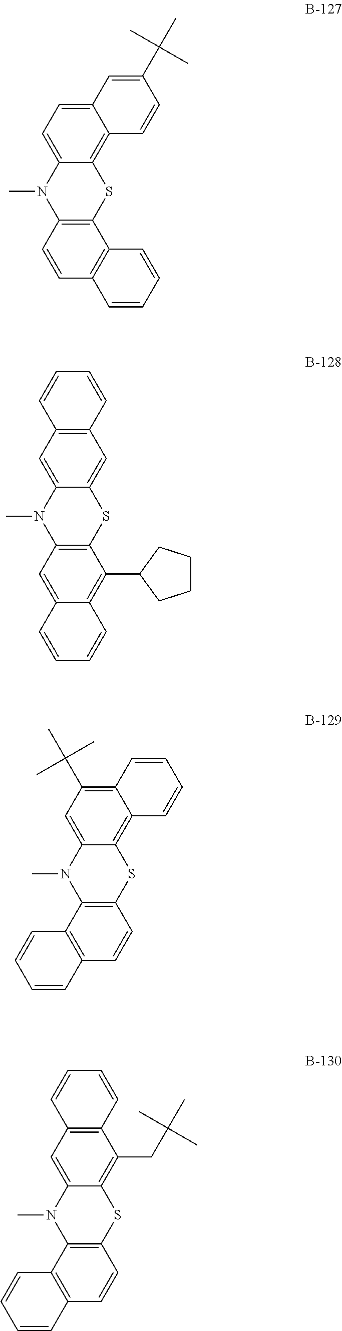

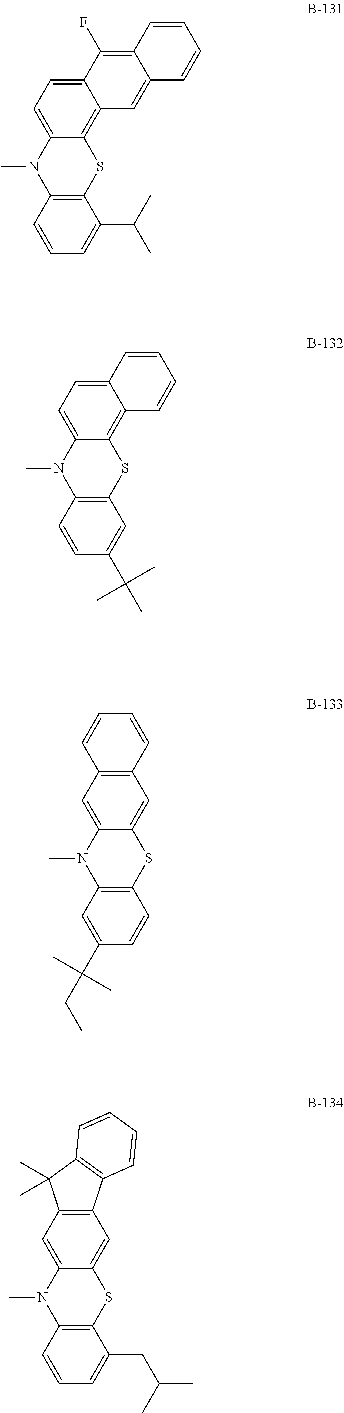 [SMILES: CC(C)c1cccc2c1Sc1c(ccc3c(F)c4ccccc4cc13)N2C ; CN1c2ccc(C(C)(C)C)cc2Sc2c1ccc1ccccc21 ; CCC(C)(C)c1ccc2c(c1)N(C)c1cc3ccccc3cc1S2 ; CC(C)Cc1cccc2c1Sc1cc3c(cc1N2C)C(C)(C)c1ccccc1-3]

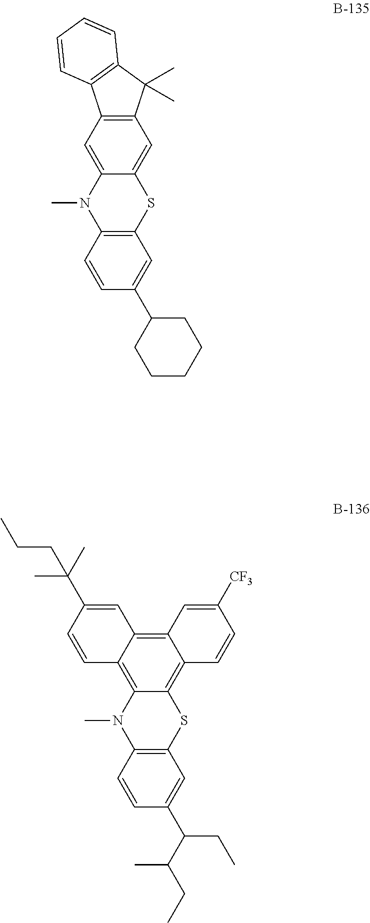 [SMILES: CN1c2ccc(C3CCCCC3)cc2Sc2cc3c(cc21)-c1ccccc1C3(C)C ; CCCC(C)(C)c1ccc2c3c(c4ccc(C(F)(F)F)cc4c2c1)Sc1cc(C(CC)C(C)CC)ccc1N3C]

| | |
|---|---|
| | (a) |
| 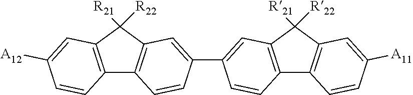 | |

| Com- | | | | | | |
|---|---|---|---|---|---|---|
| pound | | | | | | |
| No. | R₂₁ | R₂₂ | R′₂₁ | R′₂₂ | A₁₁ | A₁₂ |
| a-1 | Me | Me | Me | Me | B-1 | B-1 |
| a-2 | Me | Me | Me | Me | B-2 | B-2 |
| a-3 | Me | Me | Me | Me | B-3 | B-3 |
| a-4 | Me | Me | Me | Me | B-8 | B-8 |
| a-5 | Me | Me | Me | Me | B-9 | B-9 |
| a-6 | Me | Me | Me | Me | B-10 | B-10 |
| a-7 | Me | Me | Me | Me | B-14 | B-14 |
| a-8 | Me | Me | Me | Me | B-21 | B-21 |
| a-9 | Me | Me | Me | Me | B-23 | B-23 |
| a-10 | Me | Me | Me | Me | B-31 | B-33 |
| a-11 | Me | Me | Me | Me | B-42 | B-42 |
| a-12 | H | H | H | H | B-43 | B-43 |
| a-13 | H | H | H | Me | B-47 | B-47 |
| a-14 | Et | Et | Et | Et | B-48 | B-48 |
| a-15 | n-Bu | n-Bu | n-Bu | n-Bu | B-31 | B-33 |
| a-16 | Ph | Ph | Ph | Ph | B-4 | B-4 |
| a-17 | Me | Me | Me | Ph | B-5 | B-5 |
| a-18 | i-Pr | i-Pr | i-Pr | i-Pr | B-17 | B-17 |
| a-19 | 2-MeOEt | 2-MeOEt | 2-MeOEt | 2-MeOEt | B-1 | B-2 |
| a-20 | Me | Me | Me | Me | B-26 | B-26 |
| a-21 | Et | Et | Ph | Ph | B-8 | B-9 |
| a-22 | Me | Me | Me | Me | B-8 | B-10 |
| a-23 | Me | Me | Me | Me | B-1 | B-8 |
| a-24 | Me | Me | Me | Me | B-30 | B-10 |
| a-25 | Me | Et | Me | Ph | B-1 | B-20 |
| a-26 | Me | Me | Me | Me | B-61 | B-61 |
| a-27 | Me | Me | Me | Me | B-64 | B-64 |
| a-28 | Me | Me | Me | Me | B-66 | B-66 |
| a-29 | Me | Me | Me | Me | B-69 | B-69 |
| a-30 | Me | Me | Me | Me | B-71 | B-71 |
| a-31 | Me | Me | Me | Me | B-72 | B-72 |
| a-32 | Me | Me | Me | Me | B-74 | B-74 |
| a-33 | Me | Me | Me | Me | B-76 | B-76 |
| a-34 | Me | Me | Me | Me | B-78 | B-78 |
| a-35 | Me | Me | Me | Me | B-81 | B-81 |
| a-36 | Me | Me | Me | Me | B-84 | B-84 |
| a-37 | H | H | H | H | B-86 | B-86 |
| a-38 | H | H | H | Me | B-89 | B-89 |
| a-39 | Et | Et | Et | Et | B-93 | B-93 |
| a-40 | n-Bu | n-Bu | n-Bu | n-Bu | B-98 | B-101 |
| a-41 | Ph | Ph | Ph | Ph | B-102 | B-105 |
| a-42 | Me | Me | Me | Ph | B-106 | B-106 |
| a-43 | i-Pr | i-Pr | i-Pr | i-Pr | B-107 | B-110 |
| a-44 | 2-MeOEt | 2-MeOEt | 2-MeOEt | 2-MeOEt | B-112 | B-115 |
| a-45 | Me | Me | Me | Me | B-116 | B-119 |
| a-46 | Et | Et | Ph | Ph | B-120 | B-124 |
| a-47 | Me | Me | Me | Me | B-127 | B-131 |
| a-48 | Me | Me | Me | Me | B-132 | B-136 |
| a-49 | Me | Me | Me | Me | B-128 | B-128 |
| a-50 | Me | Et | Me | Ph | B-1 | B-61 |

(b)

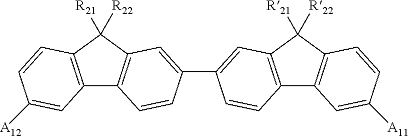

| Compound | | | | | | |
|---|---|---|---|---|---|---|
| No. | R₂₁ | R₂₂ | R′₂₁ | R′₂₂ | A₁₁ | A₁₂ |
| b-1 | Me | Me | Me | Me | B-1 | B-1 |
| b-2 | Me | Me | Me | Me | B-2 | B-2 |
| b-3 | Me | Me | Me | Me | B-3 | B-3 |
| b-4 | Me | Me | Me | Me | B-8 | B-8 |
| b-5 | Me | Me | Me | Me | B-9 | B-9 |
| b-6 | H | H | H | H | B-43 | B-43 |
| b-7 | H | H | H | Me | B-47 | B-47 |
| b-8 | Et | Et | Et | Et | B-48 | B-48 |
| b-9 | n-Bu | n-Bu | n-Bu | n-Bu | B-6 | B-6 |
| b-10 | Ph | Ph | Ph | Ph | B-11 | B-11 |
| b-11 | Me | Me | Me | Ph | B-15 | B-15 |
| b-12 | i-Pr | i-Pr | i-Pr | i-Pr | B-17 | B-17 |
| b-13 | 2-MeOEt | 2-MeOEt | 2-MeOEt | 2-MeOEt | B-23 | B-23 |
| b-14 | Et | Et | Ph | Ph | B-26 | B-26 |
| b-15 | Me | Et | Me | Ph | B-32 | B-32 |
| b-16 | Me | Me | Me | Me | B-62 | B-62 |
| b-17 | Me | Me | Me | Me | B-65 | B-65 |
| b-18 | Me | Me | Me | Me | B-73 | B-73 |
| b-19 | Me | Me | Me | Me | B-77 | B-77 |
| b-20 | Me | Me | Me | Me | B-86 | B-86 |
| b-21 | H | H | H | H | B-83 | B-83 |
| b-22 | H | H | H | Me | B-90 | B-90 |
| b-23 | Et | Et | Et | Et | B-103 | B-103 |
| b-24 | n-Bu | n-Bu | n-Bu | n-Bu | B-113 | B-113 |
| b-25 | Ph | Ph | Ph | Ph | B-118 | B-118 |
| b-26 | Me | Me | Me | Ph | B-126 | B-126 |
| b-27 | i-Pr | i-Pr | i-Pr | i-Pr | B-130 | B-130 |
| b-28 | 2-MeOEt | 2-MeOEt | 2-MeOEt | 2-MeOEt | B-133 | B-133 |
| b-29 | Et | Et | Ph | Ph | B-92 | B-92 |
| b-30 | Me | Et | Me | Ph | B-95 | B-95 |

(c)

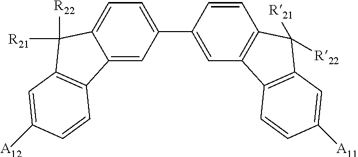

| Compound | | | | | | |
|---|---|---|---|---|---|---|
| No. | R₂₁ | R₂₂ | R′₂₁ | R′₂₂ | A₁₁ | A₁₂ |
| c-1 | Me | Me | Me | Me | B-1 | B-1 |
| c-2 | Me | Me | Me | Me | B-2 | B-2 |
| c-3 | Me | Me | Me | Me | B-3 | B-3 |
| c-4 | Me | Me | Me | Me | B-8 | B-8 |
| c-5 | Me | Me | Me | Me | B-9 | B-9 |
| c-6 | H | H | H | H | B-10 | B-10 |
| c-7 | H | H | H | Me | B-51 | B-51 |
| c-8 | Et | Et | Et | Et | B-46 | 8-44 |
| c-9 | n-Bu | n-Bu | n-Bu | n-Bu | B-37 | B-37 |
| c-10 | Ph | Ph | Ph | Ph | B-38 | B-38 |
| c-11 | Me | Me | Me | Ph | B-33 | B-35 |
| c-12 | i-Pr | i-Pr | i-Pr | i-Pr | B-27 | B-27 |
| c-13 | 2-MeOEt | 2-MeOEt | 2-MeOEt | 2-MeOEt | B-24 | B-24 |
| c-14 | Et | Et | Ph | Ph | B-7 | B-7 |
| c-15 | Me | Et | Me | Ph | B-7 | B-24 |
| c-16 | Me | Me | Me | Me | B-63 | B-63 |
| c-17 | Me | Me | Me | Me | B-67 | B-67 |
| c-18 | Me | Me | Me | Me | B-75 | B-75 |
| c-19 | Me | Me | Me | Me | B-78 | B-78 |
| c-20 | Me | Me | Me | Me | B-87 | B-87 |
| c-21 | H | H | H | H | B-91 | B-91 |
| c-22 | H | H | H | Me | B-99 | B-99 |
| c-23 | Et | Et | Et | Et | B-108 | B-108 |
| c-24 | n-Bu | n-Bu | n-Bu | n-Bu | B-111 | B-111 |
| c-25 | Ph | Ph | Ph | Ph | B-114 | B-114 |
| c-26 | Me | Me | Me | Ph | B-121 | B-121 |
| c-27 | i-Pr | i-Pr | i-Pr | i-Pr | B-125 | 8-125 |
| c-28 | 2-MeOEt | 2-MeOEt | 2-MeOEt | 2-MeOEt | B-129 | B-129 |
| c-29 | Et | Et | Ph | Ph | B-94 | B-94 |
| c-30 | Me | Et | Me | Ph | B-109 | B-109 |

(d)

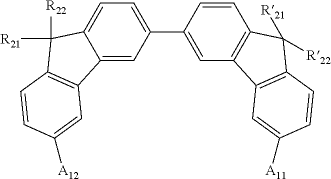

| Compound | | | | | | |
|---|---|---|---|---|---|---|
| No. | R₂₁ | R₂₂ | R′₂₁ | R′₂₂ | A₁₁ | A₁₂ |
| d-1 | Me | Me | Me | Me | B-1 | B-1 |
| d-2 | Me | Me | Me | Me | B-2 | B-2 |
| d-3 | Me | Me | Me | Me | B-3 | B-3 |
| d-4 | Me | Me | Me | Me | B-8 | B-8 |
| d-5 | Me | Me | Me | Me | B-9 | B-9 |
| d-6 | H | H | H | H | B-10 | B-10 |
| d-7 | H | H | H | Me | B-12 | B-12 |
| d-8 | Et | Et | Et | Et | B-18 | B-18 |
| d-9 | n-Bu | n-Bu | n-Bu | n-Bu | B-25 | B-25 |
| d-10 | Ph | Ph | Ph | Ph | B-31 | B-31 |
| d-11 | Me | Me | Me | Ph | B-34 | B-34 |
| d-12 | i-Pr | i-Pr | i-Pr | i-Pr | B-39 | B-39 |
| d-13 | 2-MeOEt | 2-MeOEt | 2-MeOEt | 2-MeOEt | B-49 | B-49 |
| d-14 | Et | Et | Ph | Ph | B-16 | B-22 |
| d-15 | Me | Et | Me | Ph | B-3 | B-10 |
| d-16 | Me | Me | Me | Me | B-61 | B-61 |
| d-17 | Me | Me | Me | Me | B-70 | B-70 |
| d-18 | Me | Me | Me | Me | B-72 | B-72 |
| d-19 | Me | Me | Me | Me | B-79 | B-79 |
| d-20 | Me | Me | Me | Me | B-88 | B-88 |
| d-21 | H | H | H | H | B-96 | B-96 |
| d-22 | H | H | H | Me | B-100 | B-100 |
| d-23 | Et | Et | Et | Et | B-117 | B-117 |
| d-24 | n-Bu | n-Bu | n-Bu | n-Bu | B-125 | B-125 |
| d-25 | Ph | Ph | Ph | Ph | B-131 | B-131 |
| d-26 | Me | Me | Me | Ph | B-134 | B-134 |
| d-27 | i-Pr | i-Pr | i-Pr | i-Pr | B-135 | B-135 |
| d-28 | 2-MeOEt | 2-MeOEt | 2-MeOEt | 2-MeOEt | B-14 | B-74 |
| d-29 | Et | Et | Ph | Ph | B-25 | B-86 |
| d-30 | Me | Et | Me | Ph | B-101 | B-10 |

(e)

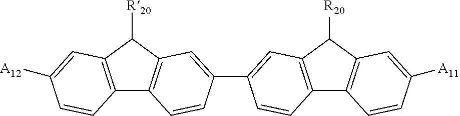

| Compound No. | R₂₀ | R′₂₀ | A₁₁ | A₁₂ |
|---|---|---|---|---|
| e-1 | Ph | Ph | B-1 | B-1 |
| e-2 | Ph | Ph | B-2 | B-2 |
| e-3 | Ph | Ph | B-3 | B-3 |
| e-4 | Ph | Ph | B-8 | B-8 |
| e-5 | Ph | Ph | B-9 | B-9 |
| e-6 | Ph | Ph | B-10 | B-10 |
| e-7 | Ph | Ph | B-14 | B-14 |
| e-8 | Ph | Ph | B-20 | B-20 |
| e-9 | Ph | Ph | B-21 | B-21 |
| e-10 | 2-tol | 2-tol | B-1 | B-1 |
| e-11 | 3-tol | 3-tol | B-2 | B-2 |
| e-12 | 4-tol | 4-tol | B-3 | B-3 |
| e-13 | 2-Np | 2-Np | B-8 | B-8 |
| e-14 | 1-Np | 1-Np | B-9 | B-9 |
| e-15 | 2-An | 2-An | B-10 | B-10 |
| e-16 | 2-Fn | 2-Fn | B-4 | B-4 |
| e-17 | Me | Me | B-28 | B-28 |
| e-18 | i-Pr | i-Pr | B-36 | B-36 |
| e-19 | Et | Et | B-40 | B-40 |
| e-20 | Ph | 2-tol | B-45 | B-50 |
| e-21 | 3-tol | Ph | B-8 | B-9 |
| e-22 | 2-Fn | Ph | B-8 | B-10 |
| e-23 | t-Bu | t-Bu | B-1 | B-1 |
| e-24 | t-Bu | t-Bu | B-3 | B-3 |
| e-25 | 2-Np | Ph | B-1 | B-8 |
| e-26 | Ph | Ph | B-62 | B-62 |
| e-27 | Ph | Ph | B-66 | B-66 |
| e-28 | Ph | Ph | B-73 | B-73 |
| e-29 | Ph | Ph | B-77 | B-77 |
| e-30 | Ph | Ph | B-82 | B-82 |
| e-31 | Ph | Ph | B-84 | B-84 |
| e-32 | Ph | Ph | B-85 | B-85 |
| e-33 | Ph | Ph | B-86 | B-86 |
| e-34 | Ph | Ph | B-90 | B-90 |
| e-35 | 2-tol | 2-tol | B-97 | B-97 |
| e-36 | 3-tol | 3-tol | B-99 | B-99 |
| e-37 | 4-tol | 4-tol | B-104 | B-104 |
| e-38 | 2-Np | 2-Np | B-109 | B-109 |
| e-39 | 1-Np | 1-Np | B-111 | B-111 |
| e-40 | 2-An | 2-An | B-112 | B-112 |
| e-41 | 2-Fn | 2-Fn | B-116 | B-116 |
| e-42 | Me | Me | B-123 | B-123 |
| e-43 | i-Pr | i-Pr | B-126 | B-126 |
| e-44 | Et | Et | B-127 | B-131 |
| e-45 | Ph | 2-tol | B-45 | B-50 |
| e-46 | 3-tol | Ph | B-8 | B-9 |
| e-47 | 2-Fn | Ph | B-8 | B-10 |
| e-48 | t-Bu | t-Bu | B-72 | B-17 |
| e-49 | t-Bu | t-Bu | B-3 | B-68 |
| e-50 | 2-Np | Ph | B-8 | B-68 |

(f)

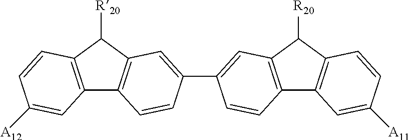

| Compound No. | R₂₀ | R′₂₀ | A₁₁ | A₁₂ |
|---|---|---|---|---|
| f-1 | Ph | Ph | B-1 | B-1 |
| f-2 | Ph | Ph | B-2 | B-2 |
| f-3 | Ph | Ph | B-3 | B-3 |
| f-4 | Ph | Ph | B-8 | B-8 |
| f-5 | 2-tol | 2-tol | B-9 | B-9 |
| f-6 | 3-tol | 3-tol | B-10 | B-10 |
| f-7 | 4-tol | 4-tol | B-3 | B-3 |
| f-8 | 2-Np | 2-Np | B-8 | B-8 |
| f-9 | 1-Np | 1-Np | B-9 | B-9 |
| f-10 | 2-An | 2-An | B-10 | B-10 |
| f-11 | 2-Fn | 2-Fn | B-14 | B-14 |
| f-12 | Me | Me | B-21 | B-21 |
| f-13 | i-Pr | i-Pr | B-29 | B-29 |
| f-14 | Et | Et | B-41 | B-41 |
| f-15 | Ph | 2-tol | B-45 | B-50 |
| f-16 | 3-tol | Ph | B-9 | B-2 |
| f-17 | 2-Fn | Ph | B-8 | B-3 |
| f-18 | t-Bu | t-Bu | B-3 | B-4 |
| f-19 | 2-Np | Ph | B-1 | B-9 |
| f-20 | Ph | Ph | B-63 | B-63 |
| f-21 | Ph | Ph | B-68 | B-68 |
| f-22 | Ph | Ph | B-71 | B-71 |
| f-23 | Ph | Ph | B-74 | B-74 |
| f-24 | 2-tol | 2-tol | B-76 | B-76 |
| f-25 | 3-tol | 3-tot | B-80 | B-80 |
| f-26 | 4-tol | 4-tol | B-83 | B-83 |
| f-27 | 2-Np | 2-Np | B-87 | B-87 |
| f-28 | 1-Np | 1-Np | B-93 | B-93 |
| f-29 | 2-An | 2-An | B-97 | B-97 |
| f-30 | 2-Fn | 2-En | B-100 | B-100 |
| f-31 | Me | Me | B-104 | B-104 |
| f-32 | i-Pr | i-Pr | B-111 | B-111 |
| f-33 | Et | Et | B-113 | B-113 |
| f-34 | Ph | 2-tol | B-118 | B-118 |
| f-35 | 3-tol | Ph | B-124 | B-124 |
| f-36 | 2-Fn | Ph | B-127 | B-127 |
| f-37 | t-Bu | t-Bu | B-34 | B-114 |
| f-38 | 2-Np | Ph | B-45 | B-105 |

(g)

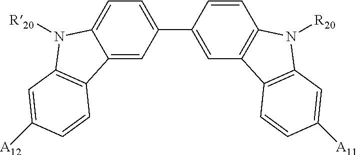

| Compound No. | R₂₀ | R′₂₀ | A₁₁ | A₁₂ |
|---|---|---|---|---|
| g-1 | Ph | Ph | B-2 | B-2 |
| g-2 | Ph | Ph | B-3 | B-3 |
| g-3 | Ph | Ph | B-9 | B-9 |
| g-4 | Ph | Ph | B-10 | B-10 |
| g-10 | 2-tol | 2-tol | B-9 | B-9 |
| g-11 | 3-tol | 3-tol | B-14 | B-14 |
| g-12 | 4-tol | 4-tol | B-10 | B-10 |
| g-13 | 2-Np | 2-Np | B-8 | B-8 |
| g-14 | 1-Np | 1-Np | B-9 | B-9 |
| g-15 | 2-An | 2-An | B-10 | B-10 |
| g-16 | 2-En | 2-Fn | B-21 | B-21 |
| g-17 | Me | Me | B-26 | B-26 |
| g-18 | i-Pr | i-Pr | B-31 | B-31 |
| g-19 | Et | Et | B-37 | B-37 |
| g-20 | Ph | 2-tol | B-43 | B-43 |
| g-21 | 3-tol | Ph | B-48 | B-48 |
| g-22 | 2-Fn | Ph | B-22 | B-22 |
| g-24 | t-Bu | t-Bu | B-28 | B-28 |
| g-25 | 2-Np | Ph | B-1 | B-9 |
| g-26 | Ph | Ph | B-64 | B-64 |
| g-27 | Ph | Ph | B-67 | B-67 |
| g-28 | Ph | Ph | B-71 | B-71 |
| g-29 | Ph | Ph | B-75 | B-75 |
| g-30 | 2-tol | 2-tol | B-78 | B-78 |
| g-31 | 3-tol | 3-tol | B-81 | B-81 |
| g-32 | 4-tol | 4-tol | B-85 | B-85 |
| g-33 | 2-Np | 2-Np | B-88 | B-88 |
| g-34 | 1-Np | 1-Np | B-91 | B-91 |
| g-35 | 2-An | 2-An | B-95 | B-95 |
| g-36 | 2-Fn | 2-Fn | B-98 | B-98 |
| g-37 | Me | Me | B-101 | B-101 |
| g-38 | i-Pr | i-Pr | B-102 | B-102 |
| g-39 | Et | Et | B-106 | B-106 |
| g-40 | Ph | 2-tol | B-109 | B-109 |
| g-41 | 3-tol | Ph | B-114 | B-114 |
| g-42 | 2-Fn | Ph | B-116 | B-116 |
| g-43 | t-Bu | t-Bu | B-120 | B-120 |
| g-44 | 2-Np | Ph | B-123 | B-123 |
| g-45 | Ph | 2-tol | B-127 | B-127 |
| g-46 | Ph | Ph | B-131 | B-131 |
| g-47 | Ph | 2-tol | B-132 | B-132 |
| g-48 | 3-tol | Ph | B-135 | B-35 |
| g-49 | 2-Fn | Ph | B-22 | B-122 |
| g-50 | t-Bu | t-Bu | B-28 | B-128 |

(h)

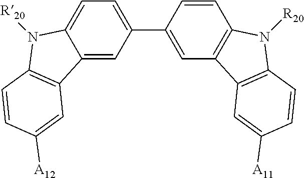

| Compound No. | R₂₀ | R′₂₀ | A₁₁ | A₁₂ |
|---|---|---|---|---|
| h-1 | Ph | Ph | B-2 | B-2 |
| h-2 | Ph | Ph | B-3 | B-3 |
| h-3 | Ph | Ph | B-9 | B-9 |
| h-4 | Ph | Ph | B-10 | B-10 |
| h-5 | 2-tol | 2-tol | B-9 | B-9 |
| h-6 | 3-tol | 3-tol | B-14 | B-14 |
| h-7 | 4-tol | 4-tol | B-10 | B-10 |
| h-8 | 2-Np | 2-Np | B-8 | B-8 |
| h-9 | 1-Np | 1-Np | B-9 | B-9 |
| h-10 | 2-An | 2-An | B-10 | B-10 |
| h-11 | 2-Fn | 2-Fn | B-21 | B-21 |
| h-12 | Me | Me | B-26 | B-26 |
| h-13 | i-Pr | i-Pr | B-31 | B-31 |
| h-14 | Et | Et | B-38 | B-38 |
| h-15 | Ph | 2-tol | B-42 | B-42 |
| h-16 | 3-tol | Ph | B-51 | B-51 |
| h-17 | 2-Fn | Ph | B-3 | B-4 |
| h-18 | t-Bu | t-Bu | B-5 | B-5 |
| h-19 | 2-Np | Ph | B-3 | B-10 |
| h-20 | Ph | Ph | B-65 | B-65 |
| h-21 | Ph | Ph | B-68 | B-68 |
| h-22 | Ph | Ph | B-79 | B-79 |
| h-23 | Ph | Ph | B-80 | B-80 |
| h-24 | 2-tol | 2-tol | B-86 | B-86 |
| h-25 | 3-tol | 3-tol | B-89 | B-89 |
| h-26 | 4-tol | 4-tol | B-103 | B-103 |
| h-27 | 2-Np | 2-Np | B-105 | B-105 |
| h-28 | 1-Np | 1-Np | B-107 | B-107 |
| h-29 | 2-An | 2-An | B-115 | B-115 |
| h-30 | 2-Fn | 2-Fn | B-119 | B-119 |
| h-31 | Me | Me | B-125 | B-125 |
| h-32 | i-Pr | i-Pr | B-128 | B-128 |
| h-33 | Et | Et | B-133 | B-133 |
| h-34 | Ph | 2-tol | B-42 | B-142 |
| h-35 | 3-tol | Ph | B-51 | B-115 |
| h-36 | 2-Fn | Ph | B-13 | B-67 |
| h-37 | t-Bu | t-Bu | B-5 | B-65 |
| h-38 | 2-Np | Ph | B-3 | B-73 |

(i)

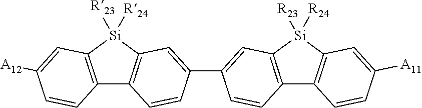

| Compound | | | | | | |
|---|---|---|---|---|---|---|
| No. | R₂₃ | R₂₄ | R′₂₃ | R′₂₄ | A₁₁ | A₁₂ |
| i-1 | Me | Me | Me | Me | B-1 | B-1 |
| i-2 | Me | Me | Me | Me | B-2 | B-2 |
| i-3 | Me | Me | Me | Me | B-3 | B-3 |
| i-4 | Me | Me | Me | Me | B-8 | B-8 |
| i-5 | Me | Me | Me | Me | B-9 | B-9 |
| i-6 | Me | Me | Me | Me | B-10 | B-10 |
| i-7 | Me | Me | Me | Me | B-14 | B-14 |
| i-8 | Me | Me | Me | Me | B-22 | B-22 |
| i-9 | Me | Me | Me | Me | B-27 | B-27 |
| i-10 | Me | Me | Me | Me | B-33 | B-33 |
| i-11 | Me | Me | Me | Me | B-42 | B-42 |
| i-12 | H | H | H | H | B-43 | B-43 |
| i-13 | H | H | H | Me | B-44 | B-44 |
| i-14 | Et | Et | Et | Et | B-45 | B-45 |
| i-15 | n-Bu | n-Bu | n-Bu | n-Bu | B-31 | B-33 |
| i-16 | Ph | Ph | Ph | Ph | B-4 | B-4 |
| i-17 | Me | Me | Me | Ph | B-5 | B-5 |
| i-18 | i-Pr | i-Pr | i-Pr | i-Pr | B-17 | B-17 |
| i-19 | 2-MeOEt | 2-MeOEt | 2-MeOEt | 2-MeOEt | B-1 | B-2 |
| i-20 | 3-tol | Me | 3-tol | Me | B-1 | B-3 |
| i-21 | Et | Et | Ph | Ph | B-8 | B-9 |
| i-22 | 4-tol | Ph | 4-tol | Me | B-8 | B-10 |
| i-23 | Me | Me | Me | Me | B-1 | B-8 |
| i-24 | 2-tol | Me | 2-tol | Me | B-30 | B-10 |
| i-25 | Me | Et | Me | Ph | B-1 | B-20 |
| i-26 | Me | Me | Me | Me | B-65 | B-65 |
| i-27 | Me | Me | Me | Me | B-67 | B-67 |
| i-28 | Me | Me | Me | Me | B-62 | B-62 |
| i-29 | Me | Me | Me | Me | B-72 | B-72 |
| i-30 | Me | Me | Me | Me | B-77 | B-77 |
| i-31 | Me | Me | Me | Me | B-84 | B-84 |
| i-32 | Me | Me | Me | Me | B-85 | B-85 |
| i-33 | Me | Me | Me | Me | B-86 | B-86 |
| i-34 | Me | Me | Me | Me | B-90 | B-90 |
| i-35 | Me | Me | Me | Me | B-94 | B-94 |
| i-36 | Me | Me | Me | Me | B-96 | B-96 |
| i-37 | H | H | H | H | B-103 | B-103 |
| i-38 | H | H | H | Me | B-108 | B-108 |
| i-39 | Et | Et | Et | Et | B-110 | B-110 |
| i-40 | n-Bu | n-Bu | n-Bu | n-Bu | B-117 | B-117 |
| i-41 | Ph | Ph | Ph | Ph | B-121 | B-121 |
| i-42 | Me | Me | Me | Ph | B-126 | B-126 |
| i-43 | i-Pr | i-Pr | 1-Pr | i-Pr | B-129 | B-129 |
| i-44 | 2-MeOEt | 2-MeOEt | 2-MeOEt | 2-MeOEt | B-130 | B-130 |
| i-45 | 3-tol | Me | 3-tol | Me | B-133 | B-133 |
| i-46 | Et | Et | Ph | Ph | B-134 | B-134 |
| i-47 | 4-tol | Ph | 4-tol | Me | B-136 | B-136 |
| i-48 | Me | Me | Me | Me | B-1 | B-71 |
| i-49 | 2-tol | Me | 2-tol | Me | B-30 | B-90 |
| i-50 | Me | Et | Me | Ph | B-1 | B-66 |

(j)

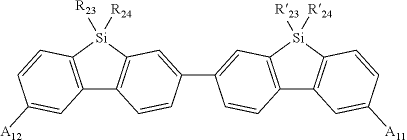

| Compound | | | | | | |
|---|---|---|---|---|---|---|
| No. | R₂₃ | R₂₄ | R′₂₃ | R′₂₄ | A₁₁ | A₁₂ |
| j-1 | Me | Me | Me | Me | B-1 | B-1 |
| j-2 | Me | Me | Me | Me | B-2 | B-2 |
| j-3 | Me | Me | Me | Me | B-3 | B-3 |
| j-4 | Me | Me | Me | Me | B-8 | B-8 |
| j-5 | Me | Me | Me | Me | B-9 | B-9 |
| j-6 | Me | Me | Me | Me | B-10 | B-10 |
| j-7 | Me | Me | Me | Me | B-14 | B-14 |
| j-8 | Me | Me | Me | Me | B-21 | B-21 |
| j-9 | Me | Me | Me | Me | B-31 | B-31 |
| j-10 | Me | Me | Me | Me | B-33 | B-33 |
| j-11 | Me | Me | Me | Me | B-42 | B-42 |
| j-12 | H | H | H | H | B-43 | B-43 |
| j-13 | H | H | H | Me | B-44 | B-44 |
| j-14 | Et | Et | Et | Et | B-45 | B-45 |
| j-15 | n-Bu | n-Bu | n-Bu | n-Bu | B-31 | B-31 |
| j-16 | Ph | Ph | Ph | Ph | B-4 | B-4 |
| j-17 | Me | Me | Me | Ph | B-5 | B-5 |
| j-18 | i-Pr | i-Pr | i-Pr | i-Pr | B-18 | B-18 |
| j-19 | 2-MeOEt | 2-MeOEt | 2-MeOEt | 2-MeOEt | B-1 | B-2 |
| j-20 | 3-tol | Me | 3-tol | Me | B-4 | B-3 |
| j-21 | Et | Et | Ph | Ph | B-8 | B-9 |
| j-22 | 4-tol | Ph | 4-tol | Me | B-8 | B-10 |
| j-23 | Me | Me | Me | Me | B-4 | B-5 |
| j-24 | 2-tol | Me | 2-tol | Me | B-31 | B-10 |
| j-25 | Me | Et | Me | Ph | B-3 | B-20 |
| j-26 | Me | Me | Me | Me | B-61 | B-61 |
| j-27 | Me | Me | Me | Me | B-64 | B-64 |
| j-28 | Me | Me | Me | Me | B-66 | B-66 |
| j-29 | Me | Me | Me | Me | B-69 | B-69 |
| j-30 | Me | Me | Me | Me | B-71 | B-71 |
| j-31 | Me | Me | Me | Me | B-72 | B-72 |
| j-32 | Me | Me | Me | Me | B-74 | B-74 |
| j-33 | Me | Me | Me | Me | B-76 | B-76 |
| j-34 | Me | Me | Me | Me | B-78 | B-78 |
| j-35 | Me | Me | Me | Me | B-81 | B-81 |
| j-36 | Me | Me | Me | Me | B-84 | B-84 |
| j-37 | H | H | H | H | B-86 | B-86 |
| j-38 | H | H | H | Me | B-89 | B-89 |
| j-39 | Et | Et | Et | Et | B-93 | B-93 |
| j-40 | n-Bu | n-Bu | n-Bu | n-Bu | B-98 | B-101 |
| j-41 | Ph | Ph | Ph | Ph | B-61 | B-61 |
| j-42 | Me | Me | Me | Ph | B-64 | B-64 |
| j-43 | i-Pr | i-Pr | i-Pr | i-Pr | B-66 | B-66 |
| j-44 | 2-MeOEt | 2-MeOEt | 2-MeOEt | 2-MeOEt | B-91 | B-91 |
| j-45 | 3-tol | Me | 3-tol | Me | B-99 | B-99 |
| j-46 | Et | Et | Ph | Ph | B-108 | B-108 |
| j-47 | 4-tol | Ph | 4-tol | Me | B-111 | B-111 |
| j-48 | Me | Me | Me | Me | B-114 | B-114 |
| j-49 | 2-tol | Me | 2-tol | Me | B-121 | B-121 |
| j-50 | Me | Et | Me | Ph | B-125 | B-125 |

(k)

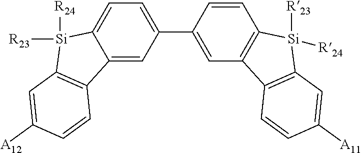

| Compound | | | | | | |
|---|---|---|---|---|---|---|
| No. | R₂₃ | R₂₄ | R′₂₃ | R′₂₄ | A₁₁ | A₁₂ |
| k-1 | Me | Me | Me | Me | B-1 | B-1 |
| k-2 | Me | Me | Me | Me | B-2 | B-2 |
| k-3 | Me | Me | Me | Me | B-3 | B-3 |
| k-4 | Me | Me | Me | Me | B-8 | B-8 |
| k-5 | Me | Me | Me | Me | B-9 | B-9 |
| k-6 | Me | Me | Me | Me | B-10 | B-10 |
| k-7 | Me | Me | Me | Me | B-14 | B-14 |
| k-8 | Me | Me | Me | Me | B-25 | B-25 |
| k-9 | Me | Me | Me | Me | B-22 | B-22 |
| k-10 | Me | Me | Me | Me | B-29 | B-29 |
| k-11 | Me | Me | Me | Me | B-33 | B-33 |
| k-12 | H | H | H | H | B-42 | B-42 |
| k-13 | H | H | H | Me | B-45 | B-45 |
| k-14 | Et | Et | Et | Et | B-50 | B-50 |
| k-15 | n-Bu | n-Bu | n-Bu | n-Bu | B-31 | B-31 |
| k-16 | Ph | Ph | Ph | Ph | B-3 | B-3 |
| k-17 | Me | Me | Me | Ph | B-9 | B-9 |
| k-18 | i-Pr | i-Pr | i-Pr | i-Pr | B-17 | B-18 |
| k-19 | 2-MeOEt | 2-MeOEt | 2-MeOEt | 2-MeOEt | B-3 | B-2 |
| k-20 | 3-tol | Me | 3-tol | Me | B-4 | B-3 |
| k-21 | Et | Et | Ph | Ph | B-8 | B-9 |
| k-22 | 4-tol | Ph | Ph | Me | B-8 | B-10 |
| k-23 | Me | Me | Me | Me | B-4 | B-5 |
| k-24 | 3-tol | Me | 2-tol | Me | B-31 | B-10 |
| k-25 | Me | Et | Me | Ph | B-3 | B-21 |
| k-26 | Me | Me | Me | Me | B-64 | B-64 |
| k-27 | Me | Me | Me | Me | B-67 | B-67 |
| k-28 | Me | Me | Me | Me | B-71 | B-71 |
| k-29 | Me | Me | Me | Me | B-75 | B-75 |
| k-30 | Me | Me | Me | Me | B-78 | B-78 |
| k-31 | Me | Me | Me | Me | B-81 | B-81 |
| k-32 | Me | Me | Me | Me | B-85 | B-85 |
| k-33 | Me | Me | Me | Me | B-88 | B-88 |
| k-34 | Me | Me | Me | Me | B-91 | B-91 |
| k-35 | Me | Me | Me | Me | B-95 | B-95 |
| k-36 | Me | Me | Me | Me | B-98 | B-98 |
| k-37 | H | H | H | H | B-101 | B-101 |
| k-38 | H | H | H | Me | B-102 | B-102 |
| k-39 | Et | Et | Et | Et | B-106 | B-106 |
| k-40 | n-Bu | n-Bu | n-Bu | n-Bu | B-109 | B-109 |
| k-41 | Ph | Ph | Ph | Ph | B-111 | B-111 |
| k-42 | Me | Me | Me | Ph | B-112 | B-112 |
| k-43 | i-Pr | i-Pr | i-Pr | i-Pr | B-116 | B-116 |
| k-44 | 2-MeOEt | 2-MeOEt | 2-MeOEt | 2-MeOEt | B-123 | B-123 |
| k-45 | 3-tol | Me | 3-tol | Me | B-126 | B-126 |
| k-46 | Et | Et | Ph | Ph | B-127 | B-131 |
| k-47 | 4-tol | Ph | Ph | Me | B-45 | B-50 |
| k-48 | Me | Me | Me | Me | B-8 | B-9 |
| k-49 | 3-tol | Me | 2-tol | Me | B-8 | B-10 |
| k-50 | Me | Et | Me | Ph | B-72 | B-17 |

(l)

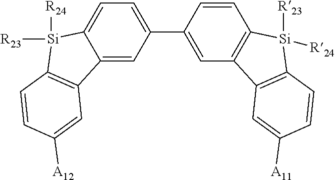

| Compound | | | | | | |
|---|---|---|---|---|---|---|
| No. | R₂₃ | R₂₄ | R′₂₃ | R′₂₄ | A₁₁ | A₁₂ |
| l-1 | Me | Me | Me | Me | B-1 | B-1 |
| l-2 | Me | Me | Me | Me | B-2 | B-2 |
| l-3 | Me | Me | Me | Me | B-3 | B-3 |
| l-4 | Me | Me | Me | Me | B-8 | B-8 |
| l-5 | Me | Me | Me | Me | B-9 | B-9 |
| l-6 | Me | Me | Me | Me | B-10 | B-10 |
| l-7 | Me | Me | Me | Me | B-14 | B-14 |
| l-8 | Me | Me | Me | Me | B-22 | B-22 |
| l-9 | Me | Me | Me | Me | B-27 | B-27 |
| l-10 | Me | Me | Me | Me | B-33 | B-33 |
| l-11 | Me | Me | Me | Me | B-42 | B-42 |
| l-12 | H | H | H | H | B-43 | B-43 |
| l-13 | H | H | H | Me | B-44 | B-44 |
| l-14 | Et | Et | Et | Et | B-45 | B-45 |
| l-15 | n-Bu | n-Bu | n-Bu | n-Bu | B-31 | B-33 |
| l-16 | Ph | Ph | Ph | Ph | B-4 | B-4 |
| l-17 | Me | Me | Me | Ph | B-5 | B-5 |
| l-18 | i-Pr | i-Pr | i-Pr | i-Pr | B-17 | B-17 |
| l-19 | 2-MeOEt | 2-MeOEt | 2-MeOEt | 2-MeOEt | B-1 | B-2 |
| l-20 | 3-tol | Me | 3-tol | Me | B-1 | B-3 |
| l-21 | Et | Et | Ph | Ph | B-8 | B-9 |
| l-22 | 4-tol | Ph | 4-tol | Me | B-8 | B-10 |
| l-23 | Me | Me | Me | Me | B-1 | B-8 |
| l-24 | 2-tol | Me | 2-tol | Me | B-30 | B-10 |
| l-25 | Me | Et | Me | Ph | B-1 | B-20 |
| l-26 | Me | Me | Me | Me | B-62 | B-62 |
| l-27 | Me | Me | Me | Me | B-65 | B-65 |
| l-28 | Me | Me | Me | Me | B-73 | B-73 |
| l-29 | Me | Me | Me | Me | B-77 | B-77 |
| l-30 | Me | Me | Me | Me | B-86 | B-86 |
| l-31 | Me | Me | Me | Me | B-83 | B-83 |
| l-32 | Me | Me | Me | Me | B-90 | B-90 |
| l-33 | Me | Me | Me | Me | B-93 | B-93 |
| l-34 | Me | Me | Me | Me | B-98 | B-101 |
| l-35 | Me | Me | Me | Me | B-102 | B-105 |
| l-36 | Me | Me | Me | Me | B-106 | B-106 |
| l-37 | H | H | H | H | B-107 | B-110 |
| l-38 | H | H | H | Me | B-112 | B-115 |
| l-39 | Et | Et | Et | Et | B-116 | B-119 |
| l-40 | n-Bu | n-Bu | n-Bu | n-Bu | B-120 | B-124 |
| l-41 | Ph | Ph | Ph | Ph | B-127 | B-131 |
| l-42 | Me | Me | Me | Ph | B-132 | B-136 |
| l-43 | i-Pr | i-Pr | i-Pr | i-Pr | B-128 | B-128 |
| l-44 | 2-MeOEt | 2-MeOEt | 2-MeOEt | 2-MeOEt | B-1 | B-61 |
| l-45 | 3-tol | Me | 3-tot | Me | B-1 | B-71 |
| l-46 | Et | Et | Ph | Ph | B-30 | B-90 |
| l-47 | 4-tol | Ph | 4-tol | Me | B-1 | B-66 |
| l-48 | Me | Me | Me | Me | B-5 | B-66 |
| l-49 | 2-tol | Me | 2-tol | Me | B-70 | B-71 |
| l-50 | Me | Et | Me | Ph | B-80 | B-81 |

(m)

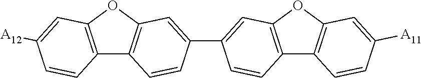

| Compound No. | A₁₁ | A₁₂ |
|---|---|---|
| m-1 | B-1 | B-1 |
| m-2 | B-2 | B-2 |
| m-3 | B-3 | B-3 |
| m-4 | B-8 | B-8 |
| m-5 | B-9 | B-9 |
| m-6 | B-10 | B-10 |
| m-7 | B-14 | B-14 |
| m-8 | B-25 | B-25 |
| m-9 | B-22 | B-22 |
| m-10 | B-29 | B-29 |
| m-11 | B-33 | B-33 |
| m-12 | B-42 | B-42 |
| m-13 | B-45 | B-45 |
| m-14 | B-50 | B-50 |
| m-15 | B-31 | B-31 |
| m-16 | B-3 | B-3 |
| m-17 | B-9 | B-9 |
| m-18 | B-17 | B-18 |
| m-19 | B-3 | B-2 |
| m-20 | B-4 | B-3 |
| m-21 | B-63 | B-63 |
| m-22 | B-68 | B-68 |
| m-23 | B-71 | B-71 |
| m-24 | B-74 | B-74 |
| m-25 | B-76 | B-76 |
| m-26 | B-80 | B-80 |
| m-27 | B-83 | B-83 |
| m-28 | B-88 | B-88 |
| m-29 | B-96 | B-96 |
| m-30 | B-100 | B-100 |
| m-31 | B-117 | B-117 |
| m-32 | B-125 | B-125 |
| m-33 | B-131 | B-131 |
| m-34 | B-134 | B-134 |
| m-35 | B-135 | B-135 |
| m-36 | B-14 | B-74 |
| m-37 | B-25 | B-86 |
| m-38 | B-101 | B-10 |
| m-39 | B-6 | B-66 |
| m-40 | B-16 | B-73 |

(n)

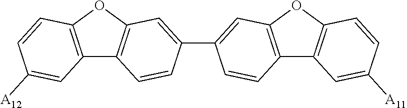

| Compound No. | A₁₁ | A₁₂ |
|---|---|---|
| n-1 | B-2 | B-2 |
| n-2 | B-3 | B-3 |
| n-3 | B-9 | B-9 |
| n-4 | B-10 | B-10 |
| n-10 | B-9 | B-9 |
| n-11 | B-14 | B-14 |
| n-12 | B-10 | B-10 |
| n-13 | B-8 | B-8 |
| n-14 | B-9 | B-9 |
| n-15 | B-10 | B-10 |
| n-16 | B-21 | B-21 |
| n-17 | B-26 | B-26 |
| n-18 | B-31 | B-31 |
| n-19 | B-38 | B-38 |
| n-20 | B-42 | B-42 |
| n-21 | B-51 | B-51 |
| n-22 | B-3 | B-4 |
| n-24 | B-5 | B-5 |
| n-25 | B-3 | B-10 |
| n-26 | B-64 | B-64 |
| n-27 | B-67 | B-67 |
| n-28 | B-71 | B-71 |
| n-29 | B-75 | B-75 |
| n-30 | B-78 | B-78 |
| n-31 | B-81 | B-81 |
| n-32 | B-85 | B-85 |
| n-33 | B-88 | B-88 |
| n-34 | B-91 | B-91 |
| n-35 | B-95 | B-95 |
| n-36 | B-98 | B-98 |
| n-37 | B-101 | B-101 |
| n-38 | B-104 | B-104 |
| n-39 | B-109 | B-109 |
| n-40 | B-111 | B-111 |
| n-41 | B-112 | B-112 |
| n-42 | B-116 | B-116 |
| n-43 | B-123 | B-123 |
| n-44 | B-126 | B-126 |
| n-45 | B-127 | B-131 |
| n-46 | B-45 | B-50 |
| n-47 | B-8 | B-9 |
| n-48 | B-8 | B-10 |
| n-49 | B-72 | B-17 |
| n-50 | B-3 | B-68 |

(o)

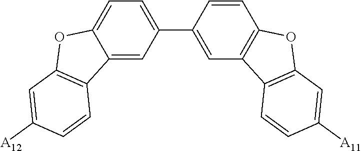

| Compound No. | A₁₁ | A₁₂ |
|---|---|---|
| o-1 | B-2 | B-2 |
| o-2 | B-3 | B-3 |
| o-3 | B-9 | B-9 |
| o-4 | B-10 | B-10 |
| o-10 | B-9 | B-9 |
| o-11 | B-14 | B-14 |
| o-12 | B-10 | B-10 |
| o-13 | B-8 | B-8 |
| o-14 | B-9 | B-9 |
| o-15 | B-10 | B-10 |
| o-16 | B-21 | B-21 |
| o-17 | B-26 | B-26 |
| o-18 | B-31 | B-31 |
| o-19 | B-37 | B-37 |
| o-20 | B-43 | B-43 |
| o-21 | B-48 | B-48 |
| o-22 | B-22 | B-22 |
| o-24 | B-28 | B-28 |
| o-25 | B-1 | B-9 |
| o-26 | B-62 | B-62 |
| o-27 | B-66 | B-66 |
| o-28 | B-73 | B-73 |
| o-29 | B-77 | B-77 |
| o-30 | B-82 | B-82 |
| o-31 | B-84 | B-84 |
| o-32 | B-85 | B-85 |
| o-33 | B-86 | B-86 |
| o-34 | B-87 | B-87 |
| o-35 | B-89 | B-89 |
| o-36 | B-93 | B-93 |
| o-37 | B-98 | B-101 |
| o-38 | B-102 | B-105 |
| o-39 | B-106 | B-106 |
| o-40 | B-107 | B-110 |
| o-41 | B-112 | B-115 |
| o-42 | B-116 | B-119 |
| o-43 | B-120 | B-124 |
| o-44 | B-127 | B-131 |
| o-45 | B-132 | B-136 |
| o-46 | B-128 | B-128 |
| o-47 | B-1 | B-61 |
| o-48 | B-66 | B-70 |
| o-49 | B-72 | B-75 |
| o-50 | B-67 | B-76 |

(p)

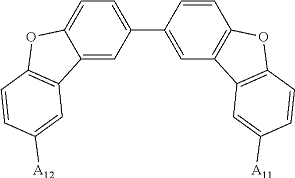

| Compound No. | A₁₁ | A₁₂ |
|---|---|---|
| p-1 | B-1 | B-1 |
| p-2 | B-2 | B-2 |
| p-3 | B-3 | B-3 |
| p-4 | B-8 | B-8 |
| p-5 | B-9 | B-9 |
| p-6 | B-10 | B-10 |
| p-7 | B-14 | B-14 |
| p-8 | B-22 | B-22 |
| p-9 | B-27 | B-27 |
| p-10 | B-33 | B-33 |
| p-11 | B-42 | B-42 |
| p-12 | B-43 | B-43 |
| p-13 | B-44 | B-44 |
| p-14 | B-45 | B-45 |
| p-15 | B-31 | B-33 |
| p-16 | B-4 | B-4 |
| p-17 | B-5 | B-5 |
| p-18 | B-17 | B-17 |
| p-19 | B-1 | B-2 |
| p-20 | B-1 | B-3 |
| p-21 | B-8 | B-9 |
| p-22 | B-8 | B-10 |
| p-23 | B-1 | B-8 |
| p-24 | B-30 | B-10 |
| p-25 | B-1 | B-20 |
| p-26 | B-63 | B-63 |
| p-27 | B-68 | B-68 |
| p-28 | B-71 | B-71 |
| p-29 | B-74 | B-74 |
| p-30 | B-76 | B-76 |
| p-31 | B-80 | B-80 |
| p-32 | B-83 | B-83 |
| p-33 | B-87 | B-87 |
| p-34 | B-93 | B-93 |
| p-35 | B-95 | B-95 |
| p-36 | B-98 | B-98 |
| p-37 | B-101 | B-101 |
| p-38 | B-102 | B-102 |
| p-39 | B-106 | B-106 |
| p-40 | B-109 | B-109 |
| p-41 | B-111 | B-111 |
| p-42 | B-112 | B-112 |
| p-43 | B-116 | B-116 |
| p-44 | B-123 | B-123 |
| p-45 | B-126 | B-126 |
| p-46 | B-127 | B-131 |
| p-47 | B-45 | B-50 |
| p-48 | B-8 | B-9 |
| p-49 | B-8 | B-10 |
| p-50 | B-72 | B-17 |

(q)

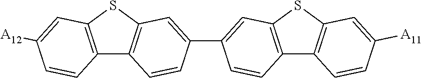

| Compound No. | A₁₁ | A₁₂ |
|---|---|---|
| q-1 | B-1 | B-1 |
| q-2 | B-2 | B-2 |
| q-3 | B-3 | B-3 |
| q-4 | B-8 | B-8 |
| q-5 | B-9 | B-9 |
| q-6 | B-10 | B-10 |
| q-7 | B-14 | B-14 |
| q-8 | B-22 | B-22 |
| q-9 | B-27 | B-27 |
| q-10 | B-33 | B-33 |
| q-11 | B-42 | B-42 |
| q-12 | B-43 | B-43 |
| q-13 | B-44 | B-44 |
| q-14 | B-45 | B-45 |
| q-15 | B-31 | B-33 |
| q-16 | B-4 | B-4 |
| q-17 | B-5 | B-5 |
| q-18 | B-17 | B-17 |
| q-19 | B-1 | B-2 |
| q-20 | B-1 | B-3 |
| q-21 | B-8 | B-9 |
| q-22 | B-8 | B-10 |
| q-23 | B-1 | B-8 |
| q-24 | B-30 | B-10 |
| q-25 | B-1 | B-20 |
| q-26 | B-64 | B-64 |
| q-27 | B-67 | B-67 |
| q-28 | B-71 | B-71 |
| q-29 | B-75 | B-75 |
| q-30 | B-78 | B-78 |
| q-31 | B-81 | B-81 |
| q-32 | B-85 | B-85 |
| q-33 | B-88 | B-88 |
| q-34 | B-91 | B-91 |
| q-35 | B-95 | B-95 |
| q-36 | B-98 | B-98 |
| q-37 | B-100 | B-100 |
| q-38 | B-101 | B-101 |
| q-39 | B-102 | B-102 |
| q-40 | B-104 | B-104 |
| q-41 | B-106 | B-106 |
| q-42 | B-109 | B-109 |
| q-43 | B-111 | B-111 |
| q-44 | B-113 | B-113 |
| q-45 | B-114 | B-114 |
| q-46 | B-118 | B-118 |
| q-47 | B-124 | B-124 |
| q-48 | B-127 | B-127 |
| q-49 | B-34 | B-114 |
| q-50 | B-45 | B-105 |

(r)

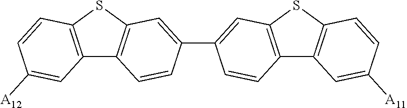

| Compound No. | A₁₁ | A₁₂ |
|---|---|---|
| r-1 | B-1 | B-1 |
| r-2 | B-2 | B-2 |
| r-3 | B-3 | B-3 |
| r-4 | B-8 | B-8 |
| r-5 | B-9 | B-9 |
| r-6 | B-10 | B-10 |
| r-7 | B-14 | B-14 |
| r-8 | B-21 | B-21 |
| r-9 | B-23 | B-23 |
| r-10 | B-31 | B-33 |
| r-11 | B-42 | B-42 |
| r-12 | B-43 | B-43 |
| r-13 | B-47 | B-47 |
| r-14 | B-48 | B-48 |
| r-15 | B-31 | B-33 |
| r-16 | B-4 | B-4 |
| r-17 | B-5 | B-5 |
| r-18 | B-17 | B-17 |
| r-19 | B-1 | B-2 |
| r-20 | B-1 | B-3 |
| r-21 | B-8 | B-9 |
| r-22 | B-8 | B-10 |
| r-23 | B-1 | B-8 |
| r-24 | B-30 | B-10 |
| r-25 | B-1 | B-20 |
| r-26 | B-64 | B-64 |
| r-27 | B-67 | B-67 |
| r-28 | B-71 | B-71 |
| r-29 | B-75 | B-75 |
| r-30 | B-78 | B-78 |
| r-31 | B-81 | B-81 |
| r-32 | B-85 | B-85 |
| r-33 | B-88 | B-88 |
| r-34 | B-91 | B-91 |
| r-35 | B-95 | B-95 |
| r-36 | B-98 | B-98 |
| r-37 | B-101 | B-101 |
| r-38 | B-102 | B-102 |
| r-39 | B-106 | B-106 |
| r-40 | B-109 | B-109 |
| r-41 | B-111 | B-111 |
| r-42 | B-112 | B-112 |
| r-43 | B-116 | B-116 |
| r-44 | B-123 | B-123 |
| r-45 | B-126 | B-126 |
| r-46 | B-127 | B-131 |
| r-47 | B-45 | B-50 |
| r-48 | B-8 | B-9 |
| r-49 | B-8 | B-10 |
| r-50 | B-72 | B-17 |

(s)

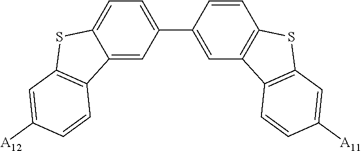

| Compound No. | A₁₁ | A₁₂ |
|---|---|---|
| s-1 | B-1 | B-1 |
| s-2 | B-2 | B-2 |
| s-3 | B-3 | B-3 |
| s-4 | B-8 | B-8 |
| s-5 | B-9 | B-9 |
| s-6 | B-10 | B-10 |
| s-7 | B-51 | B-51 |
| s-8 | B-46 | B-44 |
| s-9 | B-37 | B-37 |
| s-10 | B-38 | B-38 |
| s-11 | B-33 | B-35 |
| s-12 | B-27 | B-27 |
| s-13 | B-24 | B-24 |
| s-14 | B-7 | B-7 |
| s-15 | B-7 | B-24 |
| s-16 | B-63 | B-63 |
| s-17 | B-68 | B-68 |
| s-18 | B-71 | B-71 |
| s-19 | B-76 | B-76 |
| s-20 | B-80 | B-80 |
| s-21 | B-83 | B-83 |
| s-22 | B-87 | B-87 |
| s-23 | B-93 | B-93 |
| s-24 | B-94 | B-94 |
| s-25 | B-99 | B-99 |
| s-26 | B-108 | B-108 |
| s-27 | B-114 | B-114 |
| s-28 | B-121 | B-121 |
| s-29 | B-125 | B-125 |
| s-30 | B-129 | B-129 |

(t)

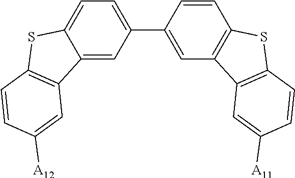

| Compound No. | A₁₁ | A₁₂ |
|---|---|---|
| t-1 | B-1 | B-1 |
| t-2 | B-2 | B-2 |
| t-3 | B-3 | B-3 |
| t-4 | B-8 | B-8 |
| t-5 | B-9 | B-9 |
| t-6 | B-10 | B-10 |
| t-7 | B-14 | B-14 |
| t-8 | B-20 | B-20 |
| t-9 | B-21 | B-21 |
| t-10 | B-1 | B-1 |
| t-11 | B-2 | B-2 |
| t-12 | B-3 | B-3 |
| t-13 | B-8 | B-8 |
| t-14 | B-9 | B-9 |
| t-15 | B-10 | B-10 |
| t-16 | B-4 | B-4 |
| t-17 | B-28 | B-28 |
| t-18 | B-36 | B-36 |
| t-19 | B-40 | B-40 |
| t-20 | B-45 | B-50 |
| t-21 | B-8 | B-9 |
| t-22 | B-8 | B-10 |
| t-23 | B-1 | B-1 |
| t-24 | B-3 | B-3 |
| t-25 | B-1 | B-8 |
| t-26 | B-62 | B-62 |
| t-27 | B-66 | B-66 |
| t-28 | B-73 | B-73 |
| t-29 | B-77 | B-77 |
| t-30 | B-82 | B-82 |
| t-31 | B-84 | B-84 |
| t-32 | B-85 | B-85 |
| t-33 | B-86 | B-86 |
| t-34 | B-90 | B-90 |
| t-35 | B-97 | B-97 |
| t-36 | B-99 | B-99 |
| t-37 | B-104 | B-104 |
| t-38 | B-109 | B-109 |
| t-39 | B-111 | B-111 |
| t-40 | B-112 | B-112 |
| t-41 | B-102 | B-102 |
| t-42 | B-106 | B-106 |
| t-43 | B-109 | B-109 |
| t-44 | B-111 | B-111 |
| t-45 | B-112 | B-112 |
| t-46 | B-116 | B-116 |
| t-47 | B-123 | B-123 |
| t-48 | B-126 | B-126 |
| t-49 | B-127 | B-131 |
| t-50 | B-45 | B-50 |

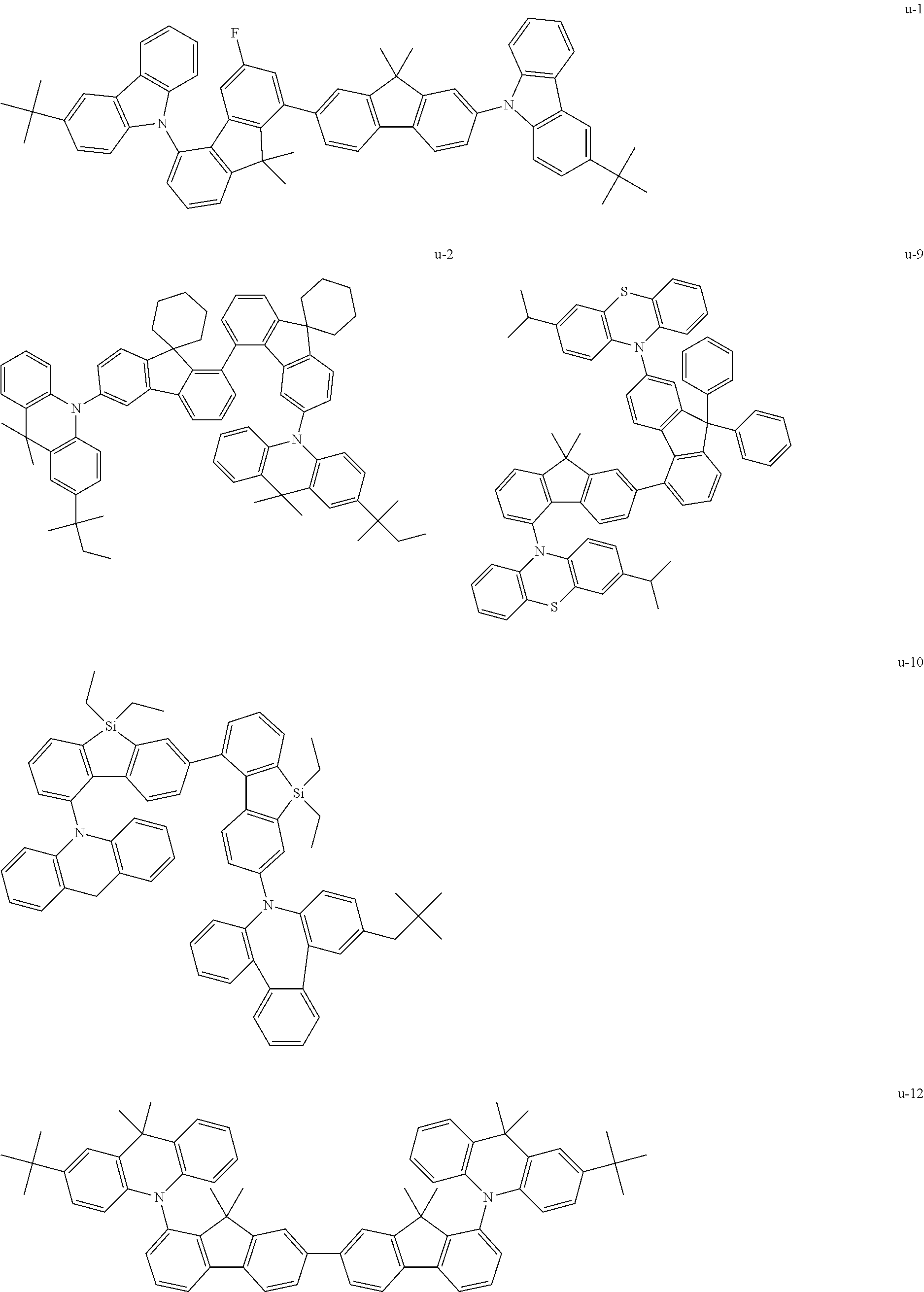

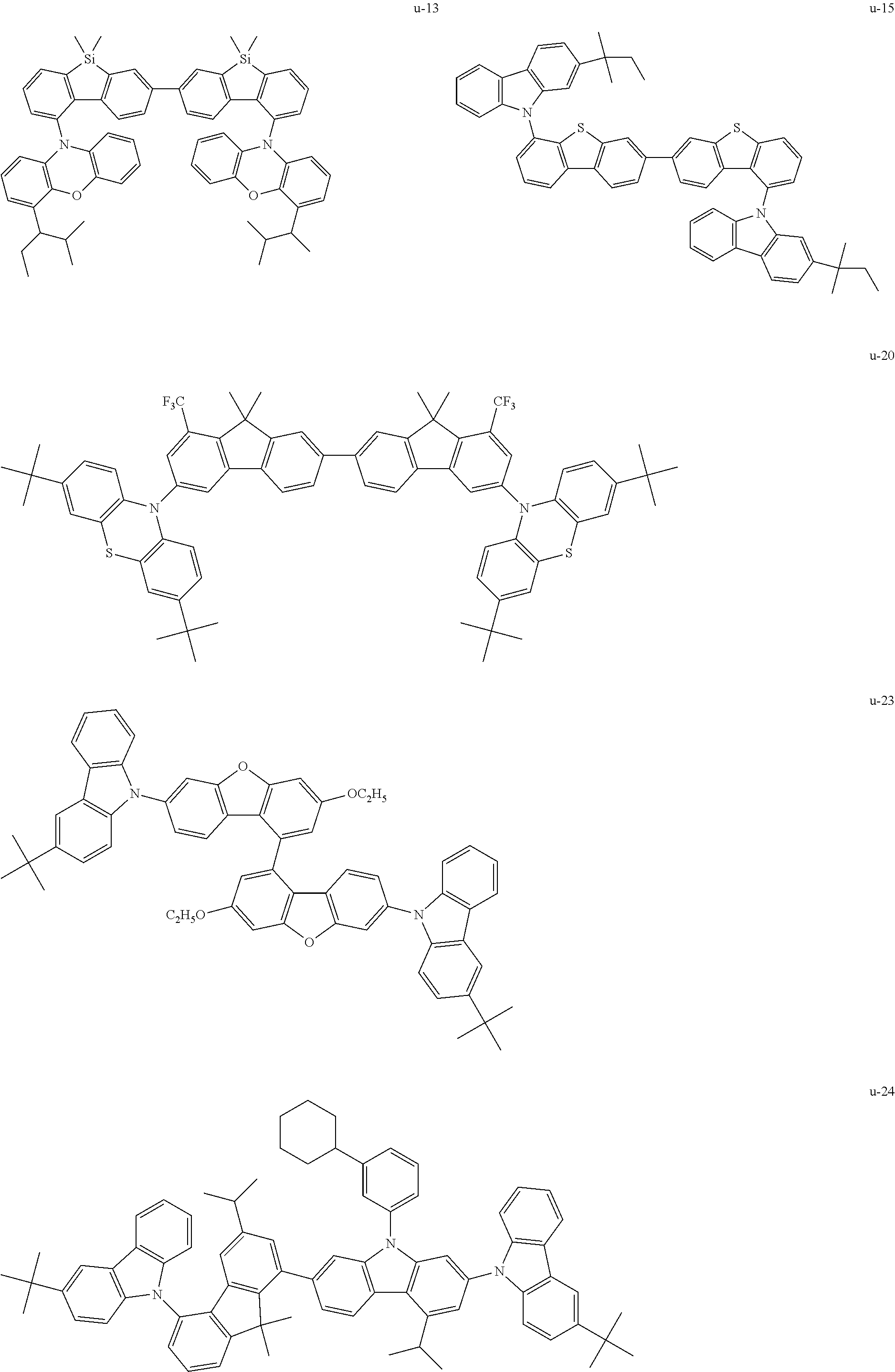

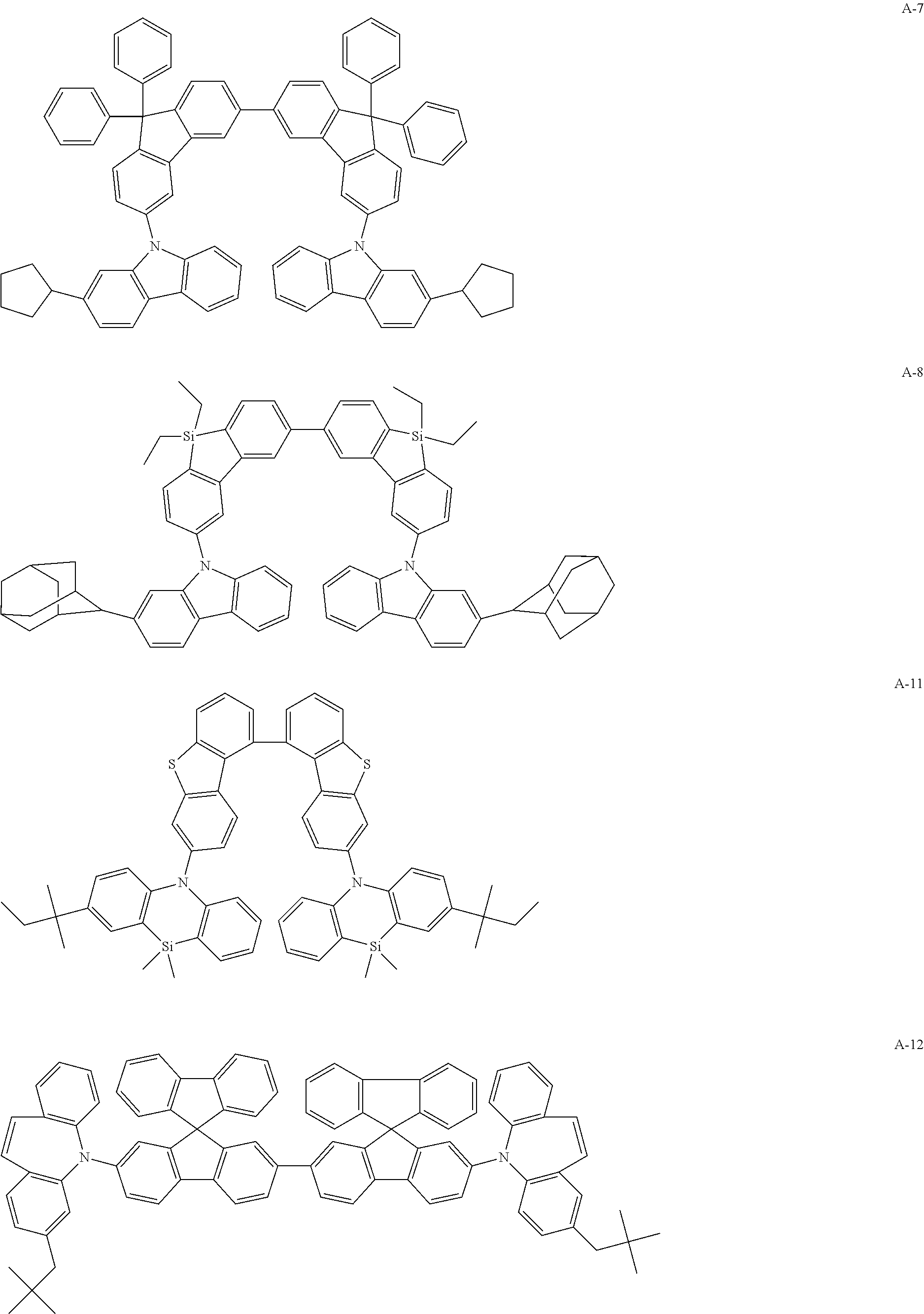

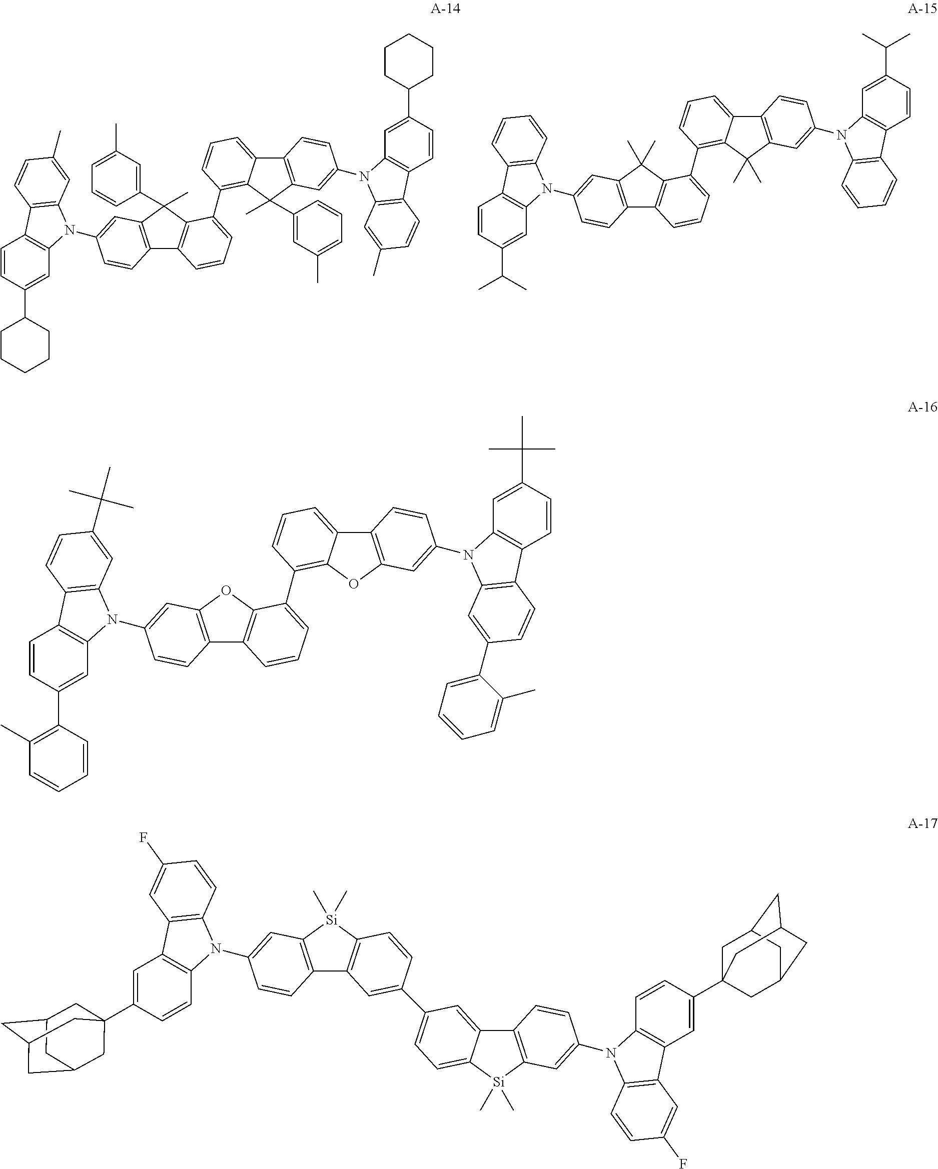

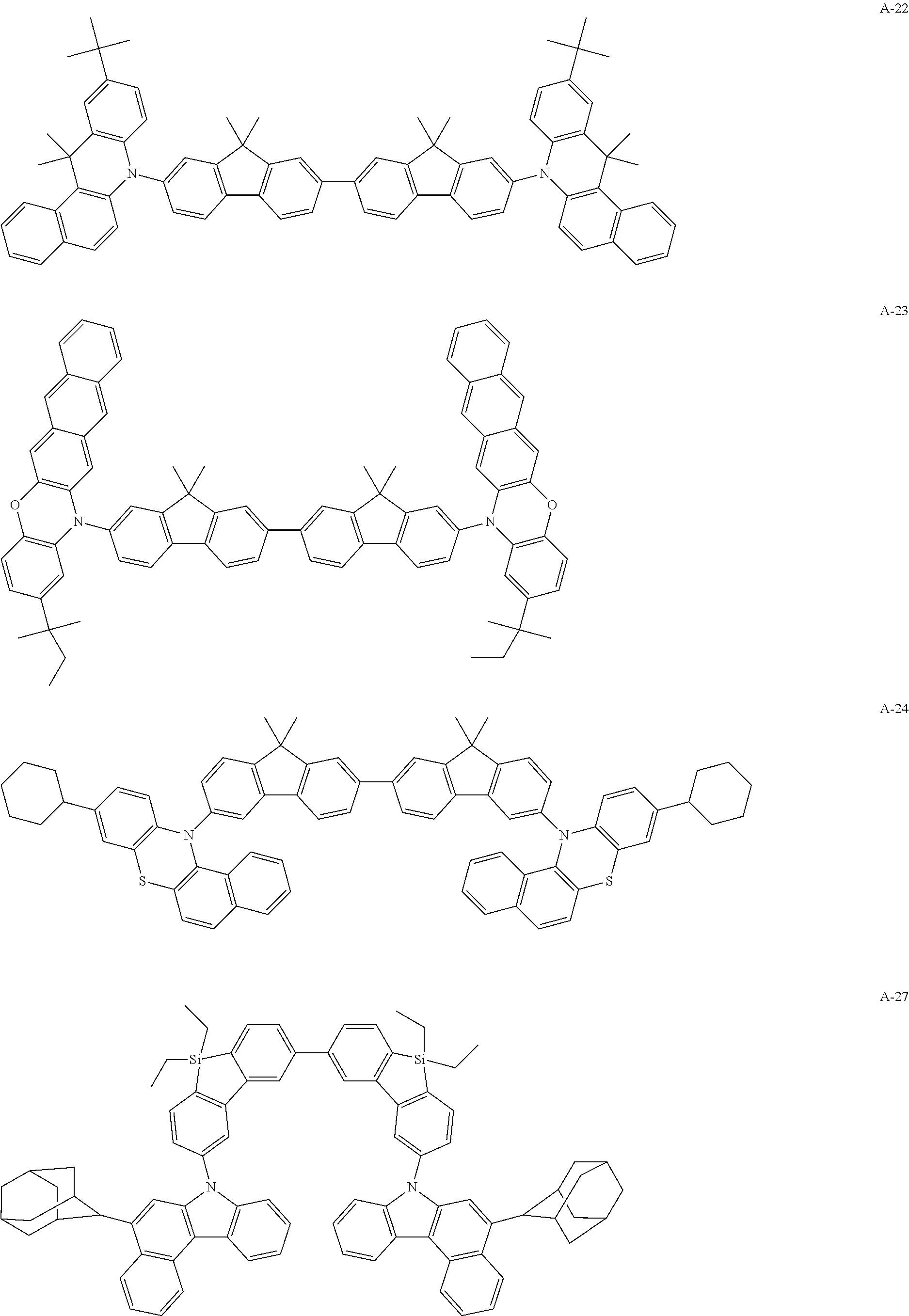

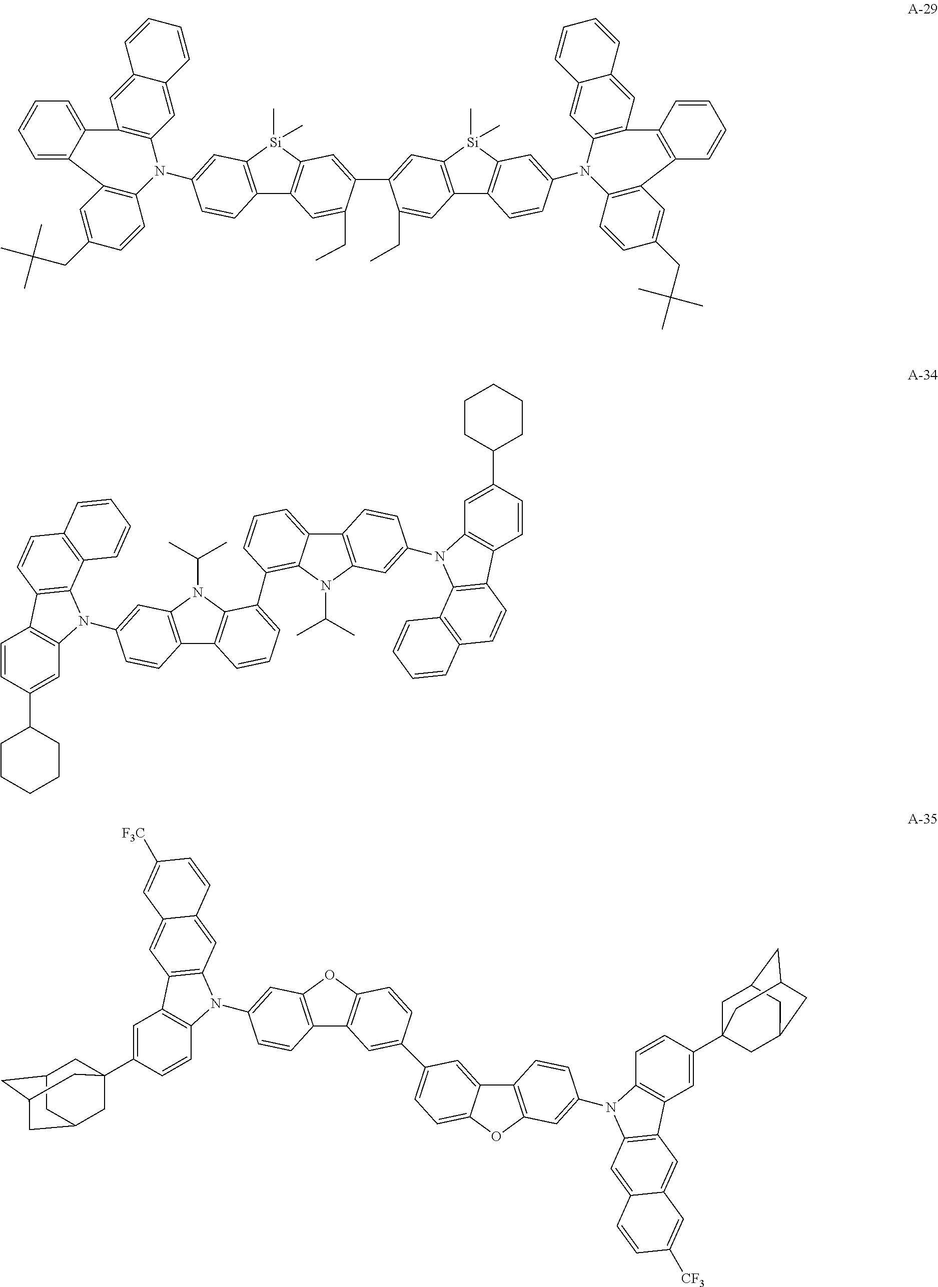 [SMILES: CCc1cc2c(cc1-c1cc3c(cc1CC)-c1ccc(N4c5ccc(CC(C)(C)C)cc5-c5ccccc5-c5cc6ccccc6cc54)cc1[Si]3(C)C)[Si](C)(C)c1cc(N3c4ccc(CC(C)(C)C)cc4-c4ccccc4-c4cc5ccccc5cc43)ccc1-2 ; CC(C)n1c2cc(-n3c4cc(C5CCCCC5)ccc4c4ccc5ccccc5c43)ccc2c2cccc(-c3cccc4c5ccc(-n6c7cc(C8CCCCC8)ccc7c7ccc8ccccc8c76)cc5n(C(C)C)c34)c21 ; FC(F)(F)c1ccc2cc3c(cc2c1)c1cc(C24CC5CC(CC(C5)C2)C4)ccc1n3-c1ccc2c(c1)oc1ccc(-c3ccc4oc5cc(-n6c7ccc(C89CC%10CC(CC(C%10)C8)C9)cc7c7cc8cc(C(F)(F)F)ccc8cc76)ccc5c4c3)cc12]

The molecular weight of the compound represented by Formula (1), (2), (F-1) or (F-2) is preferably 500 to 2,000, more preferably 500 to 1,500, still more preferably 700 to 1,500, and among them, the molecular weight is preferably 800 to 1,500, particularly preferably 900 to 1,500, and most preferably 940 to 1,500. By having a molecular weight from 500 to 2,000, the material may be deposited, thereby further increasing heat resistance.

When used in an organic electronics device, these compounds preferably have fewer impurities such as halogen ions and metal ions from the viewpoint of device performance thereof.

Further, it is possible to synthesize the compound represented by Formula (1), (2), (F-1) or (F-2) by applying already known methods. After the synthesis, a high-purity material for organic electronics may be obtained with high yield in a short period of time by purification using the purification method of the present invention.

Since the material for organic electronics of the present invention may be used in an organic electronic device such as a photoelectric conversion device, an organic electroluminescence device, an organic semiconductor device such as an organic thin film transistor, and is a high-purity material, an organic electronics device having excellent device performance may be obtained.

Among them, it is preferred that the material for organic electrics of the present invention is used in a photoelectric conversion device or an organic electroluminescence device.

Hereinafter, a photoelectric conversion device using the material for organic electronics of the present invention, an optical sensor and an imaging device, which use the photoelectric conversion device, and an organic electroluminescence device using the material for organic electronics of the present invention will be described.

[Photoelectric Conversion Device]

A photoelectric conversion device according to the present invention includes the material for organic electronics of the present invention. Since the material for organic electronics according to the present invention is a high-purity material, it is possible to obtain a photoelectric conversion device having high sensitivity and low dark current.

A preferred aspect of the photoelectric conversion device is an aspect in which the photoelectric conversion device has a transparent conductive film, a photoelectric conversion film and a conductive film in this order, the photoelectric conversion film includes a photoelectric conversion layer and a charge blocking layer, and the charge blocking layer includes the compound for organic electronics of the present invention. In addition, an aspect in which the conductive film, the charge blocking layer, the photoelectric conversion layer and the transparent conductive film are laminated in this order is a more preferred aspect. From the viewpoint of response speed, sensitivity and heat resistance of the device, the charge blocking layer includes preferably the compound represented by Formula (1) or (2), more preferably the compound represented by Formula (1), still more preferably the compound represented by Formula (F-1), and particularly preferably the compound represented by Formula (F-2).

Further, it is preferred that the photoelectric conversion layer also includes the material for organic electronics of the present invention, and examples of the material for organic electronics and for photoelectric conversion layer of the present invention include a compound represented by Formula (I) to be described below.

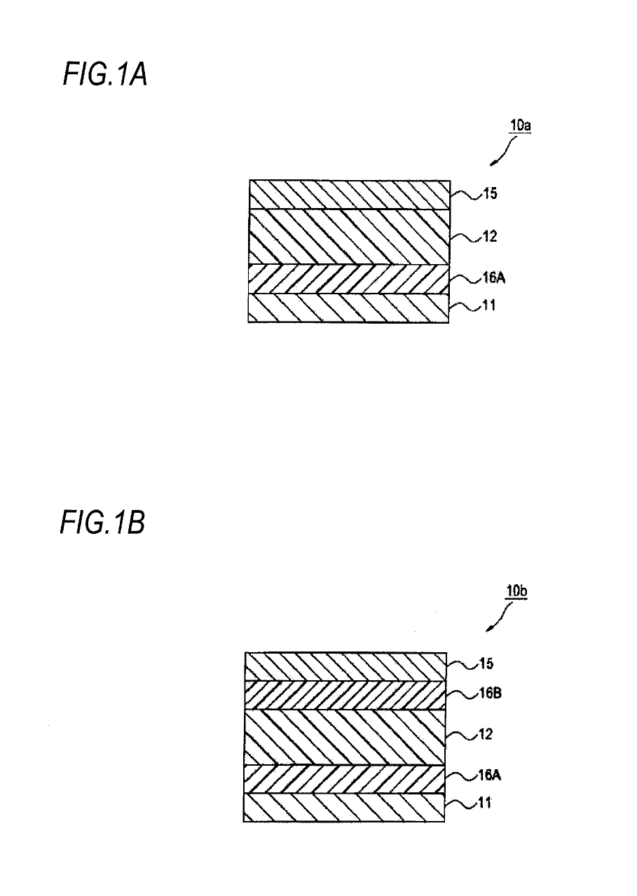

FIG. 1 illustrates a configuration example of a photoelectric conversion device according to exemplary embodiments of the present invention.

A photoelectric conversion device 10a illustrated in FIG. 1(a) has a configuration in which on a conductive film (hereinafter, referred to as a lower electrode) 11 serving as a lower electrode, a photoelectric conversion film (an electron blocking layer 16A and a photoelectric conversion layer 12 formed on the electron blocking layer 16A) formed on the lower electrode 11, and a transparent conductive film (hereinafter, referred to as an upper electrode) 15 serving as an upper electrode are laminated in this order.

FIG. 1(b) illustrates a configuration example of another photoelectric conversion device. A photoelectric conversion device 10b illustrated in FIG. 1(b) has a configuration in which a photoelectric conversion film (the electron blocking layer 16A, the photoelectric conversion layer 12, and a hole blocking layer 16B), and the upper electrode 15 are laminated in this order on the lower electrode 11. Further, the lamination order of the charge blocking layer, the photoelectric conversion layer, and the hole blocking layer in FIGS. 1(a) and (1b) may be reversed according to the use and the characteristics.

In these configurations, it is preferred that light is incident to the photoelectric conversion film through the transparent conductive layer.

Further, when these photoelectric devices are used, electric field may be applied thereto. In this case, the conductive film and the transparent conductive film may form a pair of electrodes, and an electric field, for example, from 1×10⁻⁴ V/cm to 1×10⁷ V/cm may be applied between the pair of electrodes. It is preferred that an electrode brought into contact with the charge blocking layer is used as a cathode, and the other electrode is used as an anode.

Elements constituting the photoelectric conversion device according to the present exemplary embodiment will be described.

(Electrode)

The electrodes (the upper electrode (transparent conductive film) 15 and the lower electrode (conductive film) 11)) are formed of a conductive material. As a conductive material, a metal, an alloy, a metal oxide, an electroconductive compound or a mixture thereof and the like may be used.

Since light is incident from the upper electrode 15, the upper electrode 15 needs to be sufficiently transparent with respect to light to be detected. Specific examples thereof include a conductive metal oxide such as tin oxide (ATO or FTO) doped with antimony or fluorine, and the like, tin oxide, zinc oxide, indium oxide, indium tin oxide (ITO) and indium zinc oxide (IZO), a metal thin film such as gold, silver, chromium, and nickel, a mixture or laminate of these metals with the conductive metal oxides, an inorganic conductive material such as copper iodide and copper sulfide, an organic conductive material such as polyaniline, polythiophene and polypyrrole, a laminate of these with ITO, and the like. Among the materials, in views of high conductivity and transparency, the transparent conductive metal oxide is preferred. It is preferred that the transparent conductive film is directly formed on the photoelectric conversion film. Since the upper electrode 15 is film-formed on the photoelectric conversion layer 12, it is preferred that the upper electrode 15 is film-formed by a method which does not degrade characteristics of the photoelectric conversion layer 12.

The lower electrode 11 may have transparency or not have transparency and use a material reflecting light according to the use thereof. Specific examples thereof include a conductive metal oxide such as tin oxide (ATO or FTO) doped with antimony or fluorine, and the like, tin oxide, zinc oxide, indium oxide, indium tin oxide (ITO) and indium zinc oxide (IZO), a metal such as gold, silver, chromium, nickel, titanium, tungsten and aluminum, a conductive compound (an example thereof includes titanium nitride (TiN)) such as oxides or nitrides of these metals, a mixture or a laminate of these metals with the conductive metal oxides, an inorganic conductive material such as copper iodide and copper sulfide, an organic conductive material such as polyaniline, polythiophene and polypyrrole, a laminate of these with ITO or titanium nitride, and the like.

A method for forming the electrode is not particularly limited, but may be appropriately selected in consideration of suitability with an electrode material. Specifically, the electrode may be formed by a wet method such as a printing method and a coating method, a physical method such as a vacuum deposition method, a sputtering method, and an ion plating method, and a chemical method such as CVD, and a plasma CVD method.

When the material of the electrode is ITO, the electrode may be formed by an electron beam method, a sputtering method, a resistance heating deposition method, a chemical reaction method (sol-gel method and the like), and a method such as coating of dispersion materials of indium tin oxide. In addition, a film manufactured by using ITO may be subjected to UV-ozone treatment, plasma treatment and the like. When the material of the electrode is TiN, various methods including a reactive sputtering method are used, and UV-ozone treatment, plasma treatment and the like may also be performed.

In consideration of suppression of leakage current, an increase in resistance value of a thin film, and an increase in transmittance, which are accompanied by the formation of the thin film, the film thickness of the upper electrode 15 is preferably 5 to 100 nm, and more preferably 5 to 20 nm.

[Photoelectric Conversion Layer]

In the present invention, an organic material constituting the photoelectric conversion layer (12 in FIG. 1) includes preferably at least one of a p-type organic semiconductor and an n-type organic semiconductor, and more preferably both the p-type organic semiconductor and the n-type organic semiconductor. Further, the effects of the present invention are particularly greatly exhibited when the photoelectric conversion layer includes a material having an electron affinity of 4.0 eV or more. Examples of the material having an electron affinity of 4.0 eV or more include an n-type organic semiconductor to be described below.

[p-Type Organic Semiconductor]

A p-type organic semiconductor (compound) is a donor-type organic semiconductor (compound) and refers to an organic compound having a property of easily donating electrons, usually typified by a hole transportable organic compound. More specifically, the p-type organic semiconductor material refers to an organic compound having a smaller ionization potential when two organic materials are brought into contact with each other and used. Accordingly, for the donor-type organic compound, any organic compound may be used as long as the organic compound is an organic compound having an electron donating property. For example, it is possible to use a metal complex having a triarylamine compound, a benzidine compound, a pyrazoline compound, a styrylamine compound, a hydrazone compound, a triphenylmethane compound, a carbazole compound, a polysilane compound, a thiophene compound, a phthalocyanine compound, a cyanine compound, merocyanine compound, an oxonol compound, a polyamine compound, indole compound, a pyrrole compound, a pyrazole compound, a polyarylene compound, a condensed aromatic carbon ring compound (a naphthalene derivative, an anthracene derivative, a phenanthrene derivative, a tetracene derivative, a pyrene derivative, a perylene derivative, and a fluoranthene derivative), a nitrogen-containing heterocyclic compound as a ligand, and the like. Further, the p-type organic semiconductor is not limited thereto, and as described above, an organic compound may be used as the donor-type organic semiconductor as long as the organic compound is an organic compound having an ionization potential smaller than that of the organic compound used as an n-type (acceptor property) compound. Among the aforementioned compounds, a triarylamine compound is preferred.

As the p-type organic semiconductor, a compound represented by the following Formula (I) is more preferred.

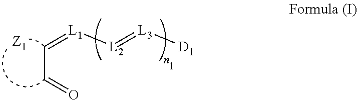

In the formula, Z₁ is a ring including at least two carbon atoms, and represents a 5-membered ring, a 6-membered ring or a condensed ring including at least one of the 5-membered ring and the 6-membered ring. L₁, L₂ and L₃ each independently represent an unsubstituted methine group or a substituted methine group. D₁ represents an atom group. n₁ represents an integer of 0 or more.

Formula (I) will be described.

Z₁ represents an atom group necessary for forming a 5- or 6-membered ring. L₁, L₂ and L₃ each independently represent an unsubstituted methine group or a substituted methine group. D₁ represents an atom group. n₁ represents an integer of 0 or more.

Z₁ is a ring including at least two carbon atoms, and represents a 5-membered ring, a 6-membered ring or a condensed ring including at least one of the 5-membered ring and the 6-membered ring. As the 5-membered ring, the six-membered ring or the condensed ring including at least one of the 5-membered ring and the 6-membered ring, those usually used as an acidic nucleus in a merocyanine pigment are preferred, and specific examples thereof include, for example, those described below.

(a) A 1,3-dicarbonyl nucleus: for example, a 1,3-indandione nucleus, 1,3-cyclohexanedione, 5,5-dimethyl-1,3-cyclohexanedione, 1,3-dioxane-4,6-dione and the like.

(b) A pyrazolinone nucleus: for example, 1-phenyl-2-pyrazolin-5-one, 3-methyl-1-phenyl-2-pyrazolin-5-one, 1-(2-benzothiazoyl)-3-methyl-2-pyrazolin-5-one and the like.

(c) An isoxazolinone nucleus: for example, 3-phenyl-2-isoxazolin-5-one, 3-methyl-2-isoxazolin-5-one and the like.

(d) A oxyindole nucleus: for example, 1-alkyl-2,3-dihydro-2-oxyindole and the like.

(e) a 2,4,6-triketohexahydropyrimidine nucleus: for example, barbituric acid, 2-thiobarbituric acid, derivatives thereof and the like. Examples of the derivatives include a 1-alkyl form such as 1-methyl and 1-ethyl, a 1,3-dialkyl form such as 1,3-dimethyl, 1,3-diethyl and 1,3-dibutyl, a 1,3-diaryl form such as 1,3-diphenyl, 1,3-di(p-chlorophenyl) and 1,3-di(p-ethoxycarbonylphenyl), a 1-alkyl-3-aryl form such as 1-ethyl-3-phenyl, a 1,3 position-diheterocyclic ring substitution product such as 1,3-di(2-pyridyl) and the like.

(f) A 2-thio-2,4-thiazolidinedione nucleus: for example, rhodanine and derivatives thereof. Examples of the derivatives include 3-alkylrhodanine such as 3-methylrhodanine, 3-ethylrhodanine and 3-allylrhodanine, 3-arylrhodanine such as 3-phenylrhodanine, 3 position-heterocyclic group-substituted rhodanine such as 3-(2-pyridyl)rhodanine, and the like.

(g) A 2-thio-2,4-oxazolidinedion (2-thio-2,4-(3H,5H)-oxazoledione nucleus: for example, 3-ethyl-2-thio-2,4-oxazolidinedione and the like.

(h) A thianaphthenone nucleus: for example, 3-(2H)-thianaphthenon-1,1-dioxide and the like.

(i) A 2-thio-2,5-thiazolidinedione nucleus: for example, 3-ethyl-2-thio-2,5-thiazolidinedione and the like.

(j) A 2,4-thiazolidinedione nucleus: for example, 2,4-thiazolidinedione, 3-ethyl-2,4-thiazolidinedione, 3-phenyl-2,4-thiazolidinedione and the like.

(k) A thiazolin-4-one nucleus: for example, 4-thiazolinone, 2-ethyl-4-thiazolinone and the like.

(l) A 2,4-imidazolidinedione (hydantoin) nucleus: for example, 2,4-imidazolidinedione, 3-ethyl-2,4-imidazolidinedione and the like.

(m) A 2-thio-2,4-imidazolidinedione (2-thiohydantoin) nucleus: for example, 2-thio-2,4-imidazolidinedione, 3-ethyl-2-thio-2,4-imidazolidinedione and the like.

(n) An 2-imidazolin-5-one nucleus: for example, 2-n-propylmercapto-2-imidazolin-5-one and the like.

(o) A 3,5-pyrazolidinedione nucleus: for example, 1,2-diphenyl-3,5-pyrazolidinedione, 1,2-dimethyl-3,5-pyrazolidinedione and the like.

(p) A benzothiophen-3-one nucleus: for example, benzothiophen-3-one, oxobenzothiophen-3-one, dioxobenzothiophen-3-one and the like.

(q) An indanone nucleus: for example, 1-indanone, 3-phenyl-1-indanone, 3-methyl-1-indanone, 3,3-diphenyl-1-indanone, 3,3-dimethyl-1-indanone and the like.

The ring represented by Z₁ is preferably a 1,3-dicarbonyl nucleus, a pyrazolinone nucleus, a 2,4,6-triketohexahydropyrimidine nucleus (including a thioketone form, for example, a barbituric acid nucleus and a 2-thiobarbituric acid nucleus), a 2-thio-2,4-thiazolidinedione nucleus, a 2-thio-2,4-oxazolidinedione nucleus, a 2-thio-2,5-thiazolidinedione nucleus, a 2,4-thiazolidinedione nucleus, a 2,4-imidazolidinedione nucleus, a 2-thio-2,4-imidazolidinedione nucleus, a 2-imidazoline-5-one nucleus, a 3,5-pyrazolidinedione nucleus, a benzothiophen-3-one nucleus, and an indanone nucleus, and more preferably a 1,3-dicarbonyl nucleus, a 2,4,6-triketohexahydropyrimidine nucleus (including a thioketone form, for example, a barbituric acid nucleus and a 2-thiobarbituric acid nucleus), a 3,5-pyrazolidinedione nucleus, a benzothiophen-3-one nucleus, and an indanone nucleus, still more preferably a 1,3-dicarbonyl nucleus, a 2,4,6-triketohexahydropyrimidine nucleus (including a thioketone form, for example, a barbituric acid nucleus and a 2-thiobarbituric acid nucleus), and particularly preferably a 1,3-indandione nucleus, a barbituric acid nucleus, a 2-thiobarbituric acid nucleus and a derivative thereof.

A preferred ring represented by Z₁ is represented by the following Formula.

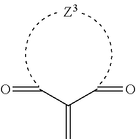

Z³ is a ring including at least three carbon atoms, and represents a 5-membered ring, a 6-membered ring or a condensed ring including at least one of the 5-membered ring and the 6-membered ring. Z³ may be selected among the rings formed by Z₁, and is preferably a 1,3-dicarbonyl nucleus or a 2,4,6-triketohexahydropyrimidine nucleus (including a thioketone form), and particularly preferably a 1,3-indandione nucleus, a barbituric acid nucleus, a 2-thiobarbituric acid nucleus and a derivative thereof.

It has been found that in the compound represented by Formula (I), a structure represented by D₁ and a structure represented by Z₁ serve as a donor part and an acceptor part, respectively, and the compound is useful as a photoelectric conversion material by linking both the structures through L1 and the like.

Further, it has been found that when used in combination with an n-type semiconductor material (acceptor property) such as C₆₀, the compound may exhibit high hole transportability by controlling the interaction between acceptor parts, when forming a co-deposition film with C₆₀.

Here, the interaction may be controlled by the structure of the acceptor part and the introduction of a substituent to be a steric hindrance. In the barbituric acid nucleus and 2-thiobarbituric acid nucleus, intermolecular interaction may be preferably controlled by substituting both hydrogens at two N-positions preferably with a substituent, and examples of the substituent include the substituent W to be described below, but the substituent is preferably an alkyl group, and more preferably a methyl group, an ethyl group, a propyl group or a butyl group.

When the ring represented by Z₁ is a 1,3-indandione nucleus, a group represented by the following Formula (IV) or a group represented by the following Formula (V) is preferred.

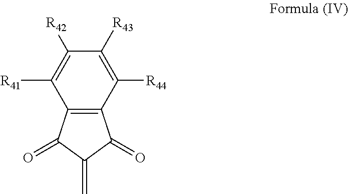

R₄₁ to R₄₄ each independently represent a hydrogen atom or a substituent.

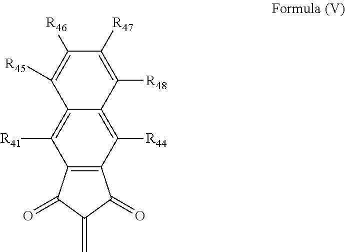

R₄₁, R₄₄ and R₄₅ to R₄₈ each independently represent a hydrogen atom or a substituent.

In the case of the group represented by Formula (IV), R₄₁ to R₄₄ each independently represent a hydrogen atom or a substituent. As the substituent, for example, those exemplified as the substituent W may be applied. In addition, adjacent members out of R₄₁ to R₄₄ may be bound with each other to form a ring (examples of the ring formed include the ring R to be described below), and it is preferred that R₄₂ and R₄₃ are bound with each other to form a ring (for example, a benzene ring, a pyridine ring and a pyrazine ring). It is preferred that all of R₄₁ to R₄₄ are a hydrogen atom.

It is preferred that the group represented by Formula (IV) is the group represented by Formula (V).

In the case of the group represented by Formula (V), R₄₁, R₄₄ and R₄₅ to R₄₈ each independently represent a hydrogen atom or a substituent. As the substituent, for example, those exemplified as the substituent W may be applied. It is preferred that all of R₄₁, R₄₄ and R₄₅ to R₄₈ are a hydrogen atom.

When the ring formed by Z₁ is a 2,4,6-triketohexahydropyrimidine nucleus (including a thioketone form), a group represented by the following Formula (VI) is preferred.

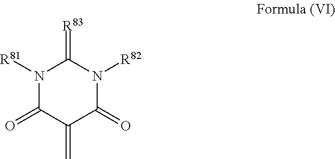

R₈₁ and R₈₂ each independently represent a hydrogen atom or a substituent. R₈₃ represents an oxygen atom, a sulfur atom or a substituent.

In the case of the group represented by Formula (VI), R₈₁ and R₈₂ each independently represent a hydrogen atom or a substituent. As the substituent, for example, those exemplified as the substituent W may be applied. R₈₁ and R₈₂ are each independently, preferably an alkyl group, an aryl group or a heterocyclic group (2-pyridyl and the like), and more preferably an alkyl group having 1 to 6 carbon atoms (for example, methyl, ethyl, n-propyl and t-butyl).

R₈₃ represents an oxygen atom, a sulfur atom or a substituent, but R₈₃ preferably represents an oxygen atom or a sulfur atom. The substituent is preferably a substituent with the bonding part being a nitrogen atom or a carbon atom, and when the bonding part is a nitrogen atom, an alkyl group (having 1 to 12 carbon atoms) or an aryl group (having a carbon number of 6 to 12) is preferred, and specific examples thereof include a methylamino group, an ethylamino group, a butylamino group, a hexylamino group, a phenylamino group or a naphthylamino group. When the bonding part is a carbon atom, it is sufficient if at least one electron-attracting group is substituted, and examples of the electron-attracting group includes a carbonyl group, a cyano group, a sulfoxide group, a sulfonyl group or a phosphoryl group, and it is preferred that the electron-attracting group further has a substituent. Examples of this substituent include the substituent W. It is preferred that R₈₃ preferably forms a 5- or 6-membered ring containing a carbon atom of the bonding part, and specific examples thereof include those having the following structures.

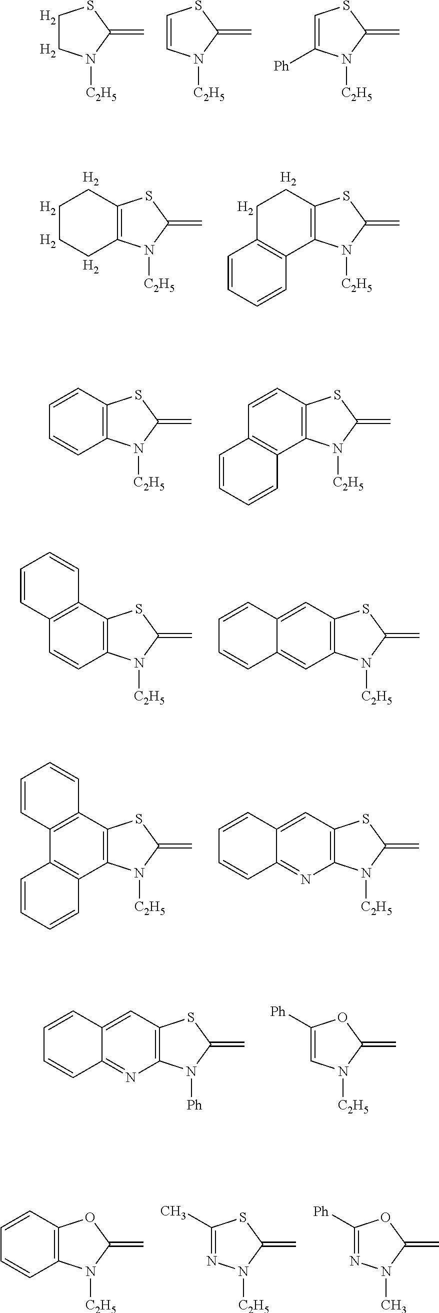

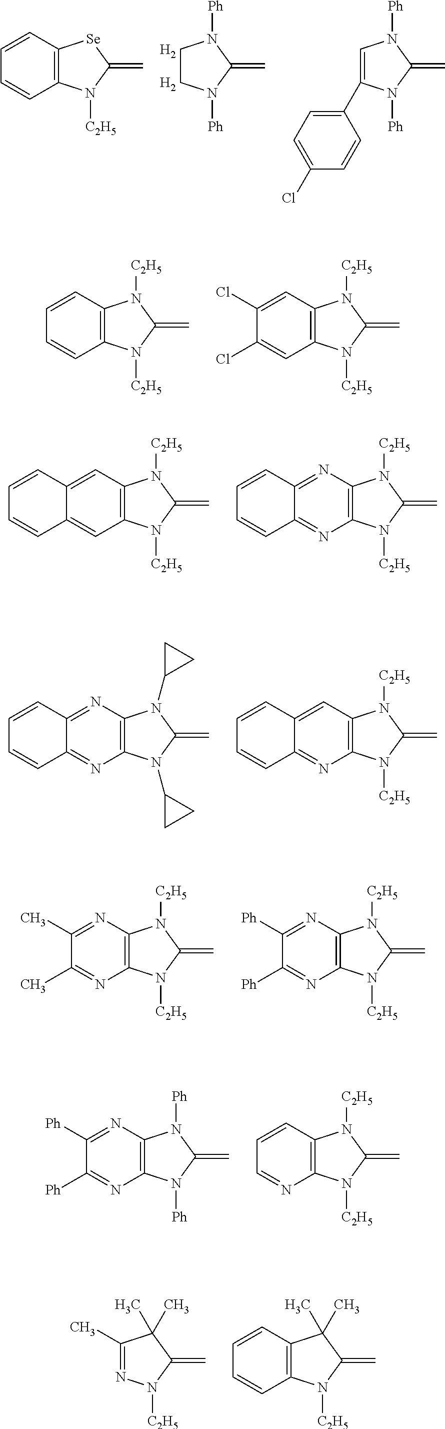

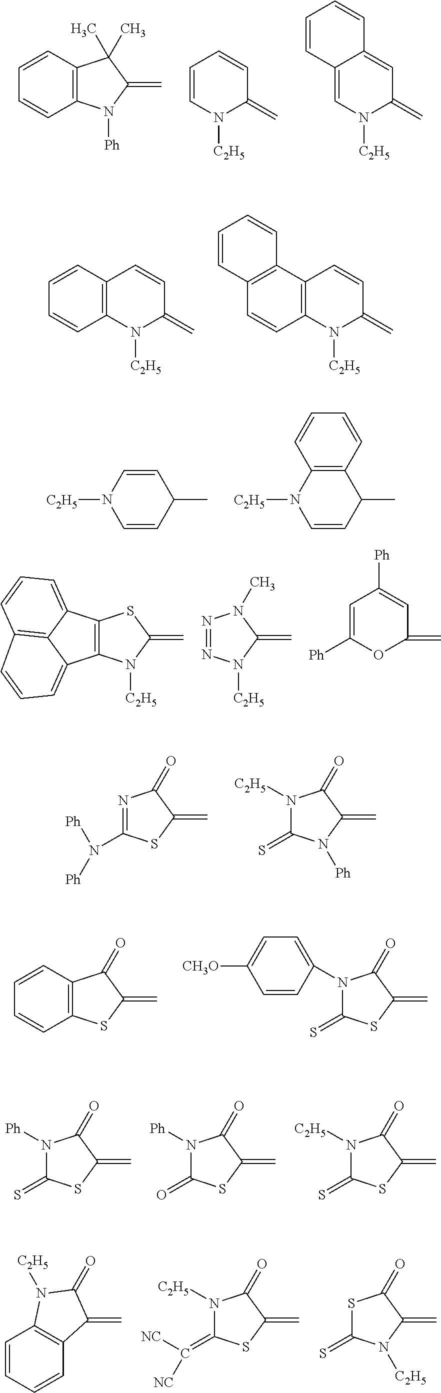

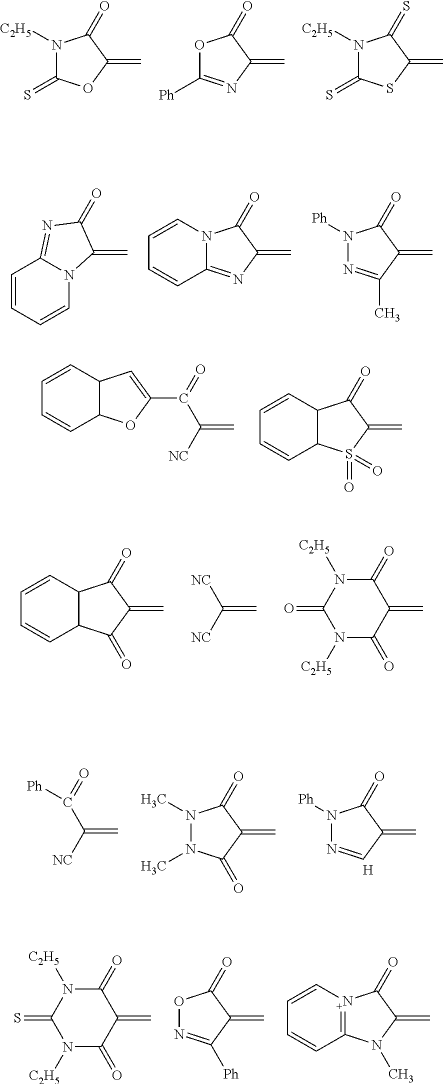

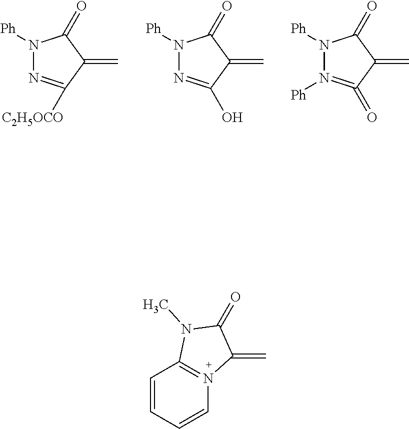

In the aforementioned groups, Ph indicates a phenyl group.

In Formula (I), L₁, L₂ and L₃ each independently represent an unsubstituted methine group or a substituted methine group. The substituted methine groups may be bound with each other to form a ring (for example, a 6-membered ring, for example, a benzene ring). Examples of the substituent of the substituted methine group include the substituent W, and it is preferred that all of L₁, L₂, and L₃ are an unsubstituted methine group.

In Formula (I), n₁ represents an integer of 0 or more, preferably an integer of 0 to 3, and more preferably 0. In the case where n₁ is increased, an absorption wavelength region may be a long wavelength, or a decomposition temperature by heat is decreased. From the viewpoint of providing appropriate absorption in a visible range and suppressing heat decomposition during the deposition and film formation, it is preferred that n₁=0.

In Formula (I), D₁ represents an atom group.

D₁ is preferably a group including —NR^{a}(R^{b}), and it is also preferred that D₁ represents an aryl group with —NR^{a}(R^{b}) being substituted (preferably, a phenyl group or a naphthyl group, which may have a substituent).

R^{a} and R^{b} each independently represent a hydrogen atom or a substituent, and examples of the substituent represented by R^{a} and R^{b} include the substituent W, but the substituent is preferably an aliphatic hydrocarbon group (preferably, an alkyl group and an alkenyl group, which may have a substituent), an aryl group (preferably, a phenyl group which may have a substituent) or a heterocyclic group, which may have a substituent. As the heterocyclic ring, a 5-membered ring such as furan, thiophene, pyrrole and oxadiazole is preferred.

When R^{a} and R^{b} are an aliphatic hydrocarbon group, an aryl group or a heterocyclic group, the substituent is preferably an alkyl group, an alkenyl group, an aryl group, an alkoxy group, an aryloxy group, an acyl group, an alkoxycarbonyl group, an aryloxycarbonyl group, an acylamino group, a sulfonylamino group, a sulfonyl group, a silyl group and an aromatic heterocyclic group, more preferably an alkyl group, an alkenyl group, an aryl group, an alkoxy group, an aryloxy group, a silyl group and an aromatic heterocyclic group, and still more preferably an alkyl group, an aryl group, an alkoxy group, an aryloxy group, a silyl group and an aromatic heterocyclic group. As specific examples thereof, those exemplified for the substituent W may be applied.

R^{a} and R^{b} are preferably an alkyl group, an aryl group or an aromatic heterocyclic group. R^{a} and R^{b} are particularly preferably an alkyl group, an alkylene group capable of forming a ring upon being linked to L or an aryl group, more preferably an alkyl group having 1 to 8 carbon atoms, an alkylene group capable of forming a 5-membered to 6-membered ring upon being linked to L or a substituted or unsubstituted phenyl group, and still more preferably an alkyl group having 1 to 8 carbon atom, or a substituted or unsubstituted phenyl group.

When R^{a} and R^{b} are a substituent (preferably, an alkyl group, an alkenyl group or a group having these groups as a substituent), these groups may be combined with a hydrogen atom or a substituent of an aromatic ring (preferably a benzene ring) structure of an aryl group in which —NR^{a}(R^{b}) is substituted, to form a ring (preferably a 6-membered ring). In this case, it is preferred that the substituent is represented by Formula (VIII), (IX) or (X) to be described below.

Substituents out of R^{a} and R^{b} may be bound with each other to form a ring (examples of the ring formed include the ring R to be described below. Preferably a 5-membered ring or a 6-membered ring, and more preferably a 6-membered ring), and R^{a} and R^{b} each may be bound with a substituent in L (representing one of L₁, L₂ and L₃) to form a ring (preferably a 5-membered ring or a 6-membered ring, and more preferably a 6-membered ring).

It is preferred that D₁ is an aryl group (preferably a phenyl group) of which the para-position is substituted with an amino group. In this case, it is preferred that D₁ is represented by the following Formula (II). The amino group may be substituted. Examples of the amino group include the substituent W, and the amino group is preferably an aliphatic hydrocarbon group (preferably an alkyl group which may have a substituent), an aryl group (preferably a phenyl group which may have a substituent) or a heterocyclic group. The amino group is preferably a so-called diaryl group-substituted amino group, which is substituted with two aryl groups, and in this case, is preferably represented by the following Formula (III). Further, the substituent (preferably an alkyl group or an alkenyl group which may have a substituent) of the amino group may be bound with a hydrogen atom or a substituent of the aromatic ring (preferably a benzene ring) structure of an aryl group to form a ring (Examples of the ring formed include the ring R to be described. Preferably a 6-membered ring).

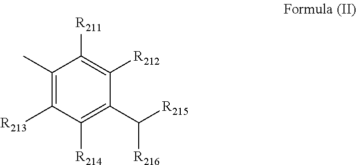

In the formula, R₂₁₁ to R₂₁₆ each independently represent a hydrogen atom or a substituent. Further, R₂₁₁ and R₂₁₂, R₂₁₃ and R₂₁₄, R₂₁₅ and R₂₁₆, R₂₁₂ and R₂₁₅, and R₂₁₄ and R₂₁₆ may be bound with each other to form a ring.

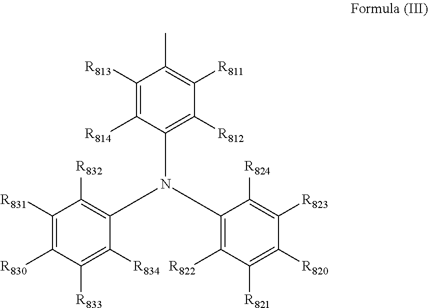

In the formula, R₈₁₁ to R₈₁₄, R₈₂₀ to R₈₂₄ and R₈₃₀ to R₈₃₄ each independently represent a hydrogen atom or a substituent. In addition, at least two of R₈₁₁ to R₈₁₄, R₈₂₀ to R₈₂₄ and R₈₃₀ to R₈₃₄ may be bound with each other to form a ring.

It is also preferred that D₁ is represented by the following Formula (VII).

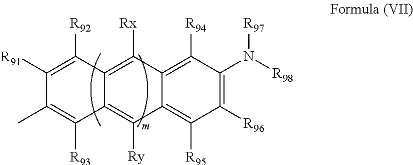

In the formula, R₉₁ to R₉₈ each independently represent a hydrogen atom or a substituent. m represents an integer of 0 or more. Rx and Ry each independently represent a hydrogen atom or a substituent, and when m is 2 or more, Rx and Ry, each of which is bonded to a 6-membered ring, may be other substituents. Further, R₉₁ and R₉₂, R₉₂ and Rx, Rx and R₉₄, R₉₄ and R₉₇, R₉₃ and Ry, Ry and R₉₅, R₉₅ and R₉₆, and R₉₇ and R₉₈ may each independently form a ring. Further, the bonding part with L₃ (L₁ when n₁ is 0) may be at the position of R₉₁, R₉₂ and R₉₃, and in that case, a substituent corresponding to each of R₉₁, R₉₂ and R₉₃ or a hydrogen atom may be bonded to a site which is indicated as the bonding part with L₃ in Formula (VII), and adjacent R's may be bound with each other to form a ring. Here, “adjacent R's may be bound with each other to form a ring” means that for example, when R₉₁ is a bonding part with L₃ (L₁ when n₁ is 0), R₉₀ and R₉₃ may be bound with each other to form a ring if R₉₀ is bonded to a bonding part in Formula (VII), when R₉₂ is a bonding part with L₃ (L₁ when n₁ is 0), R₉₀ and R₉₁, and R₉₀ and R₉₃ may be bound with each other to form a ring, respectively, if R₉₀ is bonded to a bonding part in Formula (VIII), and when R₉₃ is a bonding part with L₃ (L₁ when n₁ is 0), R₉₀ and R₉₁, and R₉₁ and R₉₂ may be bound with each other to form a ring, respectively, if R₉₀ is bonded to a bonding part in Formula (VII).

It is preferred that the aforementioned ring is a benzene ring.

Examples of the substituent of R₉₁ to R₉₈, Rx and Ry include the substituent W. It is preferred that any one of R₉₁ to R₉₆ is a hydrogen atom, and it is preferred that any one of Rx and Ry is a hydrogen atom. It is preferred that R₉₁ to R₉₆ are a hydrogen atom, and Rx and Ry are also a hydrogen atom.

It is preferred that R₉₇ and R₉₈ each independently represent a phenyl group which may be substituted, and examples of the substituent include the substituent W, and the substituent is preferably an unsubstituted phenyl group.

m represents an integer of 0 or more, and is preferably 0 or 1.

It is also preferred that D₁ is a group represented by Formula (VIII), (IX) or (X).

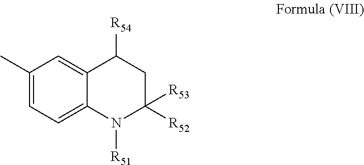

In the formula, R₅₁ to R₅₄ each independently represent hydrogen or a substituent. Examples of the substituent include the substituent W. R₅₂ and R₅₃, and R₅₁ and R₅₂ may be linked to each other to form a ring, respectively.

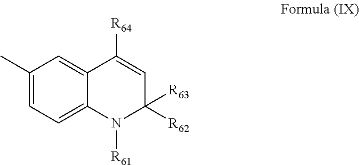

In the formula, R₆₁ to R₆₄ each independently represent hydrogen or a substituent. Examples of the substituent include the substituent W. R₆₂ and R₆₃, and R₆₁ and R₆₂ may be linked to each other to form a ring, respectively.

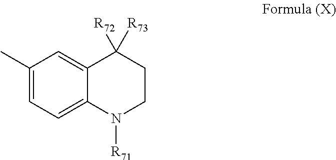

In the formula, R₇₁ to R₇₃ each independently represent hydrogen or a substituent. Examples of the substituent include the substituent W. R₇₂ and R₇₃ may be linked to each other to form a ring.

In D₁, the group represented by Formula (II) or (III) is more preferably used.

In Formula (II), R₂₁₁ to R₂₁₆ each independently represent a hydrogen atom or a substituent. In addition, R₂₁₁ and R₂₁₂, R₂₁₃ and R₂₁₄, R₂₁₅ and R₂₁₆, R₂₁₂ and R₂₁₅, and R₂₁₄ and R₂₁₆ may be bound with each other to form a ring, respectively. Examples of the ring formed include the ring R to be described below.

Examples of the substituent in R₂₁₁ to R₂₁₄ include the substituent W, and it is preferred that R₂₁₁ to R₂₁₄ are a hydrogen atom, or R₂₁₂ and R₂₁₅ or R₂₁₄ and R₂₁₆ form a 5-membered ring, and it is more preferred that any one of R₂₁₁ to R₂₁₄ is a hydrogen atom.

Examples of the substituent in R₂₁₅ and R₂₁₆ include the substituent W, and among the substituent, a substituted or unsubstituted aryl group is preferred, and as a substituent of the substituted aryl group, an alkyl group (for example, a methyl group and an ethyl group) and an aryl group (for example, a phenyl group, a naphthalene group, a phenanthryl group and an anthryl group) are preferred. R₂₁₅ and R₂₁₆ are preferably a phenyl group, an alkyl substituted phenyl group, a phenyl substituted phenyl group, a naphthyl group, a phenanthryl group, an anthryl group or a fluorenyl group (preferably a 9,9′-dimethyl-2-fluorenyl group).

In Formula (III), R₈₁₁ to R₈₁₄, R₈₂₀ to R₈₂₄ and R₈₃₀ to R₈₃₄ independently represent a hydrogen atom or a substituent. Further, R₈₁₁ to R₈₁₄, R₈₂₀ to R₈₂₄ and R₈₃₀ to R₈₃₄ may be bound with each other to form a ring. Examples of the ring formed include the ring R to be described below. Examples of forming the ring include the case where R₈₁₁ and R₈₁₂, and R₈₁₃ and R₈₁₄ are bound with each other to form a benzene ring, adjacent two (R₈₂₄ and R₈₂₃, R₈₂₃ and R₈₂₀, R₈₂₀ and R₈₂₁, and R₈₂₁ and R₈₂₂) of R₈₂₀ to R₈₂₄ are bound with each other to form a benzene ring, adjacent two (R₈₃₄ and R₈₃₃, R₈₃₃ and R₈₃₀, R₈₃₀ and R₈₃₁, and R₈₃₁ and R₈₃₂) of R₈₃₀ to R₈₃₄ are bound with each other to form a benzene ring, and R₈₂₂ and R₈₃₄ are bound with each other to form a 5-membered ring with an N atom.

Examples of the substituent represented by R₈₁₁ to R₈₁₄, R₈₂₀ to R₈₂₄, and R₈₃₀ to R₈₃₄ include the substituent W, and the substituent is preferably an alkyl group (for example, a methyl group and an ethyl group), an aryl group (for example, a phenyl group and a naphthyl group), and the substituent W (preferably an aryl group) in these groups may also be substituted. Among them, it is preferred that R₈₂₀ and R₈₃₀ are a substituent, and it is more preferred that the other R₈₁₁ to R₈₁₄, R₈₂₁ to R₈₂₄, and R₈₃₁ to R₈₃₄ are a hydrogen atom.

The compound represented by Formula (I) is preferably a compound represented by the following Formula (pI).

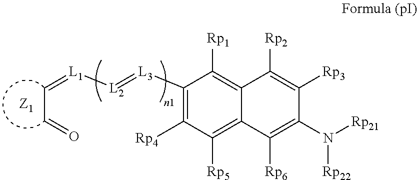

In the formula, Z₁ is a ring including at least two carbon atoms, and represents a 5-membered ring, a 6-membered ring or a condensed ring including at least one of the 5-membered ring and the 6-membered ring. L₁, L₂ and L₃ each independently represent an unsubstituted methine group or a substituted methine group. n₁ represents an integer of 0 or more. Rp₁, Rp₂, Rp₃, Rp₄, Rp₅ and Rp₆ each independently represent a hydrogen atom or a substituent. Rp₁ and Rp₂, Rp₂ and Rp₃, Rp₄ and Rp₅, and Rp₅ and Rp₆ may be bound with each other to form a ring, respectively. Rp₂₁ and Rp₂₂ each independently represent a substituted aryl group, an unsubstituted aryl group, a substituted heteroaryl group or an unsubstituted heteroaryl group.

It is possible to obtain photoelectric conversion device having excellent heat resistance and fast responsiveness by using a compound in which a naphthylene group is disposed for a linking part of a donor part (the moiety of (—NRp₂₁Rp₂₂)/acceptor part (the moiety bonded to a naphthalene group through L₁ to L₃) as a photoelectric conversion material together with fullerenes, as described above. It is thought that by disposing a naphthylene group for the linking part of donor part/acceptor part, interaction with fullerenes is enhanced and the response speed is improved. Further, the compound has sufficient sensitivity.

In Formula (pI), Z₁, L₁, L₂, L₃ and n₁ have the same meaning as Z₁, L₁, L₂, L₃ and n₁ in Formula (I), and preferred ranges thereof are also the same.

Rp₁ to Rp₆ independently represent a hydrogen atom or a substituent. When Rp₁ to Rp₆ represent a substituent, examples of the substituent which Rp₁ to Rp₆ represent include the substituent W to be described below, and a halogen atom, an alkyl group, an aryl group, a heterocyclic group, a hydroxyl group, a nitro group, an alkoxy group, an aryloxy group, a heterocyclic oxy group, an amino group, an alkylthio group, an arylthio group, an alkenyl group, a cyano group and a heterocyclic thio group are preferred.

Rp₁ to Rp₆ are preferably independently a hydrogen atom, a halogen atom, an alkyl group, an aryl group, a heterocyclic group, a hydroxyl group, a nitro group, an alkoxy group, an aryloxy group, a heterocyclic oxy group, an amino group, an alkylthio group, an arylthio group, an alkenyl group, a cyano group or a heterocyclic thio group, more preferably a hydrogen atom, an alkyl group, an aryl group and a heterocyclic group, still more preferably a hydrogen atom, an alkyl group having 1 to 20 carbon atoms, an aryl group having 6 to 20 carbon atoms and a heterocyclic group having 4 to 16 carbon atoms, further preferably a hydrogen atom, an alkyl group having 1 to 12 carbon atoms and an aryl group having 6 to 14 carbon atoms, further more preferably a hydrogen atom, an alkyl group having 1 to 6 carbon atoms and an aryl group having 6 to 10 carbon atoms, and particularly preferably a hydrogen atom. The alkyl group may be branched. In addition, when Rp₁ to Rp₆ are a substituent, Rp₁ to Rp₆ may further have a substituent. Examples of the further substituent include the substituent to be described below. In the case of a plurality of the further substituents, the plurality of the substituents may be linked to each other to form a ring. Examples of the ring formed include the ring R to be described below.

Preferred specific examples of Rp₁ to Rp₆ include a hydrogen atom, a methyl group, an ethyl group, a propyl group, a butyl group, a hexyl group, a cyclohexyl group, a phenyl group and a naphthyl group.

Rp₁ and Rp₂, Rp₂ and Rp₃, Rp₄ and Rp₅, and Rp₅ and Rp₆ may be bound with each other to form a ring. Examples of the ring formed include the ring R to be described below. The ring is preferably a benzene ring, a naphthalene ring, an anthracene ring, a pyridine ring, a pyrimidine ring and the like.

Rp₂₁ and Rp₂₂ each independently represent a substituted aryl group, an unsubstituted aryl group, a substituted heteroaryl group or an unsubstituted heteroaryl group. It is preferred that both Rp21 and Rp22 are not an unsubstituted phenyl group.

The aryl group represented by Rp₂₁ and Rp₂₂ is preferably an aryl group having 6 to 30 carbon atoms, and more preferably an aryl group having 6 to 20 carbon atoms. Specific examples of the aryl group include a phenyl group, a naphthyl group, a biphenyl group, a terphenyl group, an anthryl group and a fluorenyl group.

The substituent of the substituted aryl group in Rp₂₁ and Rp₂₂ is preferably an alkyl group (for example, a methyl group, an ethyl group and a t-butyl group), an alkoxy group (for example, a methoxy group, an ethoxy group and an isopropoxy group), an aryl group (for example, a phenyl group, a naphthyl group, a phenanthryl group and an anthryl group), and a heteroaryl group (for example, a thienyl group, a furanyl group, a pyridyl group and a carbazolyl group).

The aryl group or the substituted aryl group, which Rp₂₁ and Rp₂₂ represent, is preferably a phenyl group, a substituted phenyl group, a biphenyl group, a naphthyl group, a phenanthryl group, an anthryl group, a fluorenyl group and a substituted fluorenyl group (preferably a 9,9′-dialkyl-2-fluorenyl group).

When Rp₂₁ and Rp₂₂ are a heteroaryl group, the heteroaryl group is preferably a heteroaryl group composed of a 5-, 6- or 7-membered ring or a condensed ring thereof. Examples of the heteroatom included in the heteroaryl group include an oxygen atom, a sulfur atom and a nitrogen atom. Specific examples of the ring constituting the heteroaryl group include a furan ring, a thiophene ring, a pyrrole ring, a pyrroline ring, a pyrrolidine ring, an oxazole ring, an isoxazole ring, a thiazole ring, an isothiazole ring, an imidazole ring, an imidazoline ring, an imidazolidine ring, a pyrazole ring, a pyrazoline ring, a pyrazolidine ring, a triazole ring, a furazan ring, a tetrazole ring, a pyran ring, a thiin ring, a pyridine ring, a piperidine ring, an oxazine ring, a morpholine ring, a thiazine ring, a pyridazine ring, a pyrimidine ring, a pyrazine ring, a piperazine ring, a triazine ring and the like.

Examples of the condensed ring include a benzofuran ring, an isobenzofuran ring, a benzothiophene ring, an indole ring, an indoline ring, an isoindole ring, a benzoxazole ring, a benzothiazole ring, an indazole ring, a benzimidazole ring, a quinoline ring, an isoquinoline ring, a cinnoline ring, a phthalazine ring, a quinazoline ring, a quinoxaline ring, a dibenzofuran ring, a carbazole ring, a xanthene ring, an acridine ring, a phenanthridine ring, a phenanthroline ring, a phenazine ring, a phenoxazine ring, a thianthrene ring, a thienothiophene ring, an indolizine ring, a quinolizine ring, a quinuclidine ring, a naphthyridine ring, a purine ring, a pteridine ring and the like.

The substituent of the substituted heteroaryl group in Rp₂₁ and Rp₂₂ is preferably an alkyl group (for example, a methyl group, an ethyl group and a t-butyl group), an alkoxy group (for example, a methoxy group, an ethoxy group and an isopropoxy group), an aryl group (for example, a phenyl group, a naphthyl group, a phenanthryl group and an anthryl group), and a heteroaryl group (for example, a thienyl group, a furanyl group, a pyridyl group and a carbazolyl group).

The ring constituting the heteroaryl group or the substituted heteroaryl group represented by Rp₂₁ and Rp₂₂ is preferably a thiophene ring, a substituted thiophene ring, a furan ring, a substituted furan ring, a thienothiophene ring, a substituted thienothiophene ring and a carbazolyl group.

Rp₂₁ and Rp₂₂ are each independently, preferably a phenyl group, a naphthyl group, a fluorenyl group, a biphenyl group, an anthracenyl group and a phenanthrenyl group, and more preferably a phenyl group, a naphthyl group or a fluorenyl group. When Rp₂₁ and Rp₂₂ has a substituent, the substituent is preferably an alkyl group, an alkyl halide group, an alkoxy group, an aryl group or a heteroaryl group, and more preferably a methyl group, an isopropyl group, a t-butyl group, a trifluoromethyl group, a phenyl group or a carbazolyl group.

When Z₁ is the group represented by Formula (VI) or the group represented by Formula (VII), the compound represented by Formula (pI) is a compound represented by the following Formula (pII) or a compound represented by the following Formula (pIII), respectively.

The compound represented by Formula (pI) is preferably a compound represented by the following Formula (pII) or a compound represented by the following Formula (pIII).

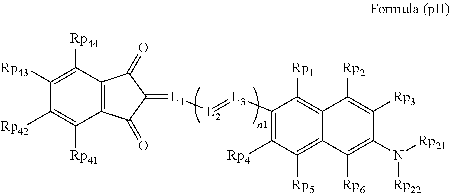

In the formula, L₁, L₂, L₃, n₁, Rp₁, Rp₂, Rp₃, Rp₄, Rp₅, Rp₆, Rp₂₁ and Rp₂₂ have the same meaning as in Formula (pI), and preferred ranges are also the same. Rp₄₁, Rp₄₂, Rp₄₃ and Rp₄₄ have the same meaning as R₄₁, R₄₂, R₄₃ and R₄₄ in Formula (IV), and preferred ranges are also the same.

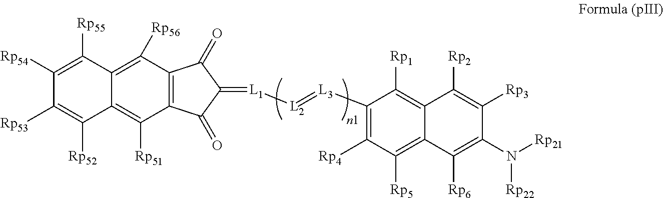

In the formula, L₁, L₂, L₃, n₁, Rp₁, Rp₂, Rp₃, Rp₄, Rp₅, Rp₆, Rp₂₁ and Rp₂₂ have the same meaning as in Formula (pI), and preferred ranges are also the same. Rp₅₁, Rp₅₂, Rp₅₃, Rp₅₄, Rp₅₅ and Rp₅₆ have the same meaning in R₄₁, R₄₄, R₄₅, R₄₆, R₄₇ and R₄₈ in Formula (V), and preferred ranges thereof are also the same.

The compound represented by Formula (pI) is preferably a compound represented by the following Formula (pIV).

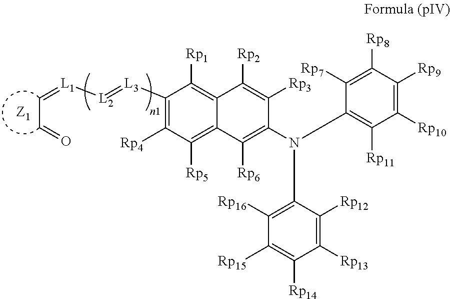

In the formula, Z₁, L₁, L₂, L₃, n₁, Rp₁, Rp₂, Rp₃, Rp₄, Rp₅ and Rp₆ have the same meaning as in Formula (pI), and preferred ranges are also the same.

Rp₇ to Rp₁₁ and Rp₁₂ to Rp₁₆ each independently represent a hydrogen atom or a substituent. However, the case where all of Rp₇ to Rp₁₁ and Rp₁₂ to Rp₁₆ are a hydrogen atom is excluded. Further, adjacent members out of Rp₇ to Rp₁₁ and Rp₁₂ to Rp₁₆ may be bound with each other to form a ring. In addition, Rp₃ and Rp₇, and Rp₆ and Rp₁₆ may be linked to each other, respectively.

In Formula (pIV), Rp₇ to Rp₁₁ and Rp₁₂ to Rp₁₆ each independently represent a hydrogen atom or a substituent. However, there is no case where all of Rp₇ to Rp₁₁ and Rp₁₂ to Rp₁₆ are a hydrogen atom. Further, when Rp₃ and Rp₇, or Rp₆ and Rp₁₆ are linked, all the other members Rp₈ to Rp₁₁ and Rp₁₂ to Rp₁₅ may be a hydrogen atom.

When Rp₇ to Rp₁₁ and Rp₁₂ to Rp₁₆ represent a substituent, examples of the substituent which Rp₇ to Rp₁₁, and Rp₁₂ to Rp₁₆ represent include the substituent W to be described below, and particularly, a halogen atom, an alkyl group, an aryl group, a heterocyclic group, a hydroxyl group, a nitro group, an alkoxy group, an aryloxy group, a heterocyclic oxy group, an amino group, an alkylthio group, an arylthio group, an alkenyl group, a cyano group and a heterocyclic thio group are preferred.

Rp₇ to Rp₁₁ and Rp₁₂ to Rp₁₆ are each independently, preferably a hydrogen atom, a halogen atom, an alkyl group, an aryl group, a heterocyclic group, a hydroxyl group, a nitro group, an alkoxy group, an aryloxy group, a heterocyclic oxy group, an amino group, an alkylthio group, an arylthio group, an alkenyl group, a cyano group or a heterocyclic thio group, more preferably a hydrogen atom, an alkyl group, an alkenyl group, an alkoxy group, an aryl group, an aryloxy group and a heterocyclic group, more preferably a hydrogen atom, an alkyl group having 1 to 20 carbon atoms, an alkenyl group having 2 to 20 carbon atoms, an alkoxy group having 1 to 20 carbon atoms, an aryl group having 6 to 20 carbon atoms, an aryloxy group having 6 to 20 carbon atoms, and a heterocyclic group composed of a 5-, 6- or 7-membered ring or a condensed ring thereof, and still more preferably a hydrogen atom, an alkyl group having 1 to 12 carbon atoms, an alkenyl group having 2 to 12 carbon atoms, an alkyloxy group having 1 to 12 carbon atoms, an aryl group having 6 to 10 carbon atoms, an aryloxy group having 6 to 10 carbon atoms and a heterocyclic group composed of a 5- or 6-membered ring or a condensed ring thereof.

In the case of an alkyl group, the alkyl group may be either straight or branched. Examples of the heteroatom included in the heterocyclic group include an oxygen atom, a sulfur atom, a nitrogen atom and the like.

Specific examples of an alkyl group, an alkenyl group, an aryl group and the like include the groups illustrated in an alkyl group, an alkenyl group and an aryl group of the substituent W to be described below.

Further, adjacent members out of Rp₇ to Rp₁₁ and Rp₁₂ to Rp₁₆ may be bound with each other to form a ring. Examples of the ring formed include the ring R to be described below. The ring formed is preferably a benzene ring, a naphthalene ring, an anthracene ring, a pyridine ring, a pyrimidine ring and the like.

In addition, Rp₃ and Rp₇, or Rp₆ and Rp₁₆ may be linked to each other. When Rp₃ and Rp₇, or Rp₆ and Rp₁₆ are linked, a condensed ring composed of four or more rings including a naphthylene group and a phenyl group is formed. The linkage between Rp₃ and Rp₇ or between Rp₆ and Rp₁₆ may be a single bond.

The compound represented by Formula (I) may be prepared in accordance with the synthesis method described in the Japanese Patent Application Laid-Open No. 2000-297068. After the synthesis, a high-purity material for organic electronics (here, a photoelectric conversion material) may be obtained with high yield in a short period of time by purification using the purification method of the present invention.

Hereinafter, specific examples of the compound represented by Formula (I) will be described, but the present invention is not limited thereto.

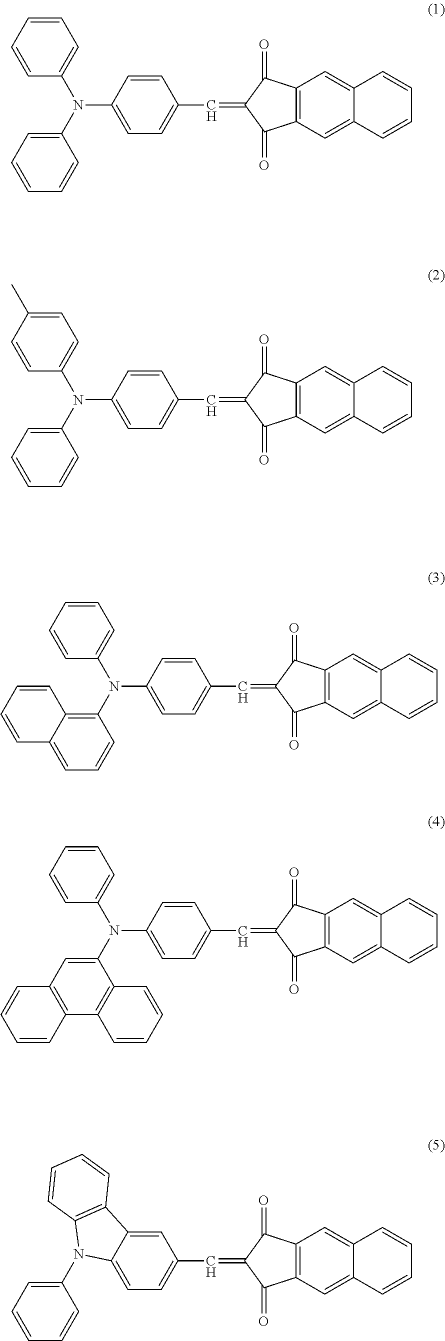 [SMILES: O=C1C(=Cc2ccc(N(c3ccccc3)c3ccccc3)cc2)C(=O)c2cc3ccccc3cc21 ; Cc1ccc(N(c2ccccc2)c2ccc(C=C3C(=O)c4cc5ccccc5cc4C3=O)cc2)cc1 ; O=C1C(=Cc2ccc(N(c3ccccc3)c3cccc4ccccc34)cc2)C(=O)c2cc3ccccc3cc21 ; O=C1C(=Cc2ccc(N(c3ccccc3)c3cc4ccccc4c4ccccc34)cc2)C(=O)c2cc3ccccc3cc21 ; O=C1C(=Cc2ccc3c(c2)c2ccccc2n3-c2ccccc2)C(=O)c2cc3ccccc3cc21]

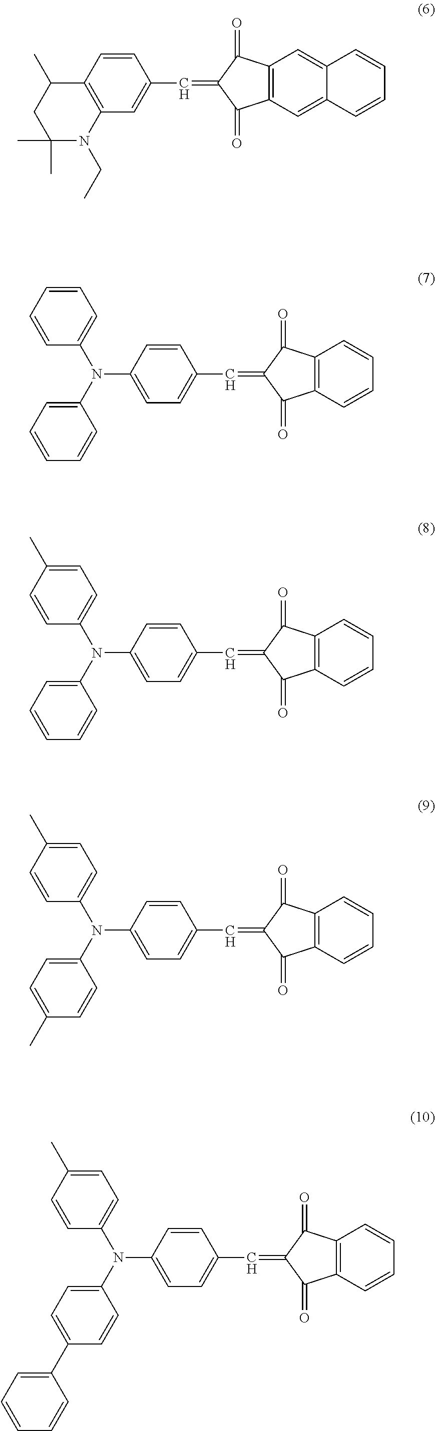 [SMILES: CCN1c2cc(C=C3C(=O)c4cc5ccccc5cc4C3=O)ccc2C(C)CC1(C)C ; O=C1C(=Cc2ccc(N(c3ccccc3)c3ccccc3)cc2)C(=O)c2ccccc21 ; Cc1ccc(N(c2ccccc2)c2ccc(C=C3C(=O)c4ccccc4C3=O)cc2)cc1 ; Cc1ccc(N(c2ccc(C)cc2)c2ccc(C=C3C(=O)c4ccccc4C3=O)cc2)cc1 ; Cc1ccc(N(c2ccc(C=C3C(=O)c4ccccc4C3=O)cc2)c2ccc(-c3ccccc3)cc2)cc1]

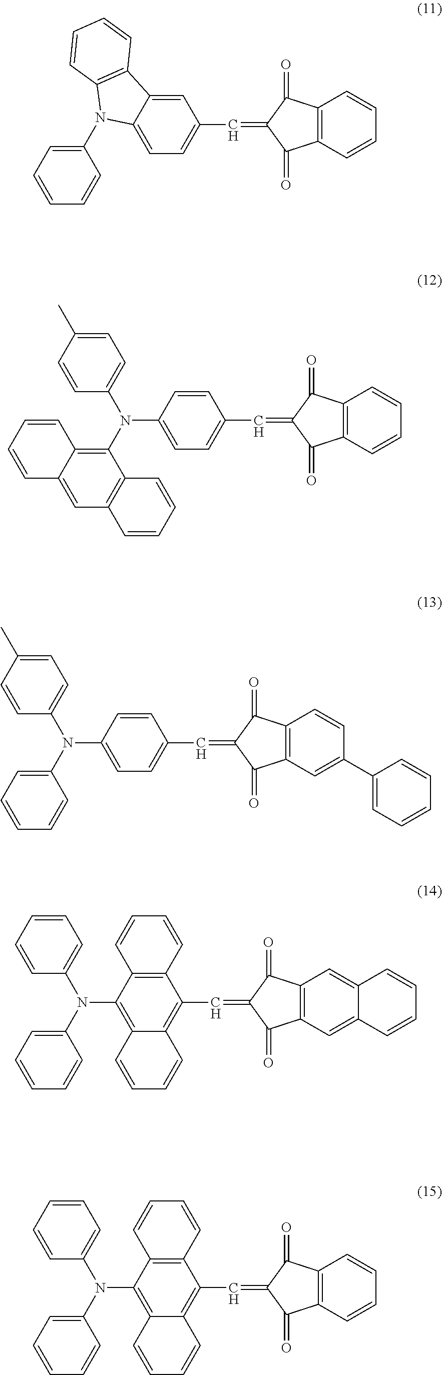 [SMILES: O=C1C(=Cc2ccc3c(c2)c2ccccc2n3-c2ccccc2)C(=O)c2ccccc21 ; Cc1ccc(N(c2ccc(C=C3C(=O)c4ccccc4C3=O)cc2)c2c3ccccc3cc3ccccc23)cc1 ; Cc1ccc(N(c2ccccc2)c2ccc(C=C3C(=O)c4ccc(-c5ccccc5)cc4C3=O)cc2)cc1 ; O=C1C(=Cc2c3ccccc3c(N(c3ccccc3)c3ccccc3)c3ccccc23)C(=O)c2cc3ccccc3cc21 ; O=C1C(=Cc2c3ccccc3c(N(c3ccccc3)c3ccccc3)c3ccccc23)C(=O)c2ccccc21]

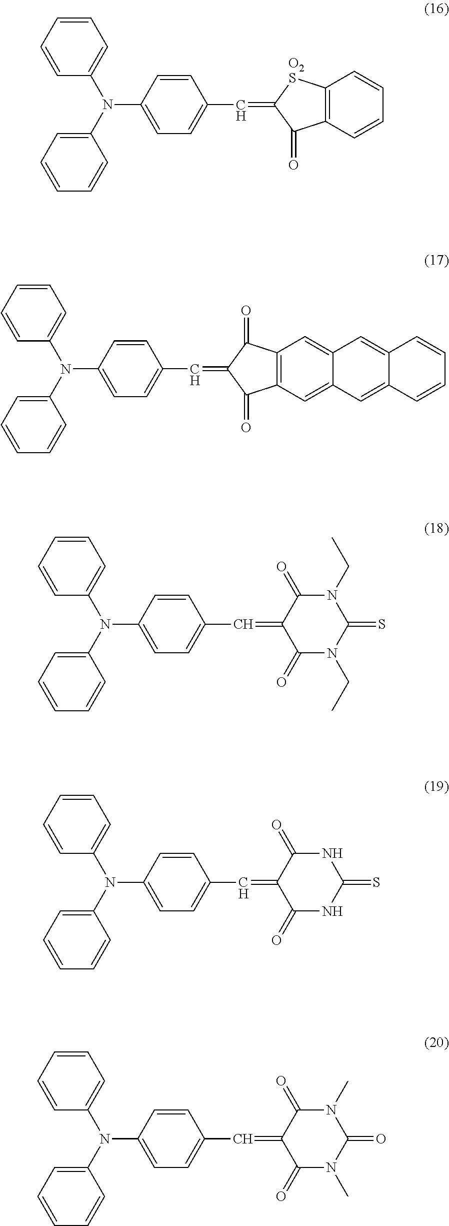 [SMILES: O=C1C(=Cc2ccc(N(c3ccccc3)c3ccccc3)cc2)S(=O)(=O)c2ccccc21 ; O=C1C(=Cc2ccc(N(c3ccccc3)c3ccccc3)cc2)C(=O)c2cc3cc4ccccc4cc3cc21 ; CCN1C(=O)C(=Cc2ccc(N(c3ccccc3)c3ccccc3)cc2)C(=O)N(CC)C1=S ; O=C1NC(=S)NC(=O)C1=Cc1ccc(N(c2ccccc2)c2ccccc2)cc1 ; CN1C(=O)C(=Cc2ccc(N(c3ccccc3)c3ccccc3)cc2)C(=O)N(C)C1=O]

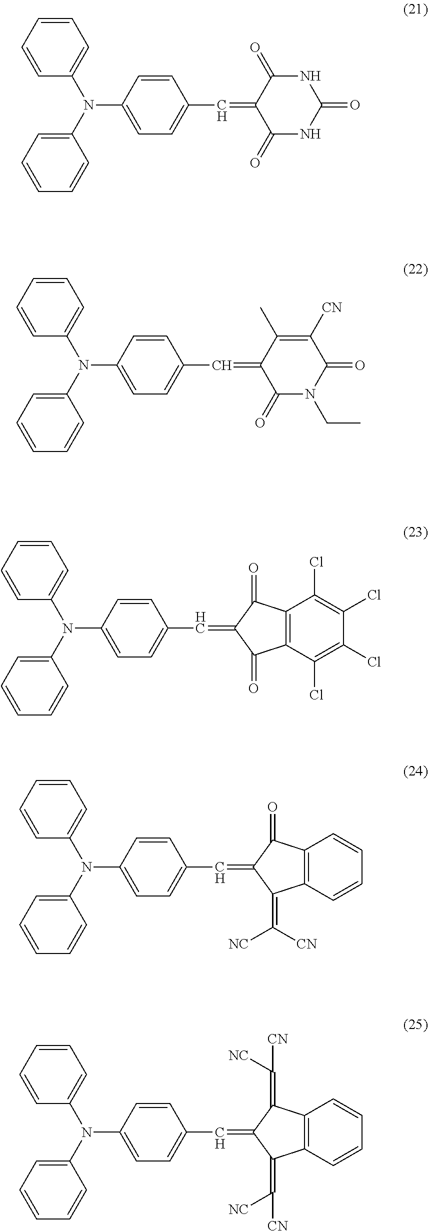 [SMILES: O=C1NC(=O)C(=Cc2ccc(N(c3ccccc3)c3ccccc3)cc2)C(=O)N1 ; CCN1C(=O)C(=Cc2ccc(N(c3ccccc3)c3ccccc3)cc2)C(C)=C(C#N)C1=O ; O=C1C(=Cc2ccc(N(c3ccccc3)c3ccccc3)cc2)C(=O)c2c(Cl)c(Cl)c(Cl)c(Cl)c21 ; N#CC(C#N)=C1C(=Cc2ccc(N(c3ccccc3)c3ccccc3)cc2)C(=O)c2ccccc21 ; N#CC(C#N)=C1C(=Cc2ccc(N(c3ccccc3)c3ccccc3)cc2)C(=C(C#N)C#N)c2ccccc21]

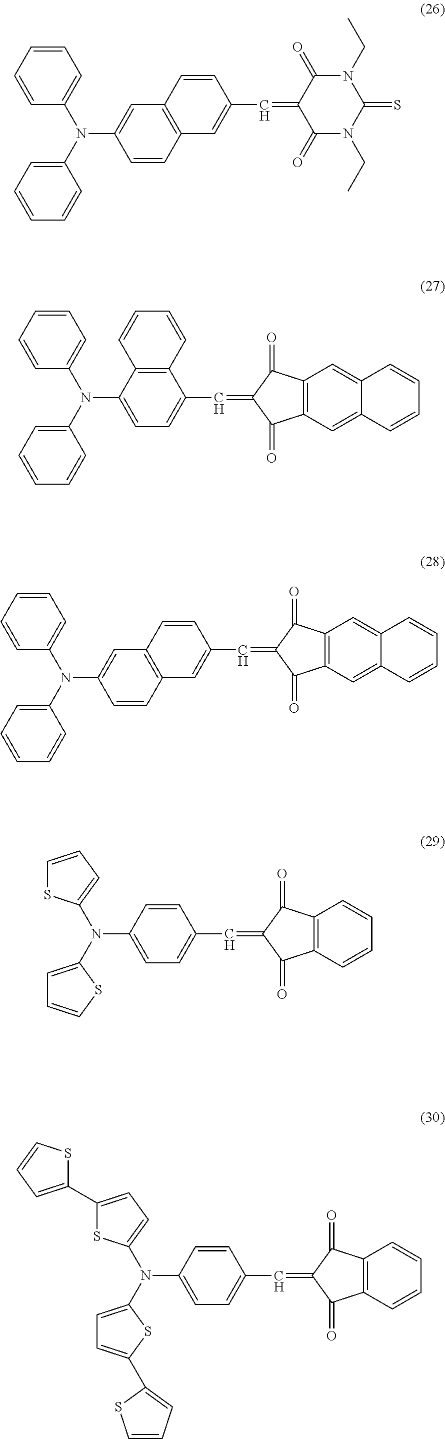 [SMILES: CCN1C(=O)C(=Cc2ccc3cc(N(c4ccccc4)c4ccccc4)ccc3c2)C(=O)N(CC)C1=S ; O=C1C(=Cc2ccc(N(c3ccccc3)c3ccccc3)c3ccccc23)C(=O)c2cc3ccccc3cc21 ; O=C1C(=Cc2ccc3cc(N(c4ccccc4)c4ccccc4)ccc3c2)C(=O)c2cc3ccccc3cc21 ; O=C1C(=Cc2ccc(N(c3cccs3)c3cccs3)cc2)C(=O)c2ccccc21 ; O=C1C(=Cc2ccc(N(c3ccc(-c4cccs4)s3)c3ccc(-c4cccs4)s3)cc2)C(=O)c2ccccc21]

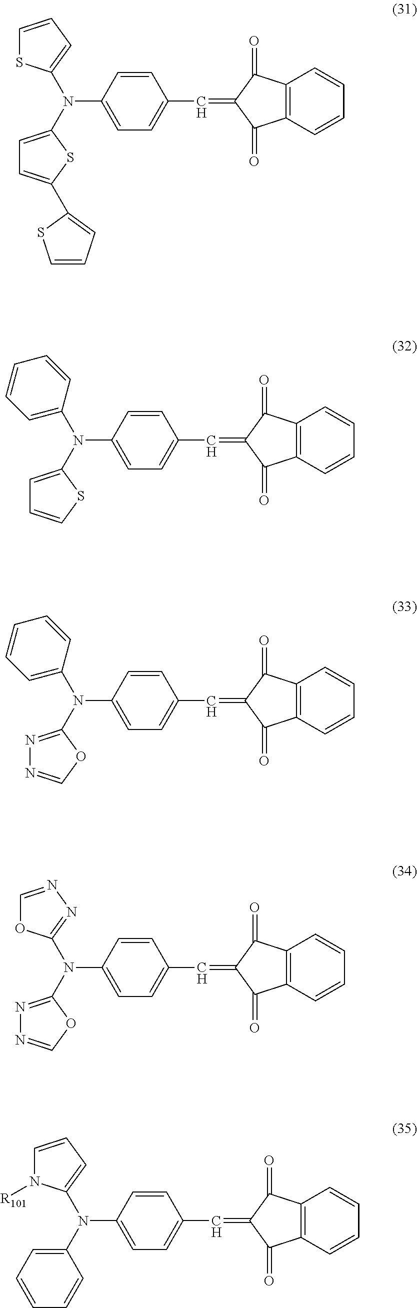

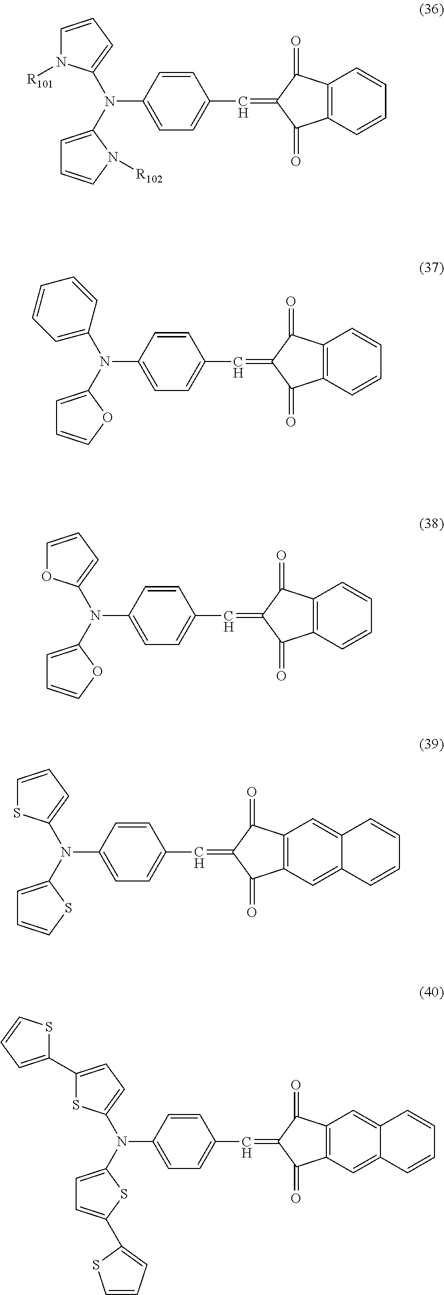

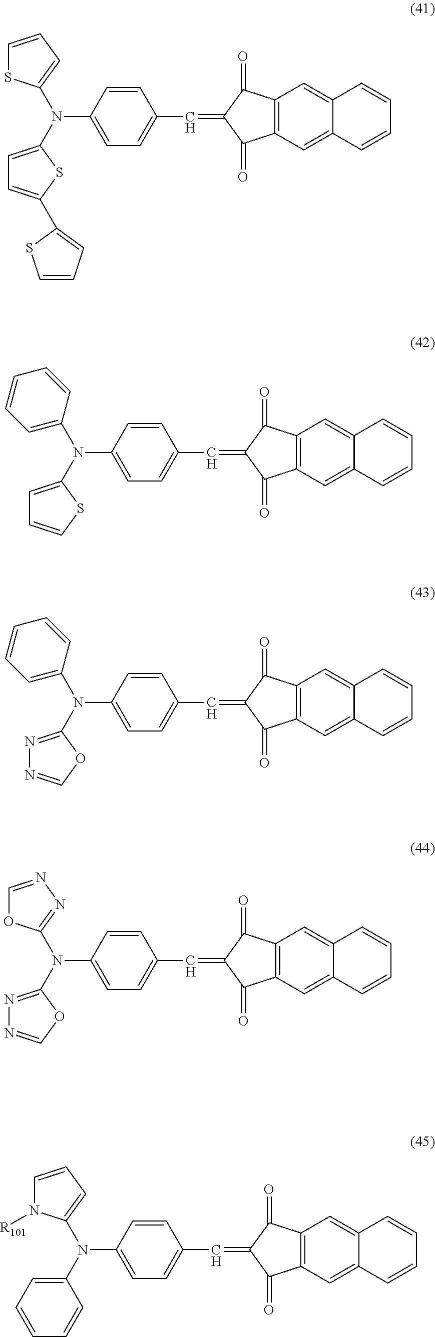

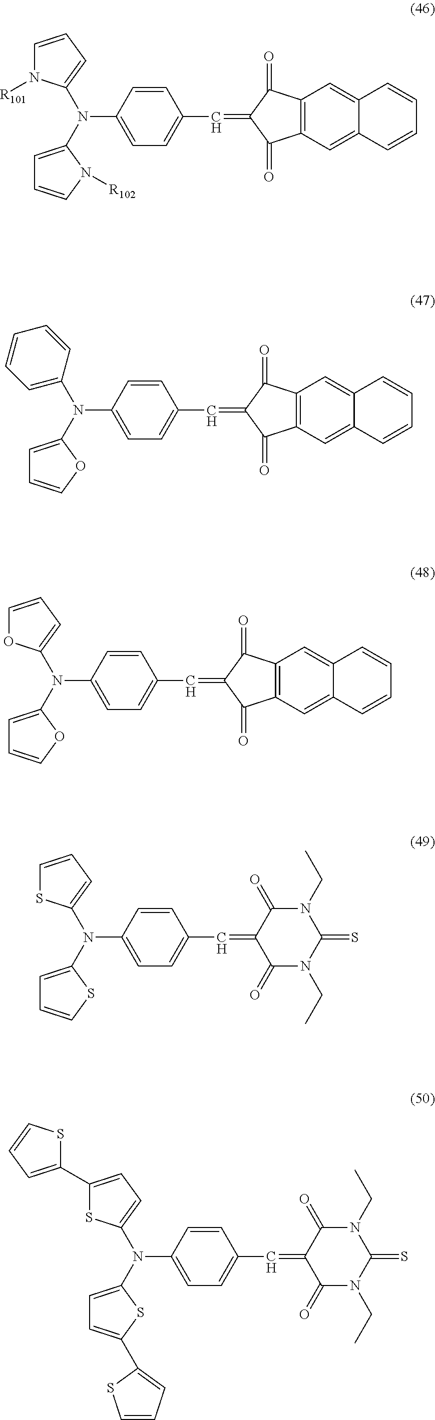

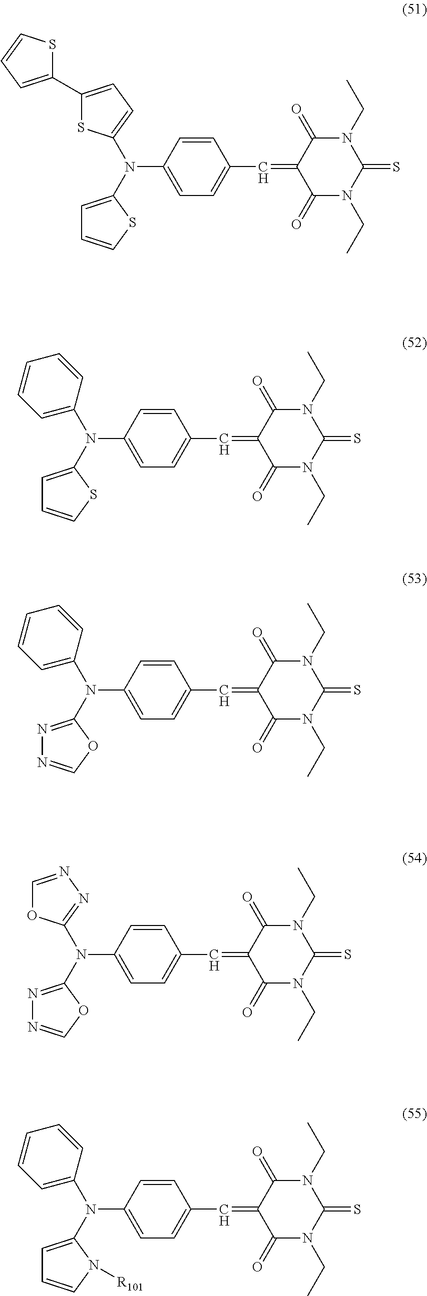

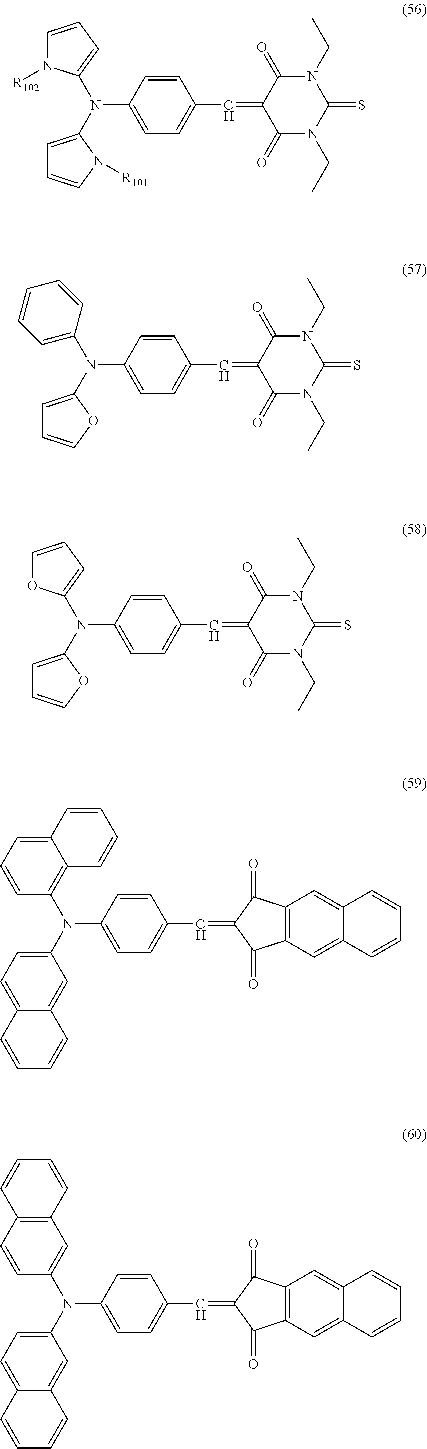

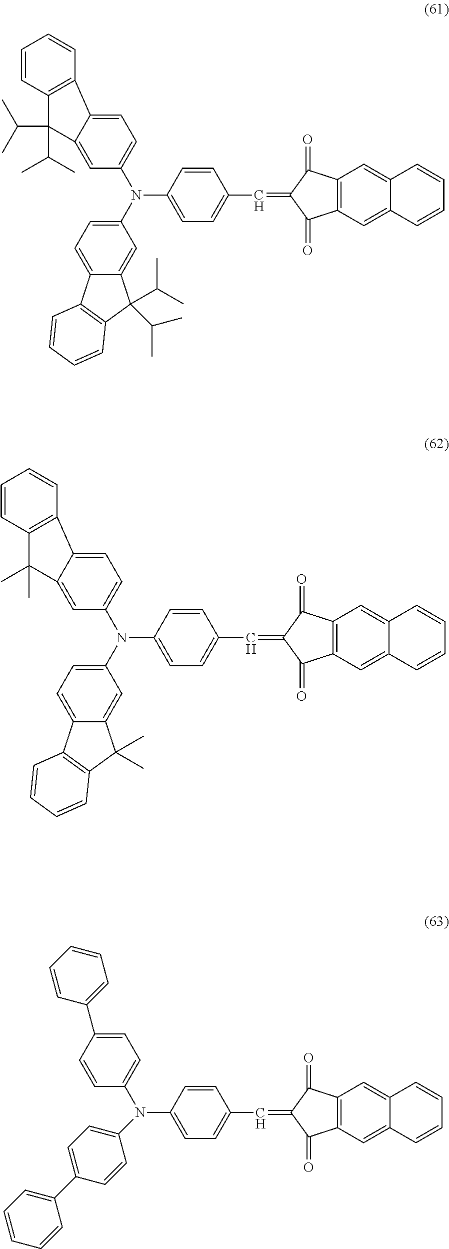 [SMILES: CC(C)C1(C(C)C)c2ccccc2-c2ccc(N(c3ccc(C=C4C(=O)c5cc6ccccc6cc5C4=O)cc3)c3ccc4c(c3)C(C(C)C)(C(C)C)c3ccccc3-4)cc21 ; CC1(C)c2ccccc2-c2ccc(N(c3ccc(C=C4C(=O)c5cc6ccccc6cc5C4=O)cc3)c3ccc4c(c3)C(C)(C)c3ccccc3-4)cc21 ; O=C1C(=Cc2ccc(N(c3ccc(-c4ccccc4)cc3)c3ccc(-c4ccccc4)cc3)cc2)C(=O)c2cc3ccccc3cc21]

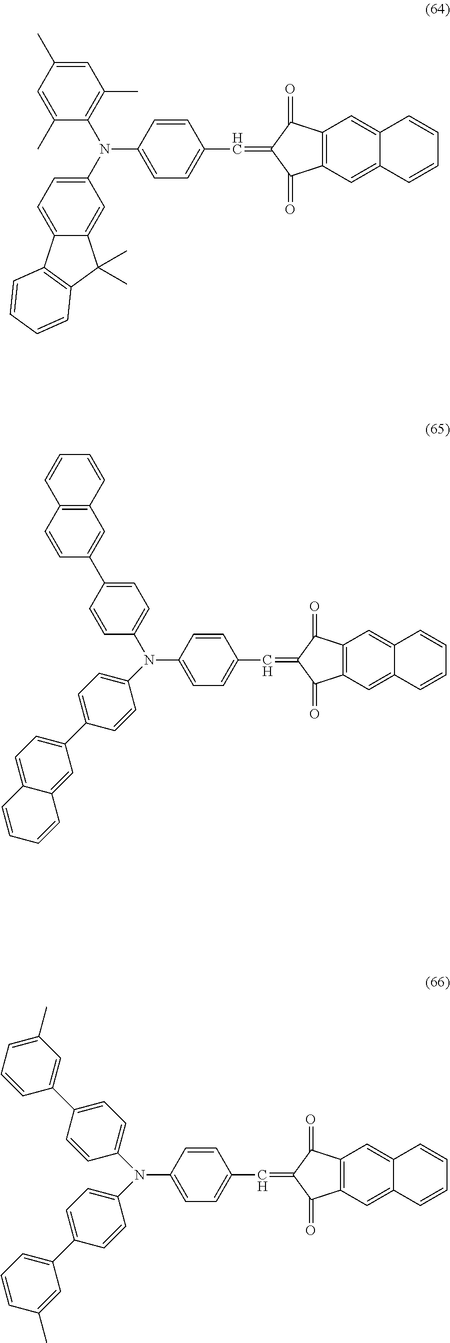 [SMILES: Cc1cc(C)c(N(c2ccc(C=C3C(=O)c4cc5ccccc5cc4C3=O)cc2)c2ccc3c(c2)C(C)(C)c2ccccc2-3)c(C)c1 ; O=C1C(=Cc2ccc(N(c3ccc(-c4ccc5ccccc5c4)cc3)c3ccc(-c4ccc5ccccc5c4)cc3)cc2)C(=O)c2cc3ccccc3cc21 ; Cc1cccc(-c2ccc(N(c3ccc(C=C4C(=O)c5cc6ccccc6cc5C4=O)cc3)c3ccc(-c4cccc(C)c4)cc3)cc2)c1]

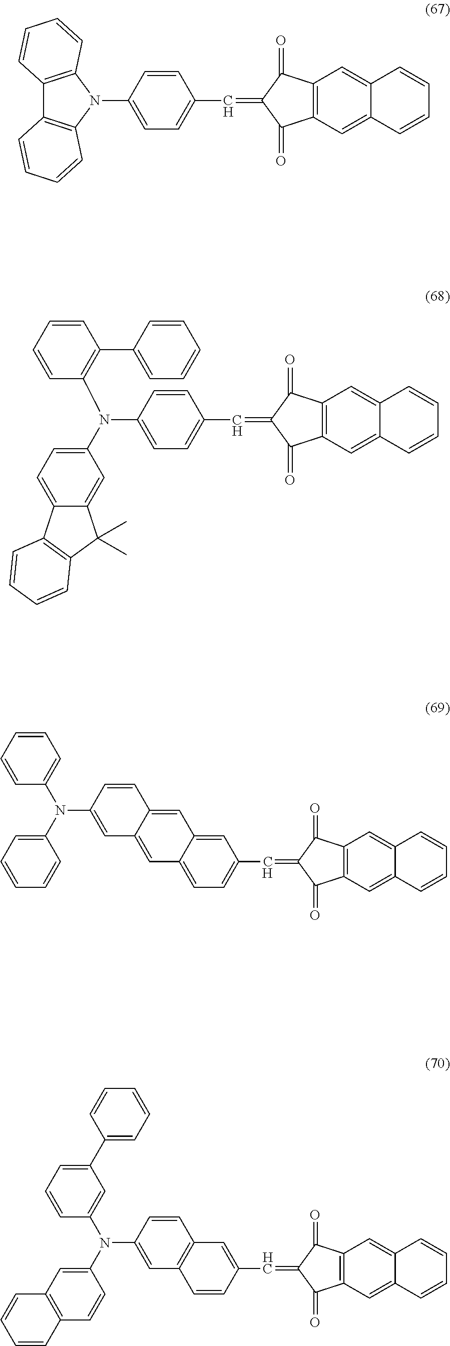 [SMILES: O=C1C(=Cc2ccc(-n3c4ccccc4c4ccccc43)cc2)C(=O)c2cc3ccccc3cc21 ; CC1(C)c2ccccc2-c2ccc(N(c3ccc(C=C4C(=O)c5cc6ccccc6cc5C4=O)cc3)c3ccccc3-c3ccccc3)cc21 ; O=C1C(=Cc2ccc3cc4cc(N(c5ccccc5)c5ccccc5)ccc4cc3c2)C(=O)c2cc3ccccc3cc21 ; O=C1C(=Cc2ccc3cc(N(c4cccc(-c5ccccc5)c4)c4ccc5ccccc5c4)ccc3c2)C(=O)c2cc3ccccc3cc21]

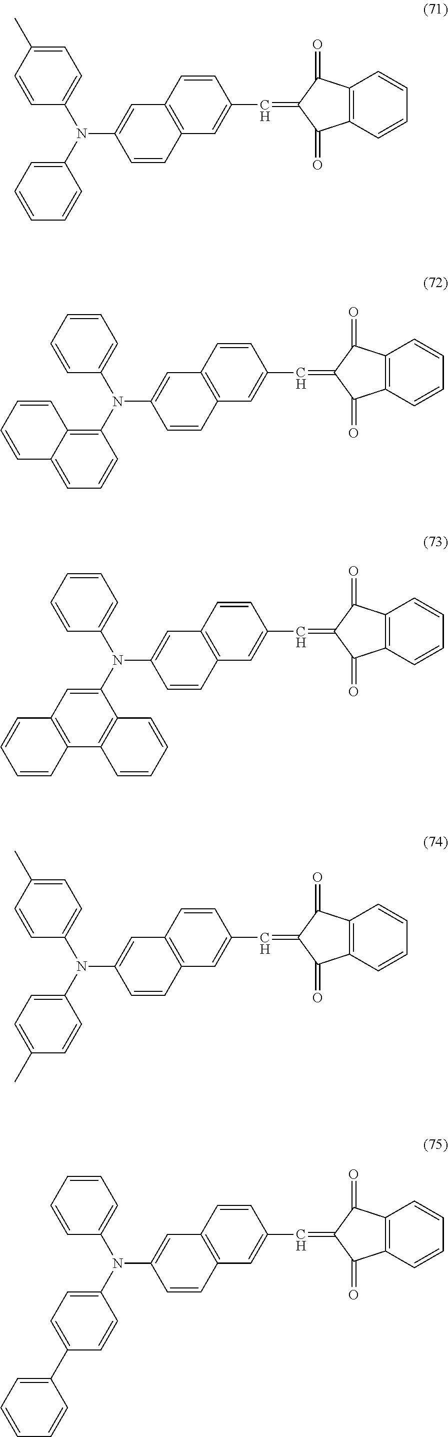 [SMILES: Cc1ccc(N(c2ccccc2)c2ccc3cc(C=C4C(=O)c5ccccc5C4=O)ccc3c2)cc1 ; O=C1C(=Cc2ccc3cc(N(c4ccccc4)c4cccc5ccccc45)ccc3c2)C(=O)c2ccccc21 ; O=C1C(=Cc2ccc3cc(N(c4ccccc4)c4cc5ccccc5c5ccccc45)ccc3c2)C(=O)c2ccccc21 ; Cc1ccc(N(c2ccc(C)cc2)c2ccc3cc(C=C4C(=O)c5ccccc5C4=O)ccc3c2)cc1 ; O=C1C(=Cc2ccc3cc(N(c4ccccc4)c4ccc(-c5ccccc5)cc4)ccc3c2)C(=O)c2ccccc21]

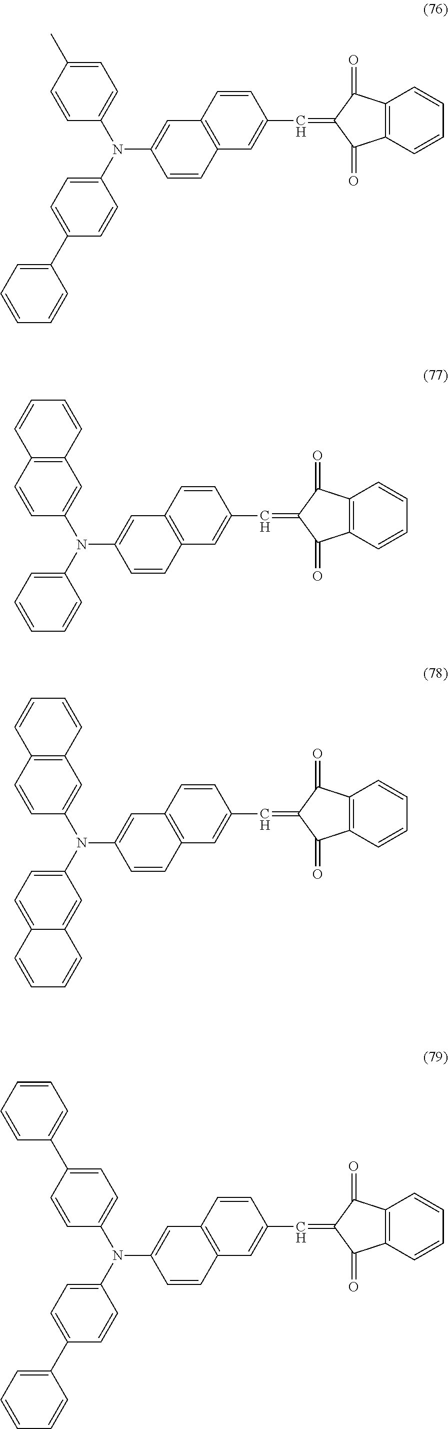 [SMILES: Cc1ccc(N(c2ccc(-c3ccccc3)cc2)c2ccc3cc(C=C4C(=O)c5ccccc5C4=O)ccc3c2)cc1 ; O=C1C(=Cc2ccc3cc(N(c4ccccc4)c4ccc5ccccc5c4)ccc3c2)C(=O)c2ccccc21 ; O=C1C(=Cc2ccc3cc(N(c4ccc5ccccc5c4)c4ccc5ccccc5c4)ccc3c2)C(=O)c2ccccc21 ; O=C1C(=Cc2ccc3cc(N(c4ccc(-c5ccccc5)cc4)c4ccc(-c5ccccc5)cc4)ccc3c2)C(=O)c2ccccc21]

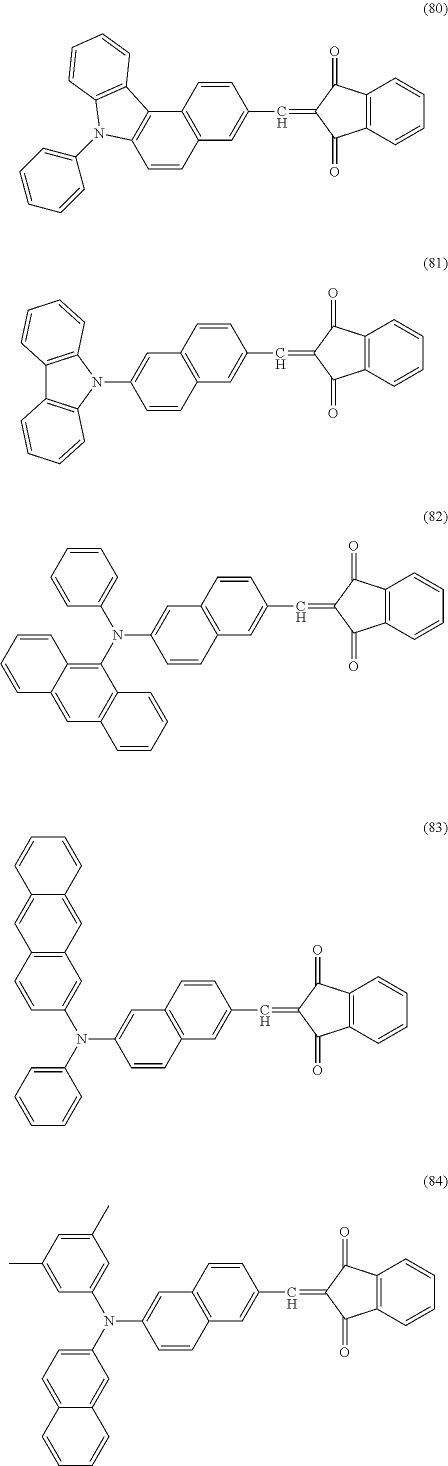 [SMILES: O=C1C(=Cc2ccc3c(ccc4c3c3ccccc3n4-c3ccccc3)c2)C(=O)c2ccccc21 ; O=C1C(=Cc2ccc3cc(-n4c5ccccc5c5ccccc54)ccc3c2)C(=O)c2ccccc21 ; O=C1C(=Cc2ccc3cc(N(c4ccccc4)c4c5ccccc5cc5ccccc45)ccc3c2)C(=O)c2ccccc21 ; O=C1C(=Cc2ccc3cc(N(c4ccccc4)c4ccc5cc6ccccc6cc5c4)ccc3c2)C(=O)c2ccccc21 ; Cc1cc(C)cc(N(c2ccc3ccccc3c2)c2ccc3cc(C=C4C(=O)c5ccccc5C4=O)ccc3c2)c1]

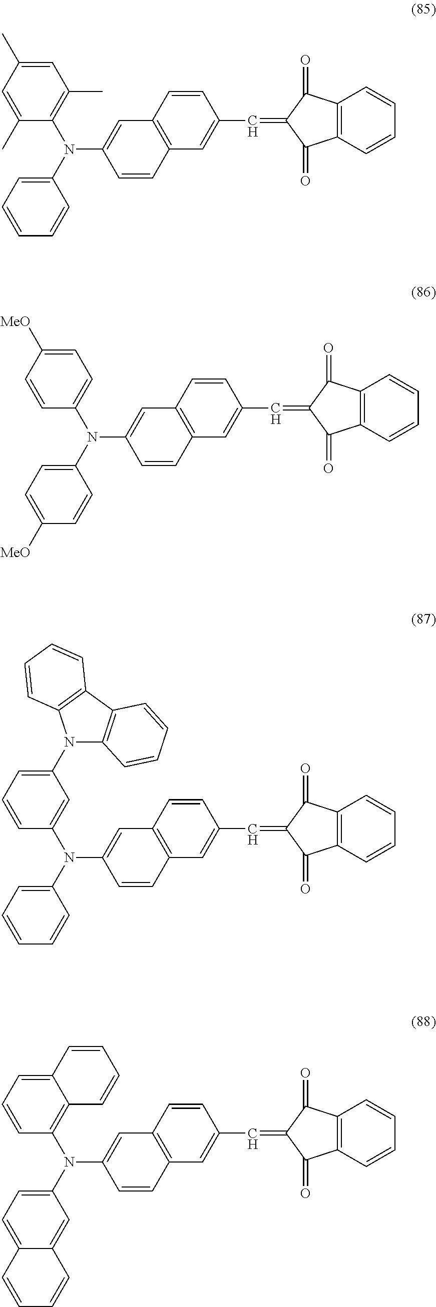 [SMILES: Cc1cc(C)c(N(c2ccccc2)c2ccc3cc(C=C4C(=O)c5ccccc5C4=O)ccc3c2)c(C)c1 ; COc1ccc(N(c2ccc(OC)cc2)c2ccc3cc(C=C4C(=O)c5ccccc5C4=O)ccc3c2)cc1 ; O=C1C(=Cc2ccc3cc(N(c4ccccc4)c4cccc(-n5c6ccccc6c6ccccc65)c4)ccc3c2)C(=O)c2ccccc21 ; O=C1C(=Cc2ccc3cc(N(c4ccc5ccccc5c4)c4cccc5ccccc45)ccc3c2)C(=O)c2ccccc21]

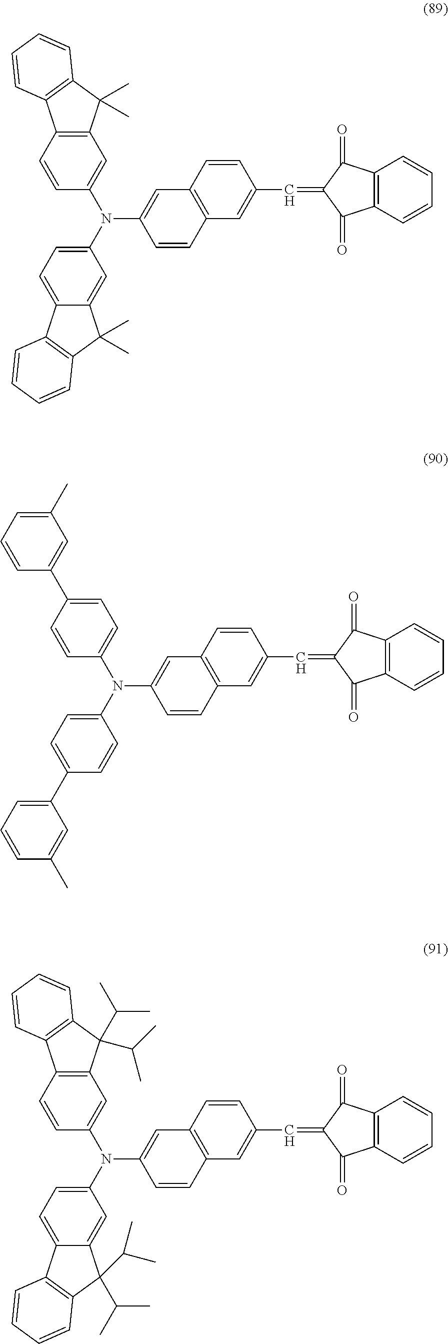 [SMILES: CC1(C)c2ccccc2-c2ccc(N(c3ccc4c(c3)C(C)(C)c3ccccc3-4)c3ccc4cc(C=C5C(=O)c6ccccc6C5=O)ccc4c3)cc21 ; Cc1cccc(-c2ccc(N(c3ccc(-c4cccc(C)c4)cc3)c3ccc4cc(C=C5C(=O)c6ccccc6C5=O)ccc4c3)cc2)c1 ; CC(C)C1(C(C)C)c2ccccc2-c2ccc(N(c3ccc4c(c3)C(C(C)C)(C(C)C)c3ccccc3-4)c3ccc4cc(C=C5C(=O)c6ccccc6C5=O)ccc4c3)cc21]

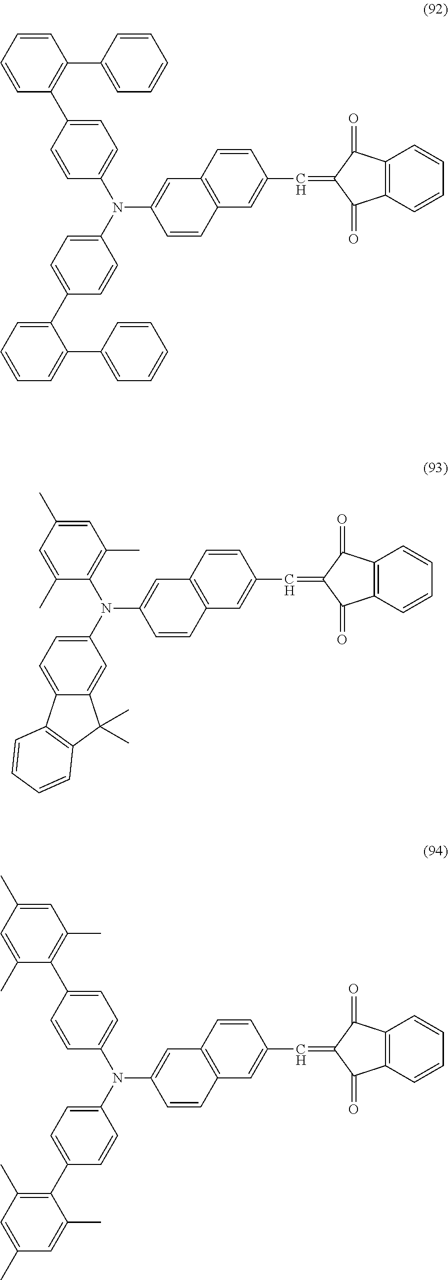 [SMILES: O=C1C(=Cc2ccc3cc(N(c4ccc(-c5ccccc5-c5ccccc5)cc4)c4ccc(-c5ccccc5-c5ccccc5)cc4)ccc3c2)C(=O)c2ccccc21 ; Cc1cc(C)c(N(c2ccc3c(c2)C(C)(C)c2ccccc2-3)c2ccc3cc(C=C4C(=O)c5ccccc5C4=O)ccc3c2)c(C)c1 ; Cc1cc(C)c(-c2ccc(N(c3ccc(-c4c(C)cc(C)cc4C)cc3)c3ccc4cc(C=C5C(=O)c6ccccc6C5=O)ccc4c3)cc2)c(C)c1]

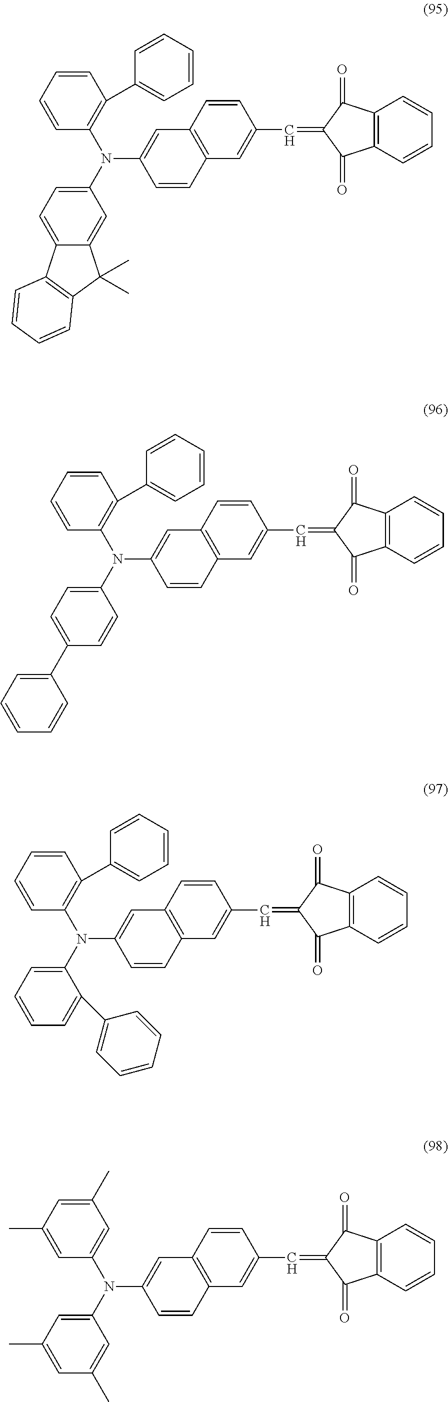 [SMILES: CC1(C)c2ccccc2-c2ccc(N(c3ccc4cc(C=C5C(=O)c6ccccc6C5=O)ccc4c3)c3ccccc3-c3ccccc3)cc21 ; O=C1C(=Cc2ccc3cc(N(c4ccc(-c5ccccc5)cc4)c4ccccc4-c4ccccc4)ccc3c2)C(=O)c2ccccc21 ; O=C1C(=Cc2ccc3cc(N(c4ccccc4-c4ccccc4)c4ccccc4-c4ccccc4)ccc3c2)C(=O)c2ccccc21 ; Cc1cc(C)cc(N(c2cc(C)cc(C)c2)c2ccc3cc(C=C4C(=O)c5ccccc5C4=O)ccc3c2)c1]

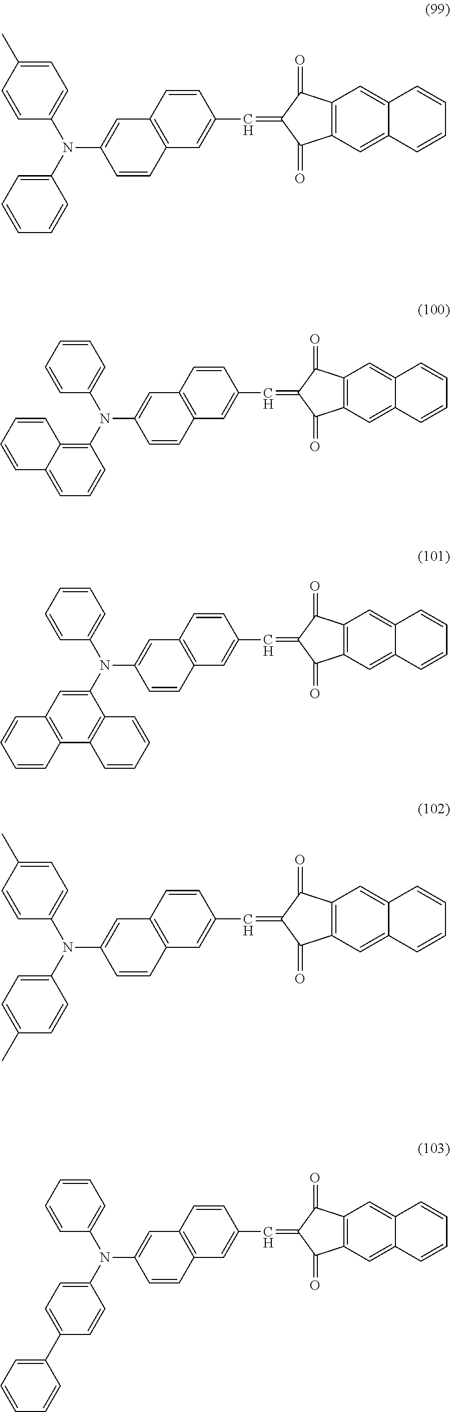 [SMILES: Cc1ccc(N(c2ccccc2)c2ccc3cc(C=C4C(=O)c5cc6ccccc6cc5C4=O)ccc3c2)cc1 ; O=C1C(=Cc2ccc3cc(N(c4ccccc4)c4cccc5ccccc45)ccc3c2)C(=O)c2cc3ccccc3cc21 ; O=C1C(=Cc2ccc3cc(N(c4ccccc4)c4cc5ccccc5c5ccccc45)ccc3c2)C(=O)c2cc3ccccc3cc21 ; Cc1ccc(N(c2ccc(C)cc2)c2ccc3cc(C=C4C(=O)c5cc6ccccc6cc5C4=O)ccc3c2)cc1 ; O=C1C(=Cc2ccc3cc(N(c4ccccc4)c4ccc(-c5ccccc5)cc4)ccc3c2)C(=O)c2cc3ccccc3cc21]

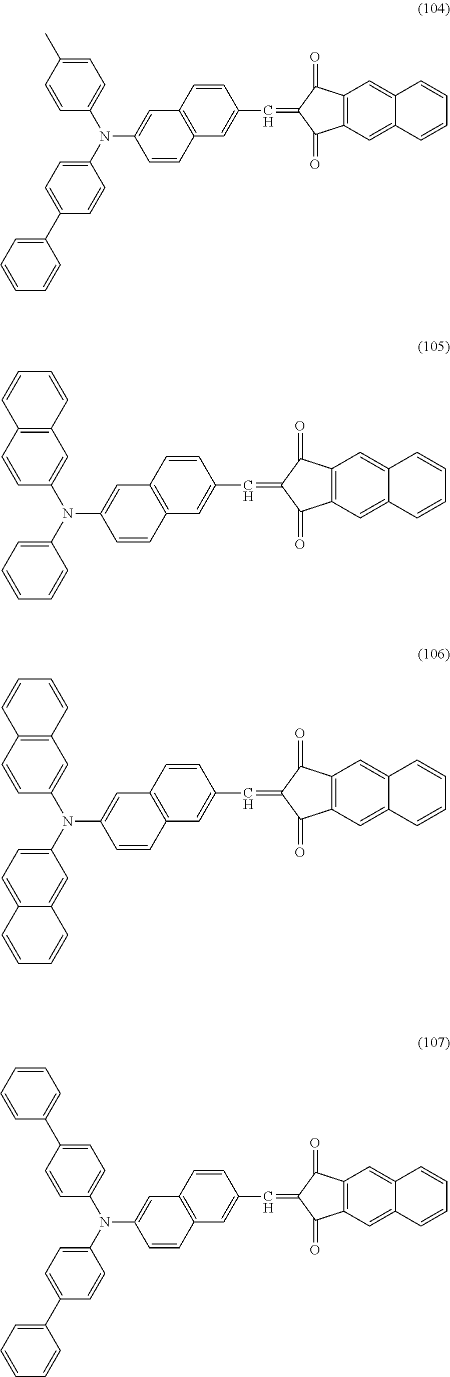 [SMILES: Cc1ccc(N(c2ccc(-c3ccccc3)cc2)c2ccc3cc(C=C4C(=O)c5cc6ccccc6cc5C4=O)ccc3c2)cc1 ; O=C1C(=Cc2ccc3cc(N(c4ccccc4)c4ccc5ccccc5c4)ccc3c2)C(=O)c2cc3ccccc3cc21 ; O=C1C(=Cc2ccc3cc(N(c4ccc5ccccc5c4)c4ccc5ccccc5c4)ccc3c2)C(=O)c2cc3ccccc3cc21 ; O=C1C(=Cc2ccc3cc(N(c4ccc(-c5ccccc5)cc4)c4ccc(-c5ccccc5)cc4)ccc3c2)C(=O)c2cc3ccccc3cc21]

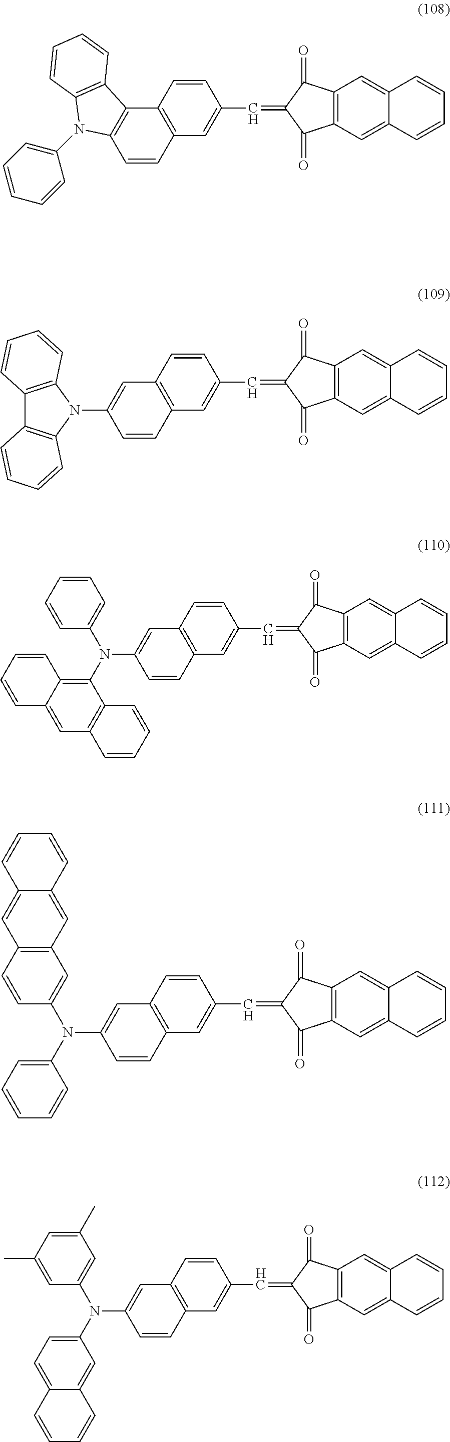 [SMILES: O=C1C(=Cc2ccc3c(ccc4c3c3ccccc3n4-c3ccccc3)c2)C(=O)c2cc3ccccc3cc21 ; O=C1C(=Cc2ccc3cc(-n4c5ccccc5c5ccccc54)ccc3c2)C(=O)c2cc3ccccc3cc21 ; O=C1C(=Cc2ccc3cc(N(c4ccccc4)c4c5ccccc5cc5ccccc45)ccc3c2)C(=O)c2cc3ccccc3cc21 ; O=C1C(=Cc2ccc3cc(N(c4ccccc4)c4ccc5cc6ccccc6cc5c4)ccc3c2)C(=O)c2cc3ccccc3cc21 ; Cc1cc(C)cc(N(c2ccc3ccccc3c2)c2ccc3cc(C=C4C(=O)c5cc6ccccc6cc5C4=O)ccc3c2)c1]

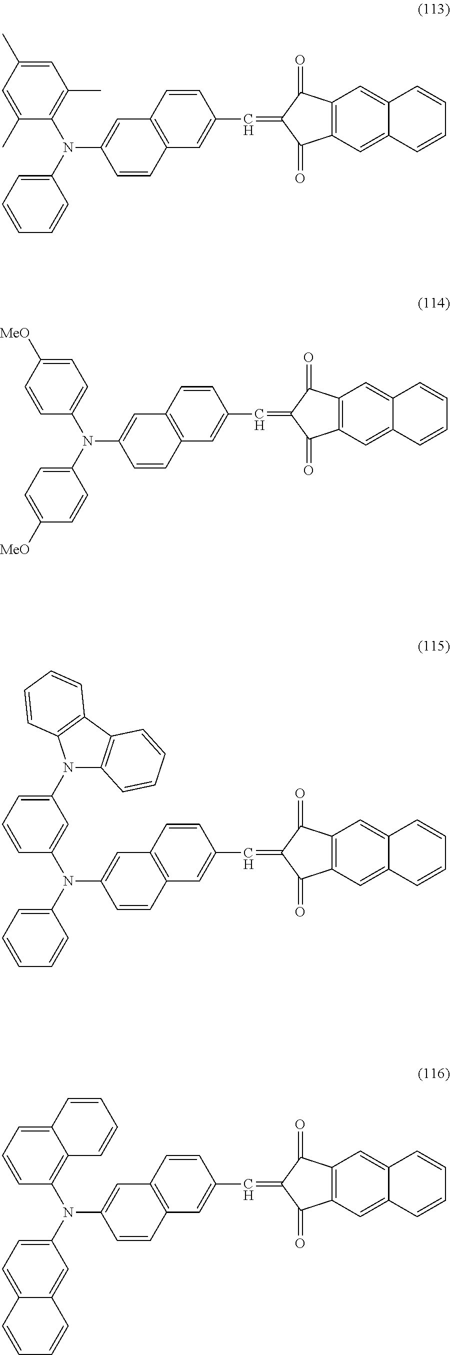 [SMILES: Cc1cc(C)c(N(c2ccccc2)c2ccc3cc(C=C4C(=O)c5cc6ccccc6cc5C4=O)ccc3c2)c(C)c1 ; COc1ccc(N(c2ccc(OC)cc2)c2ccc3cc(C=C4C(=O)c5cc6ccccc6cc5C4=O)ccc3c2)cc1 ; O=C1C(=Cc2ccc3cc(N(c4ccccc4)c4cccc(-n5c6ccccc6c6ccccc65)c4)ccc3c2)C(=O)c2cc3ccccc3cc21 ; O=C1C(=Cc2ccc3cc(N(c4ccc5ccccc5c4)c4cccc5ccccc45)ccc3c2)C(=O)c2cc3ccccc3cc21]

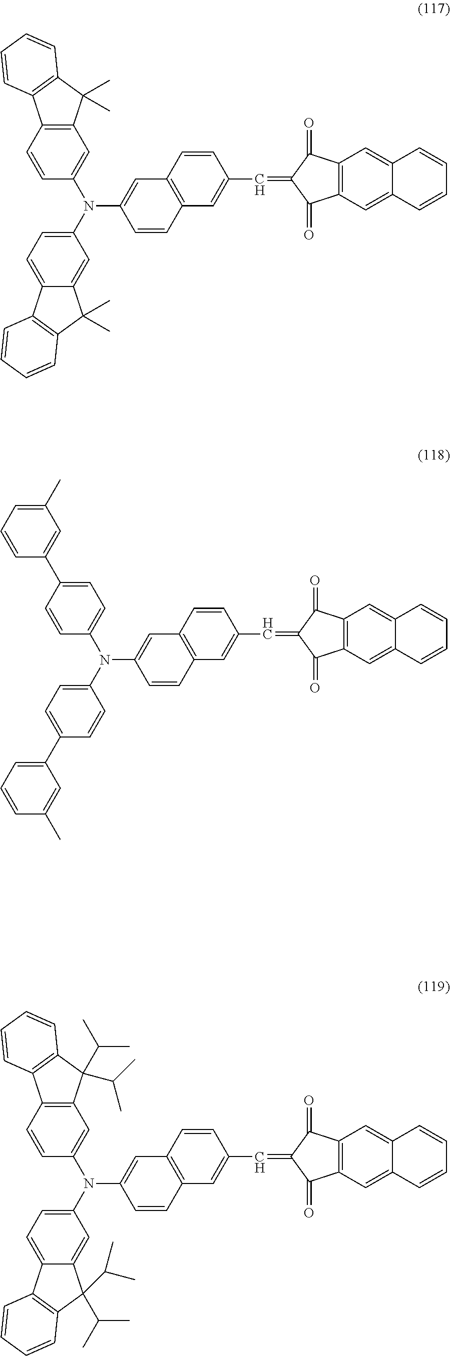 [SMILES: CC1(C)c2ccccc2-c2ccc(N(c3ccc4c(c3)C(C)(C)c3ccccc3-4)c3ccc4cc(C=C5C(=O)c6cc7ccccc7cc6C5=O)ccc4c3)cc21 ; Cc1cccc(-c2ccc(N(c3ccc(-c4cccc(C)c4)cc3)c3ccc4cc(C=C5C(=O)c6cc7ccccc7cc6C5=O)ccc4c3)cc2)c1 ; CC(C)C1(C(C)C)c2ccccc2-c2ccc(N(c3ccc4c(c3)C(C(C)C)(C(C)C)c3ccccc3-4)c3ccc4cc(C=C5C(=O)c6cc7ccccc7cc6C5=O)ccc4c3)cc21]

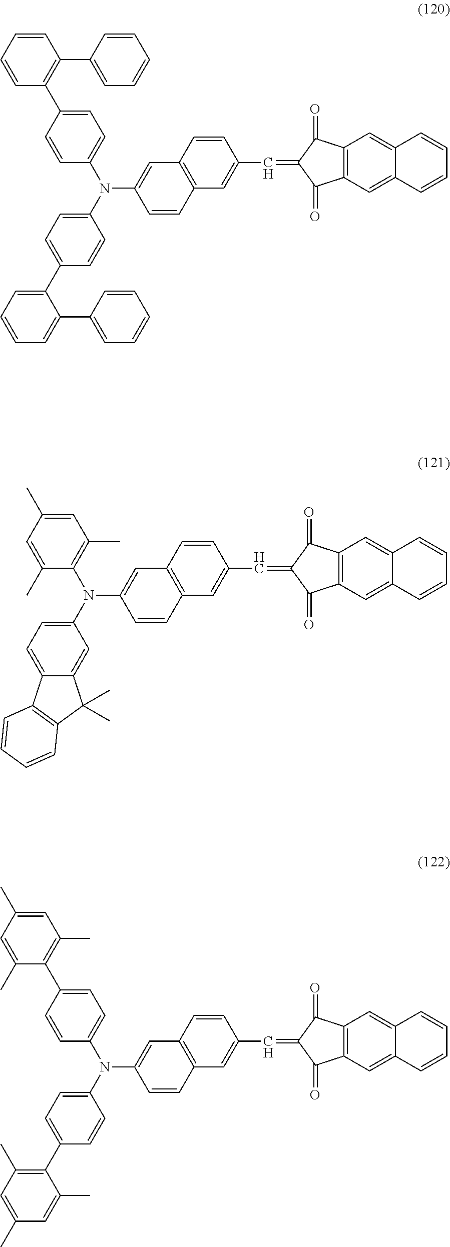 [SMILES: O=C1C(=Cc2ccc3cc(N(c4ccc(-c5ccccc5-c5ccccc5)cc4)c4ccc(-c5ccccc5-c5ccccc5)cc4)ccc3c2)C(=O)c2cc3ccccc3cc21 ; Cc1cc(C)c(N(c2ccc3c(c2)C(C)(C)c2ccccc2-3)c2ccc3cc(C=C4C(=O)c5cc6ccccc6cc5C4=O)ccc3c2)c(C)c1 ; Cc1cc(C)c(-c2ccc(N(c3ccc(-c4c(C)cc(C)cc4C)cc3)c3ccc4cc(C=C5C(=O)c6cc7ccccc7cc6C5=O)ccc4c3)cc2)c(C)c1]

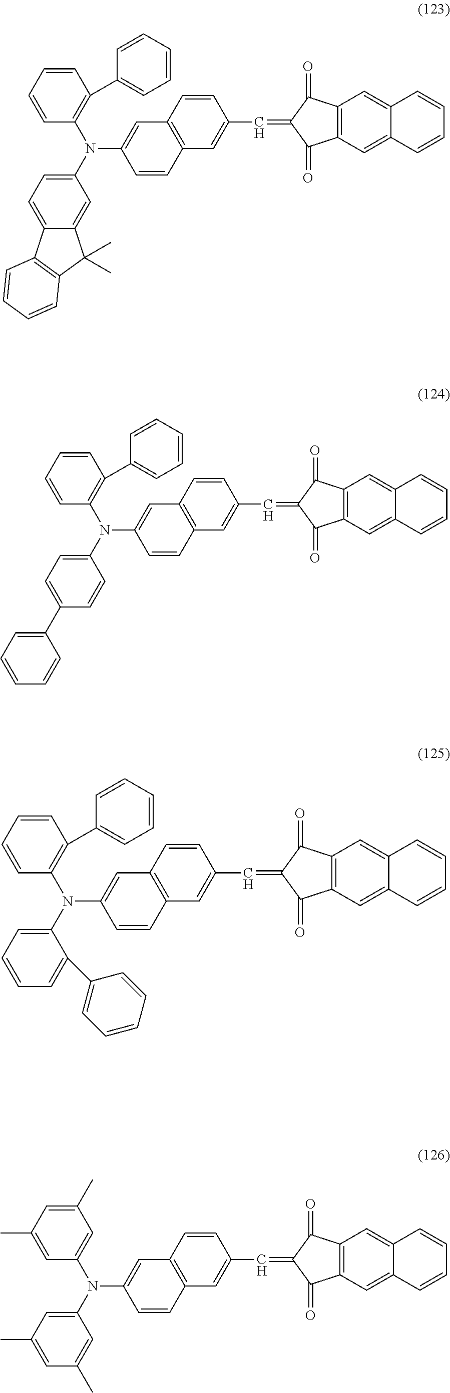 [SMILES: CC1(C)c2ccccc2-c2ccc(N(c3ccc4cc(C=C5C(=O)c6cc7ccccc7cc6C5=O)ccc4c3)c3ccccc3-c3ccccc3)cc21 ; O=C1C(=Cc2ccc3cc(N(c4ccc(-c5ccccc5)cc4)c4ccccc4-c4ccccc4)ccc3c2)C(=O)c2cc3ccccc3cc21 ; O=C1C(=Cc2ccc3cc(N(c4ccccc4-c4ccccc4)c4ccccc4-c4ccccc4)ccc3c2)C(=O)c2cc3ccccc3cc21 ; Cc1cc(C)cc(N(c2cc(C)cc(C)c2)c2ccc3cc(C=C4C(=O)c5cc6ccccc6cc5C4=O)ccc3c2)c1]

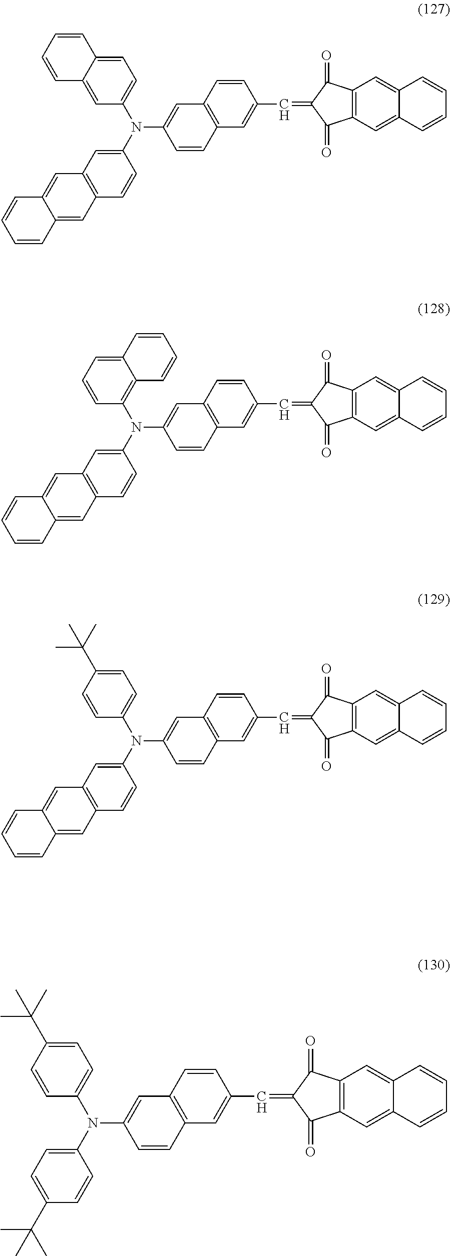 [SMILES: O=C1C(=Cc2ccc3cc(N(c4ccc5ccccc5c4)c4ccc5cc6ccccc6cc5c4)ccc3c2)C(=O)c2cc3ccccc3cc21 ; O=C1C(=Cc2ccc3cc(N(c4ccc5cc6ccccc6cc5c4)c4cccc5ccccc45)ccc3c2)C(=O)c2cc3ccccc3cc21 ; CC(C)(C)c1ccc(N(c2ccc3cc(C=C4C(=O)c5cc6ccccc6cc5C4=O)ccc3c2)c2ccc3cc4ccccc4cc3c2)cc1 ; CC(C)(C)c1ccc(N(c2ccc(C(C)(C)C)cc2)c2ccc3cc(C=C4C(=O)c5cc6ccccc6cc5C4=O)ccc3c2)cc1]

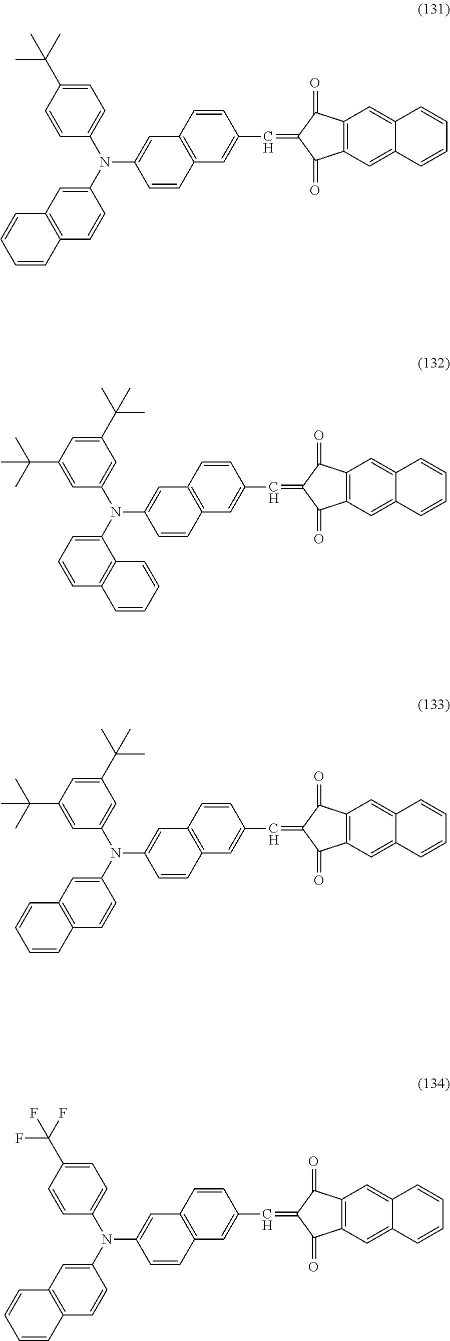 [SMILES: CC(C)(C)c1ccc(N(c2ccc3ccccc3c2)c2ccc3cc(C=C4C(=O)c5cc6ccccc6cc5C4=O)ccc3c2)cc1 ; CC(C)(C)c1cc(N(c2ccc3cc(C=C4C(=O)c5cc6ccccc6cc5C4=O)ccc3c2)c2cccc3ccccc23)cc(C(C)(C)C)c1 ; CC(C)(C)c1cc(N(c2ccc3ccccc3c2)c2ccc3cc(C=C4C(=O)c5cc6ccccc6cc5C4=O)ccc3c2)cc(C(C)(C)C)c1 ; O=C1C(=Cc2ccc3cc(N(c4ccc(C(F)(F)F)cc4)c4ccc5ccccc5c4)ccc3c2)C(=O)c2cc3ccccc3cc21]

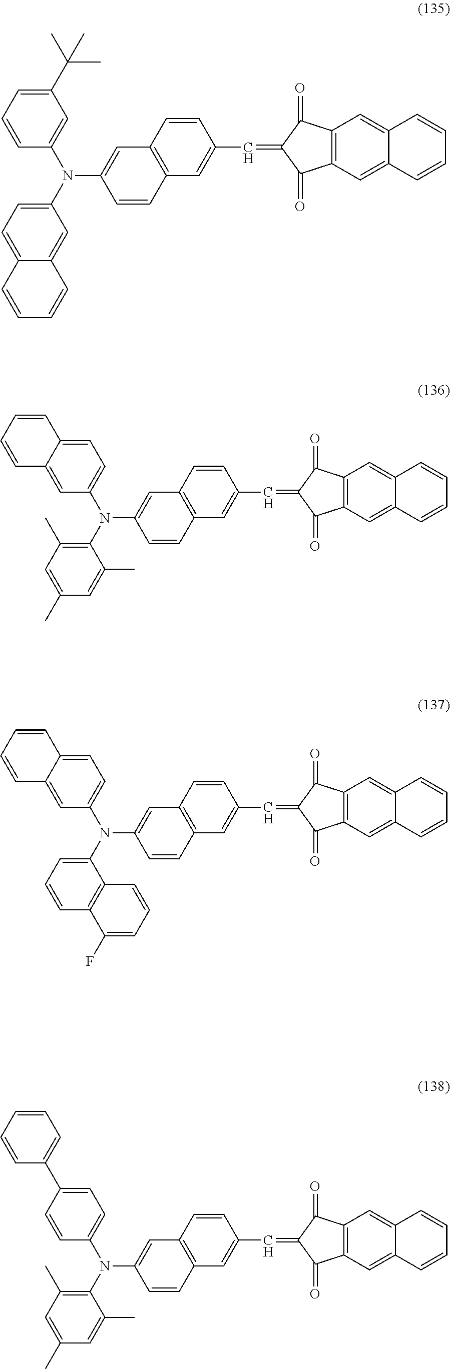 [SMILES: CC(C)(C)c1cccc(N(c2ccc3ccccc3c2)c2ccc3cc(C=C4C(=O)c5cc6ccccc6cc5C4=O)ccc3c2)c1 ; Cc1cc(C)c(N(c2ccc3ccccc3c2)c2ccc3cc(C=C4C(=O)c5cc6ccccc6cc5C4=O)ccc3c2)c(C)c1 ; O=C1C(=Cc2ccc3cc(N(c4ccc5ccccc5c4)c4cccc5c(F)cccc45)ccc3c2)C(=O)c2cc3ccccc3cc21 ; Cc1cc(C)c(N(c2ccc(-c3ccccc3)cc2)c2ccc3cc(C=C4C(=O)c5cc6ccccc6cc5C4=O)ccc3c2)c(C)c1]

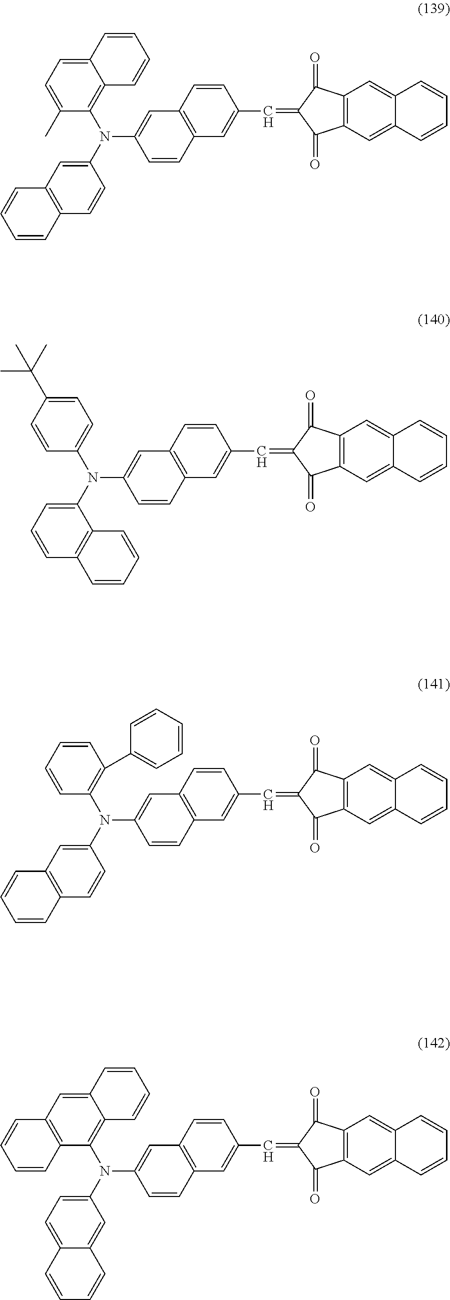 [SMILES: Cc1ccc2ccccc2c1N(c1ccc2ccccc2c1)c1ccc2cc(C=C3C(=O)c4cc5ccccc5cc4C3=O)ccc2c1 ; CC(C)(C)c1ccc(N(c2ccc3cc(C=C4C(=O)c5cc6ccccc6cc5C4=O)ccc3c2)c2cccc3ccccc23)cc1 ; O=C1C(=Cc2ccc3cc(N(c4ccc5ccccc5c4)c4ccccc4-c4ccccc4)ccc3c2)C(=O)c2cc3ccccc3cc21 ; O=C1C(=Cc2ccc3cc(N(c4ccc5ccccc5c4)c4c5ccccc5cc5ccccc45)ccc3c2)C(=O)c2cc3ccccc3cc21]

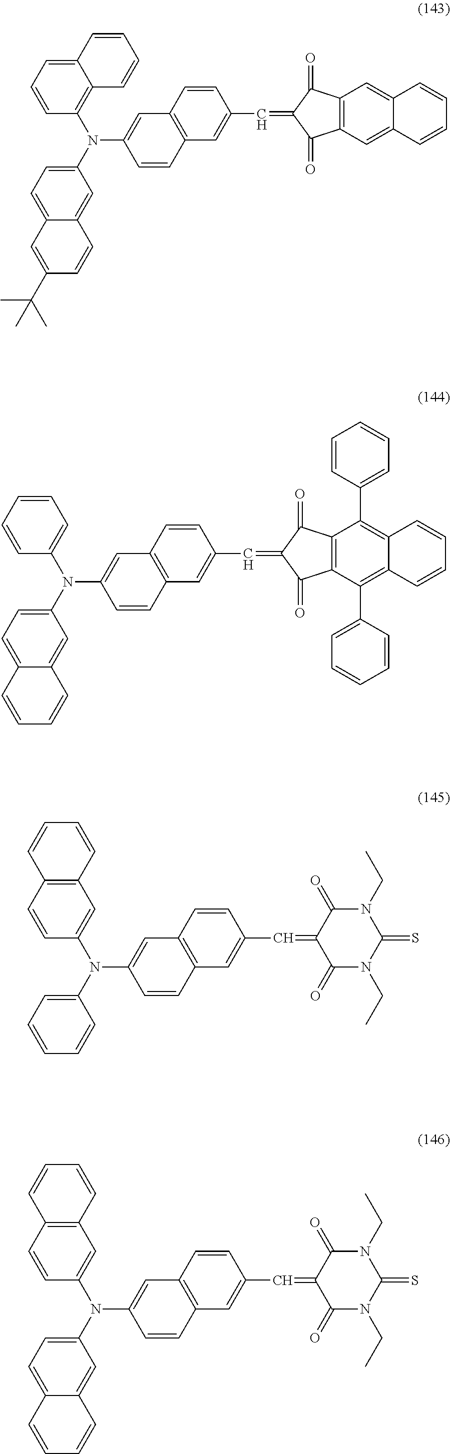 [SMILES: CC(C)(C)c1ccc2cc(N(c3ccc4cc(C=C5C(=O)c6cc7ccccc7cc6C5=O)ccc4c3)c3cccc4ccccc34)ccc2c1 ; O=C1C(=Cc2ccc3cc(N(c4ccccc4)c4ccc5ccccc5c4)ccc3c2)C(=O)c2c1c(-c1ccccc1)c1ccccc1c2-c1ccccc1 ; CCN1C(=O)C(=Cc2ccc3cc(N(c4ccccc4)c4ccc5ccccc5c4)ccc3c2)C(=O)N(CC)C1=S ; CCN1C(=O)C(=Cc2ccc3cc(N(c4ccc5ccccc5c4)c4ccc5ccccc5c4)ccc3c2)C(=O)N(CC)C1=S]

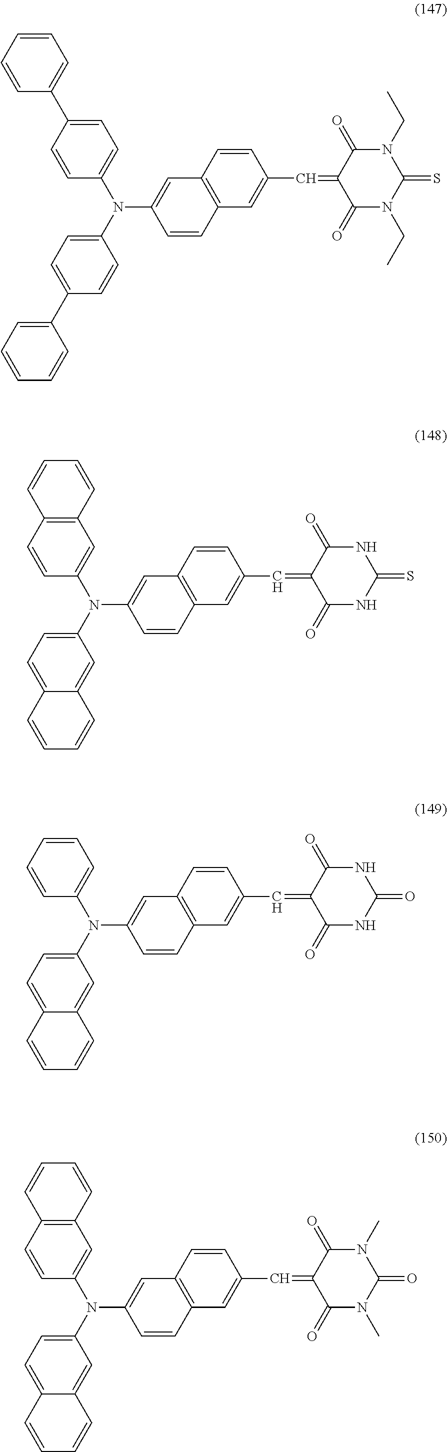 [SMILES: CCN1C(=O)C(=Cc2ccc3cc(N(c4ccc(-c5ccccc5)cc4)c4ccc(-c5ccccc5)cc4)ccc3c2)C(=O)N(CC)C1=S ; O=C1NC(=S)NC(=O)C1=Cc1ccc2cc(N(c3ccc4ccccc4c3)c3ccc4ccccc4c3)ccc2c1 ; O=C1NC(=O)C(=Cc2ccc3cc(N(c4ccccc4)c4ccc5ccccc5c4)ccc3c2)C(=O)N1 ; CN1C(=O)C(=Cc2ccc3cc(N(c4ccc5ccccc5c4)c4ccc5ccccc5c4)ccc3c2)C(=O)N(C)C1=O]

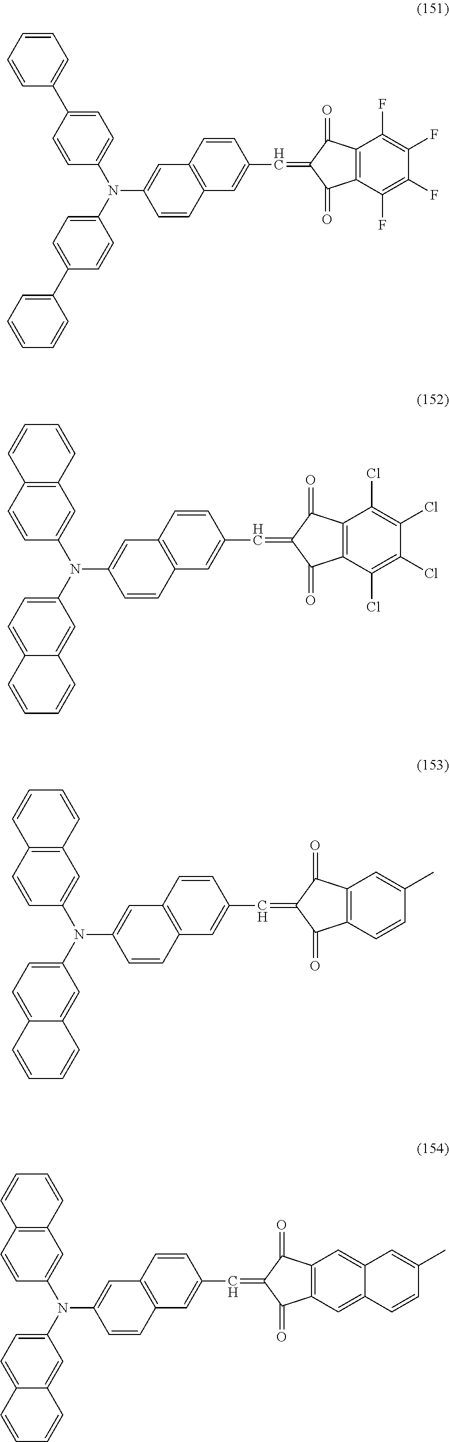 [SMILES: O=C1C(=Cc2ccc3cc(N(c4ccc(-c5ccccc5)cc4)c4ccc(-c5ccccc5)cc4)ccc3c2)C(=O)c2c(F)c(F)c(F)c(F)c21 ; O=C1C(=Cc2ccc3cc(N(c4ccc5ccccc5c4)c4ccc5ccccc5c4)ccc3c2)C(=O)c2c(Cl)c(Cl)c(Cl)c(Cl)c21 ; Cc1ccc2c(c1)C(=O)C(=Cc1ccc3cc(N(c4ccc5ccccc5c4)c4ccc5ccccc5c4)ccc3c1)C2=O ; Cc1ccc2cc3c(cc2c1)C(=O)C(=Cc1ccc2cc(N(c4ccc5ccccc5c4)c4ccc5ccccc5c4)ccc2c1)C3=O]

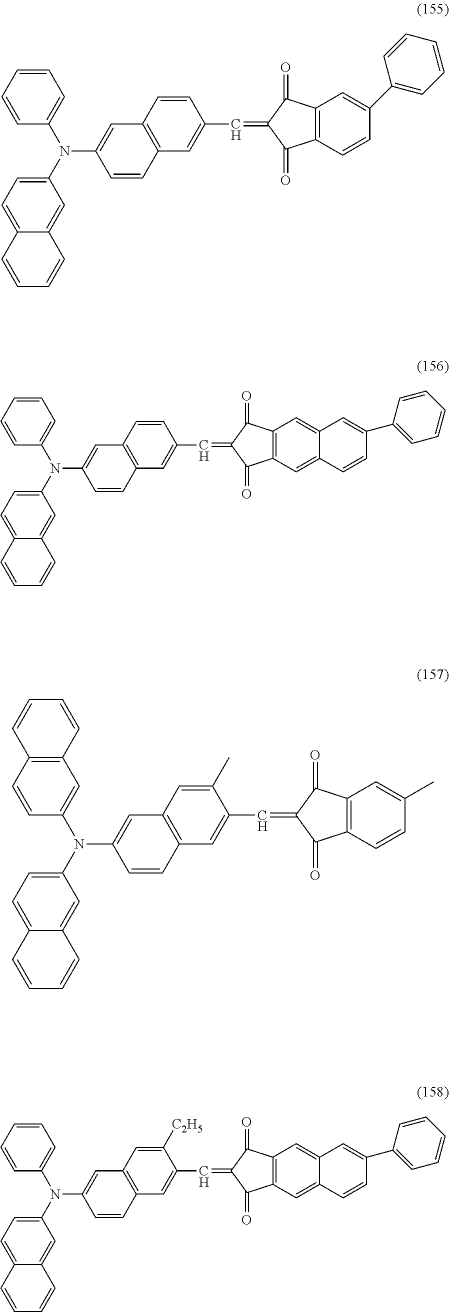 [SMILES: O=C1C(=Cc2ccc3cc(N(c4ccccc4)c4ccc5ccccc5c4)ccc3c2)C(=O)c2cc(-c3ccccc3)ccc21 ; O=C1C(=Cc2ccc3cc(N(c4ccccc4)c4ccc5ccccc5c4)ccc3c2)C(=O)c2cc3cc(-c4ccccc4)ccc3cc21 ; Cc1ccc2c(c1)C(=O)C(=Cc1cc3ccc(N(c4ccc5ccccc5c4)c4ccc5ccccc5c4)cc3cc1C)C2=O ; CCc1cc2cc(N(c3ccccc3)c3ccc4ccccc4c3)ccc2cc1C=C1C(=O)c2cc3ccc(-c4ccccc4)cc3cc2C1=O]

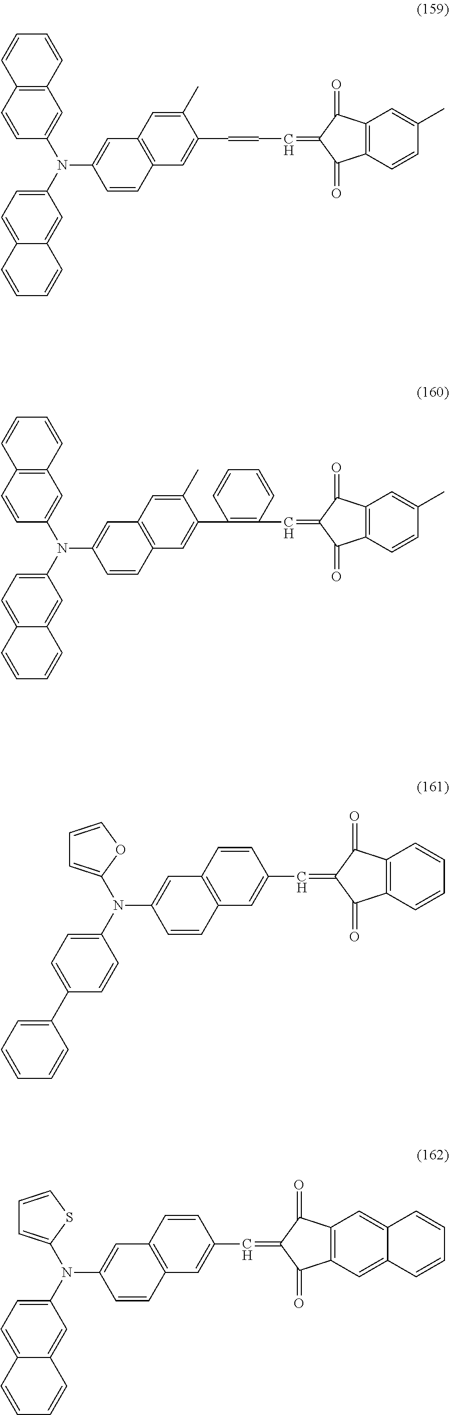 [SMILES: Cc1ccc2c(c1)C(=O)C(=CC=Cc1cc3ccc(N(c4ccc5ccccc5c4)c4ccc5ccccc5c4)cc3cc1C)C2=O ; Cc1ccc2c(c1)C(=O)C(=Cc1ccccc1-c1cc3ccc(N(c4ccc5ccccc5c4)c4ccc5ccccc5c4)cc3cc1C)C2=O ; O=C1C(=Cc2ccc3cc(N(c4ccc(-c5ccccc5)cc4)c4ccco4)ccc3c2)C(=O)c2ccccc21 ; O=C1C(=Cc2ccc3cc(N(c4ccc5ccccc5c4)c4cccs4)ccc3c2)C(=O)c2cc3ccccc3cc21]

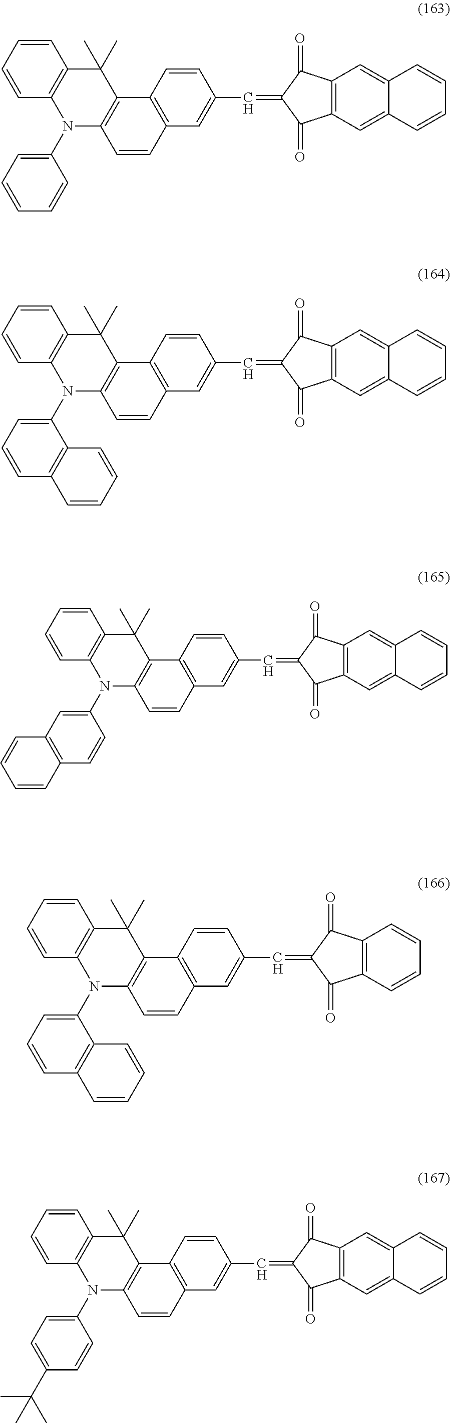 [SMILES: CC1(C)c2ccccc2N(c2ccccc2)c2ccc3cc(C=C4C(=O)c5cc6ccccc6cc5C4=O)ccc3c21 ; CC1(C)c2ccccc2N(c2cccc3ccccc23)c2ccc3cc(C=C4C(=O)c5cc6ccccc6cc5C4=O)ccc3c21 ; CC1(C)c2ccccc2N(c2ccc3ccccc3c2)c2ccc3cc(C=C4C(=O)c5cc6ccccc6cc5C4=O)ccc3c21 ; CC1(C)c2ccccc2N(c2cccc3ccccc23)c2ccc3cc(C=C4C(=O)c5ccccc5C4=O)ccc3c21 ; CC(C)(C)c1ccc(N2c3ccccc3C(C)(C)c3c2ccc2cc(C=C4C(=O)c5cc6ccccc6cc5C4=O)ccc32)cc1]

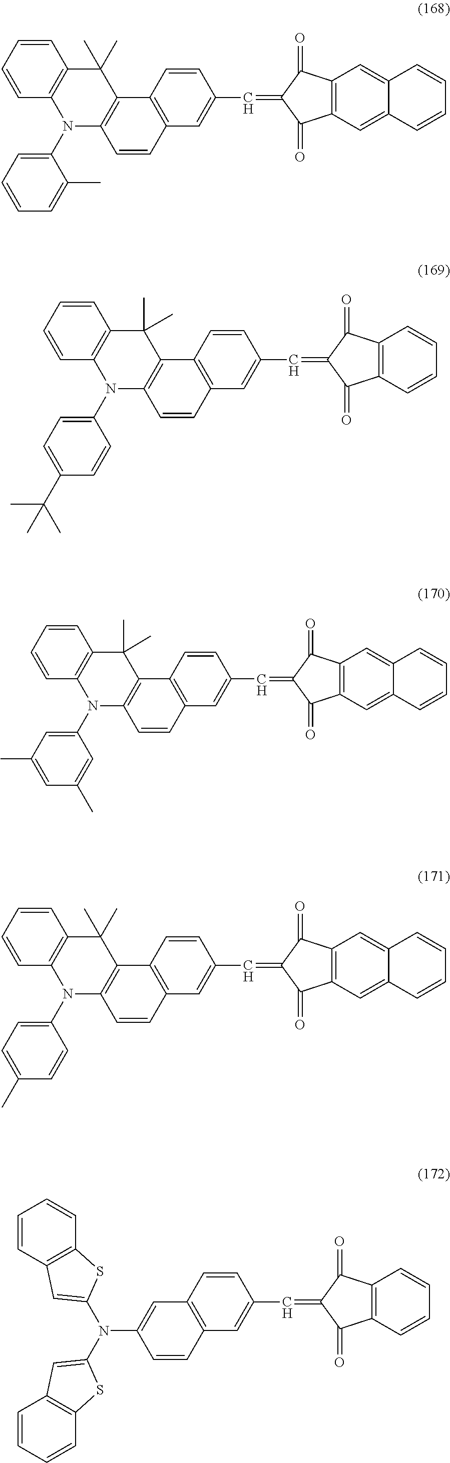 [SMILES: Cc1ccccc1N1c2ccccc2C(C)(C)c2c1ccc1cc(C=C3C(=O)c4cc5ccccc5cc4C3=O)ccc21 ; CC(C)(C)c1ccc(N2c3ccccc3C(C)(C)c3c2ccc2cc(C=C4C(=O)c5ccccc5C4=O)ccc32)cc1 ; Cc1cc(C)cc(N2c3ccccc3C(C)(C)c3c2ccc2cc(C=C4C(=O)c5cc6ccccc6cc5C4=O)ccc32)c1 ; Cc1ccc(N2c3ccccc3C(C)(C)c3c2ccc2cc(C=C4C(=O)c5cc6ccccc6cc5C4=O)ccc32)cc1 ; O=C1C(=Cc2ccc3cc(N(c4cc5ccccc5s4)c4cc5ccccc5s4)ccc3c2)C(=O)c2ccccc21]

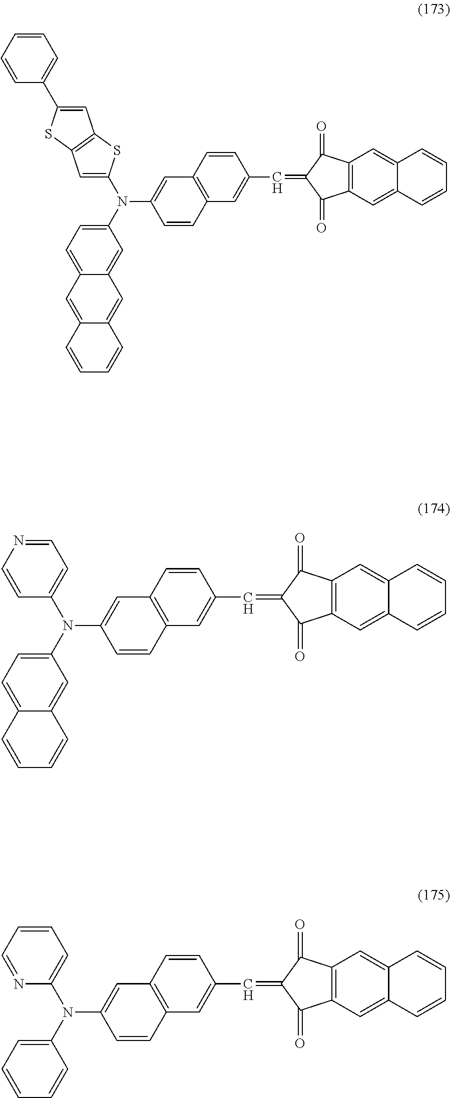 [SMILES: O=C1C(=Cc2ccc3cc(N(c4ccc5cc6ccccc6cc5c4)c4cc5sc(-c6ccccc6)cc5s4)ccc3c2)C(=O)c2cc3ccccc3cc21 ; O=C1C(=Cc2ccc3cc(N(c4ccncc4)c4ccc5ccccc5c4)ccc3c2)C(=O)c2cc3ccccc3cc21 ; O=C1C(=Cc2ccc3cc(N(c4ccccc4)c4ccccn4)ccc3c2)C(=O)c2cc3ccccc3cc21]

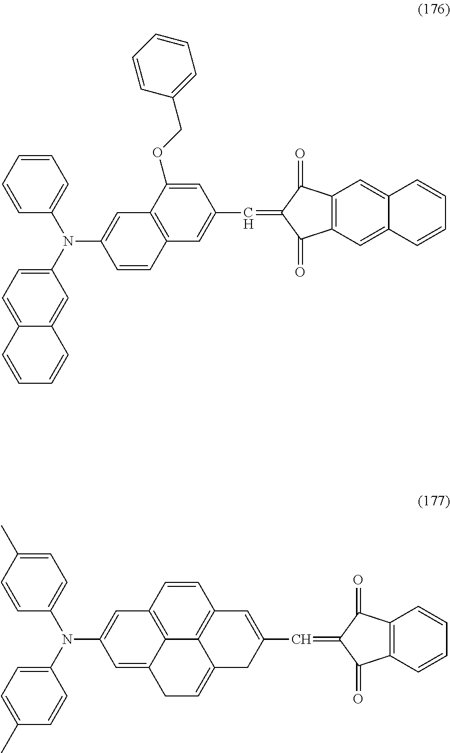 [SMILES: O=C1C(=Cc2cc(OCc3ccccc3)c3cc(N(c4ccccc4)c4ccc5ccccc5c4)ccc3c2)C(=O)c2cc3ccccc3cc21 ; Cc1ccc(N(c2ccc(C)cc2)c2cc3c4c5c(ccc4c2)C=C(C=C2C(=O)c4ccccc4C2=O)CC5=CC3)cc1]

In the compounds exemplified above, R₁₀₁ and R₁₀₂ each independently represent a hydrogen atom or a substituent. Examples of the substituent include the substituent W, and the substituent is preferably an alkyl group or an aryl group.

[n-Type Organic Semiconductor]

An n-type organic semiconductor (compound) is an acceptor-type organic semiconductor (compound) and refers to an organic compound usually typified by an electron transporting organic compound and having a property of easily accepting electrons. More specifically, the n-type organic semiconductor material refers to an organic compound having higher electron affinity when two organic compounds are brought into contact with each other and used.

Accordingly, for the acceptor-type organic compound, any organic compound can be used as long as the organic compound is an organic compound having an electron accepting property. Examples thereof include a metal complex having a condensed aromatic carbocyclic compound (naphthalene, anthracene, fullerene, phenanthrene, tetracene, pyrene, perylene, fluoranthene or derivatives thereof), a 5- to 7-membered heterocyclic compound containing a nitrogen atom, an oxygen atom or a sulfur atom (for example, pyridine, pyrazine, pyrimidine, pyridazine, triazine, quinoline, quinoxaline, quinazoline, phthalazine, cinnoline, isoquinoline, pteridine, acridine, phenazine, phenanthroline, tetrazole, pyrazole, imidazole, thiazole, oxazole, indazole, benzimidazole, benzotriazole, benzoxazole, benzothiazole, carbazole, purine, triazolopyridazine, triazolopyrimidine, tetrazaindene, oxadiazole, imidazopyridine, pyralidine, pyrrolopyridine, thiadiazolopyridine, dibenzazepine, tribenzazepine and the like), a polyarylene compound, a fluorene compound, a cyclopentadiene compound, a silyl compound and a nitrogen-containing heterocyclic compound as a ligand, and the like. Further, the acceptor-type organic semiconductor is not limited thereto, and any organic compound may be used as an acceptor-type organic semiconductor as long as the organic compound is an organic compound having electron affinity larger than that of an organic compound used as the donor-type organic compound as described above.

As the n-type organic semiconductor, fullerene or a fullerene derivative is preferably used.

The fullerene indicates fullerene C₆₀, fullerene C₇₀, fullerene C₇₆, fullerene C₇₈, fullerene C₈₀, fullerene C₈₂, fullerene C₈₄, fullerene C₉₀, fullerene C₉₆, fullerene C₂₄₀, fullerene C₅₄₀, a mixed fullerene or a fullerene nanotube, and the fullerene derivative indicates a compound obtained by adding a substituent to the fullerenes. As the substituent group, an alkyl group, an aryl group or a heterocyclic group is preferred.

The following compounds are preferred as the fullerene derivative.

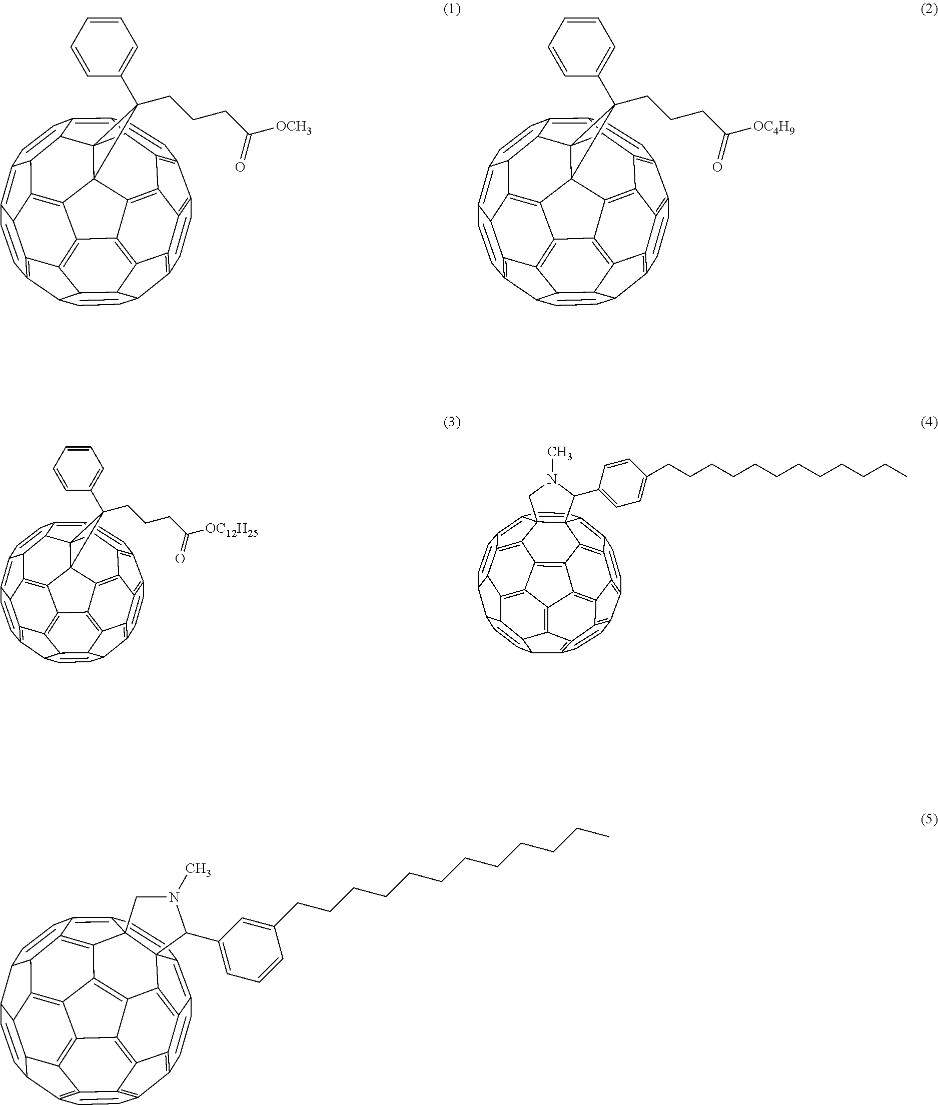

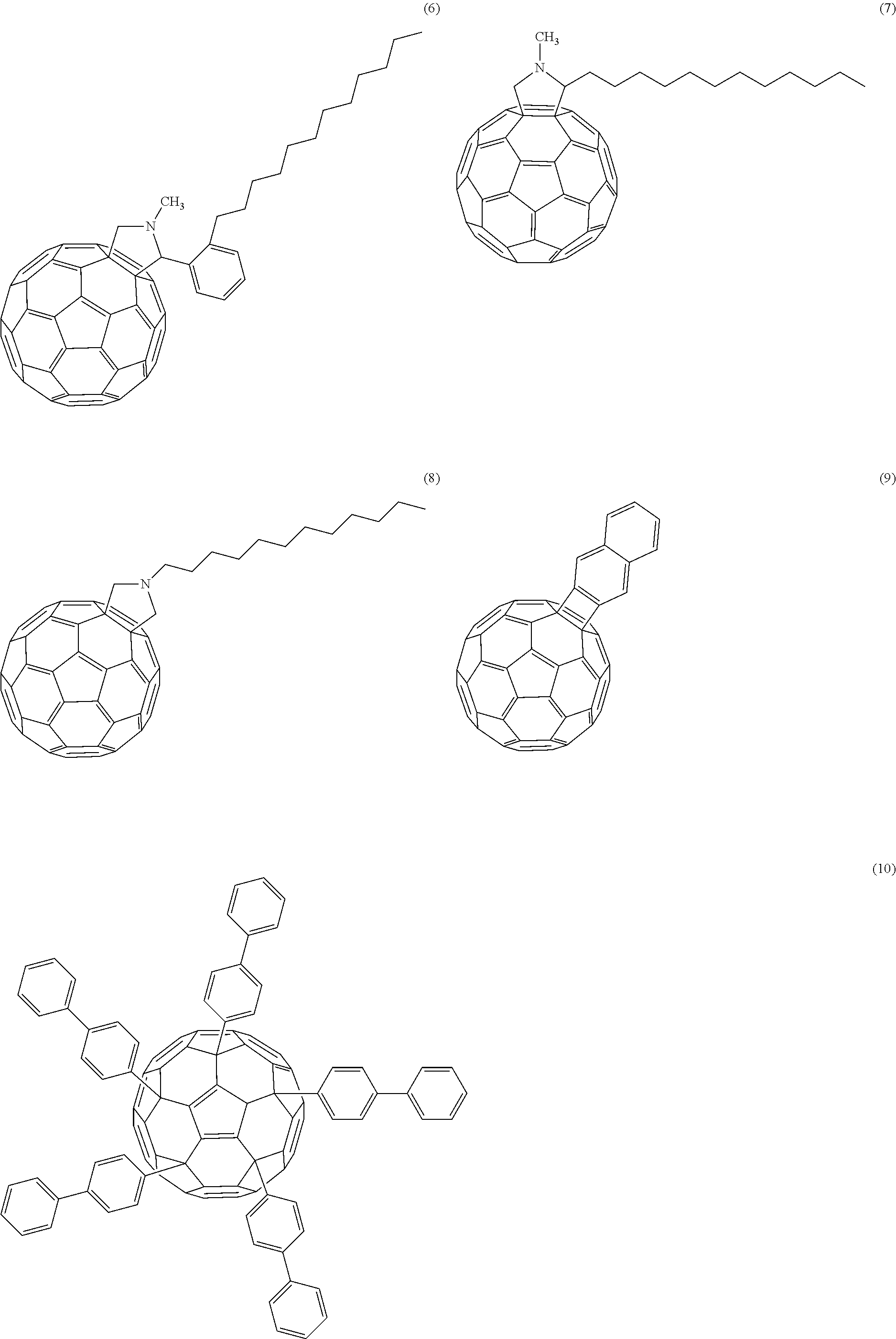

As the fullerene and fullerene derivative, it is also possible to use the compounds described in Scientific Review Quarterly No. 43 (1999), edited by The Chemical Society of Japan (1999), and the Japanese Patent Application Laid-Open Nos. H10-167994, H11-255508, H11-255509, 2002-241323 and 2003-196881.

The content of the fullerene or fullerene derivative in a mixed layer with a p-type material is preferably 50% or more (by mole), more preferably 200% or more (by mole), and particularly preferably 300% or more (by mole), based on the amount of other materials forming the mixed film.

The photoelectric conversion layer may be formed by deposition. The deposition may be any of physical vapor deposition (PVD) and chemical vapor deposition (CVD), but physical vapor deposition such as vacuum deposition is preferred. When a film is formed by the vacuum deposition method, manufacturing conditions such as vacuum degree and deposition temperature may be adjusted according to a typical method.

A thickness of the photoelectric conversion layer is preferably 10 nm to 1,000 nm, more preferably 50 nm to 800 nm, and particularly preferably 100 nm to 500 nm. It is possible to obtain a suitable dark current suppression effect by adjusting the thickness to 10 nm or more, and it is possible to obtain a suitable photoelectric conversion efficiency by adjusting the thickness to 1000 nm or less.

In the method for manufacturing a photoelectric conversion device of the present invention, it is also preferred that the method includes film-forming each of a photoelectric conversion layer and a charge blocking layer by vacuum heating deposition (vacuum deposition).

(Charge Blocking Layer)

An electron donating organic material may be used in the charge blocking layer. Specifically, it is possible to use an aromatic diamine compound such as N,N′-bis(3-methylphenyl)-(1,1′-biphenyl)-4,4′-diamine (TPD) or 4,4′-bis[N-(naphthyl)-N-phenyl-amino]biphenyl (α-NPD), a porphyrin compound such as oxazole, oxadiazole, triazole, imidazole, imidazolone, a stilbene derivative, a pyrazoline derivative, tetrahydroimidazole, polyarylalkane, butadiene, 4,4′,4″-tris(N-(3-methylphenyl)N-phenylamino)triphenylamine (m-MTDATA), porphin, tetraphenylporphin copper, phthalocyanine, copper phthalocyanine, and titanium phthalocyanine oxide, a triazole derivative, an oxadiazole derivative, an imidazole derivative, a polyarylalkane derivative, a pyrazoline derivative, a pyrazolone derivative, a phenylenediamine derivative, an anileamine derivative, an amino substituted chalcone derivative, an oxazole derivative, a styrylanthracene derivative, a fluorenone derivative, a hydrazone derivative, a silazane derivative and the like as a low molecular material, and a polymer such as phenylenevinylene, fluorene, carbazole, indole, pyrene, pyrrole, picoline, thiophene, acetylene, and diacetylene or a derivative thereof may be used as a polymer material. Any compound may be used as long as the compound is not an electron donating compound, but has a sufficient hole transporting property.

Specifically, the compounds, which are described in paragraph Nos. [0083] to [0089] of the Japanese Patent Application Laid-Open No. 2008-72090, are preferred.

In the present invention, particularly, the charge blocking layer contains preferably a compound represented by Formula (1) or (2), more preferably a compound represented by Formula (1), still more preferably a compound represented by Formula (F-1), and particularly preferably a compound represented by Formula (F-1).

(Hole Blocking Layer)

An electron accepting organic material may be used in the hole blocking layer. As the electron accepting material, it is possible to use an oxadiazole derivative such as 1,3-bis(4-tert-butylphenyl-1,3,4-oxadiazolyl)phenylene (OXD-7), an anthraquinodimethane derivative, a diphenylquinone derivative, bathocuproine, bathophenanthroline and a derivative thereof, a triazole compound, a tris(8-hydroxyquinolinate)aluminum complex, a bis(4-methyl-8-quinolinate)aluminum complex, a distyrylarylene derivative, a sylol compound and the like. In addition, any material may be used as long as the material is not an electron accepting organic material, but has a sufficient electron transporting property. A porphyrin-based compound, a styryl-based compound such as DCM (4-dicyanomethylene-2-methyl-6-(4-(dimethylaminostyryl))-4H pyran), and a 4H pyran-based compound may be used.

[Optical Sensor]

The photoelectric conversion device may be divided roughly into a photoelectric cell and an optical sensor, but the photoelectric conversion device of the present invention is useful to the optical sensor. The optical sensor may have a form of using the photoelectric conversion device alone, and may be in the form of a line sensor where the photoelectric conversion devices are linearly disposed, or the form of a two-dimensional sensor where the photoelectric conversion devices are disposed on a plane. The photoelectric conversion device of the present invention serves as an imaging device by converting optical image information into an electric signal using an optical system and a driving unit, such as a scanner, in a line sensor, and converting light imaging information into an electric signal by image-forming the optical image information using an optical system on a sensor, such as an imaging module, in a two-dimensional sensor.

Since a photoelectric cell is a power generation apparatus, an efficiency for converting light energy into electric energy is an important performance, and dark current that is current in a dark place is not considered as a problem in terms of a function. Further, when installing a color filter, a heating process in the subsequent stage is not required. Since an important performance of the optical sensor is conversion of a brightness signal into an electric signal with a high precision, efficiency for converting a light quantity into current is an important performance, but when output in a dark place, the signal becomes a noise and therefore, low dark current is required. Further, resistance to a step in the subsequent stage is also important.

[Imaging Device]

Next, configuration examples of an imaging device including the photoelectric conversion device of the present invention will be described. Further, in the configuration examples to be described below, for the members and the like having the configuration action equivalent to those of the members and the like previously described, the description thereof will be simplified or omitted by imparting the same or like reference numerals in the drawing.

The imaging device is a device of converting light information of an image into an electric signal, in which a plurality of photoelectric conversion devices is disposed on a matrix in the same plane form and light signals may be converted into electric signals in each photoelectric conversion device (pixel) and the electric signals may be output for each pixel to the outside of a sequential imaging device. Accordingly, the imaging device is configured of one photoelectric conversion device and one or more transistors per one pixel.

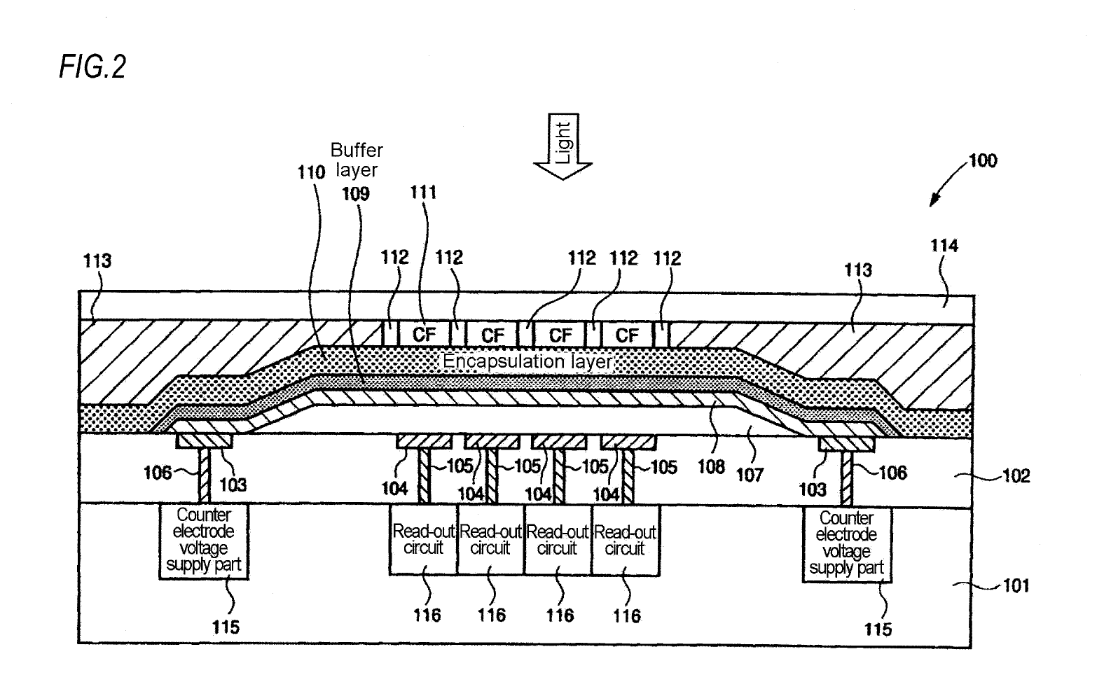

FIG. 2 is a cross-sectional schematic view illustrating a schematic configuration of an imaging device for describing an exemplary embodiment of the present invention. The imaging device is used by being mounted in an imaging apparatus such as a digital camera and a digital video camera, an imaging module such as an electronic endoscope and a mobile phone, and the like.

This imaging device includes a plurality of photoelectric conversion devices which is configured as illustrated in FIG. 1, and a circuit board formed with a read-out circuit for reading out signals according to the charges generated in the photoelectric conversion film of each photoelectric conversion device, in which the plurality of photoelectric conversion devices is one-dimensionally or two-dimensionally arranged on the same surface at an upper side of the circuit board.

An imaging device 100 illustrated in FIG. 2 includes a substrate 101, an insulating layer 102, a connection electrode 103, a pixel electrode (lower electrode) 104, a connection part 105, a connection part 106, a photoelectric conversion film 107, a counter electrode (upper electrode) 108, a buffer layer 109, an encapsulation layer 110, a color filter (CF) 111, a partition 112, a light-shielding layer 113, a protective layer 114, a counter electrode voltage supply part 115, and a read-out circuit 116.

The pixel electrode 104 has the same function as the electrode 11 of the photoelectric conversion device 10a illustrated in FIG. 1. The counter electrode 108 has the same function as the electrode 15 of the photoelectric conversion device 10a illustrated in FIG. 1. The photoelectric conversion film 107 has the same configuration as the layer formed between the electrode 11 and the electrode 15 of the photoelectric conversion device 10a illustrated in FIG.

The substrate 101 is a glass substrate or a semiconductor substrate such as Si. The insulating layer 102 is formed on the substrate 101. The plurality of pixel electrodes 104 and the plurality of connection electrodes 103 are formed on the surface of the insulating layer 102.

The photoelectric conversion film 107 is a common layer in all the photoelectric conversion devices provided on the plurality of pixel electrodes 104 by covering the plurality of pixel electrodes 104.

The counter electrode 108, which is provided on the photoelectric conversion film 107, is a common electrode in all the photoelectric conversion devices. The counter electrode 108 is formed even on the connection electrode 103 disposed at the outer side of the photoelectric conversion film 107, and is electrically connected to the connection electrode 103.

The connection part 106 is buried in the insulating layer 102, and is a plug and the like for electrically connecting the connection electrode 103 and the counter electrode voltage supply part 115. The counter electrode voltage supply part 115 is formed on the substrate 101, and applies a predetermined voltage to the counter electrode 108 through the connection part 106 and connection electrode 103. When the voltage to be applied to the counter electrode 108 is higher than a power source voltage of the imaging device, the predetermined voltage is supplied by increasing a power source voltage using a booster circuit such as a charge pump.

The read-out circuit 116 is provided on the substrate 101 to correspond to each of the plurality of pixel electrodes 104, and reads out the signal according to the electric charge collected in the corresponding pixel electrode 104. The read-out circuit 116 is composed of, for example, CCD, a CMOS circuit or a TFT circuit and the like, and is light-shielded by a light-shielding layer disposed in the insulating layer 102, which is not illustrated. The read-out circuit 116 is electrically connected to the pixel electrode 104 corresponding thereto through the connection part 105.

The buffer layer 109 is formed on the counter electrode 108 while covering the counter electrode 108. The encapsulation layer 110 is formed on the buffer layer 109 while covering the buffer layer 109. The color filter 111 is formed at a position facing each the pixel electrode 104 on the encapsulation layer 110. The partition 112 is provided between the color filters 111, and is for enhancing light transmittance efficiency of the color filter 111.

The light-shielding layer 113 is formed on the encapsulation layer 110 in a region other than a region in which the color filter 111 and the partition 112 are provided, and prevents light from entering the photoelectric conversion film 107 formed in a region other than an effective pixel region. The protective layer 114 is formed on the color filter 111, the partition 112 and the light-shielding layer 113, and protects the entire imaging device 100.

In the imaging device 100 as configured above, upon light being incident, light enters the photoelectric conversion film 107, so that an electric charge is generated. Out of the electric charges generated, holes are collected in the pixel electrode 104, and voltage signals according to the amount thereof are output by the read-out circuit 116 to the outside of the imaging device 100.

The manufacturing method of the imaging device 100 is as follows.

On the circuit substrate on which the counter electrode voltage supply part 115 and the read-out circuit 116 are formed, the connection parts 105 and 106, the plurality of connection electrode 103, the plurality of pixel electrodes 104 and the insulating layer 102 are formed. The plurality of pixel electrodes 104 is disposed on the surface of the insulating layer 102, for example, in a square lattice form.

Next, the photoelectric conversion film 107 is formed on a plurality of pixel electrodes 104, for example, by using a vacuum heating deposition method. Subsequently, the counter electrode 108 is formed on the photoelectric conversion film 107, for example, by a sputter method under the vacuum. Next, the buffer layer 109 and the encapsulation layer 110 are sequentially formed on the counter electrode 108, for example, by a vacuum heating deposition method. Subsequently, after the color filter 111, the partition 112 and the light-shielding layer 113 are formed, the protective layer 114 is formed, thereby completing the imaging device 100.

Even in the manufacturing method of the imaging device 100, even though a step of placing the imaging device 100 during the manufacturing in a non-vacuum atmosphere is added between a step of forming the photoelectric conversion layer included in the photoelectric conversion film 107 and a step of forming the encapsulation layer 110, deterioration in performance of a plurality of photoelectric conversion devices may be prevented. It is possible to suppress the manufacturing cost by adding this step while preventing the deterioration in performance of the imaging device 100.

Hereinafter, details of the encapsulation layer 110 as a constituent element of the above-described imaging device 100 will be described.

[Encapsulation Layer]

The following conditions are required for the encapsulation layer 110.

Firstly, the encapsulation layer 110 needs to protect the photoelectric conversion layer by blocking invasion of a factor degrading the organic photoelectric conversion material included in a solution, plasma and the like in each manufacturing step of the device.

Secondly, after the device is manufactured, the encapsulation layer 110 needs to prevent deterioration in the photoelectric conversion film 107 during storage and use for a long period of time by blocking invasion of the factor degrading the organic photoelectric conversion material such as water molecules.

Thirdly, when the encapsulation layer 110 is formed, the encapsulation layer 110 need not degrade the photoelectric conversion layer formed in advance.

Fourthly, since incident light reaches the photoelectric conversion film 107 through the encapsulation layer 110, with respect to light of a wavelength detected in the photoelectric conversion film 107, the encapsulation layer 110 needs to be transparent.

The encapsulation layer 110 may also be configured of a thin film made of a single material, but may have a multilayer configuration so as to impart different functions to respective layers, thereby expecting effects of relieving the stress of the entire encapsulation layer 110, suppressing the generation of defects such as cracks and pinholes due to dust generation or like in the manufacturing process, facilitating the optimization of material development and the like. For example, the encapsulation layer 110 may have a two-layered structure in which an “encapsulation auxiliary layer” having a function that is difficult to be accomplished by a layer, which is used for the original purpose of blocking permeation of deterioration factors such as water molecules, is laminated on the layer. A configuration of three or more layers may be feasible, but in consideration of the manufacturing costs, the number of layers is preferably small.

[Organic Electroluminescence Device]

An organic electroluminescence device using the material for organic electronic of the present invention will be described in detail.

The organic electroluminescence device according to the present invention is an organic electroluminescence device having at least one organic layer including a light emitting layer between a pair of electrodes, in which the organic layer contains the material for organic electronics of the present invention. Here, the material for organic electronics of the present invention may be any one of a light emitting material, a host material, an electron transporting material, a hole transporting material, a charge blocking material and a hole blocking material, but is preferably a light emitting material, a host material, a hole transporting material and a charge blocking material, and more preferably a light emitting material, a host material and a hole transporting material.

After compounds for all of the respective materials are synthesized, a high-purity material may be obtained with high yield in a short period of time by purification using the purification method of the present invention.

<Configuration of Organic Layer>

The layer configuration of the organic layer is not particularly limited and may be appropriately selected according to the use and purpose of the organic electroluminescence device, but is preferably formed on the transparent electrode or on the back electrode. In this case, the organic layer is formed on the front surface or one surface on the transparent electrode or the back electrode.

The shape, size, thickness and the like of the organic layer are not particularly limited and may be appropriately selected according to the purpose thereof.

Examples of the specific layer configuration of the organic electroluminescence device according to the present invention include the following configurations, but the present invention is not limited to these configurations.
Anode/hole transporting layer/light emitting layer/electron transporting layer/cathode
Anode/hole transporting layer/light emitting layer/hole blocking layer/electron transporting layer/cathode
Anode/hole transporting layer/light emitting layer/hole blocking layer/electron transporting layer/electron injection layer/cathode
Anode/hole injection layer/hole transporting layer/light emitting layer/hole blocking layer/electron transporting layer/cathode
Anode/hole transporting layer/charge blocking layer/light emitting layer/electron transporting layer/cathode
Anode/hole transporting layer/charge blocking layer/light emitting layer/electron transporting layer/electron injection layer/cathode
Anode/hole injection layer/hole transporting layer/charge blocking layer/light emitting layer/electron transporting layer/cathode
Anode/hole injection layer/hole transporting layer/charge blocking layer/light emitting layer/electron transporting layer/electron injection layer/cathode
Anode/hole injection layer/hole transporting layer/charge blocking layer/light emitting layer/hole blocking layer/electron transporting layer/electron injection layer/cathode
Anode/hole injection layer/hole transporting layer/charge blocking layer/light emitting layer/hole blocking layer/electron injection layer/cathode
Anode/hole injection layer/hole transporting layer/charge blocking layer/light emitting layer/hole blocking layer/electron transporting layer/cathode
Anode/hole injection layer/hole transporting layer/light emitting layer/blocking layer/electron transporting layer/electron injection layer/cathode
Anode/hole injection layer/hole transporting layer/charge blocking layer/light emitting layer/hole blocking layer/electron transporting layer/electron injection layer/cathode
Anode/hole injection layer/hole transporting layer/light emitting layer/electron transporting layer/electron injection layer/cathode

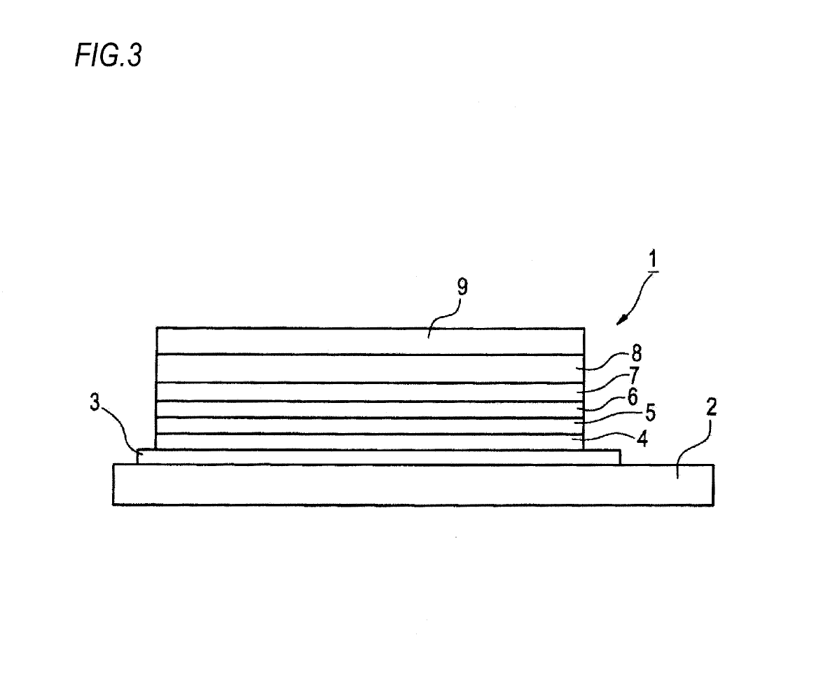

FIG. 3 illustrates an example of the configuration of the organic electroluminescence device according to the present invention. In an organic electroluminescence device 1 according to the present invention, which is illustrated in FIG. 3, a light emitting layer 6 is interposed between an anode 3 and a cathode 9 on a supporting substrate 2. Specifically, a hole injection layer 4, a hole transporting layer 5, a light emitting layer 6, a hole blocking layer 7 and an electron transporting layer 8 are laminated in this order between the anode 3 and the cathode 9.

Hereinafter, each element constituting the organic electroluminescence device according to the present invention will be described in detail.

<Substrate>

A substrate which is used in the present invention is preferably a substrate which does not scatter or decay light generated from the organic layer. In the case of an organic material, it is preferred that the organic material is excellent in heat resistance, dimensional stability, solvent resistance, electrical insulation properties and processability.

<Anode>

Typically, an anode is not particularly limited with respect to the shape, structure, size and the like thereof as long as the anode has a function as an electrode for supplying an organic layer with holes, and a material may be appropriately selected among the known electrode materials according to the use or purpose of the luminescence device. As described above, the anode is usually provided as a transparent anode.

<Cathode>

Typically, a cathode is not particularly limited with respect to the shape, structure, size and the like thereof as long as the cathode has a function as an electrode for injecting electrons into the organic layer, and a material may be appropriately selected among the known electrode materials according to the use or purpose of the luminescence device.

With respect to the substrate, the anode and the cathode, the matters described in paragraph Nos. [0070] to [0089] of the Japanese Patent Application Laid-Open No. 2008-270736 may be applied to the present invention.

<Organic Layer>

An organic layer in the present invention will be described.

The organic layer includes a light emitting layer, and examples of an organic layer other than the light emitting layer include the hole transporting layer, the electron transporting layer, the hole blocking layer, the charge blocking layer, the hole injection layer, the electron injection layer and the like.

Formation of Organic Layer

In the organic electroluminescence device of the present invention, each organic layer may be appropriately formed by any one of a dry film forming method such as a deposition method or a sputtering method, a wet film forming method such as solution application, a transfer method, a printing method and the like.

Light Emitting Layer

A light emitting layer is a layer having functions, when an electric field is applied, of accepting holes from the anode, the hole injection layer or the hole transporting layer and accepting electrons from the cathode, the electron injection layer or the electron transporting layer to provide a site of recombination of the holes and the electrons, thereby achieving light emission.

The light emitting layer in the present invention may be composed only of a light emitting material and may be composed of a mixed layer of a host material and a light emitting material. As the light emitting material, a fluorescent light emitting material or phosphorescent light emitting material may be used, and a dopant may be used either alone or in combination of two or more thereof. The host material is preferably a charge transporting material. The host material may be used either alone or in combination of two or more thereof, and examples thereof include a configuration of a mixture of an electron transporting host material and a hole transporting host material. In addition, the light emitting layer may include a material (binder material) which does not have a charge transporting property and does not emit light.

Further, the light emitting layer may have a single layer or a multi layer of two or more layers. In addition, each light emitting layer may emit light with different light emission colors.

(Fluorescent Light Emitting Material)

Examples of the fluorescent light emitting material which may be used in the present invention include a benzoxazole derivative, a benzimidazole derivative, a benzothiazole derivative, a styrylbenzene derivative, a polyphenyl derivative, a diphenylbutadiene derivative, a tetraphenylbutadiene derivative, a naphthalimide derivative, a coumarin derivative, a condensed aromatic compound, a perynone derivative, an oxadiazole derivative, an oxazine derivative, an aldazine derivative, a pyralidine derivative, a cyclopentadiene derivative, a bisstyrylanthracene derivative, a quinacridone derivative, a pyrrolopyridine derivative, a thiadiazolopyridine derivative, a cyclopentadiene derivative, a styrylamine derivative, a diketopyrrolopyrole derivative, an aromatic dimethylidine compound, various complexes represented by a complex of an 8-quinolinol derivative or a complex of a pyromethene derivative, and the like, a polymer compound such as polythiophene, polyphenylene and polyphenylenevinylene, a compound such as an organic silane derivative, and the like.

(Phosphorescent Light Emitting Material)

Examples of the phosphorescent light emitting material which may be used in the present invention include a phosphorescent light emitting compound and the like described in the patent documents such as U.S. Pat. No. 6,303,238B1, U.S. Pat. No. 6,097,147, WO00/57676, WO00/70655, WO01/08230, WO01/39234A2, WO01/41512A1, WO02/02714A2, WO02/15645A1, WO02/44189A1, WO05/19373A2, Japanese Patent Application Laid-Open Nos. 2001-247859, 2002-302671, 2002-117978, 2003-133074, 2002-235076, 2003-123982 and 2002-170684, EP1211257, Japanese Patent Application Laid-Open Nos. 2002-226495, 2002-234894, 2001-247859, 2001-298470, 2002-173674, 2002-203678, 2002-203679, 2004-357791, 2006-256999, 2007-19462, 2007-84635, and 2007-96259 and the like, and among them, examples of more preferred light emitting dopants include an Ir complex, a Pt complex, a Cu complex, a Re complex, a W complex, a Rh complex, a Ru complex, a Pd complex, an Os complex, an Eu complex, a Tb complex, a Gd complex, a Dy complex and a Ce complex. An Ir complex, a Pt complex or a Re complex is particularly preferred, and among them, an Ir complex, a Pt complex, or a Re complex, including at least one coordination mode of a metal-carbon bond, a metal-nitrogen bond, a metal-oxygen bond and a metal-sulfur bond, is preferred. In addition, from the viewpoint of light emission efficiency, driving durability, chromaticity and the like, an Ir complex, a Pt complex, or a Re complex including a tridentate or higher polydentate ligand, is particularly preferred.

The content of the light emitting material which may be used in the present invention is preferably 0.1% by mass to 50% by mass, more preferably 1% by mass to 40% by mass, and most preferably 5% by mass to 30% by mass, based on the total mass of the light emitting layer. Particularly, in the range of 5% by mass to 30% by mass, dependency of the chromaticity of light emission of the organic electroluminescence device on the addition concentration of the light emitting material is small.

(Host Material)

A host material refers to a compound which is usually responsible for injecting and transporting electric charges in a light emitting layer, and does not substantially emit light in itself. As used herein, “not substantially emit light” means that an amount of light emission from the compound that does not substantially emit light is preferably 5% or less, more preferably 3% or less, and still more preferably 1% or less, based on the total amount of light emission in the entire device.

In the present invention, it is preferred that a light emitting layer includes a host material.

Examples of the host material include a hole-transporting host material, an electron-transporting host material, or a so-called bipolar host material including both the materials, and the bipolar host material is preferred.

A concentration of the host material in the light emitting layer is not particularly limited, but is preferably a main component (a component having the largest content thereof) in the light emitting layer, more preferably 50% by mass to 99.9% by mass, still more preferably 50% by mass to 99.8% by mass, particularly preferably 60% by mass to 99.7% by mass, and most preferably 70% by mass to 95% by mass.

The glass transition point (Tg) of the host material is preferably 60° C. to 500° C., more preferably 90° C. to 250° C., and still more preferably 130° C. to 250° C., and among them, Tg is more preferably 175° C. to 250° C., particularly preferably 200° C. to 250° C. and most preferably 220° C. to 250° C.

In the light emitting layer, it is preferred that the lowest triplet excitation energy (T₁ energy) of the host material is higher than the T₁ energy of the light emitting material in terms of light emission efficiency and driving durability.

A partial structure thereof may contain the following compounds as the host material used in the present invention. Examples thereof include pyrrole, indole, carbazole (for example, CBP (4,4′-di(9-carbazoyl)biphenyl)), azaindole, azacarbazole, triazole, oxazole, oxadiazole, pyrazole, imidazole, thiophene, polyarylalkane, pyrazoline, pyrazolone, phenylenediamine, arylamine, amino substituted chalcone, styrylanthracene, fluorenone, hydrazone, stilbene, silazane, an aromatic tertiary amine compound, a styrylamine compound, a porphyrin-based compound, a polysilane-based compound, poly(N-vinylcarbazole), an aniline-based copolymer, an electrically conductive high-molecular oligomer such as a thiophene oligomer and polythiophene, organosilane, a carbon film, pyridine, pyrimidine, triazine, imidazole, pyrazole, triazole, oxazole, oxadiazole, fluorenone, anthraquinodimethane, anthrone, diphenylquinone, thiopyran dioxide, carbodiimide, fluorenylidenemethane, distyrylpyrazine, a fluorine-substituted aromatic compound, a heterocyclic tetracarboxylic anhydride such as naphthalene perylene, phthalocyanine, and various metal complexes represented by a metal complex of a 8-quinolinol derivative, metal phthalocyanine, and a metal complex including benzoxazole or benzothiazole as the ligand thereof and a derivative thereof (which may have a substituent or a condensed ring), or a material exemplified in the paragraph of a hole injection layer, a hole transporting layer, an electron injection layer and an electron transporting layer to be described below.

Further, as the host material used in the present invention, it is possible to suitably use, for example, a compound described in paragraph Nos. [0113] to [0161] of the Japanese Patent Application Laid-Open No. 2002-100476 and a compound described in paragraph Nos. [0087] to [0098] of the Japanese Patent Application Laid-Open No. 2004-214179, but the host material is not limited thereto.

A thickness of the light emitting layer is not particularly limited, but, usually, preferably 1 nm to 500 nm, more preferably 5 nm to 200 nm, and still more preferably 10 nm to 100 nm.

Electron Injection Layer and Electron Transporting Layer

A hole injection layer and a hole transporting layer are provided between an anode and a light emitting layer, and are a layer having a function of accepting holes from the anode or the anode side to transport the holes into the cathode side. It is preferred that the hole injection layer and the hole transporting layer are specifically a layer that contains a carbazole derivative, a triazole derivative, an oxazole derivative, an oxadiazole derivative, an imidazole derivative, a polyarylalkane derivative, a pyrazoline derivative, a pyrazolone derivative, a phenylenediamine derivative, an arylamine derivative, an amino substituted chalcone derivative, a styrylanthracene derivative, a fluorenone derivative, a hydrazone derivative, a stilbene derivative, a silazane derivative, an aromatic tertiary amine compound, a styrylamine compound, a porphyrin-based compound, an organic silane derivative, carbon and the like.

A thickness of the hole injection layer and the hole transporting layer is each preferably 500 nm or less from the viewpoint of decreasing the driving voltage.

The thickness of the hole transporting layer is preferably 1 nm to 500 nm, more preferably 5 nm to 200 nm, and still more preferably 5 nm to 100 nm. Further, the thickness of the hole injection layer is preferably 0.1 nm to 500 nm, more preferably 0.5 nm to 300 nm, and still more preferably 1 nm to 200 nm.

The hole injection layer and the hole transporting layer may have a single layer structure composed of one or two or more kinds of the above-described materials, or may have a multilayered structure composed of a plurality of layers having the same or different compositions.

Electron Injection Layer and Electron Transporting Layer

An electron injection layer and the electron transporting layer are provided between a cathode and a light emitting layer, and are a layer having a function of accepting electrons from the cathode or the cathode side to transport the electron into the anode side. It is preferred that the electron injection layer and the electron transporting layer are specifically a layer that contains a triazole derivative, an oxazole derivative, an oxadiazole derivative, an imidazole derivative, a fluorenone derivative, an anthraquinodimethane derivative, an anthrone derivative, a diphenylquinone derivative, a thiopyran dioxide derivative, a carbodiimide derivative, a fluorenylidenemethane derivative, a distyrylpyrazine derivative, a tetracarboxylic acid anhydride of an aromatic ring, such as naphthalene and perylene, a phthalocyanine derivative, a phenanthrene derivative, a phenanthroline derivative, various complexes represented by a complex of an 8-quinolinol derivative or a complex having metal phthalocyanine, benzoxazole or benzothiazole as a ligand, an organic silane derivative, and the like.

A thickness of the electron injection layer and the electron transporting layer is each preferably 100 nm or less from the viewpoint of decreasing the driving voltage.

The thickness of the electron transporting layer is preferably 1 nm to 100 nm, more preferably 5 nm to 50 nm, and still more preferably 10 nm to 30 nm. Further, the thickness of the electron injection layer is preferably 0.1 nm to 100 nm, more preferably 0.2 nm to 80 nm, and still more preferably 0.5 nm to 50 nm.

The electron injection layer and the electron transporting layer may have a single layer structure composed of one or two or more kinds of the above-described materials, or may have a multilayered structure composed of a plurality of layers having the same or different compositions.

Hole Blocking Layer

A hole blocking layer is provided between a cathode and a light emitting layer, and is a layer having a function of preventing holes transported from the anode side into the light emitting layer from going therethrough into the cathode side. In the present invention, the hole blocking layer may be formed as an organic layer adjacent to the light emitting layer at the cathode side.

Examples of the organic compound constituting the hole blocking layer include an aluminum complex such as aluminum(III)bis(2-methyl-8-quinolinato)4-phenylphenolate (abbreviated as BAlq), a carbazole derivative, a triazole derivative, a phenanthroline derivative such as 2,9-dimethyl-4,7-diphenyl-1,10-phenanthroline (abbreviated as BCP), and the like.

The thickness of the hole blocking layer is preferably 1 nm to 500 nm, more preferably 5 nm to 200 nm, and still more preferably 10 nm to 100 nm.

The hole blocking layer may have a single layer structure composed of one or two or more kinds of the above-described materials, or may have a multilayered structure composed of a plurality of layers having the same or different compositions.

Charge Blocking Layer

A charge blocking layer is provided between an anode and a light emitting layer, and is a layer having a function of preventing electrons transported from the cathode side into the light emitting layer from going therethrough into the anode side. In the present invention, the charge blocking layer may be formed as an organic layer adjacent to the light emitting layer at the anode side.

As an example of the organic compound constituting the charge blocking layer, those exemplified as the above-described hole transporting material may be applied.

The thickness of the charge blocking layer is preferably 1 nm to 500 nm, more preferably 5 nm to 200 nm, and still more preferably 10 nm to 100 nm.

The charge blocking layer may have a single layer structure composed of one or two or more kinds of the above-described materials or may have a multilayered structure composed of a plurality of layers having the same or different compositions.

<Protective Layer>

In the present invention, the entire organic EL device may be protected by a protective layer.

With respect to the protective layer, the matters described in paragraph Nos. [0169] and [0170] of the Japanese Patent Application Laid-Open No. 2008-270736 may be applied to the present invention.

<Sealing Container>

The entire device of the present invention may be sealed using a sealing container.

With respect to the sealing container, the matters described in paragraph No. [0171] of the Japanese Patent Application Laid-Open No. 2008-270736 may be applied to the present invention.

(Driving)

The organic electroluminescence device according to the present invention may achieve light emission by applying a voltage (typically from 2 volts to 15 volts) of direct current (may include an alternating current component if necessary) or a current of direct current between the anode and the cathode.

With respect to a driving method of the organic electroluminescence device according to the present invention, it is possible to apply driving methods described in each Japanese Patent Application Laid-Open Nos. H2-148687, 6-301355, 5-29080, 7-134558, 8-234685 and 8-241047, each Japanese Patent No. 2784615, U.S. Pat. Nos. 5,828,429 and 6,023,308, and the like.

(Use of Organic Electroluminescence Device)

The organic electroluminescence device according to the present invention may be suitably used for a display device, a display, a backlight, electrophotography, an illumination light source, a recording light source, an exposure light source, a reading light source, an indicator, a signboard, interiors or optical communications, and the like. In particular, the organic electroluminescence device according to the present invention is preferably used for a device that is driven in a region with high light emission luminance intensity, such as an illumination apparatus and a display apparatus.

[Substituent W]

The substituent W will be described.

Examples of the substituent group W include a halogen atom, an alkyl group (including a cycloalkyl group, a bicycloalkyl group and a tricycloalkyl group), an alkenyl group (including a cycloalkenyl group and a bicycloalkenyl group), an alkynyl group, an aryl group, a heterocyclic group (may also be called a heterocyclic ring group), a cyano group, a hydroxyl group, a nitro group, a carboxyl group, an alkoxy group, an aryloxy group, a sylyloxy group, a heterocyclic oxy group, an acyloxy group, a carbamoyloxy group, an alkoxycarbonyloxy group, an aryloxycarbonyloxy group, an amino group (including an anilino group), an ammonio group, an acylamino group, an aminocarbonylamino group, an alkoxycarbonylamino group, an aryloxycarbonylamino group, a sulfamoylamino group, an alkyl and arylsulfonylamino group, a mercapto group, an alkylthio group, an arylthio group, a heterocyclic thio group, a sulfamoyl group, a sulfo group, an alkyl and arylsulfinyl group, an alkyl and arylsulfonyl group, an acyl group, an aryloxycarbonyl group, an alkoxycarbonyl group, a carbamoyl group, an aryl and heterocyclic azo group, an imido group, a phosphino group, a phosphinyl group, a phosphinyloxy group, a phosphinylamino group, a phosphono group, a sylyl group, a hydrazino group, a ureido group, a boric acid group (—B(OH)₂), a phosphato group (—OPO(OH)₂), a sulfato group (—OSO₃H), and other known substituents.

More specifically, W represents the following (1) to (48).

(1) A Halogen Atom

For example, a fluorine atom, a chlorine atom, a bromine atom and an iodine atom

(2) An Alkyl Group

represents a straight, branched, or cyclic substituted or unsubstituted alkyl group. also includes (2-a) to (2-e).

(2-a) An Alkyl Group

preferably an alkyl group having 1 to 30 carbon atoms (for example, methyl, ethyl, n-propyl, isopropyl, t-butyl, n-octyl, eicosyl, 2-chloroethyl, 2-cyanoethyl and 2-ethylhexyl)

(2-b) A Cycloalkyl Group

preferably a substituted or unsubstituted cycloalkyl group having 3 to 30 carbon atoms (for example, cyclohexyl, cyclopentyl and 4-n-dodecylcyclohexyl)

(2-c) A Bicycloalkyl Group

preferably a substituted or unsubstituted bicycloalkyl group having 5 to 30 carbon atoms (for example, bicycle[1,2,2]heptan-2-yl and bicycle[2,2,2]octan-3-yl)

(2-d) A Tricycloalkyl Group

preferably a substituted or unsubstituted tricycloalkyl group having 7 to 30 carbon atoms (for example, 1-adamantyl)

(2-e) A Polycyclic Cycloalkyl Group Having a Larger Number of Ring Structures

Further, the alkyl group in the substituents described below (for example, the alkyl group in an alkylthio group) represents an alkyl group having such a concept and further includes an alkenyl group and an alkynyl group.

(3) An Alkenyl Group

represents a straight, branched, or cyclic substituted or unsubstituted alkenyl group. also includes (3-a) to (3-c).

(3-a) An Alkenyl Group

preferably a substituted or unsubstituted alkenyl group having 2 to 30 carbon atoms (for example, vinyl, allyl, prenyl, geranyl and oleyl)

(3-b) A Cycloalkenyl Group

preferably a substituted or unsubstituted cycloalkenyl group having 3 to 30 carbon atoms (for example, 2-cyclopenten-1-yl and 2-cyclohexen-1-yl)

(3-c) A Bicycloalkenyl Group

a substituted or unsubstituted bicycloalkenyl group, preferably a substituted or unsubstituted bicycloalkenyl group having 5 to 30 carbon atoms (for example, bicyclo[2,2,1]hept-2-en-1-yl and bicyclo[2,2,2]oct-2-en-4-yl)

(4) An Alkynyl Group

preferably a substituted or unsubstituted alkynyl group having 2 to 30 carbon atoms (for example, ethynyl, propargyl, and a trimethylsilylethynyl group)

(5) An Aryl Group

preferably a substituted or unsubstituted aryl group having 6 to 30 carbon atoms (for example, phenyl, p-tolyl, naphthyl, m-chlorophenyl, o-hexadecanoylaminophenyl and ferrocenyl)

(6) A Heterocyclic Group

preferably a monovalent group, obtained by removing one hydrogen atom from a 5- or 6-membered substituted or unsubstituted, aromatic or non-aromatic heterocyclic compound, more preferably a 5- or 6-membered aromatic heterocyclic group having 2 to 50 carbon atoms (for example, 2-furyl, 2-thienyl, 2-pyrimidinyl, 2-benzothiazolyl, 2-carbazolyl, 3-carbazolyl, 9-carbazolyl. Further, the heterocyclic group may also be a cationic heterocyclic group such as 1-methyl-2-pyridinio and 1-methyl-2-quinolino)

(7) A Cyano Group

(8) A Hydroxyl Group

(9) A Nitro Group

(10) A Carboxyl Group

(11) An Alkoxy Group

preferably a substituted or unsubstituted alkoxy group having 1 to 30 carbon atoms (for example, methoxy, ethoxy, isopropoxy, t-butoxy, n-octyloxy and 2-methoxyethoxy)

(12) An Aryloxy Group

preferably a substituted or unsubstituted aryloxy group having 6 to 30 carbon atoms (for example, phenoxy, 2-methylphenoxy, 4-t-butylphenoxy, 3-nitrophenoxy, 2-tetradecanoylaminophenoxy)

(13) A Silyloxy Group

preferably a silyloxy group having 3 to 20 carbon atoms (for example, trimethylsilyloxy, t-butyldimethylsilyloxy)

(14) A Heterocyclic Oxy Group

preferably a substituted or unsubstituted heterocyclic oxy group having 2 to 30 carbon atoms (for example, 1-phenyltetrazol-5-oxy and 2-tetrahydropyranyloxy)

(15) An Acyloxy Group

preferably a formyloxy group, a substituted or unsubstituted alkylcarbonyloxy group having 2 to 30 carbon atoms, or a substituted or unsubstituted arylcarbonyloxy group having 6 to 30 carbon atoms (for example, formyloxy, acetyloxy, pivaloyloxy, stearoyloxy, benzoyloxy and p-methoxyphenylcarbonyloxy)

(16) A Carbamoyloxy Group

preferably a substituted or unsubstituted carbamoyloxy group having 1 to 30 carbon atoms (for example, N,N-dimethylcarbamoyloxy, N,N-diethylcarbamoyloxy, morpholinocarbonyloxy, N,N-di-n-octylaminocarbonyloxy and N-n-octylcarbamoyloxy)

(17) An Alkoxycarbonyloxy Group

preferably a substituted or unsubstituted alkoxycarbonyloxy group having 2 to 30 carbon atoms (for example, methoxycarbonyloxy, ethoxycarbonyloxy, t-butoxycarbonyloxy and n-octylcarbonyloxy)

(18) An Aryloxycarbonyloxy Group

preferably a substituted or unsubstituted aryloxycarbonyloxy group having 7 to 30 carbon atoms (for example, phenoxycarbonyloxy, p-methoxyphenoxycarbonyloxy and p-n-hexadecyloxyphenoxycarbonyloxy)

(19) An Amino Group

preferably an amino group, a substituted or unsubstituted alkylamino group having 1 to 30 carbon atoms, or a substituted or unsubstituted anilino group having 6 to 30 carbon atoms (for example, amino, methylamino, dimethylamino, anilino, N-methyl-anilino and diphenylamino)

(20) An Ammonio Group

preferably an ammonio group or an ammonio group substituted with a substituted or unsubstituted alkyl, aryl or heterocyclic group having 1 to 30 carbon atoms (for example, trimethylammonio, triethylammonio and diphenylmethylammonio)

(21) An Acylamino Group

preferably a formylamino group, a substituted or unsubstituted alkylcarbonylamino group having 1 to 30 carbon atoms, or a substituted or unsubstituted arylcarbonylamino group having 6 to 30 carbon atoms (for example, formylammo, acetylammo, pivaloylamino, lauroylamino, benzoylamino and 3,4,5-tri-n-octyloxyphenylcarbonylamino)

(22) An Aminocarbonylamino Group

preferably a substituted or unsubstituted aminocarbonylamino group having 1 to 30 carbon atoms (for example, carbamoylamino, N,N-dimethylaminocarbonylamino, N,N-diethylaminocarbonylamino and morpholinocarbonylamino)

(23) An Alkoxycarbonylamino Group

preferably a substituted or unsubstituted alkoxycarbonylamino group having 2 to 30 carbon atoms (for example, methoxycarbonylamino, ethoxycarbonylamino, t-butoxycarbonylamino, n-octadecyloxycarbonylamino and N-methyl-methoxycarbonylamino)

(24) An Aryloxycarbonylamino Group

preferably a substituted or unsubstituted aryloxycarbonylamino group having form 7 to 30 carbon atoms (for example, phenoxycarbonylamino, p-chlorophenoxycarbonylamino and m-n-octyloxyphenoxycarbonylamino)

(25) A Sulfamoylamino Group

preferably a substituted or unsubstituted sulfamoylamino group having 0 to 30 carbon atoms (for example, sulfamoylamino, N,N-dimethylaminosulfonylamino and N-n-octylaminosulfonylamino)

(26) An Alkyl or Arylsulfonylamino Group

preferably a substituted or unsubstituted alkylsulfonylamino group having 1 to 30 carbon atoms, or a substituted or unsubstituted arylsulfonylamino group having 6 to 30 carbon atoms (for example, methylsulfonylamino, butylsulfonylamino, phenylsulfonylamino, 2,3,5-trichlorophenylsulfonylamino and p-methylphenylsulfonylamino)

(27) A Mercapto Group

(28) An Alkylthio Group

preferably a substituted or unsubstituted alkylthio group having 1 to 30 carbon atoms (for example, methylthio, ethylthio and n-hexadecylthio)

(29) An Arylthio Group

preferably a substituted or unsubstituted arylthio group having 6 to 30 carbon atoms (for example, phenylthio, p-chlorophenylthio and m-methoxyphenylthio)

(30) A Heterocyclic Thio Group

preferably a substituted or unsubstituted heterocyclic thio group having 2 to 30 carbon atoms (for example, 2-benzothiazolylthio and 1-phenyltetrazol-5-ylthio)

(31) A Sulfamoyl Group

preferably a substituted or unsubstituted sulfamoyl group having 0 to 30 carbon atoms (for example, N-ethylsulfamoyl, N-(3-dodecyloxypropyl)sulfamoyl, N,N-dimethylsulfamoyl, N-acetylsulfamoyl, N-benzoylsulfamoyl and N—(N′-phenylcarbamoyl)sulfamoyl)

(32) A Sulfo Group

(33) An Alkyl or Arylsulfinyl Group

preferably a substituted or unsubstituted alkylsulfinyl group having 1 to 30 carbon atoms, or a substituted or unsubstituted arylsulfinyl group having 6 to 30 carbon atoms (for example, methylsulfinyl, ethylsulfinyl, phenylsulfinyl and p-methylphenylsulfinyl)

(34) An Alkyl or Arylsulfonyl Group

preferably a substituted or unsubstituted alkylsulfonyl group having 1 to 30 carbon atoms, or a substituted or unsubstituted arylsulfonyl group having 6 to 30 carbon atoms (for example, methylsulfonyl, ethylsulfonyl, phenylsulfonyl and p-methylphenylsulfonyl)

(35) An Acyl Group

preferably a formyl group, a substituted or unsubstituted alkylcarbonyl group having 2 to 30 carbon atoms, a substituted or unsubstituted arylcarbonyl group having 7 to 30 carbon atoms, or a substituted or unsubstituted heterocyclic carbonyl group having 4 to 30 carbon atoms and being bonded to a carbonyl group through a carbon atom (for example, acetyl, pivaloyl, 2-chloroacetyl, stearoyl, benzoyl, p-n-octyloxyphenylcarbonyl, 2-pyridylcarbonyl and 2-furylcarbonyl)

(36) An Aryloxycarbonyl Group

preferably a substituted or unsubstituted aryloxycarbonyl group having 7 to 30 carbon atoms (for example, phenoxycarbonyl, o-chlorophenoxycarbonyl, m-nitrophenoxycarbonyl and p-t-butylphenoxycarbonyl)

(37) An Alkoxycarbonyl Group

preferably a substituted or unsubstituted alkoxycarbonyl group having 2 to 30 carbon atoms (for example, methoxycarbonyl, ethoxycarbonyl, t-butoxycarbonyl and n-octadecyloxycarbonyl)

(38) A Carbamoyl Group

preferably a substituted or unsubstituted carbamoyl group having 1 to 30 carbon atoms (for example, carbamoyl, N-methylcarbamoyl, N,N-dimethylcarbamoyl, N,N-di-n-octylcarbamoyl and N-(methylsulfonyl)carbamoyl)

(39) An Aryl and Heterocyclic Azo Group

preferably a substituted or unsubstituted arylazo group having 6 to 30 carbon atoms, or a substituted or unsubstituted heterocyclic azo group having 3 to 30 carbon atoms (for example, phenylazo, p-chlorophenylazo and 5-ethylthio-1,3,4-thiadiazol-2-ylazo)

(40) An Imido Group

preferably N-succinimido and N-phthalimido

(41) A Phosphino Group

preferably a substituted or unsubstituted phosphino group having 2 to 30 carbon atoms (for example, dimethylphosphino, diphenylphosphino and methylphenoxyphosphino)

(42) A Phosphinyl Group

preferably a substituted or unsubstituted phosphinyl group having 2 to 30 carbon atoms (for example, phosphinyl, dioctyloxyphosphinyl and diethoxyphosphinyl)

(43) A Phosphinyloxy Group

preferably a substituted or unsubstituted phosphinyloxy group having 2 to 30 carbon atoms (for example, diphenoxyphosphinyloxy and dioctyloxyphosphinyloxy)

(44) A Phosphinylamino Group

preferably a substituted or unsubstituted phosphinylamino group having 2 to 30 carbon atoms (for example, dimethoxyphosphinylamino and dimethylaminophosphinylamino)

(45) A Phospho Group

(46) A Silyl Group

preferably a substituted or unsubstituted silyl group having 3 to 30 carbon atoms (for example, trimethylsilyl, triethylsilyl, triisopropylsilyl, t-butyldimethylsilyl and phenyldimethylsilyl)

(47) A Hydrazino Group

preferably a substituted or unsubstituted hydrazino group having 0 to 30 carbon atoms (for example, trimethylhydrazino)

(48) A Ureido Group

preferably a substituted or unsubstituted ureido group having 0 to 30 carbon atoms (for example, N,N-dimethylureido)

Among the aforementioned substituents W, those having a hydrogen atom may be deprived of the hydrogen atom and further substituted with the aforementioned group. Examples of such a substituent include a —CONHSO₂— group (a sulfonylcarbamoyl group or a carbonylsulfamoyl group), a —CONHCO— group (a carbonylcarbamoyl group) and an —SO₂NHSO₂— group (a sulfonylsulfamoyl group). More specific examples thereof include an alkylcarbonylaminosulfonyl group (for example, acetylaminosulfonyl), an arylcarbonylaminosulfonyl group (for example, a benzoylaminosulfonyl group), an alkylsulfonylaminocarbonyl group (for example, methylsulfonylaminocarbonyl) and an arylsulfonylaminocarbonyl group (for example, p-methylphenylsulfonylaminocarbonyl).

[Ring R]

Examples of the ring R include an aromatic or non-aromatic hydrocarbon ring or heterocyclic ring or a polycyclic condensed ring formed by further combining these rings. Examples thereof include a benzene ring, a naphthalene ring, an anthracene ring, a phenanthrene ring, a fluorene ring, a triphenylene ring, a naphthacene ring, a biphenyl ring, a pyrrole ring, a furan ring, a thiophene ring, an imidazole ring, an oxazole ring, a thiazole ring, a pyridine ring, a pyrazine ring, a pyrimidine ring, a pyridazine ring, an indolizine ring, an indole ring, a benzofuran ring, a benzothiophene ring, an isobenzofuran ring, a quinolizine ring, a quinoline ring, a phthalazine ring, a naphthyridine ring, a quinoxaline ring, a quinoxazoline ring, an isoquinoline ring, a carbazole ring, a phenanthridine ring, an acridine ring, a phenanthroline ring, a thianthrene ring, a chromene ring, a xanthene ring, a phenoxathiin ring, a phenothiazine ring and a phenazine ring. The ring R may further have the substituent of the substituent W.

### EXAMPLE

Hereinafter, the present invention will be described in detail with reference to Examples, but the present invention is not limited thereto.

### Examples 1 to 38 and Comparative Examples 1 to 30

Hereinafter, the structure of the exemplified compound as an organic material used in the Examples and the Comparative Examples will be illustrated.

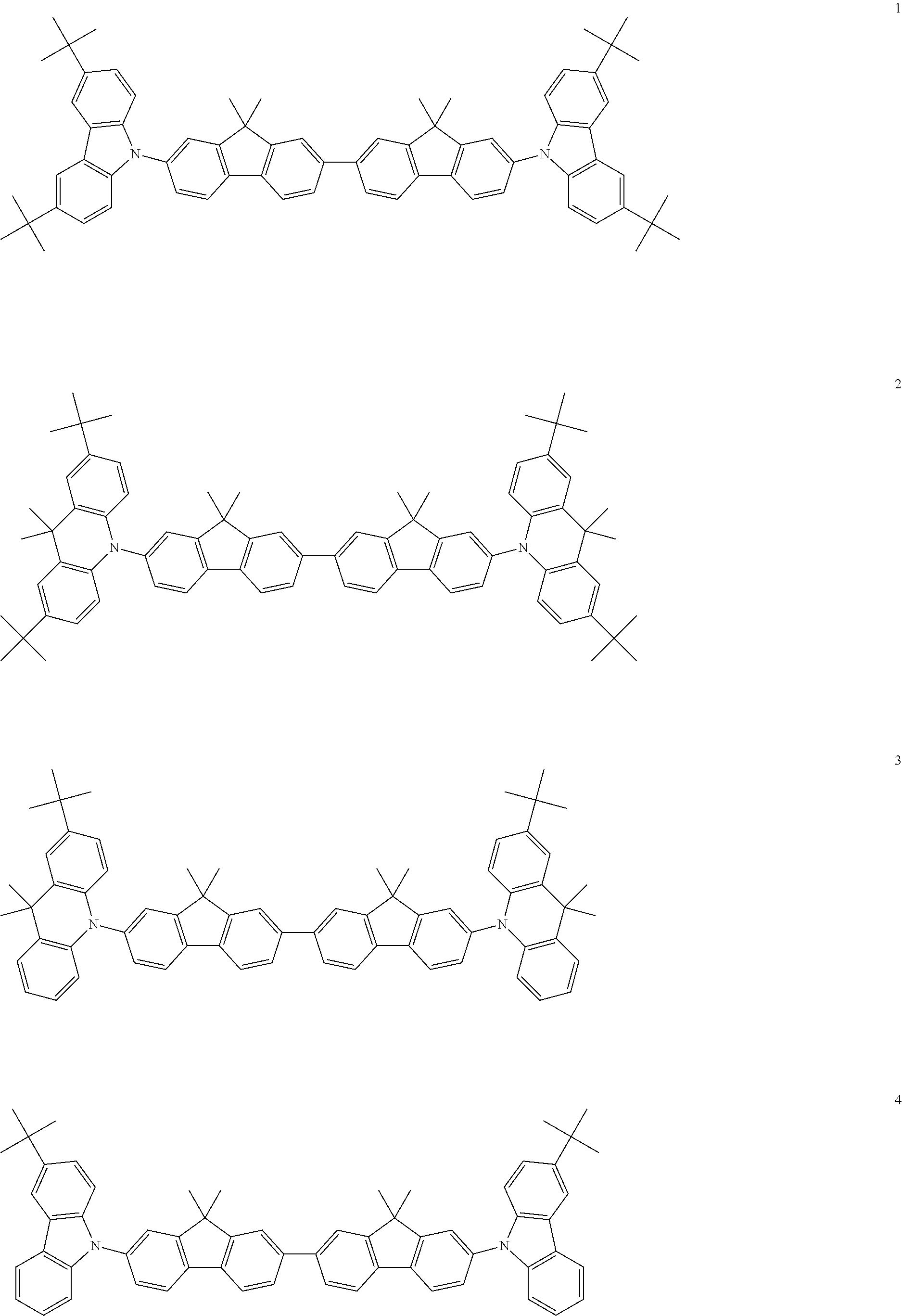

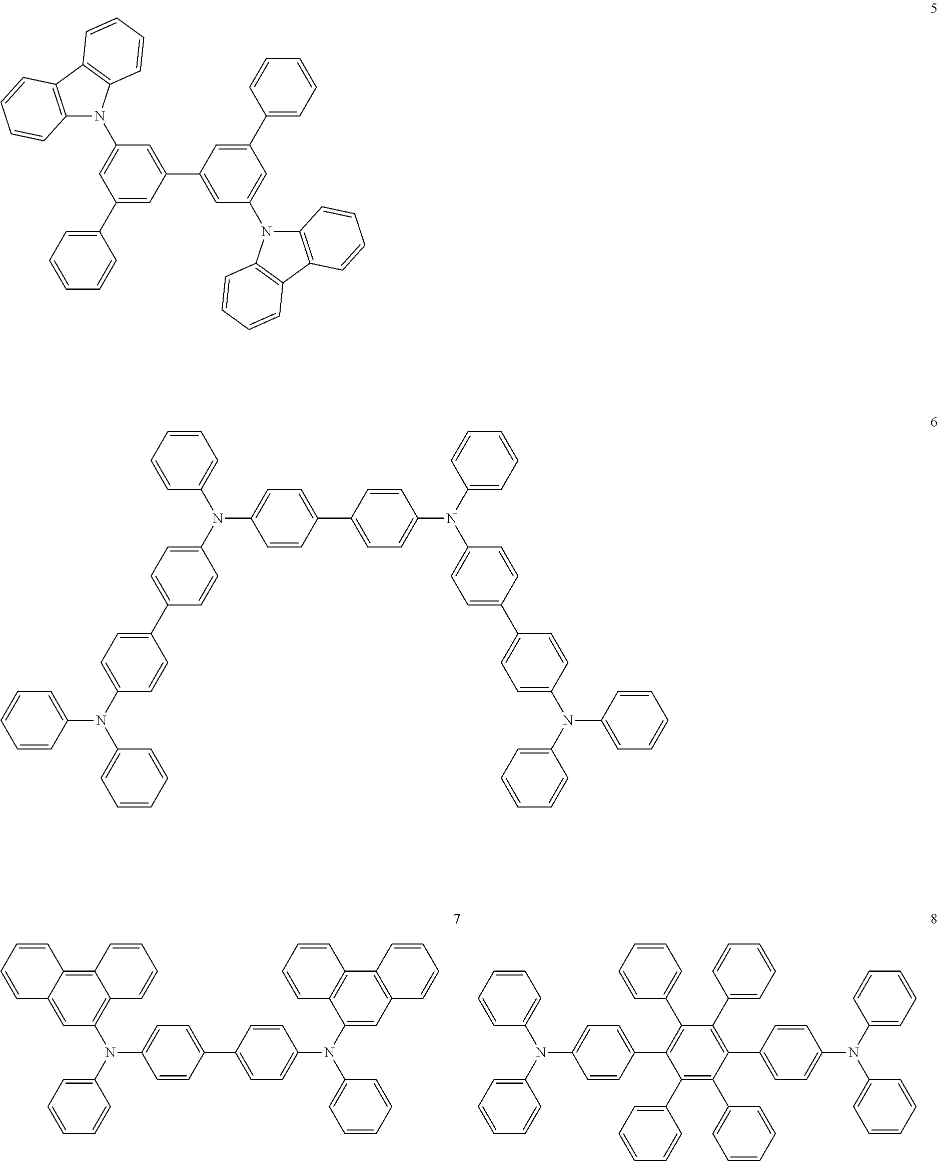 [SMILES: c1ccc(-c2cc(-c3cc(-c4ccccc4)cc(-n4c5ccccc5c5ccccc54)c3)cc(-n3c4ccccc4c4ccccc43)c2)cc1 ; c1ccc(N(c2ccccc2)c2ccc(-c3ccc(N(c4ccccc4)c4ccc(-c5ccc(N(c6ccccc6)c6ccc(-c7ccc(N(c8ccccc8)c8ccccc8)cc7)cc6)cc5)cc4)cc3)cc2)cc1 ; c1ccc(N(c2ccc(-c3ccc(N(c4ccccc4)c4cc5ccccc5c5ccccc45)cc3)cc2)c2cc3ccccc3c3ccccc23)cc1 ; c1ccc(-c2c(-c3ccccc3)c(-c3ccc(N(c4ccccc4)c4ccccc4)cc3)c(-c3ccccc3)c(-c3ccccc3)c2-c2ccc(N(c3ccccc3)c3ccccc3)cc2)cc1]

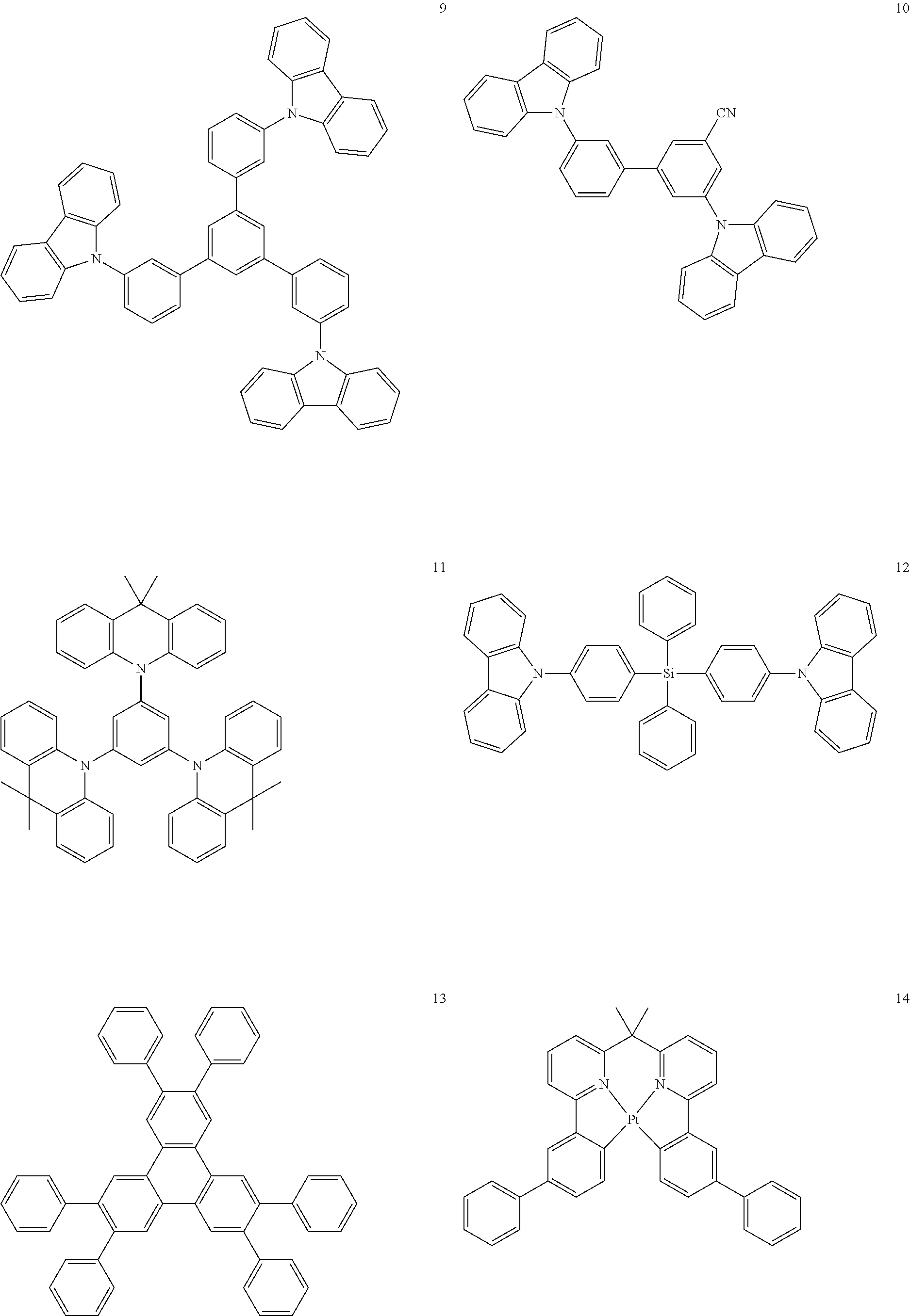

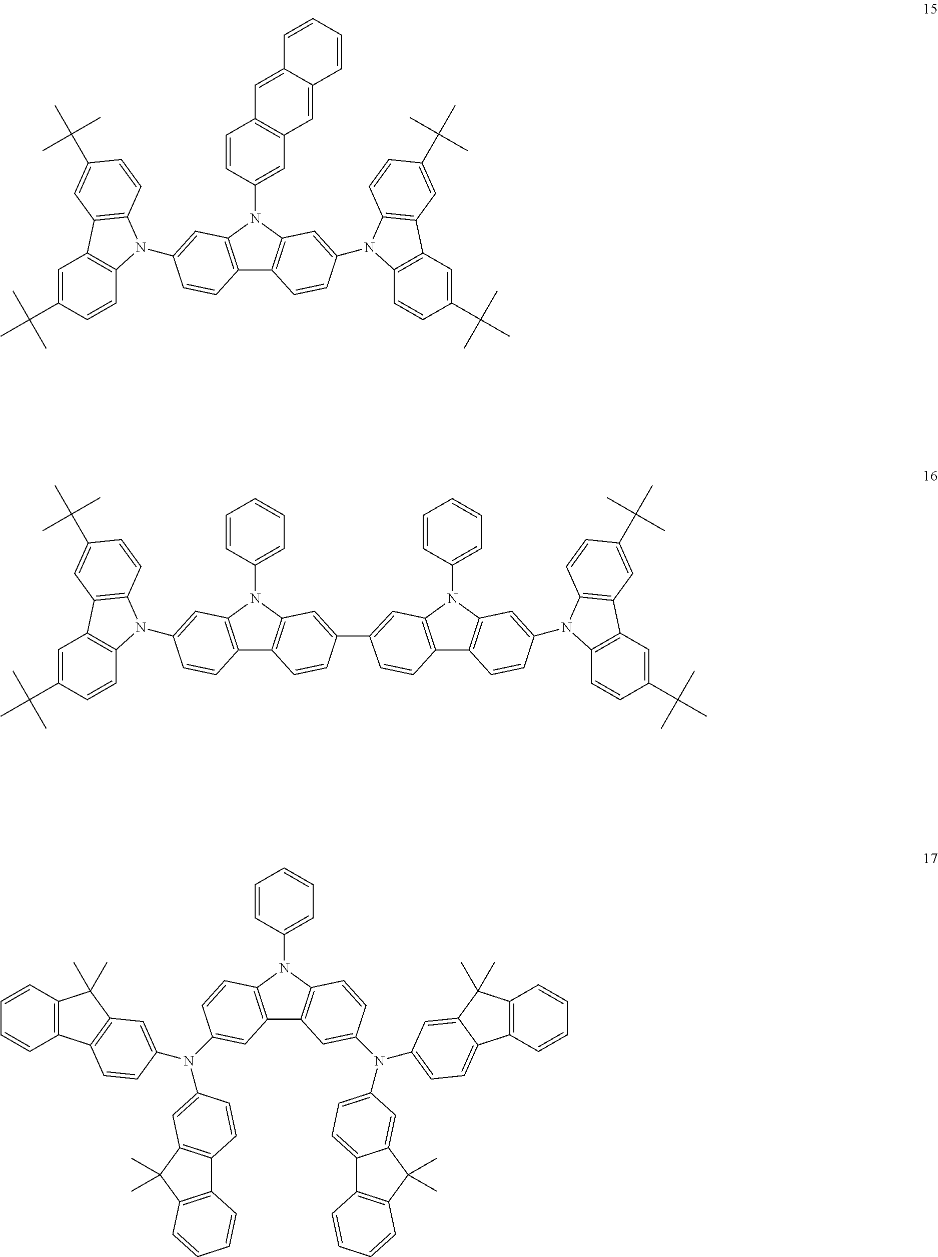

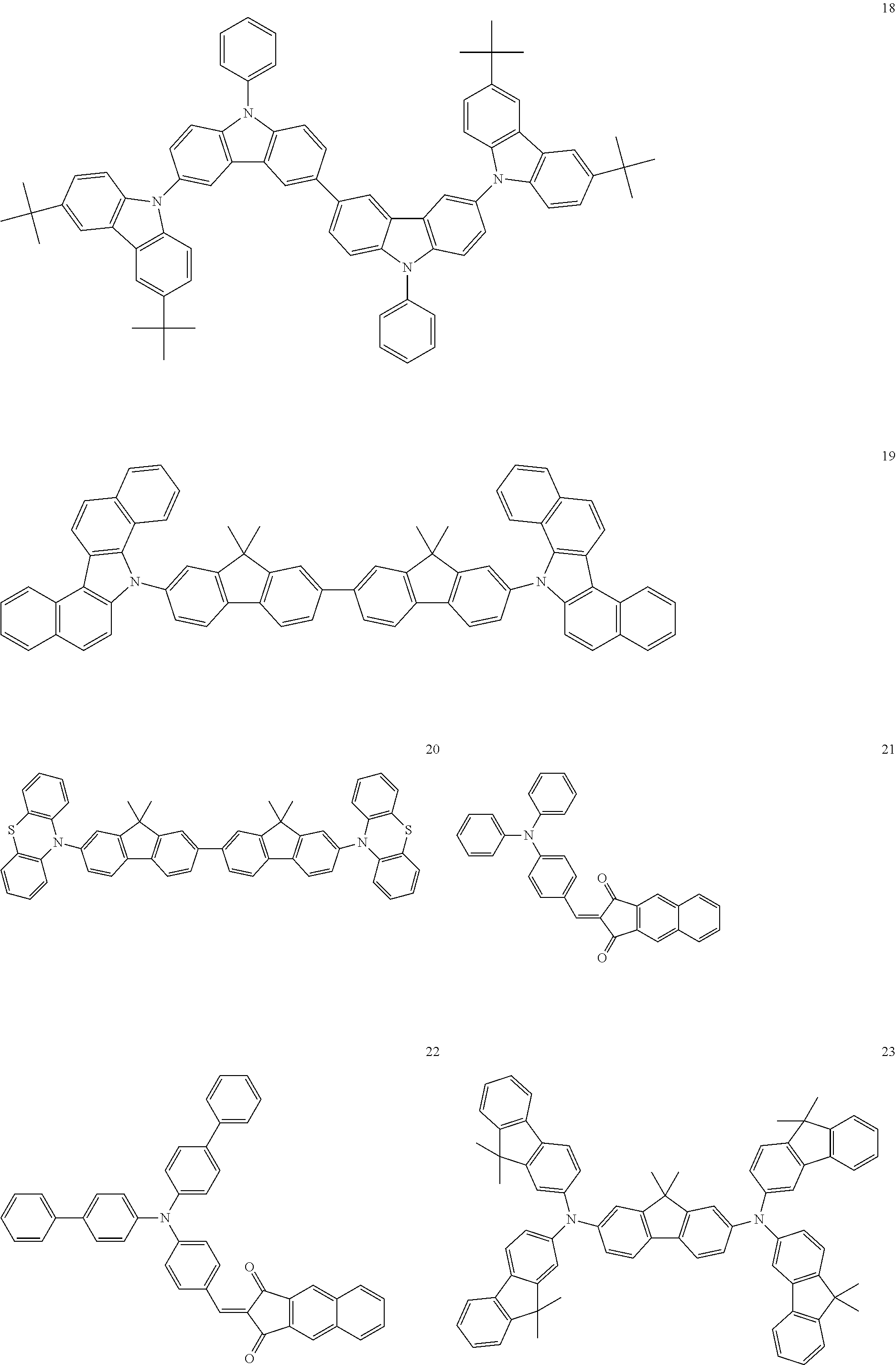

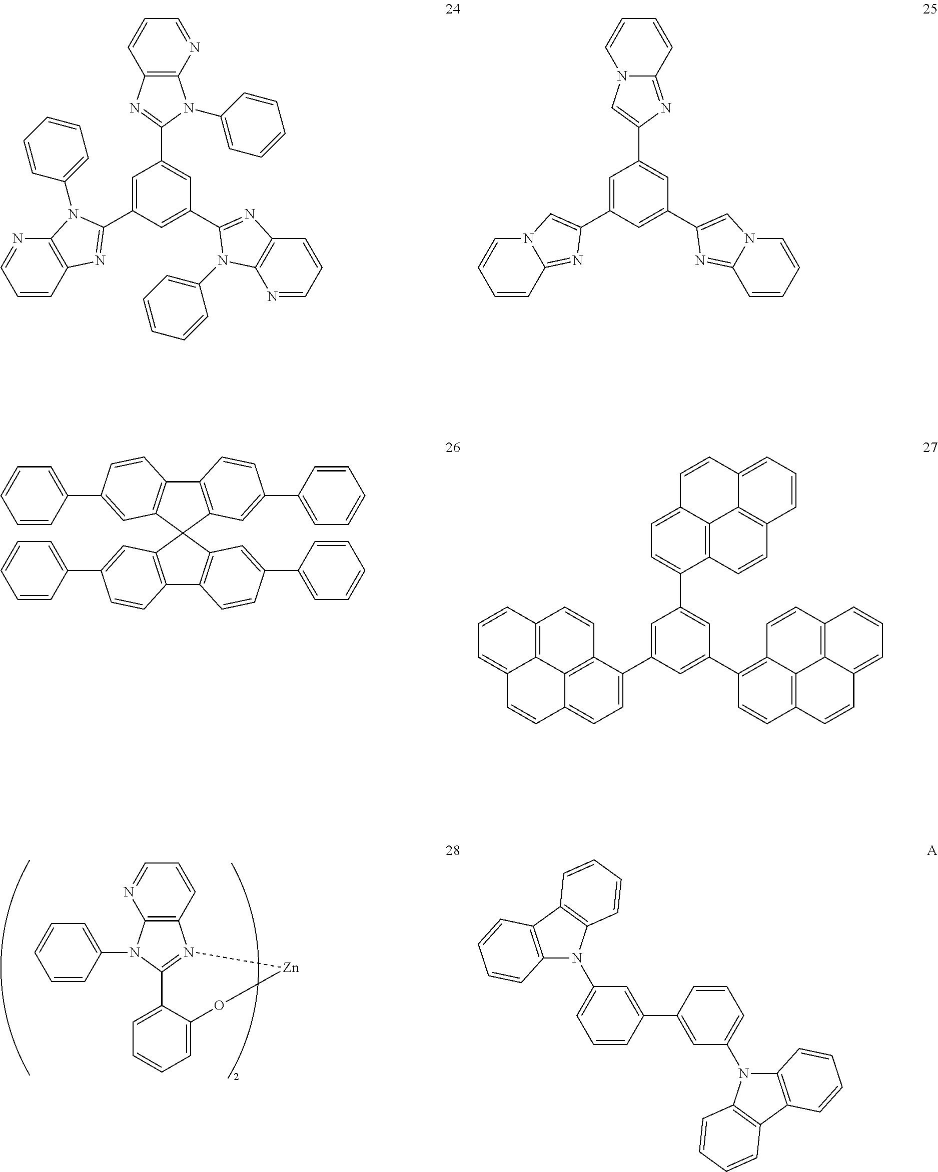

[Synthesis of Compound]

(Synthesis of Exemplified Compound 4)

Exemplified Compound 4 may be prepared by the following reaction formula.

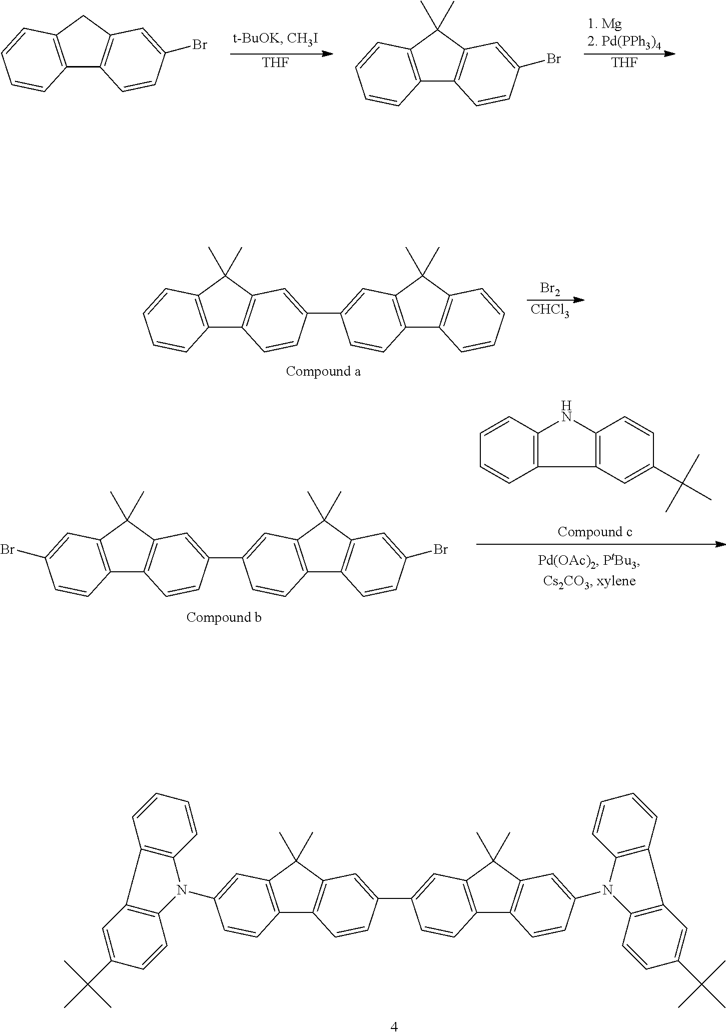

2-bromofluorene (89.0 g, 0.363 mol) was dissolved in 1.31 of tetrahydrofuran (THF) and cooled to 5° C., and potassium-tert-butoxide (102 g, 0.908 mol) was added thereto. Methyl iodide (565 ml, 0.908 mol) was added dropwise thereto at 5° C. After the dropwise addition was completed, 2-bromo-9,9-dimethyl-fluorene was obtained at a yield of 87% by stirring the mixture at room temperature for 5 hours. Magnesium powder (3.51 g, 0.144 mol) was added to 50 ml of THF under a nitrogen atmosphere, the resulting mixture was refluxed at a boiling temperature, a 250-ml THF solution of 2-bromo-9,9-dimethyl-fluorene (75.0 g, 0.275 mol) was added dropwise thereto, and the resulting mixture was stirred for 1 hour. Thereafter, Compound a was obtained at a yield of 82% by adding tetrakis(triphenylphosphine)palladium (1.59 g, 1.38 mol) thereto and refluxing the resulting mixture at a boiling temperature for 2 hours. Compound b was synthesized at a yield of 78% by adding bromine (39.8 g, 0.249 mol) dropwise to a 500-ml chloroform solution of Compound a (43.8 g, 0.113 mol) and stirring the resulting solution. Compound b (1.10 g, 2.02 mmol), palladium acetate (22.7 mg, 0.101 mmol), tri(t-butyl)phosphine (61.3 mg, 0.303 mmol), cesium carbonate (2.63 g, 8.08 mmol) and Compound c (991 mg, 4.44 mmol) were dissolved in 11 ml of xylene, and the resulting mixture was reacted by a boiling temperature reflux under a nitrogen atmosphere for 4 hours. An organic phase was separated by adding ethyl acetate and water to the reaction mixture, was washed with water and a saturated saline solution, and then concentrated under reduced pressure, and the obtained reaction mixture was purified by recrystallization, thereby obtaining Exemplified Compound 4 at a yield of 77%. The NMR measurement result of the obtained Exemplified Compound 4 is as follows.

¹H-NMR (400 MHz, in CDCl₃): δ(ppm)=1.50 (s, 18H), 1.65 (s, 12H), 7.28-7.32 (m, 2H), 7.40-7.46 (m, 4H), 7.49 (d, J=8.2, 2H), 7.53 (dd, J=8.7, 1.9 Hz, 2H), 7.57 (dd, J=8.0, 1.8 Hz, 2H), 7.66 (d, J=1.8 Hz, 2H), 7.74 (dd, J=7.9, 1.6 Hz, 2H), 7.77 (s, 2H), 7.89 (d, J=7.8 Hz, 2H), 7.96 (d, J=8.0 Hz, 2H), 8.18-8.18 (m, 6H)

According to HPLC, the obtained Exemplified Compound 4 had a purity of 99.5%. In an analysis by HR-ICP-MS using ELEMNTXR manufactured by Thermo Scientific Inc., the total content of Li, Na, K, Rb, Cs, Pd, Cu and Ni atoms and ions was 7520 ppm. Inorganic impurities were removed by dissolving the obtained Exemplified Compound 4 in toluene and filtering the resulting solution (filtered with two types of filter papers in accordance with the JIS Standards. In the above-described analysis of Exemplified Compound 4 by HR-ICP-MS after the inorganic impurities were removed, the total content of Li, Na, K, Rb, Cs, Pd, Cu and Ni atoms and ions was 2,600 ppm.

(Synthesis of Exemplified Compound 1)

Exemplified Compound 1 may be prepared by the following reaction formula.

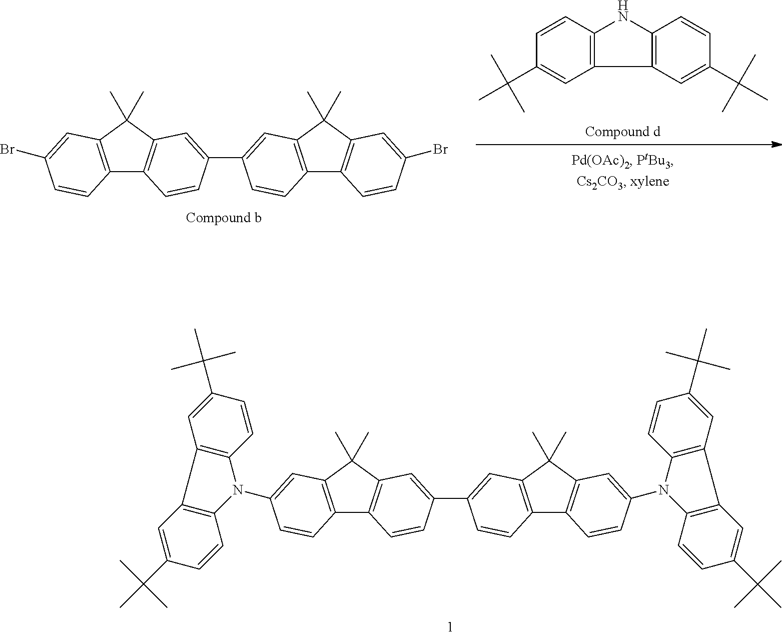 [SMILES: CC1(C)c2cc(Br)ccc2-c2ccc(-c3ccc4c(c3)C(C)(C)c3cc(Br)ccc3-4)cc21 ; CC(C)(C)c1ccc2[nH]c3ccc(C(C)(C)C)cc3c2c1 ; CC(C)(C)c1ccc2c(c1)c1cc(C(C)(C)C)ccc1n2-c1ccc2c(c1)C(C)(C)c1cc(-c3ccc4c(c3)C(C)(C)c3cc(-n5c6ccc(C(C)(C)C)cc6c6cc(C(C)(C)C)ccc65)ccc3-4)ccc1-2]

Compound b (1.10 g, 2.02 mmol), palladium acetate (22.7 mg, 0.101 mmol), tri(t-butyl)phosphine (61.3 mg, 0.303 mmol), cesium carbonate (2.63 g, 8.08 mmol) and Compound d (1.24 g, 4.44 mmol) were dissolved in 11 ml of xylene, and the resulting mixture was reacted by a boiling temperature reflux under a nitrogen atmosphere for 4 hours. An organic phase was separated by adding ethyl acetate and water to the reaction mixture, was washed with water and a saturated saline solution, and then concentrated under reduced pressure, and the obtained reaction mixture was purified by recrystallization, thereby obtaining Exemplified Compound 1 at a yield of 76%. The NMR measurement result of the obtained Exemplified Compound 1 is as follows.

¹H-NMR (400 MHz, in CDCl₃): δ(ppm)=1.49 (s, 36H), 7.44 (d, J=7.6 Hz, 4H), 7.51 (dd, J=8.4, 1.9 Hz, 4H), 7.56 (dd, J=8.0, 1.9 Hz, 2H), 7.65 (d, J=1.4 Hz, 2H), 7.73 (dd, J=7.8, 1.8 Hz, 2H), 7.77 (d, J=1.2 Hz, 2H), 7.88 (d, J=7.8 Hz, 2H), 7.95 (d, J=8.0 Hz, 2H), 8.17 (d, J=1.6 Hz, 4H) According to HPLC, the obtained Exemplified Compound 1 had a purity of 99.5%. In the above-described analysis by HR-ICP-MS, the total content of Li, Na, K, Rb, Cs, Pd, Cu and Ni atoms and ions was 5,935 ppm.

Inorganic impurities were removed by dissolving the obtained Exemplified Compound 1 in toluene and filtering the resulting solution (filtered with two types of filter papers in accordance with the JIS Standards, and then further filtered with four types of filter papers in accordance with the JIS Standards). In the above-described analysis of Exemplified Compound 1 by HR-ICP-MS after the inorganic impurities were removed, the total content of Li, Na, K, Rb, Cs, Pd, Cu and Ni atoms and ions was 911 ppm.

Exemplified Compound 1 may also be prepared by the following reaction formula.

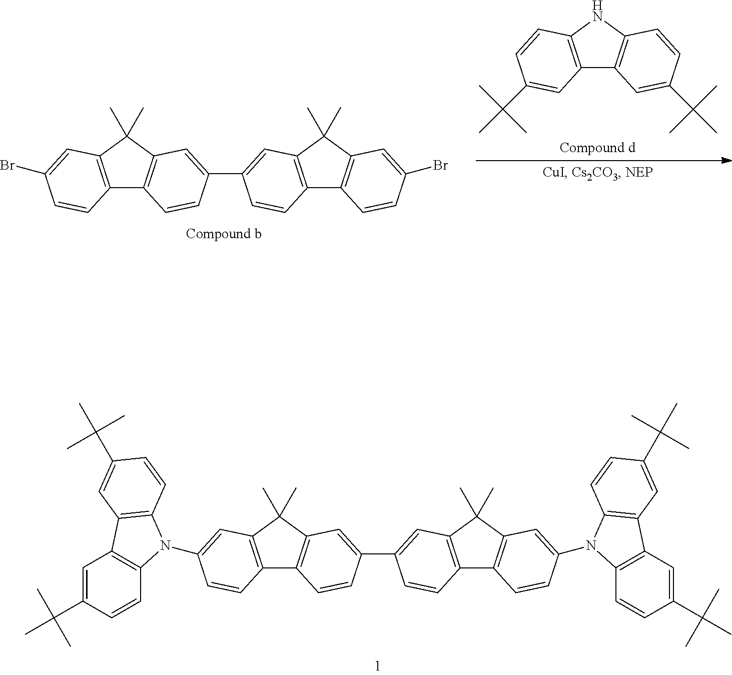 [SMILES: CC1(C)c2cc(Br)ccc2-c2ccc(-c3ccc4c(c3)C(C)(C)c3cc(Br)ccc3-4)cc21 ; CC(C)(C)c1ccc2[nH]c3ccc(C(C)(C)C)cc3c2c1 ; CC(C)(C)c1ccc2c(c1)c1cc(C(C)(C)C)ccc1n2-c1ccc2c(c1)C(C)(C)c1cc(-c3ccc4c(c3)C(C)(C)c3cc(-n5c6ccc(C(C)(C)C)cc6c6cc(C(C)(C)C)ccc65)ccc3-4)ccc1-2]

Compound b (5.00 g, 9.19 mmol), cuprous iodide (I) (1.75 g, 9.19 mmol), cesium carbonate (5.09 g, 15.6 mmol) and Compound d (5.90 g, 21.1 mmol) were dissolved in 20 ml of N-ethylpyrrolidone, and the resulting mixture was reacted by a boiling temperature reflux under a nitrogen atmosphere for 10 hours. The reaction mixture was dissolved in 100 ml of THF, filtered with celite, concentrated under reduced pressure and purified by recrystallization, thereby obtaining Exemplified Compound 1 at a yield of 61%.

According to HPLC, the obtained Exemplified Compound 1 had a purity of 99.0%. In the above-described analysis by HR-ICP-MS, the total content of Li, Na, K, Rb, Cs, Pd, Cu and Ni atoms and ions was 7,320 ppm.

(Synthesis of Exemplified Compound 2)

Exemplified Compound 2 may be prepared by the following reaction formula.

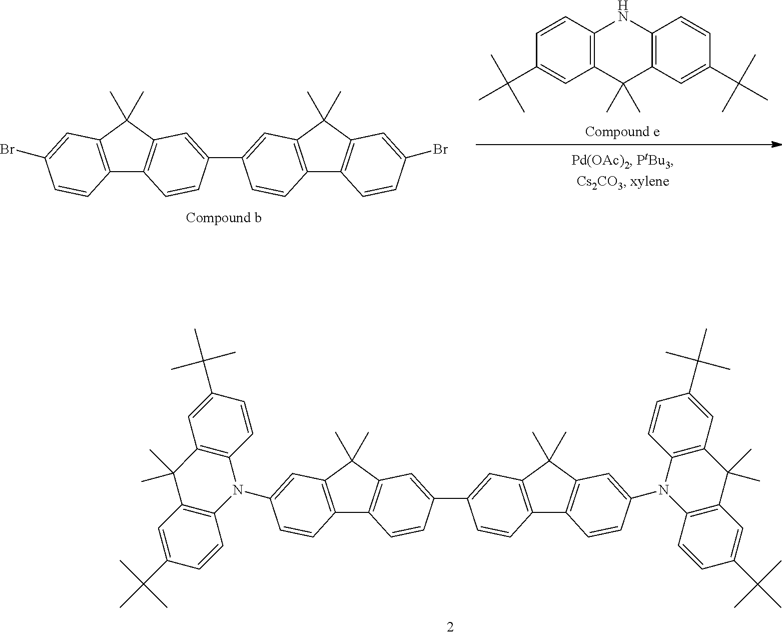 [SMILES: CC1(C)c2cc(Br)ccc2-c2ccc(-c3ccc4c(c3)C(C)(C)c3cc(Br)ccc3-4)cc21 ; CC(C)(C)c1ccc2c(c1)C(C)(C)c1cc(C(C)(C)C)ccc1N2 ; CC(C)(C)c1ccc2c(c1)C(C)(C)c1cc(C(C)(C)C)ccc1N2c1ccc2c(c1)C(C)(C)c1cc(-c3ccc4c(c3)C(C)(C)c3cc(N5c6ccc(C(C)(C)C)cc6C(C)(C)c6cc(C(C)(C)C)ccc65)ccc3-4)ccc1-2]

Compound b (1.10 g, 2.02 mmol), palladium acetate (22.7 mg, 0.101 mmol), tri(t-butyl)phosphine (61.3 mg, 0.303 mmol), cesium carbonate (2.63 g, 8.08 mmol) and Compound e (1.36 g, 4.24 mmol) were dissolved in 10 ml of xylene, and the resulting mixture was reacted by a boiling temperature reflux under a nitrogen atmosphere for 4 hours. An organic phase was separated by adding ethyl acetate and water to the reaction mixture, was washed with water and a saturated saline solution, and then concentrated under reduced pressure, and the obtained reaction mixture was purified by recrystallization, thereby obtaining Exemplified Compound 2 at a yield of 58%. The NMR measurement result of the obtained Exemplified Compound 2 was as follows.

¹H-NMR (400 MHz, in CDCl₃): δ(ppm)=1.32 (s, 18H), 1.60 (s, 12H), 1.76 (s, 12H), 6.29 (d, J=8.6 Hz, 4H), 7.00 (dd, J=8.6, 2.2 Hz, 4H), 7.31 (dd, J=7.9, 1.8 Hz, 2H), 7.43 (d, J=1.6 Hz, 2H), 7.50 (d, J=2.2 Hz, 4H), 7.73 (d, J=7.9, 1.5 Hz, 2H), 7.77 (s, 2H), 7.88 (d, J=7.8 Hz, 2H), 7.97 (d, J=7.9 Hz, 2H)

According to HPLC, the obtained Exemplified Compound 2 had a purity of 98.6%. In the above-described analysis by HR-ICP-MS, the total content of Li, Na, K, Rb, Cs, Pd, Cu and Ni atoms and ions was 5,590 ppm.

(Synthesis of Exemplified Compound 3)

Exemplified Compound 3 may be prepared by the following reaction formula.

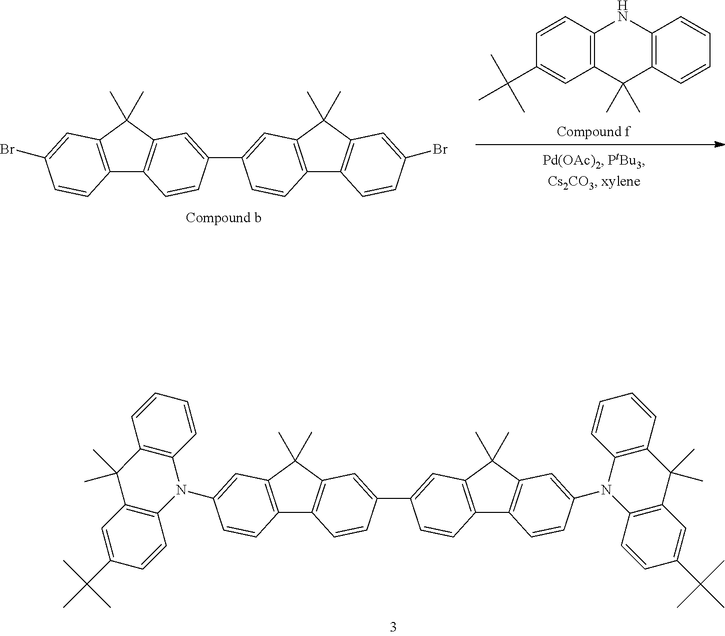 [SMILES: CC1(C)c2cc(Br)ccc2-c2ccc(-c3ccc4c(c3)C(C)(C)c3cc(Br)ccc3-4)cc21 ; CC(C)(C)c1ccc2c(c1)C(C)(C)c1ccccc1N2 ; CC(C)(C)c1ccc2c(c1)C(C)(C)c1ccccc1N2c1ccc2c(c1)C(C)(C)c1cc(-c3ccc4c(c3)C(C)(C)c3cc(N5c6ccccc6C(C)(C)c6cc(C(C)(C)C)ccc65)ccc3-4)ccc1-2]

Compound b (1.10 g, 2.02 mmol), palladium acetate (22.7 mg, 0.101 mmol), tri(t-butyl)phosphine (61.3 mg, 0.303 mmol), cesium carbonate (2.63 g, 8.08 mmol) and Compound f (1.13 g, 4.24 mmol) were dissolved in 10 ml of xylene, and the resulting mixture was reacted by a boiling temperature reflux under a nitrogen atmosphere for 4 hours. An organic phase was separated by adding ethyl acetate and water to the reaction mixture, was washed with water and a saturated saline solution, and then concentrated under reduced pressure, and the obtained reaction mixture was purified by recrystallization, thereby obtaining Exemplified Compound 3 at a yield of 64%. The NMR measurement result of the obtained Exemplified Compound 3 was as follows.

¹H-NMR (400 MHz, in CDCl₃): δ(ppm)=1.32 (s, 18H), 1.61 (s, 12H), 1.73 (s, 12H), 6.31-6.37 (m, 4H), 6.91-6.99 (m, 4H), 7.02 (dd, J=8.6, 2.1 Hz, 2H), 7.32 (dd, J=7.9, 1.6 Hz, 2H), 7.42 (d, J=1.5 Hz, 2H), 7.47 (d, J=8.5 Hz, 2H), 7.51 (d, J=2.1 Hz, 2H), 7.73 (d, J=7.8 Hz, 2H), 7.77 (s, 2H), 7.88 (d, J=7.8 Hz, 2H), 7.99 (d, J=7.9 Hz, 2H)

According to HPLC, the obtained Exemplified Compound 3 had a purity of 99.0%. In the above-described analysis by HR-ICP-MS, the total content of Li, Na, K, Rb, Cs, Pd, Cu and Ni atoms and ions was 6,670 ppm.

(Synthesis of Exemplified Compound 20)

Exemplified Compound 20 may be prepared by the following reaction formula.

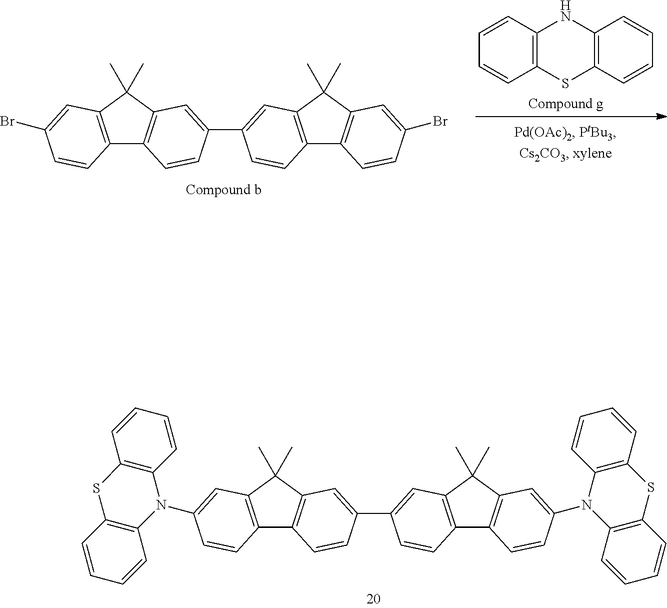 [SMILES: CC1(C)c2cc(Br)ccc2-c2ccc(-c3ccc4c(c3)C(C)(C)c3cc(Br)ccc3-4)cc21 ; c1ccc2c(c1)Nc1ccccc1S2 ; CC1(C)c2cc(-c3ccc4c(c3)C(C)(C)c3cc(N5c6ccccc6Sc6ccccc65)ccc3-4)ccc2-c2ccc(N3c4ccccc4Sc4ccccc43)cc21]

Compound b (1.10 g, 2.02 mmol), palladium acetate (22.7 mg, 0.101 mmol), tri(t-butyl)phosphine (61.3 mg, 0.303 mmol), cesium carbonate (2.63 g, 8.08 mmol) and Compound g (845 mg, 4.24 mmol) were dissolved in 10 ml of xylene, and the resulting mixture was reacted by a boiling temperature reflux under a nitrogen atmosphere for 4 hours. An organic phase was separated by adding ethyl acetate and water to the reaction mixture, was washed with water and a saturated saline solution, and then concentrated under reduced pressure, and the obtained reaction mixture was purified by recrystallization, thereby obtaining Exemplified Compound 20 at a yield of 48%. The NMR measurement result of the obtained Exemplified Compound 20 is as follows.

¹H-NMR (400 MHz, in CDCl₃): δ(ppm)=1.61 (s, 12H), 6.31 (d, J=8.0, 1.4 Hz, 4H), 6.80-6.89 (m, 8H), 7.04 (dd, J=7.3, 1.8 Hz, 4H), 7.40 (dd, J=7.9, 1.8 Hz, 2H), 7.49 (d, J=1.7 Hz, 2H), 7.72 (dd, J=7.9, 1.6 Hz, 2H), 7.76 (d, J=1.2 Hz, 2H), 7.87 (d, J=7.9 Hz, 2H), 7.98 (d, J=8.0 Hz, 2H)

According to HPLC, the obtained Exemplified Compound 20 had a purity of 98.5%. In the above-described analysis by HR-ICP-MS, the total content of Li, Na, K, Rb, Cs, Pd, Cu and Ni atoms and ions was 5,940 ppm.

(Synthesis of Exemplified Compound 19)

Exemplified Compound 19 may be prepared by the following reaction formula.

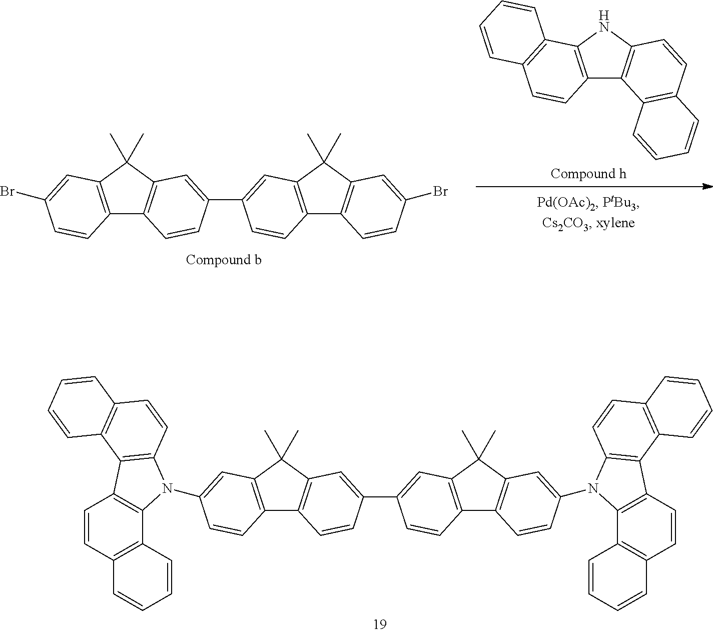 [SMILES: CC1(C)c2cc(Br)ccc2-c2ccc(-c3ccc4c(c3)C(C)(C)c3cc(Br)ccc3-4)cc21 ; c1ccc2c(c1)ccc1c2[nH]c2ccc3ccccc3c21 ; CC1(C)c2cc(-c3ccc4c(c3)C(C)(C)c3cc(-n5c6ccc7ccccc7c6c6ccc7ccccc7c65)ccc3-4)ccc2-c2ccc(-n3c4ccc5ccccc5c4c4ccc5ccccc5c43)cc21]

Compound b (1.11 g, 2.04 mmol), palladium acetate (45.8 mg, 0.204 mmol), tri(t-butyl)phosphine (82.5 mg, 0.408 mmol), cesium carbonate (2.66 g, 8.16 mmol) and Compound h (1.20 g, 4.49 mmol) were dissolved in 10 ml of xylene, and the resulting mixture was reacted by a boiling temperature reflux under a nitrogen atmosphere for 8 hours. An organic phase was separated by adding ethyl acetate and water to the reaction mixture, was washed with water and a saturated saline solution, and then concentrated under reduced pressure, and the obtained reaction mixture was purified by recrystallization, thereby obtaining Exemplified Compound 19 at a yield of 50%. The NMR measurement result of the obtained Exemplified Compound 19 is as follows.

¹H-NMR (400 MHz, in CDCl₃): δ(ppm)=1.64 (6)(s, 6H), 1.65 (4)(s, 6H), 7.20 (t, J=7.7, 2H), 7.44 (t, J=8.0, 2H), 7.48 (d, J=8.9, 2H), 7.52-7.57 (m, 4H), 7.62-7.64 (m, 4H), 7.76-7.84 (m, 8H), 7.88 (d, J=8.7, 2H), 8.01 (d, J=7.8, 2H), 8.05 (d, J=8.0, 4H), 8.09 (d, J=8.3, 2H), 8.82 (d, J=8.9, 2H), 9.01 (d, J=8.2, 2H)

According to HPLC, the obtained Exemplified Compound 19 had a purity of 98.2%. In the above-described analysis by HR-ICP-MS, the total content of Li, Na, K, Rb, Cs, Pd, Cu and Ni atoms and ions was 5,710 ppm.

(Synthesis of Exemplified Compound 5)

Exemplified Compound 5 may be prepared by the following reaction formula.

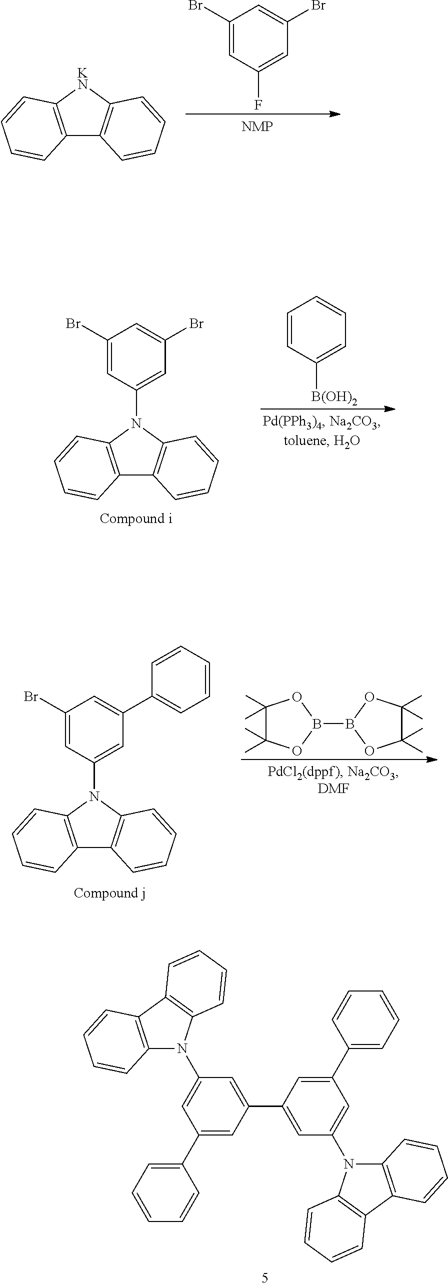

Carbazole potassium salt (17.6 g, 85.9 mol) and 1,3-dibromo-5-fluorobenzene (24.0 g, 94.5 mol) were dissolved in 150 ml of 1-methyl-2-pyrrolidone, and Compound i was obtained at a yield of 75% by stirring the resulting solution at 100° C. for 3 hours. Compound j was synthesized at a yield of 32% by dissolving Compound i (40.0 g, 99.7 mmol), phenylboronic acid (13.4 g, 110 mmol), tetrakis(triphenylphosphine)palladium (2.30 g, 1.99 mmol) and sodium carbonate (21.1 g, 199 mmol) in a mixed solvent of toluene 500 ml/H₂O 200 ml/ethanol 200 ml, and reacting the resulting mixture by a boiling temperature reflux under a nitrogen atmosphere for 2 hours. Compound j (7.00 g, 17.6 mmol), bis(pinacolato)diboron (2.23 g, 8.80 mmol), PdCl₂(dppf) (719 mg, 0.88 mmol) and sodium acetate (5.18 g, 52.8 mmol) were dissolved in 80 ml of DMF (N,N-dimethylformamide), and the resulting solution was reacted by a boiling temperature reflux under a nitrogen atmosphere for 3 hours. An organic phase was separated by adding ethyl acetate and water to the reaction mixture, was washed with water and a saturated saline solution, and then concentrated under reduced pressure, and the obtained reaction mixture was purified by recrystallization, thereby obtaining Exemplified Compound 5 at a yield of 30%.

According to HPLC, the obtained Exemplified Compound 5 had a purity of 98.9%. In the above-described analysis by HR-ICP-MS, in an ICP light emission analysis, the total content of Li, Na, K, Rb, Cs, Pd, Cu and Ni atoms and ions was 6,120 ppm.

(Synthesis of Exemplified Compound 9)

Exemplified Compound 9 may be prepared by the following reaction formula.

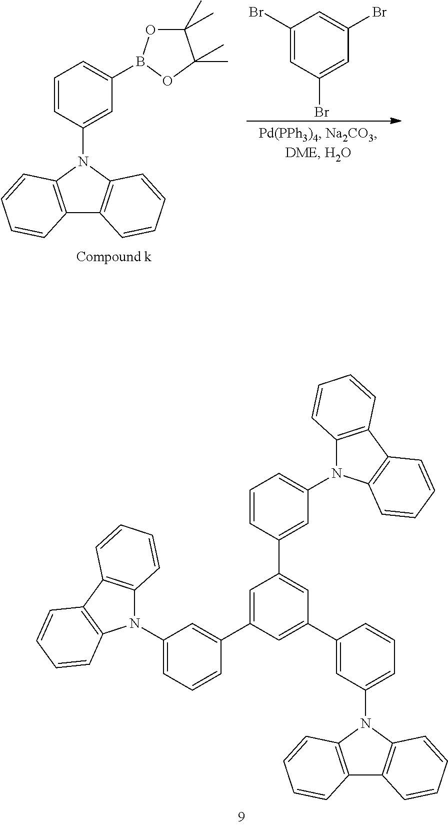 [SMILES: CC1(C)OB(c2cccc(-n3c4ccccc4c4ccccc43)c2)OC1(C)C ; Brc1cc(Br)cc(Br)c1 ; c1cc(-c2cc(-c3cccc(-n4c5ccccc5c5ccccc54)c3)cc(-c3cccc(-n4c5ccccc5c5ccccc54)c3)c2)cc(-n2c3ccccc3c3ccccc32)c1]

Compound k (7.00 g, 19.0 mol), 1,35-tribromobenzene (1.93 g, 6.13 mmol), tetrakis(triphenylphosphine)palladium (355 mg, 0.307 mmol) and sodium carbonate (3.90 g, 36.8 mmol) were dissolved in a mixed solvent of DME (1,2-dimethoxyethane) 300 ml/H₂O 80 ml, and the resulting solution was reacted by a boiling temperature reflux under a nitrogen atmosphere for 6 hours. The reaction mixture was filtered and washed with ethyl acetate and the obtained white powder was purified by recrystallization, thereby obtaining Exemplified Compound 9 at a yield of 53%.

According to HPLC, the obtained Exemplified Compound 9 had a purity of 97.5%. In the above-described analysis by HR-ICP-MS, the total content of Li, Na, K, Rb, Cs, Pd, Cu and Ni atoms and ions was 12,900 ppm.

(Synthesis of Exemplified Compound 14)

Exemplified Compound 14 may be prepared by the following reaction formula.

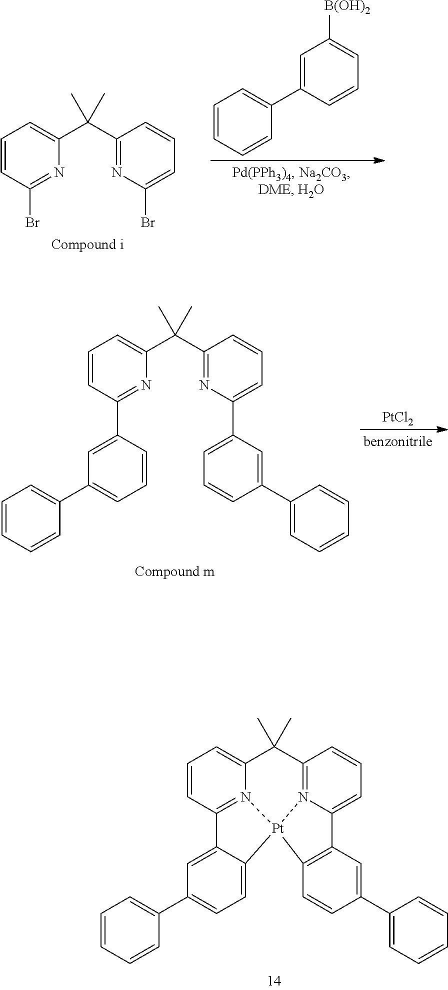

Compound 1 (2.50 g, 7.02 mol), 3-biphenylboronic acid (2.93 g, 14.8 mmol), tetrakis(triphenylphosphine)palladium (406 mg, 0.351 mmol) and sodium carbonate (5.96 g, 56.2 mmol) were dissolved in a mixed solvent of DME (1,2-dimethoxyethane) 40 ml/H₂O 40 ml, and Compound m was obtained at a yield of 72% by reacting the resulting solution by a boiling temperature reflux under a nitrogen atmosphere for 6 hours. Compound m (1.76 g, 4.39 mmol) and platinum chloride (1.17 g, 4.39 mmol) were added to 14 ml of benzonitrile, and the resulting mixture was reacted by a boiling temperature reflux under a nitrogen atmosphere for 5 hours. The reaction mixture was filtered and washed with ethyl acetate, and the obtained orange powder was purified by recrystallization using benzonitrile as a solvent, thereby obtaining Exemplified Compound 14 at a yield of 50%.

According to HPLC, the obtained Exemplified Compound 14 had a purity of 98.8%. In the above-described analysis by HR-ICP-MS, the total content of Li, Na, K, Rb, Cs, Pd, Cu and Ni atoms and ions was 6,650 ppm.

(Synthesis of Exemplified Compound 15)

Exemplified Compound 15 may be prepared by the following reaction formula.

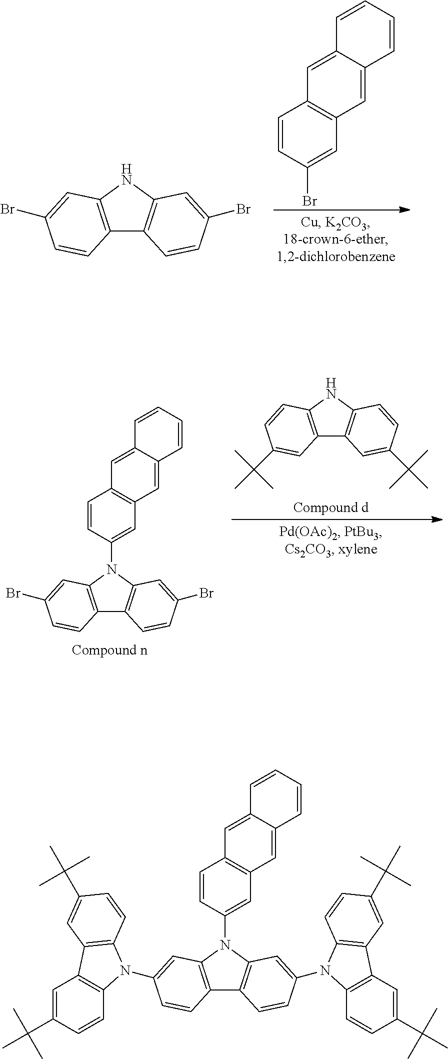 [SMILES: Brc1ccc2c(c1)[nH]c1cc(Br)ccc12 ; Brc1ccc2cc3ccccc3cc2c1 ; Brc1ccc2c3ccc(Br)cc3n(-c3ccc4cc5ccccc5cc4c3)c2c1 ; CC(C)(C)c1ccc2[nH]c3ccc(C(C)(C)C)cc3c2c1 ; CC(C)(C)c1ccc2c(c1)c1cc(C(C)(C)C)ccc1n2-c1ccc2c3ccc(-n4c5ccc(C(C)(C)C)cc5c5cc(C(C)(C)C)ccc54)cc3n(-c3ccc4cc5ccccc5cc4c3)c2c1]

2,7-dibromocarbazole is synthesized according to the Journal of Organic Chemistry, 2005, vol. 70 and paragraph 5014 to 5019, and 3.5 g of the sample, 8.3 g of 2-bromoanthracene, 0.8 g of copper powder, 3 g of potassium carbonate, 20 ml of 1,2-dichlorobenzene and 1.4 g of 18-crown-6-ether were stirred under a nitrogen atmosphere for 6 hours while being heated under reflux. The mixture was cooled to room temperature, and then 1.7 g of Compound n was obtained by purifying the reaction solution by silica-gel column chromatography using a toluene-hexane mixed solvent. The sample was reacted with Compound d, thereby obtaining Exemplified Compound 15.

According to HPLC, the obtained Exemplified Compound 15 had a purity of 98.8%. In the above-described analysis by HR-ICP-MS, the total content of Li, Na, K, Rb, Cs, Pd, Cu and Ni atoms and ions was 7,510 ppm.

(Synthesis of Exemplified Compound 16)

Exemplified Compound 16 may be prepared by the following reaction formula.

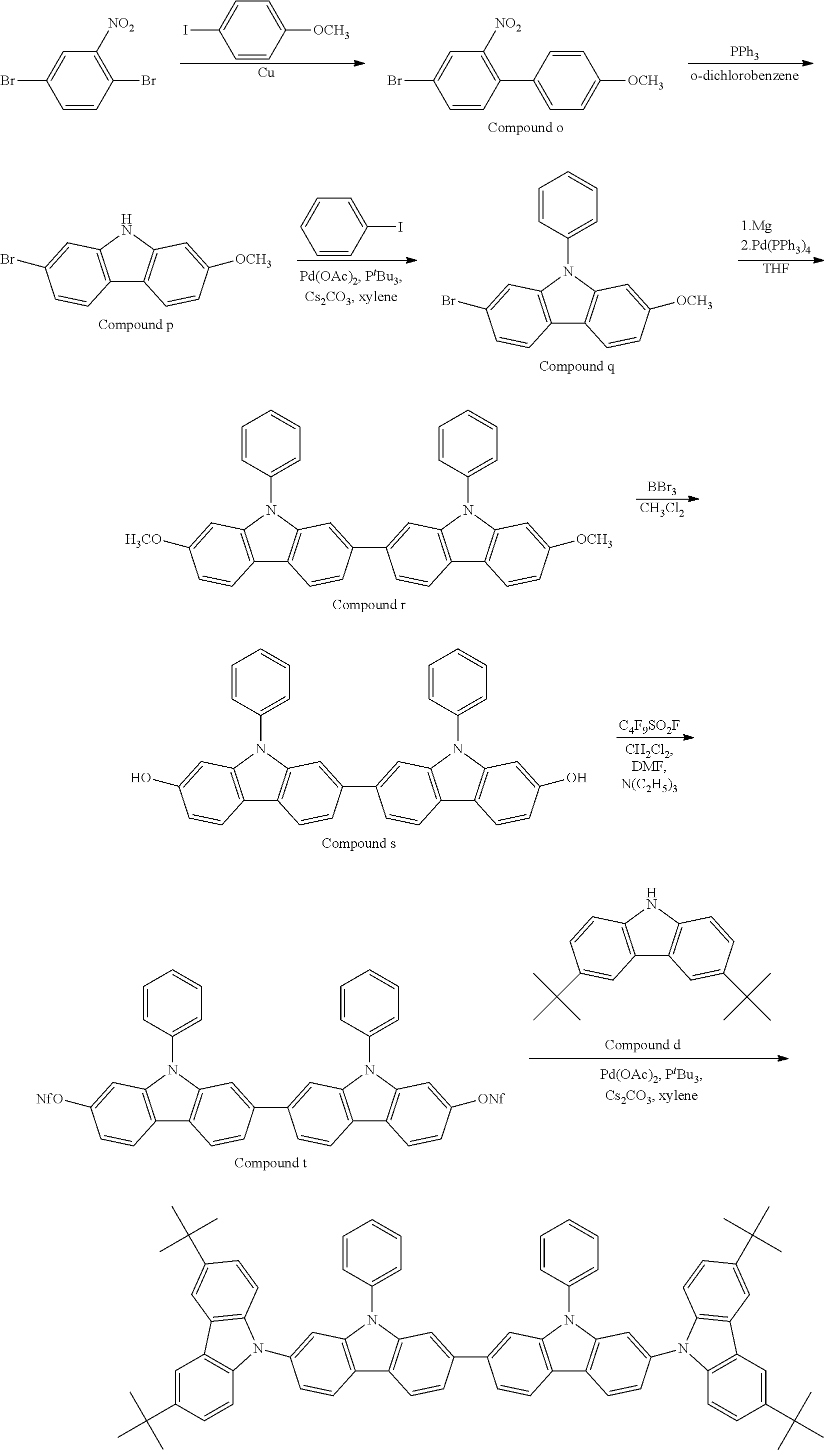

1,4-dibromo-2-nitrobenzene (23.2 g, 0.0825 mol) and copper powder (15.6 g, 0.248 mol) were added to 4-iodine anisole (25.1 g, 0.107 mol), and Compound o was obtained at a yield of 44% by stirring the resulting mixture at 175° C. for 3 hours. Compound o (11.1 g, 36.0 mmol) and triphenylphosphine (23.6 g, 90.0 mmol) were dissolved in 70 ml of o-dichlorobenzene, and Compound p was obtained at a yield of 89% by reacting the resulting solution by a boiling temperature reflux under a nitrogen atmosphere for 5 hours. Compound p (4.4 g, 0.159 mmol), palladium acetate (89.4 mg, 0.398 mmol), tri(t-butyl)phosphine (241 mg, 119 mmol), cesium carbonate (15.5 g, 47.7 mmol) and iodotoluene (16.2 g, 79.5 mmol) were dissolved in 86 ml of xylene, and Compound q was synthesized by reacting the resulting mixture by a boiling temperature reflux under a nitrogen atmosphere for 3 hours (yield 52%).

A boiling temperature reflux was performed by adding magnesium (103 mg, 4.24 mmol) to 2 ml of THF under a nitrogen atmosphere, 8 ml of the THF solution of Compound q (2.90 g, 8.23 mmol) was added dropwise thereto, and the resulting mixture was stirred for 1 hour. Thereafter, Compound r was obtained at a yield of 52% by adding tetrakis(triphenylphosphine)palladium (47.6 mg, 0.0412 mmol) thereto and refluxing the resulting mixture at a boiling temperature for 2 hours. Compound r (1.20 g, 2.20 mmol) was dissolved in 50 ml of methylene chloride, 5.5 ml of a 1 mol/lBBr₃ methylene chloride solution was added dropwise thereto at 0° C. under a nitrogen atmosphere, and the resulting solution was reacted at room temperature for 3 hours.

After the quenching reaction was completed, an organic phase was separated by adding ethyl acetate and water to the reaction mixture, was washed with water and a saturated saline solution, and then concentrated under reduced pressure. The concentrated reaction mixture (Compound s) was dissolved in 30 ml of a mixed solvent (1:1) of methylene chloride and N,N′-dimethylformamide, and triethylamine (0.92 ml, 6.60 mmol) was added thereto. Perfluorobutanesulfonyl fluoride (1.16 ml, 6.60 mmol) was added dropwise thereto at 5° C. under a nitrogen atmosphere, and Compound t was obtained at a yield of 46% by reacting the resulting mixture at room temperature for 3 hours. Compound t (1.00 g, 0.925 mmol), palladium acetate (11.3 mg, 0.0463 mmol), tri(t-butyl)phosphine (28.1 mg, 0.139 mmol), cesium carbonate (1.21 g, 3.70 mmol) and Compound d (567 mg, 2.03 mmol) were dissolved in 9 ml of xylene, and the resulting mixture was reacted by a boiling temperature reflux under a nitrogen atmosphere for 4 hours.

An organic phase was separated by adding ethyl acetate and water to the reaction mixture, was washed with water and a saturated saline solution, and then concentrated under reduced pressure, and the obtained reaction mixture was purified by recrystallization, thereby obtaining Exemplified Compound 16 at a yield of 42%.

According to HPLC, Exemplified Compound 16 had a purity of 98.0%. In the above-described analysis by HR-ICP-MS, the total content of Li, Na, K, Rb, Cs, Pd, Cu and Ni atoms and ions was 5,830 ppm.

(Synthesis of Exemplified Compound 17)

Exemplified Compound 17 may be prepared by the following reaction formula.

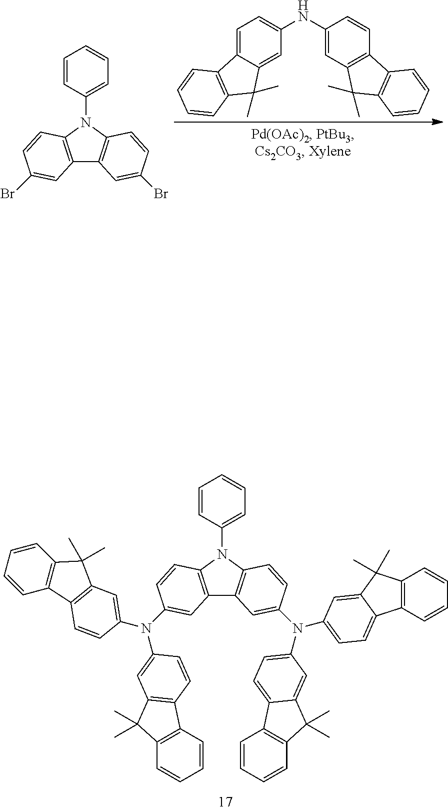 [SMILES: Brc1ccc2c(c1)c1cc(Br)ccc1n2-c1ccccc1 ; CC1(C)c2ccccc2-c2ccc(Nc3ccc4c(c3)C(C)(C)c3ccccc3-4)cc21 ; CC1(C)c2ccccc2-c2ccc(N(c3ccc4c(c3)C(C)(C)c3ccccc3-4)c3ccc4c(c3)c3cc(N(c5ccc6c(c5)C(C)(C)c5ccccc5-6)c5ccc6c(c5)C(C)(C)c5ccccc5-6)ccc3n4-c3ccccc3)cc21]

3,6-dibromo-9-phenylcarbazole (2.00 g, 4.99 mmol), palladium acetate (60.8 mg, 0.249 mmol), tri(t-butyl)phosphine (151 mg, 0.747 mmol), cesium carbonate (6.51 g, 20.0 mmol) and bis(9,9′-dimethylfluore-2-yl)amine (4.46 g, 11.0 mmol) were dissolved in 55 ml of xylene, and the resulting solution was reacted by a boiling temperature reflux under a nitrogen atmosphere for 5 hours.

An organic phase was separated by adding ethyl acetate and water to the reaction mixture, was washed with water and a saturated saline solution, and then concentrated under reduced pressure, and the obtained reaction mixture was purified by recrystallization, thereby obtaining Exemplified Compound 17 at a yield of 63%.

According to HPLC, Exemplified Compound 17 had a purity of 98.3%. In the above-described analysis by HR-ICP-MS, the total content of Li, Na, K, Rb, Cs, Pd, Cu and Ni atoms and ions was 6,210 ppm.

(Synthesis of Exemplified Compound 21)

Exemplified Compound 21 may be prepared by the following reaction formula.

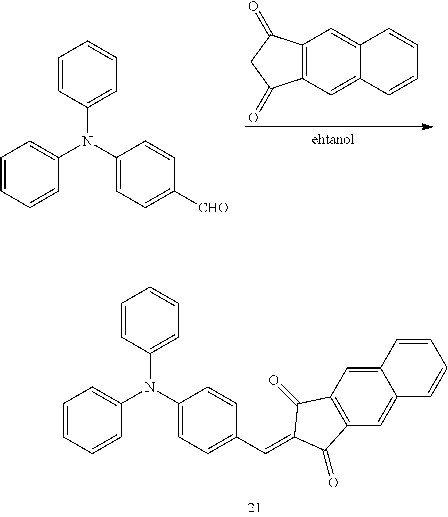 [SMILES: O=Cc1ccc(N(c2ccccc2)c2ccccc2)cc1 ; O=C1CC(=O)c2cc3ccccc3cc21 ; O=C1C(=Cc2ccc(N(c3ccccc3)c3ccccc3)cc2)C(=O)c2cc3ccccc3cc21]

Benz[f]indane-1,3-dione was synthesized according to the J. Med. Chem., 1973, vol. and paragraphs 1334 to 1339, and 2 g of the sample and 3.1 g of 4-(N,N-diphenylamino)benzaldehyde were overheating stirred for 6 hours under reflux in 20 ml of ethanol, and cooled to room temperature. The obtained crystal was separated by filtration and washed, and 4.3 g of Exemplified Compound 21 was obtained by performing recrystallization from chloroform-acetonitrile.

According to HPLC, Exemplified Compound 21 had a purity of 98.5%. In the above-described analysis by HR-ICP-MS, the total content of Li, Na, K, Rb, Cs, Pd, Cu and Ni atoms and ions was 6,620 ppm.

(Synthesis of Exemplified Compound 22)

Exemplified Compound 22 may be prepared by the following reaction formula.

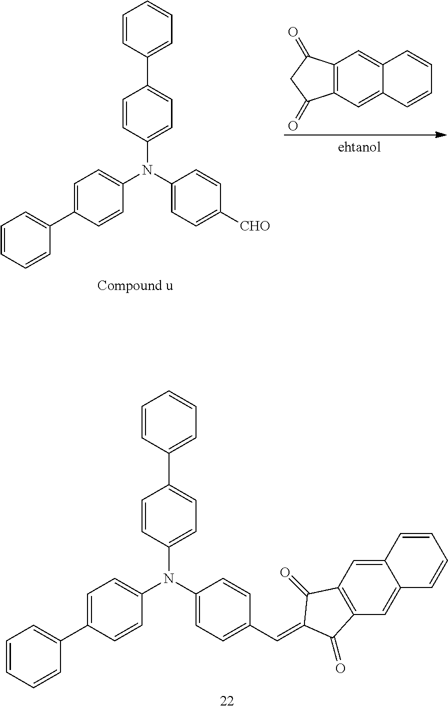 [SMILES: O=Cc1ccc(N(c2ccc(-c3ccccc3)cc2)c2ccc(-c3ccccc3)cc2)cc1 ; O=C1CC(=O)c2cc3ccccc3cc21 ; O=C1C(=Cc2ccc(N(c3ccc(-c4ccccc4)cc3)c3ccc(-c4ccccc4)cc3)cc2)C(=O)c2cc3ccccc3cc21]

Compound u is synthesized according to the J. Med. Chem., 1973, vol. 17 and pages 2088 to 2094, and Compound u (2.0, 4.70 mmol) and benz[f]indane-1,3-dione (1.01 g, 5.17 mmol) are overheating stirred for 6 hours under reflux in 20 ml of ethanol, and cooled to room temperature. The obtained crystal was separated by filtration and washed, and 1.9 g of Exemplified Compound 22 was obtained by performing recrystallization from chloroform-acetonitrile.

According to HPLC, Exemplified Compound 22 had a purity of 98.2%. In the above-described analysis by HR-ICP-MS, the total content of Li, Na, K, Rb, Cs, Pd, Cu and Ni atoms and ions was 5,520 ppm.

The other Exemplified Compounds were synthesized with reference to the aforementioned methods and the documents such as US2007/0293704, Chem. Lett., 2006, 35, 158-159, EP1559706 and WO99/40655.

Inorganic impurities included in the materials synthesized by the methods were removed by various purification methods. Specifically, materials having a content of inorganic impurities shown in the following Table 1 were prepared by performing recrystallization purification, column chromatography purification, washing with water and solvents, reslurry, and separation by filtration of impurities and precipitates after being dissolved in a solvent. Further, the sublimation purification materials in Comparative Examples were not subjected to purification for the purpose of removing inorganic impurities.

[Measurement of Content of Inorganic Impurities]

The content of inorganic impurities of the materials before sublimation purification was measured by HR-ICP-MS using ELEMNTXR manufactured by Thermo Scientific Inc. About 50 mg of the sample was put into a microwave decomposition container, 3 ml of nitric acid and 1 ml of hydrochloric acid were added thereto, the container was sealed, and then a microwave decomposition was performed. The decomposed liquid was diluted with H₂O, the volume is maintained at a constant level, and alkali metals and transition metals (Li, Na, K, Rb, Cs, Pd, Cu and Ni) were subjected to measurement by HR-ICP-MS. The content was determined by an absolute calibration curve method.

In addition, the 10% weight reduction temperature and glass transition temperature of Exemplified Compounds 1 to 28 and Compound A were measured as follows.

[Measurement of TG/DTA Under Vacuum]

For measurement of each compound, temperature was increased at 2° C./min in a range of 30° C. to 500° C. under vacuum conditions by using VAP-9000 manufactured by ULVAC-RIKO, Inc. It was confirmed that the vacuum degree was 1.0×10⁻² Pa, and temperature begins to be controlled. Temperature was increased in a range of 30° C. to 500° C. under vacuum conditions, and a temperature at which the residue of the compound reaches 90% by weight was defined as a 10% weight reduction temperature.

[Measurement of Glass Transition Point]

The glass transition point (Tg) was measured using DSC6220 manufactured by SII NanoTechnology Inc. 5 mg of the sample was placed on a pan, and the heat capacity change was measured by increasing and decreasing the temperature in a range of 30° C. to 400° C. (temperature rise: 20° C./min, temperature drop: 50° C./min and 2 cycles). Two extension lines were drawn on a caloric variation curve corresponding to the glass transition, and the glass transition point (Tg) was obtained from the intersection point of the ½ line between the extension lines and the caloric curve. In the following Tables 1 to 3, A, B, C and D indicate the following matters.

A: Tg=200° C. or more

B: 160° C. or more and less than 200° C.

C: 130° C. or more and less than 160° C.

D: Less than 130° C.

The 10% weight reduction temperatures and the glass transition temperatures of Compounds 1 to 28 and Compound A and the contents of inorganic impurities before sublimation purification in the Examples and Comparative Examples are shown in the following Tables 1 and 2.

[Sublimation Purification]

In each Example and Comparative, the sublimation purification was performed using TRS-160 manufactured by ULVAC-RIKO, Inc. Pressure was reduced to 7.0×10⁻² Pa, temperature was increased to a range of 300° C. to 400° C., and the heating temperature and heating time shown in the following Tables 1 and 2 were used. Crystals attached to a glass tube were collected as a sample subjected to sublimation purification using a spatula. The ratio of the sample before sublimation to the sample after sublimation purification was used as a sublimation purification yield.

The purity after sublimation purification was calculated by a peak area ratio of HPLC (analysis system: LC-10 A manufactured by Shimadzu Corporation, column: TSKGel-80TS manufactured by TOSOH Corporation) (detection wavelength: 254 nm).

The yield of sublimation purification and the purity after sublimation purification are shown in the following Tables 1 and 2.

TABLE 1

| | | 10% weight | Glass | Content of | | | Yield of | Sample purity |
|---|---|---|---|---|---|---|---|---|
| | Sublimation | reduction | transition | inorganic | Heating | Heating | sublimation | after sublimation |
| | purification | temp. | temp. | impurities | temp. | time | purification | purification |
| | material | (° C.) | (° C.) | (ppm) | (° C.) | (h) | (%) | (%) |
| Ex. 1 | Comp. 1 | 362 | A | 2430 | 390 | 5 | 59 | 98.7 |
| Ex. 2 | Comp. 1 | 362 | A | 911 | 390 | 5 | 83 | 99.3 |
| Ex. 3 | Comp. 1 | 362 | A | 452 | 390 | 5 | 89 | 99.4 |
| Ex. 4 | Comp. 1 | 362 | A | 182 | 390 | 3 | 89 | 99.7 |
| Ex. 5 | Comp. 2 | 343 | A | 2560 | 360 | 2.5 | 75 | 99.0 |
| Ex. 6 | Comp. 2 | 343 | A | 885 | 360 | 2.5 | 79 | 99.3 |
| Ex. 7 | Comp. 2 | 343 | A | 333 | 360 | 2.5 | 88 | 99.7 |
| Ex. 8 | Comp. 3 | 355 | A | 1860 | 380 | 2.5 | 71 | 99.0 |
| Ex. 9 | Comp. 4 | 338 | A | 2600 | 380 | 5 | 75 | 99.5 |
| Ex. 10 | Comp. 5 | 288 | C | 732 | 300 | 2 | 86 | 99.6 |
| Ex. 11 | Comp. 6 | 380 | C | 1230 | 440 | 6 | 85 | 98.9 |
| Ex. 12 | Comp. 7 | 312 | C | 1502 | 340 | 5 | 84 | 99.7 |
| Ex. 13 | Comp. 8 | 295 | D | 3460 | 320 | 2.5 | 62 | 98.6 |
| Ex. 14 | Comp. 9 | 366 | C | 980 | 380 | 6 | 62 | 99.5 |
| Ex. 15 | Comp. 9 | 366 | C | 2380 | 380 | 6 | 57 | 99.0 |
| Ex. 16 | Comp. 10 | 253 | D | 1840 | 310 | 2.5 | 74 | 99.6 |
| Ex. 17 | Comp. 11 | 306 | B | 882 | 370 | 3 | 61 | 99.8 |
| Ex. 18 | Comp. 12 | 300 | D | 1350 | 310 | 5 | 80 | 99.7 |
| Ex. 19 | Comp. 13 | 302 | B | 2510 | 340 | 3 | 93 | 99.6 |
| Ex. 20 | Comp. 14 | 341 | A | 1890 | 390 | 6 | 85 | 99.2 |
| Ex. 21 | Comp. 15 | 357 | A | 383 | 400 | 2.5 | 90 | 99.0 |
| Ex. 22 | Comp. 15 | 357 | A | 892 | 400 | 2.5 | 81 | 99.0 |
| Ex. 23 | Comp. 15 | 357 | A | 3760 | 400 | 2.5 | 75 | 98.8 |
| Ex. 24 | Comp. 16 | 374 | A | 570 | 410 | 5 | 77 | 98.7 |
| Ex. 25 | Comp. 17 | 368 | B | 790 | 390 | 3 | 79 | 98.9 |
| Ex. 26 | Comp. 18 | 372 | A | 861 | 410 | 5 | 71 | 98.6 |
| Ex. 27 | Comp. 19 | 392 | A | 395 | 440 | 7 | 58 | 98.5 |
| Ex. 28 | Comp. 19 | 392 | A | 2520 | 440 | 7 | 48 | 98.6 |
| Ex. 29 | Comp. 20 | 345 | B | 410 | 385 | 6 | 53 | 98.5 |
| Ex. 30 | Comp. 21 | 263 | D | 430 | 380 | 2.5 | 92 | 99.5 |
| Ex. 31 | Comp. 21 | 263 | D | 1820 | 380 | 2.5 | 83 | 99.1 |
| Ex. 32 | Comp. 22 | 303 | D | 936 | 325 | 4 | 73 | 99.2 |
| Ex. 33 | Comp. 23 | 361 | B | 2550 | 390 | 4 | 63 | 99.1 |
| Ex. 34 | Comp. 24 | 301 | C | 1760 | 330 | 5 | 78 | 99.3 |
| Ex. 35 | Comp. 25 | 312 | C | 480 | 350 | 5 | 61 | 99.0 |
| Ex. 36 | Comp. 26 | 298 | B | 1270 | 320 | 3 | 90 | 99.5 |
| Ex. 37 | Comp. 27 | 305 | B | 890 | 330 | 5 | 86 | 99.4 |
| Ex. 38 | Comp. 28 | 287 | B | 890 | 320 | 4 | 69 | 99.1 |

TABLE 2

| | | 10% weight | Glass | Content of | | | Yield of | Sample purity |
|---|---|---|---|---|---|---|---|---|
| | Sublimation | reduction | transition | inorganic | Heating | Heating | sublimation | after sublimation |
| | purification | temp. | temp. | impurities | temp. | time | purification | purification |
| | material | (° C.) | (° C.) | (ppm) | (° C.) | (h) | (%) | (%) |
| C. Ex. 1 | Comp. 1 | 362 | A | 7320 | 390 | 5 | 48 | 97.8 |
| C. Ex. 2 | Comp. 1 | 362 | A | 5320 | 390 | 5 | 50 | 98.1 |
| C. Ex. 3 | Comp. 2 | 343 | A | 5590 | 360 | 2.5 | 61 | 98.1 |
| C. Ex. 4 | Comp. 3 | 355 | A | 6670 | 380 | 2.5 | 55 | 98.2 |
| C. Ex. 5 | Comp. 4 | 338 | A | 7520 | 380 | 5 | 63 | 98.1 |
| C. Ex. 6 | Comp. 5 | 288 | C | 6120 | 300 | 2 | 77 | 98.3 |
| C. Ex. 7 | Comp. 6 | 380 | C | 5320 | 440 | 6 | 65 | 97.9 |
| C. Ex. 8 | Comp. 7 | 312 | C | 6200 | 340 | 5 | 66 | 97.8 |
| C. Ex. 9 | Comp. 8 | 295 | D | 7870 | 320 | 2.5 | 45 | 95.6 |
| C. Ex. 10 | Comp. 9 | 366 | C | 12900 | 380 | 6 | 44 | 98.0 |
| C. Ex. 11 | Comp. 10 | 253 | D | 7820 | 310 | 2.5 | 61 | 98.3 |
| C. Ex. 12 | Comp. 11 | 306 | B | 5690 | 370 | 3 | 55 | 98.2 |
| C. Ex. 13 | Comp. 12 | 300 | D | 7780 | 310 | 5 | 69 | 98.4 |
| C. Ex. 14 | Comp. 13 | 302 | B | 5750 | 340 | 3 | 82 | 98.2 |
| C. Ex. 15 | Comp. 14 | 341 | A | 6650 | 390 | 6 | 68 | 98.0 |
| C. Ex. 16 | Comp. 15 | 357 | A | 7510 | 400 | 2.5 | 65 | 98.1 |
| C. Ex. 17 | Comp. 16 | 374 | A | 5830 | 410 | 5 | 55 | 97.4 |
| C. Ex. 18 | Comp. 17 | 368 | B | 6210 | 390 | 3 | 62 | 97.8 |
| C. Ex. 19 | Comp. 18 | 372 | A | 5320 | 410 | 5 | 56 | 97.3 |
| C. Ex. 20 | Comp. 19 | 392 | A | 5710 | 440 | 7 | 31 | 97.0 |
| C. Ex. 21 | Comp. 20 | 345 | B | 5940 | 385 | 6 | 38 | 96.5 |
| C. Ex. 22 | Comp. 21 | 263 | D | 6620 | 380 | 2.5 | 58 | 98.1 |
| C. Ex. 23 | Comp. 22 | 303 | D | 5520 | 325 | 4 | 55 | 98.2 |
| C. Ex. 24 | Comp. 23 | 361 | B | 6820 | 390 | 4 | 48 | 97.9 |
| C. Ex. 25 | Comp. 24 | 301 | C | 7810 | 330 | 5 | 50 | 98.0 |
| C. Ex. 26 | Comp. 25 | 312 | C | 5670 | 350 | 5 | 38 | 97.4 |
| C. Ex. 26 | Comp. 26 | 298 | B | 6990 | 320 | 3 | 75 | 98.3 |
| C. Ex. 27 | Comp. 27 | 305 | B | 6370 | 330 | 5 | 78 | 98.4 |
| C. Ex. 28 | Comp. 28 | 287 | B | 5450 | 320 | 4 | 48 | 97.6 |
| C. Ex. 29 | Comp. A | 247 | D | 4830 | 270 | 5 | 93 | 99.5 |
| C. Ex. 30 | Comp. A | 247 | D | 8720 | 270 | 5 | 92 | 99.6 |

When Examples 1 to 35 were compared with Comparative Examples 1 to 28, it can be seen that in the results in which the content of inorganic impurities of the material before sublimation purification is as small as 5,000 ppm or less, the yields and purities of the samples during the sublimation purification are high. Further, in the case of comparison with the same material, it can be seen that since the yields of the samples in Examples are higher even for the same heating hours, sublimation purification is completed in a short period of time when the same yield is obtained.

In addition, in Comparative Examples 29 and 30, in the case of the materials having a 10% weight reduction temperature less than 250° C., no difference in yield and purity during the sublimation purification is observed even though the concentration of inorganic impurities before sublimation purification is 5,000 ppm or less.

Further, the purity of the sample after the sublimation purification is also slightly decreased in some cases when compared to the purity of the exemplified compound after synthesis, but since residual solvent and the like due to a solvent used during the synthesis may be removed by sublimation purification, purification is a desired method when it is considered that purification is applied to the organic electronics device. As described above, since the residual solvent is an obstacle to the manufacture of the device, sublimation purification has an overwhelming advantage over reduction in purity accompanied by sublimation purification. In addition, the reduction in purity during the sublimation purification may be decreased by adjusting the concentration of inorganic impurities before the sublimation purification to 5,000 ppm or less.

### Example 2-1

A photoelectric conversion device with the form illustrated in FIG. 1(a) was manufactured. That is, a 30-nm amorphous ITO was film-formed on a glass substrate by a sputtering method and was used as a lower electrode, and a charge blocking layer having a film thickness of 100 nm was formed by forming a film using Compound 1 after sublimation purification in Example 1 by a vacuum heating deposition method. In addition, a photoelectric conversion layer was formed by film-forming a layer, which was obtained by co-depositing Compound A-1 and fullerene (C₆₀) thereon to have a thickness of 100 nm and 300 nm, respectively in terms of single layer, by vacuum heating deposition, while the temperature of the substrate was controlled at 25° C. Further, the photoelectric conversion layer was vacuum deposited at a vacuum degree of 4×10⁻⁴ Pa or less.

In addition, a transparent conductive film was formed as an upper electrode by film-forming a 10-nm amorphous ITO thereon by a sputtering method, thereby manufacturing a photoelectric conversion device.

### Examples 2-2 to 13 and Comparative Examples 2-1 to 2-12

A photoelectric conversion device was manufactured in the same manner as in Example 2-1, except that Compound 1 used in the charge blocking layer and Compound A-1 used in the photoelectric conversion layer were changed into the compounds shown in Tables 3 and 4. The compounds shown in Tables 3 and 4 indicate compounds after sublimation purification in the respective Examples and Comparative Examples.

[Evaluation]

It was confirmed whether each device obtained serves as a photoelectric conversion device. That is, when voltage was applied to the lower electrode and the upper electrode of each device obtained so as to have an electric field intensity of 2.5×10⁵ V/cm, a dark current of 100 nA/cm² or less was exhibited in any device or dark place, whereas a dark current of 10 μA/cm² or more was exhibited in a bright place, and accordingly, it was confirmed that the photoelectric conversion device worked. Tables 3 and 4 show each dark current value (relative value when the value of the device in Example 2-1 is defined as “100” at room temperature) of each device obtained at room temperature, during the heating at 130° C., during the heating at 160° C. and during the heating at 200° C.

Further, Tables 3 and 4 show a sensitivity (a relative value when the value of the device in Example 2-1 is defined as “100”) in a region at a wavelength of 500 to 750 nm when an electric field of 2×10⁵ V/cm is applied to the photoelectric conversion device each obtained in Examples 2-1 to 2-13 and Comparative Examples 2-1 to 2-12. In addition, when the photoelectric conversion performance of each device was measured, light was incident to the upper electrode (transparent conductive film) side.

TABLE 3

| | | Charge blocking layer | | | | | | | |
|---|---|---|---|---|---|---|---|---|---|
| | | | Sample | Glass | | | | | Sensitivity in |
| | | | purity after | transi- | Dark current (Relative value) | | | | a region at a |
| | | | sublimation | tion | Room | Heat- | Heat- | Heat- | wavelength of |
| | Photoelectric | | purification | temper- | temper- | ing at | ing at | ing at | 500 nm to 750 nm |
| | conversion material | Kind of compound | (%) | ature | ature | 130° C. | 160° C. | 200° C. | (Relative value) |
| Ex. 2-1 | C₆₀/Compound A-1 | Compound 1 of Ex. 1 | 98.7 | A | 100 | 90 | 78 | 12 | 100 |
| Ex. 2-2 | C₆₀/Compound A-1 | Compound 1 of Ex. 4 | 99.7 | A | 99 | 90 | 75 | 11 | 101 |
| Ex. 2-3 | C₆₀/Compound A-1 | Compound 1 of Ex. 4 | 99.7 | A | 96 | 88 | 72 | 10 | 108 |
| Ex. 2-4 | C₆₀/Compound A-1 | Compound 2 of Ex. 5 | 99.0 | A | 330 | 260 | 123 | 17 | 103 |
| Ex. 2-5 | C₆₀/Compound A-1 | Compound 2 of Ex. 7 | 99.7 | A | 250 | 206 | 104 | 16 | 105 |
| Ex. 2-6 | C₆₀/Compound A-1 | Compound 5 of Ex. 10 | 99.6 | C | 182 | 175 | — | — | 95 |
| Ex. 2-7 | C₆₀/Compound A-1 | Compound 9 of Ex. 14 | 99.5 | C | 231 | 248 | — | — | 91 |
| Ex. 2-8 | C₆₀/Compound A-1 | Compound 11 of Ex. 17 | 99.8 | B | 293 | 299 | 187 | — | 105 |
| Ex. 2-9 | C₆₀/Compound A-1 | Compound 15 of Ex. 22 | 99.0 | A | 102 | 96 | 73 | 24 | 103 |
| Ex. 2-10 | C₆₀/Compound A-1 | Compound 16 of Ex. 24 | 98.7 | A | 185 | 156 | 101 | 38 | 99 |
| Ex. 2-11 | C₆₀/Compound A-1 | Compound 17 of Ex. 25 | 98.9 | B | 4820 | 3980 | 2860 | — | 105 |
| Ex. 2-12 | C₆₀/Compound A-1 | Compound 19 of Ex. 28 | 98.6 | A | 253 | 240 | 180 | 138 | 103 |
| Ex. 2-13 | C₆₀/Compound A-1 | Compound 23 of Ex. 33 | 99.1 | B | 4930 | 3860 | 2230 | 11300 | 108 |

“—” represents not detectable because the device was damaged by heat.

TABLE 4

| | | Charge blocking layer | | | | | | | |
|---|---|---|---|---|---|---|---|---|---|
| | | | Sample | Glass | | | | | Sensitivity in |
| | | | purity after | transi- | Dark current (Relative value) | | | | a region at a |
| | | | sublimation | tion | Room | Heat- | Heat- | Heat- | wavelength of |
| | Photoelectric | | purification | temper- | temper- | ing at | ing at | ing at | 500 nm to 750 nm |
| | conversion material | Kind of compound | (%) | ature | ature | 130° C. | 160° C. | 200° C. | (Relative value) |
| C. Ex. 2-1 | C₆₀/Compound A-1 | Compound 1 of C. Ex. 2 | 98.1 | A | 105 | 92 | 80 | 18 | 95 |
| C. Ex. 2-2 | C₆₀/Compound A-2 | Compound 1 of C. Ex. 2 | 98.1 | A | 106 | 93 | 80 | 17 | 98 |
| C. Ex. 2-3 | C₆₀/Compound A-1 | Compound 2 of C. Ex. 3 | 98.1 | A | 422 | 339 | 158 | 30 | 97 |
| C. Ex. 2-4 | C₆₀/Compound A-1 | Compound 5 of C. Ex. 6 | 98.3 | C | 232 | 195 | — | — | 91 |
| C. Ex. 2-5 | C₆₀/Compound A-1 | Compound 9 of C. Ex. 10 | 98.0 | C | 280 | 289 | — | — | 86 |
| C. Ex. 2-6 | C₆₀/Compound A-1 | Compound 11 of C. Ex. 12 | 98.2 | B | 336 | 328 | 296 | — | 100 |
| C. Ex. 2-7 | C₆₀/Compound A-1 | Compound 15 of C. Ex. 16 | 98.1 | A | 109 | 96 | 77 | 29 | 99 |
| C. Ex. 2-8 | C₆₀/Compound A-1 | Compound 16 of C. Ex. 17 | 97.4 | A | 209 | 167 | 110 | 42 | 94 |
| C. Ex. 2-9 | C₆₀/Compound A-1 | Compound 17 of C. Ex. 18 | 97.8 | B | 5820 | 4360 | 2880 | — | 99 |
| C. Ex. 2-10 | C₆₀/Compound A-1 | Compound 19 of C. Ex. 20 | 97.0 | A | 358 | 311 | 253 | 189 | 100 |
| C. Ex. 2-11 | C₆₀/Compound A-1 | Compound 23 of C. Ex. 24 | 97.9 | B | 5970 | 4930 | 2980 | 13800 | 103 |
| C. Ex. 2-12 | C₆₀/Compound A-1 | A of C. Ex. 29 | 99.5 | D | 156 | — | — | — | 94 |

“—” represents not detectable because the device is damaged by heat.

From Tables 3 and 4, it can be seen that the devices in Examples 2-1 to 2-12, in which high-purity materials after sublimation purification are used, have low dark current and high sensitivity when Examples 2-1 to 2-13 are compared to Comparative Examples 2-1 to 2-12. In addition, from the measurement result of dark current during heating, it can be seen that devices having a high glass transition temperature have high heat resistance.

Hereinafter, the structure of Compounds A-1 and A-2 will be shown.

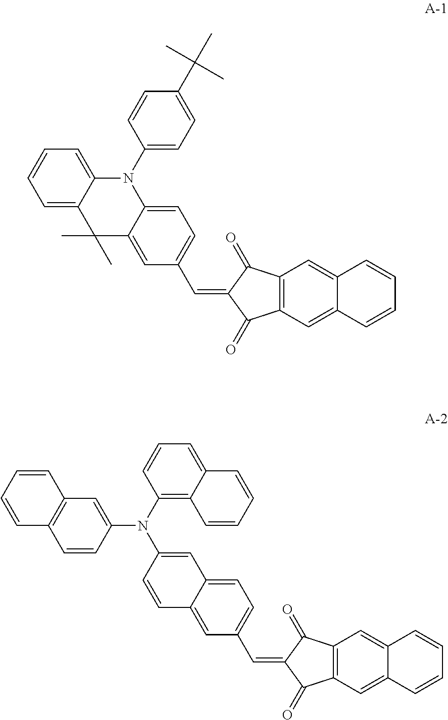 [SMILES: CC(C)(C)c1ccc(N2c3ccccc3C(C)(C)c3cc(C=C4C(=O)c5cc6ccccc6cc5C4=O)ccc32)cc1 ; O=C1C(=Cc2ccc3cc(N(c4ccc5ccccc5c4)c4cccc5ccccc45)ccc3c2)C(=O)c2cc3ccccc3cc21]

### Example 3-1

A photoelectric conversion device with the form illustrated in FIG. 1(a) was manufactured. That is, a 30-nm amorphous ITO was film-formed on a glass substrate by a sputtering method and was used as a lower electrode, and a charge blocking layer having a film thickness of 100 nm was formed by forming a film using Compound A by a vacuum heating deposition method. Further, a photoelectric conversion layer was formed by film-forming a layer, which was obtained by co-depositing Compound 21 after sublimation purification in Example 30 and fullerene (C₆₀) thereon to have a thickness of 100 nm and 300 nm, respectively in terms of single layer by vacuum heating deposition, while the temperature of the substrate was controlled at 25° C. In addition, the photoelectric conversion layer was vacuum deposited at a vacuum degree of 4×10⁻⁴ Pa or less.

Further, a transparent conductive film was formed as an upper electrode by film-forming a 10-nm amorphous ITO thereon by a sputtering method, thereby manufacturing a photoelectric conversion device.

### Examples 3-2 and 3-3 and Comparative Examples 3-1 and 3-2

A photoelectric conversion device was manufactured in the same manner as in Example 3-1, except that Compound 21 used in the photoelectric conversion layer was changed into the compound shown in Table 5. The compounds shown in Table 5 indicate compounds after sublimation purification in the Examples and Comparative Examples.

[Evaluation]

It was confirmed whether each device obtained serves as a photoelectric conversion device. That is, when voltage was applied to the lower electrode and the upper electrode of each device obtained so as to have an electric field intensity of 2.5×10⁵ V/cm, a dark current of 100 nA/cm² or less was exhibited in any device or dark place, whereas a dark current of 10 μA/cm² or more was exhibited in a bright place, and accordingly, it was confirmed that the photoelectric conversion device worked.

Table 5 shows a dark current value (a relative value when the value of the device in Example 3-1 is defined as “100”) of each device obtained. Further, Tables 5 shows a sensitivity (a relative value when the value of the device in Example 3-1 is defined as “100”) in a region at a wavelength of 500 to 750 nm when an electric field of 2×10⁵ V/cm was applied to the photoelectric conversion device each obtained in Examples 3-1 to 3-3 and Comparative Examples 3-1 and 3-2. In addition, when the photoelectric conversion performance of each device was measured, light was incident to the upper electrode (transparent conductive film) side.

TABLE 5

| | Photoelectric conversion material | | | Sensitivity in a |
|---|---|---|---|---|
| | | Sample purity | | region at a wavelength |
| | | after sublimation | Dark current | of 500 nm to 750 nm |
| | Kind of compound | purification (%) | (Relative value) | (Relative value) |
| Example 3-1 | C₆₀/Compound 21 of Example 30 | 99.5 | 100 | 100 |
| Example 3-2 | C₆₀/Compound 21 of Example 31 | 99.1 | 102 | 99 |
| Example 3-3 | C₆₀/Compound 22 of Example 32 | 99.2 | 101 | 121 |
| Comparative Example 3-1 | C₆₀/Compound 21 of C. Example 22 | 98.1 | 111 | 95 |
| Comparative Example 3-2 | C₆₀/Compound 22 of C. Example 23 | 98.2 | 109 | 107 |

From Table 5, it can be seen that the devices in Examples 3-1 to 3-3, in which high-purity materials after sublimation purification are used, have low dark current and high sensitivity when Examples 3-1 to 3-3 are compared to Comparative Examples 3-1 and 3-2.

### Example 4-1

A washed ITO substrate was put into a vapor deposition apparatus to deposit copper phthalocyanine to a thickness of 10 nm, and NPD (N,N′-di-α-naphthyl-N,N′-diphenyl)benzidine) was deposited thereon to a thickness of 40 nm. Compound 4 after sublimation purification in Example 9 and Compound B-1 were deposited thereon at a ratio (by mass) of 12:88, and the resulting layer was used as a light emitting layer. An electron transporting layer was formed by depositing BAlq [bis-(2-methyl-8-quinolinolate)-4-(phenylphenolate)aluminum][bis(6-hydroxyquinoline)-4-(phenyl-phenol)Al complex salt] to a thickness of 40 nm thereon. An organic electroluminescence device was manufactured by depositing lithium fluoride to a thickness of 3 nm thereon, and then depositing aluminum to a thickness of 60 nm.

As a result of emitting light by applying direct current constant voltage to a device, which was obtained using Source Measure Unit 2400 Type manufactured by TOYO Corporation, a phosphorescent light emission derived from B-1 was obtained.

### Examples 4-2 to 4-8 and Comparative Examples 4-1 to 4-7

Organic electroluminescence devices in Examples 4-2 to 4-8 and Comparative Examples 4-1 to 4-7 were manufactured in the same manner as in Example 4-1, except that the compound used in the light emitting layer was changed into the compound shown in Table 5. A phosphorescent light emission derived from a light emitting material used all in each device was obtained. The compounds shown in Table 6 indicate compounds after sublimation purification in the respective Examples and Comparative Examples.

Hereinafter, the structure of Compounds B-1 and B-2 used will be shown.

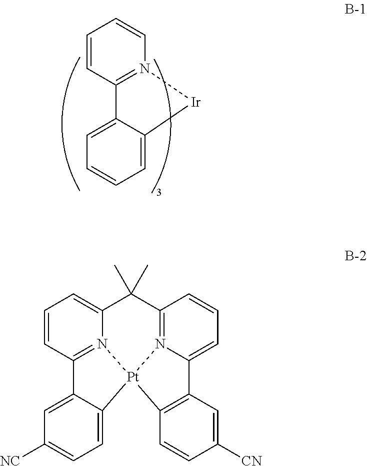

[Evaluation]

(External Quantum Efficiency)

Light was emitted by applying direct current constant voltage to each device using Source Measure Unit 2400 Type manufactured by TOYO Corporation. An external quantum efficiency (%) was calculated from the front luminance intensity at the time of 1000 cd/m². Table 6 shows the external quantum efficiency (a relative value when Example 4-1 is “1.00” as a reference) of each device.

(Driving Voltage)

An evaluation was made using a difference (ΔV) between driving voltages of an applied voltage of 1,000 cd/m² defined as a driving voltage and the driving voltage of the device in Example 4-1. A higher minus value means that the driving voltage is small and the device performance is excellent. The evaluation results are shown in Table 6.

TABLE 6

| | Light emitting layer | | | Difference in |
|---|---|---|---|---|
| | | Sample purity | External quantum | driving voltage |
| | | after sublimation | efficiency | (ΔV) from |
| | Kind of compound | purification (%) | (Relative value) | Example 4-1 |
| Example 4-1 | B-1/Compound 4 of Ex. 9 | 99.5 | 1.00 | — |
| Example 4-2 | B-2/Compound 4 of Ex. 9 | 99.5 | 0.98 | −0.52 |
| Example 4-3 | B-1/Compound 5 of Ex. 10 | 99.6 | 1.32 | −0.66 |
| Example 4-4 | B-2/Compound 5 of Ex. 10 | 99.6 | 1.25 | −1.22 |
| Example 4-5 | B-1/Compound 9 of Ex. 14 | 99.5 | 1.03 | −1.08 |
| Example 4-6 | B-1/Compound 9 of Ex. 15 | 99.0 | 1.02 | −0.03 |
| Example 4-7 | B-1/Compound 10 of Ex. 16 | 99.6 | 1.22 | −0.89 |
| Example 4-8 | B-2/Compound 12 of Ex. 18 | 99.7 | 1.14 | −0.85 |
| Comparative Example 4-1 | B-1/Compound 4 of C. Ex. 5 | 98.1 | 0.94 | +0.65 |
| Comparative Example 4-2 | B-2/Compound 4 of C. Ex. 5 | 98.1 | 0.91 | +0.11 |
| Comparative Example 4-3 | B-1/Compound 5 of C. Ex. 6 | 98.3 | 1.20 | +0.01 |
| Comparative Example 4-4 | B-2/Compound 5 of C. Ex. 6 | 98.3 | 1.11 | −0.44 |
| Comparative Example 4-5 | B-1/Compound 9 of C. Ex. 10 | 98.0 | 0.98 | +0.10 |
| Comparative Example 4-6 | B-1/Compound 10 of C. Ex. 11 | 98.3 | 1.15 | −0.69 |
| Comparative Example 4-7 | B-2/Compound 12 of C. Ex. 13 | 98.4 | 1.05 | −0.78 |

As clear from Table 6, it can be seen that the devices in Examples 4-1 to 4-8, in which high-purity materials after sublimation purification are used, have a high external quantum efficiency and a low driving voltage.

### Example 5-1

A washed ITO substrate was put into a vapor deposition apparatus to deposit copper phthalocyanine to a thickness of 10 nm, and NPD (N,N′-di-a-naphthyl-N,N′-diphenyl)benzidine) was deposited thereon to a thickness of 40 nm. Compound A and Compound 14 in Example 20 were deposited at a ratio (by mass) of 12:88 to a thickness of 20 nm, and the resulting layer was used as a light emitting layer. An electron transporting layer was formed by depositing BAlq [bis-(2-methyl-8-quinolinolate)-4-(phenylphenolate)aluminum][bis(6-hydroxyquinoline)-4-(phenyl-phenol)Al complex salt] to a thickness of 40 nm thereon. An organic electroluminescence device was manufactured by depositing lithium fluoride to a thickness of 3 nm thereon, and then depositing aluminum to a thickness of 60 nm.

As a result of emitting light by applying direct current constant voltage to a device, which was obtained using Source Measure Unit 2400 Type manufactured by TOYO Corporation, phosphorescent light emission derived from Compound 14 was obtained.

### Comparative Example 5-1

In Comparative Example 5-1, an organic electroluminescence device of Comparative Example 5-1 was manufactured in the same manner as described above, except that the compound used in the light emitting layer was changed into the compound described in Table 7. A phosphorescent light emission derived from a light emitting material used all in each device was obtained. The compounds shown in Table 7 indicate compounds after sublimation purification in the respective Examples and Comparative Examples.

[Evaluation]

(External Quantum Efficiency)

Light was emitted by applying direct current constant voltage to each device using Source Measure Unit 2400 Type manufactured by TOYO Corporation. An external quantum efficiency (%) was calculated from the front luminance intensity at the time of 1000 cd/m². Table 6 shows the external quantum efficiency (a relative value when Example 5-1 is “1.00” as a reference) of each device.

(Driving Voltage)

An evaluation was made using a difference (ΔV) between driving voltages of an applied voltage of 1,000 cd/m² defined as a driving voltage and the driving voltage of the device in Example 5-1. A higher minus value means that the driving voltage is small and the device performance is excellent. The evaluation results are shown in Table 7.

TABLE 7

| | Light emitting layer | | | Difference in |
|---|---|---|---|---|
| | | Sample purity | External quantum | driving voltage |
| | | after sublimation | efficiency | (ΔV) from |
| | Kind of compound | purification (%) | (Relative value) | Example 4-1 |
| Example 5-1 | Compound 14 of Ex. 20 | 99.2 | 1.00 | — |
| Comparative Example 5-1 | Compound 14 of C. Ex. 15 | 98.0 | 0.85 | +0.84 |

As clear from Table 7, it can be seen that the device in Example 5-1, in which a high-purity material after sublimation purification is used, has a high external quantum efficiency and a low driving voltage.

### INDUSTRIAL APPLICABILITY

The method for purifying an organic material according to the present invention may sublime and purify an organic material having high heat resistance at high sublimation temperature with high purity and high yield in a short period of time.

Further, the material for organic electronics of the present invention has high heat resistance and high purity at high sublimation temperature. In addition, the photoelectric conversion device, the optical sensor, the imaging device and the organic electroluminescence device of the present invention may use the material for organic electronics.

Although the present invention has been described with reference to detailed and specific exemplary embodiments, it is obvious to those skilled in the art that various changes or modifications may be made without departing from the spirit and scope of the present invention.

The present application is based on Japanese Patent Application (Patent Application No. 2011-086506) filed on Apr. 8, 2011 and Japanese Patent Application (Patent Application No. 2012-074554) filed on Mar. 28, 2012, the contents of which are herein incorporated by reference.

### DESCRIPTION OF SYMBOLS

1 Organic electroluminescence device
2 Substrate
3 Anode
4 Hole injection layer
5 Hole transporting layer
6 Light emitting layer
7 Hole blocking layer
8 Electron transporting layer
9 Cathode
10a, 10b Photoelectric conversion device
11 Lower electrode (conductive thin film)
12 Photoelectric conversion layer (photoelectric conversion film)
15 Upper electrode (transparent conductive thin film)
16A Electron blocking layer
16B Hole blocking layer
100 Imaging device
101 Substrate
102 Insulating layer
103 Connection electrode
104 Pixel electrode (lower electrode)
105 Connection part
106 Connection part
107 Photoelectric conversion film
108 Counter electrode (upper electrode)
109 Buffer layer
110 Encapsulation layer
111 Color filter (CF)
112 Partition
113 Light-shielding layer
114 Protective layer
115 Counter electrode voltage supply part
116 Read-out circuit

1. A method for purifying an organic material having a 10% weight reduction temperature of 250° C. or more as measured by thermogravimetry at a vacuum degree of 1×10⁻² Pa or less,
wherein the organic material is subjected to sublimation purification after a concentration of inorganic impurities in the organic material is adjusted to 5,000 ppm or less.

2. The method according to claim 1,
wherein the inorganic impurities having a concentration of 5,000 ppm or less are atoms and ions of a metal belonging to alkali metals, alkaline earth metals, transition metals, or typical metals.

3. The method according to claim 2,
wherein the inorganic impurities having a concentration of 5,000 ppm or less are atoms and ions of a metal belonging to alkali metals, or transition metals.

4. A material for organic electronics having a 10% weight reduction temperature of 250° C. or more as measured by thermogravimetry at a vacuum degree of 1×10⁻² Pa or less, wherein a purity of the material for organic electronics is 98.5% or more.

5. The material for organic electronics according to claim 4,
wherein the material for organic electronics is a compound represented by the following Formula (1): 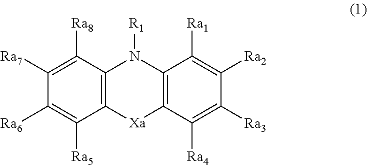
wherein in the formula, R₁ represents an alkyl group, an aryl group or a heterocyclic group, which optionally have a substituent,
Ra₁ to Ra₈ each independently represent a hydrogen atom or a substituent,
at least two of R₁ and Ra₁ to Ra₈ optionally are bound with each other to form a ring, and
Xa represents a single bond, an oxygen atom, a sulfur atom, or an alkylene group, a silylene group, an alkenylene group, a cycloalkylene group, a cycloalkenylene group, an arylene group, a divalent heterocyclic group or an imino group, which optionally has a substituent.

6. The material for organic electronics according to claim 5,
wherein the compound represented by Formula (1) is a compound represented by the following Formula (F-1): 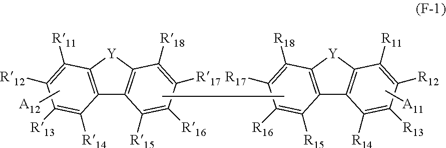
wherein in Formula (F-1), R₁₁ to R₁₈ and R′₁₁ to R′₁₈ each independently represent a hydrogen atom, a halogen atom, an alkyl group, an aryl group, a heterocyclic group, a hydroxyl group, an amino group or a mercapto group, and these groups optionally further have a substituent, provided that any one of R₁₅ to R₁₈ is linked to any one of R′₁₅ to R′₁₈ to form a single bond,
A₁₁ and A₁₂ each independently represent a substituent represented by the following Formula (A-1), and are substituted as one of R₁₁ to R₁₄ and one of R′₁₁ to R′₁₄, and
Y independently represents a carbon atom, a nitrogen atom, an oxygen atom, a sulfur atom or a silicon atom, and these groups optionally further have a substituent: 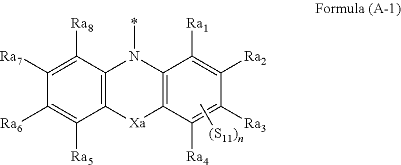
wherein in Formula (A-1), Ra₁ to Ra₈ each independently represent a hydrogen atom, a halogen atom, an alkyl group, an aryl group, a heterocyclic group or an alkoxy group, and these groups optionally further have a substituent,
at least two of Ra₁ to Ra₈ optionally are bound with each other to form a ring,
* represents a bonding position,
Xa represents a single bond, an oxygen atom, a sulfur atom, or an alkylene group, a silylene group, an alkenylene group, a cycloalkylene group, a cycloalkenylene group, an arylene group, a divalent heterocyclic group or an imino group, which optionally has a substituent,
S₁₁ independently represents the following substituent (S₁₁), and is substituted as one of Ra₁ to Ra₈, and
n independently represents an integer of 1 to 4: 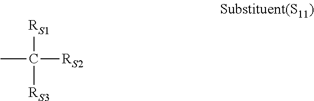
wherein R_{S1} to R_{S3} each independently represent a hydrogen atom or an alkyl group, and
at least two of R_{S1} to R_{S3} optionally are bound with each other to form a ring.

7. The material for organic electronics according to claim 6,
wherein the compound represented by Formula (F-1) is a compound represented by the following Formula (F-2): 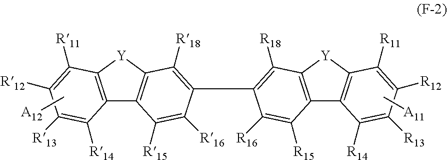
wherein in Formula (F-2), R₁₁ to R₁₆, R₁₈, R′₁₁ to R′₁₆ and R′₁₈ each independently represent a hydrogen atom, a halogen atom, an alkyl group, an aryl group, a heterocyclic group, a hydroxyl group, an amino group or a mercapto group, and these groups optionally further have a substituent,
A₁₁ and A₁₂ each independently represent the substituent represented by Formula (A-1), and are substituted as one of R₁₁ to R₁₄ and one of R′₁₁ to R′₁₄, and
Y independently represents a carbon atom, a nitrogen atom, an oxygen atom, a sulfur atom or a silicon atom, and these groups optionally further have a substituent.

8. The material for organic electronics according to claim 6,
wherein in Formula (F-1), the substituent represented by Formula (A-1) is independently substituted as R₁₂ and R′₁₂.

9. The material for organic electronics according to claim 6,
wherein n in Formula (A-1) represents 1 or 2.

10. The material for organic electronics according to claim 6,
wherein at least one of Ra₃ and Ra₆ in Formula (A-1) each independently represents the substituent (S₁₁).

11. The material for organic electronics according to claim 6,
wherein Y in Formulae (F-1) and (F-2) represents —N(R₂₀)—, and R₂₀ represents an alkyl group, an aryl group or a heterocyclic group.

12. The material for organic electronics according to claim 6,
wherein Y in Formulae (F-1) represents —C(R₂₁)(R₂₂)—, and R₂₁ and R₂₂ each independently represent an alkyl group, an aryl group or a heterocyclic group.

13. The material for organic electronics according to claim 4,
wherein the material for organic electronics is a material represented by the following Formula (2): 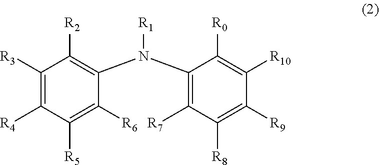
wherein in the formula, R₁ represents an alkyl group, an aryl group or a heterocyclic group, which optionally has a substituent, and
R₀ and R₂ to R₁₀ each independently represent a hydrogen atom or a substituent.

14. The material for organic electronics according to claim 13,
wherein in Formula (2), R₁ which optionally has a substituent group is an aryl group.

15. The material for organic electronics according to claim 4,
wherein a glass transition temperature (Tg) of the material for organic electronics is 130° C. or more.

16. The material for organic electronics according to claim 4,
wherein a molecular weight of the material for organic electronics is from 500 to 2,000.

17. A photoelectric conversion device comprising:
a transparent conductive film;
a photoelectric conversion film; and
a conductive film in this order,
wherein the photoelectric conversion film includes a photoelectric conversion layer and a charge blocking layer, and
the charge blocking layer contains the material for organic electronics according to claim 4.

18. The photoelectric conversion device according to claim 17,
wherein the photoelectric conversion layer includes an n-type organic semiconductor.

19. The photoelectric conversion device according to claim 18,
wherein the n-type organic semiconductor is fullerene or a fullerene derivative.

20. The photoelectric conversion device according to claim 17,
wherein the photoelectric conversion film contains a compound of the following Formula (I): 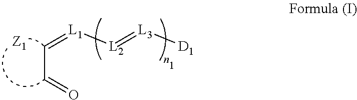
wherein in the formula, Z₁ is a ring containing at least two carbon atoms, and represents a 5-membered ring, a 6-membered ring or a condensed ring including at least one of the 5-membered ring and the 6-membered ring,
L₁, L₂ and L₃ each independently represent an unsubstituted methine group or a substituted methine group,
D₁ represents an atom group, and
n₁ represents an integer of 0 or more.

21. A method for manufacturing the photoelectric conversion device according to claim 17, the method comprising:
film-forming each of the photoelectric conversion layer and the charge blocking layer by vacuum thermal deposition.

22. An optical sensor comprising: the photoelectric conversion device according to claim 17.

23. An imaging device comprising: the photoelectric conversion device according to claim 17.

24. An organic electroluminescence device comprising: at least one organic layer including a light emitting layer between a pair of electrodes,
wherein the organic layer contains the material for organic electronics according to claim 4.
